(19) United States
(12) Patent Application Publication
(10) Pub. No.: US 2024/0148858 A1
(43) Pub. Date: May 9, 2024

(54) A THREE COMPONENT VACCINE FOR COVID-19

(71) Applicant: The Board of Trustees of the Leland Stanford Junior University, Stanford, CA (US)

(72) Inventors: Ronald Levy, Stanford, CA (US); Ole Audun Werner Haabeth, Stanford, CA (US); Adrienne Sallets, Stanford, CA (US); Timothy R. Blake, Stanford, CA (US); Paul Wender, Stanford, CA (US); Robert M. Waymouth, Stanford, CA (US); Debra Czerwinski, Stanford, CA (US); Idit Sagiv-Barfi, Stanford, CA (US); Julian Johannes Lohmeyer, Stanford, CA (US)

(73) Assignee: The Board of Trustees of the Leland Stanford Junior University, Stanford, CA (US)

(21) Appl. No.: 18/006,264

(22) PCT Filed: Jul. 26, 2021
(86) PCT No.: PCT/US21/43213

§ 371 Date: Jan. 20, 2023

Related U.S. Application Data
(60) Provisional application No. 63/056,465, filed on Jul. 24, 2020.

(51) Int. Cl. A61K 39/215; A61K 47/59; A61P 31/14

(52) U.S. Cl. CPC A61K 39/215; A61K 47/593; A61P 31/14; A61K 2039/55555

(57) ABSTRACT

There are provided herein, inter alia, complexes, compositions and methods for a vaccine for COVID-19. The cell-penetrating complexes provided herein may include a nucleic acid non-covalently bound to a cationic amphipathic polymer, the cationic amphipathic polymer including a pH-sensitive immolation domain. The cell-penetrating complexes provided herein are, inter alia, useful in vaccines, wherein the vaccine includes a ribonucleic acid including a sequence encoding a viral protein, a nucleic acid adjuvant and a cationic amphipathic polymer provided herein including embodiments thereof.

### CROSS-REFERENCE TO RELATED APPLICATIONS

This application claims priority to U.S. Provisional Application No. 63/056,465, filed Jul. 24, 2020, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 041243-561001WO_SL_ST25.txt, created on Jul. 22, 2021, 8,192 bytes, machine format IBM-PC, MS Windows operating system, is hereby incorporated by reference.

### BACKGROUND

There are currently no effective treatments or vaccines available for the disease caused by the SAR-CoV-2 virus. There exists a need to develop safe and effective vaccines that protect against COVID-19 caused by the novel coronavirus: SARS-CoV-2. Provided herein are solutions to these and other problems in the art.

### BRIEF SUMMARY

In an aspect is provided a cell-penetrating complex including a ribonucleic acid including a sequence encoding a viral protein, a nucleic acid adjuvant and a cationic amphipathic polymer.

In an aspect is provided a vaccine composition including a cell-penetrating complex, as described herein, including embodiments.

In an aspect is provided a pharmaceutical composition including a therapeutically effective amount of a cell-penetrating complex as described herein, including embodiments, and a pharmaceutically acceptable excipient.

In an aspect is provided a method of treating or preventing a viral disease in a subject in need of such treatment or prevention, the method including administering a therapeutically or prophylactically effective amount of a cell-penetrating complex as described herein, including embodiments, to the subject.

In an aspect is provided a method of treating a viral disease in a subject in need thereof, including administering to the subject a therapeutically effective amount of a cell-penetrating complex as described herein, including embodiments, and a pharmaceutical carrier, thereby treating a viral disease in the subject.

In an aspect is provided a method for immunizing a host susceptible to a viral disease, including administering a cell-penetrating complex as described herein, including embodiments, to a host under conditions such that antibodies directed to the viral protein or a functional fragment thereof are produced.

### BRIEF DESCRIPTION OF THE DRAWINGS

FIGS. 1A-1B. Example schematic of the detection step of an ELISA assay (FIG. 1A) and example protocol of ELISA assays for detecting anti-IgG or IgM antibodies (FIG. 1B).

FIGS. 2A-2E. ELISA Data D+4. Early detection of RBD-specific Antibody response in mice vaccinated with RBD mRNA-CARTs+CpG. Sample from mouse taken 4 days after administration of vaccine. Results are shown for mice vaccinated with RBD mRNA-CART (FIG. 2A), mRNA-CART+CpG (FIG. 2B), RBD mRNA-CART+agonistic CD40mAb (FIG. 2C) and RBD mRNA-CART+agonistic CD40mAb+CpG (FIG. 2D). Results from all groups of mice are overlaid for comparision (FIG. 2E).

FIG. 3. ELISA Data D+8. Superior RBD-specific Antibody response in mice vaccinated with RBD mRNA-CARTs+CpG. Samples from mouse taken 4 days and 8 days after administration of vaccine.

FIGS. 4A-4B. Specificity Test—Vaccine induced Antibodies are RBD-specific. Representative image of microplate used for detection of RBD-specific antibodies (FIG. 4A) and graph showing detection of RBD-specific actibodies (FIG. 4B). Graph further shows results for detection of Anti His (His Tag ctr.) and Anti OVA (irrelevant ctr).

FIGS. 5A-5E. Isotype Characterization of RBD-specific Antibody responses. RBD mRNA-CARTs+CpG vaccine induce robust isotype switching. Samples from mouse taken 4 days, 8 days, and 14 days after administration of vaccine. Results are shown for detection of IgG (FIG. 5A), IgG1 (FIG. 5B), IgG2a (FIG. 5C), IgG2b (FIG. 5D) and IgG3 (FIG. 5E) antibodies.

FIGS. 6A-6E. Isotype Characterization of RBD-specific Antibody responses-RBD mRNA-CARTs+CpG vaccine induce long lasting isotype switched RBD-specific antibody responses (>60 days post vaccination). Samples from mouse taken 4 days, 8 days, and 14 days after administration of vaccine. Results are shown for detection of IgG (FIG. 6A), IgG3 (FIG. 6B), IgG2a (FIG. 6C), IgG1 (FIG. 6D) and IgG2b (FIG. 6E) antibodies.

FIGS. 7A-7B. BD-specific Antibody responses in bronchoalveolar lavage (BAL). Schematic showing BAL procedure used for collecting sample from lungs for testing (FIG. 7A). RBD mRNA-CARTs+CpG vaccine induce RBD-specific antibody response that is detected in lungs of vaccinated animals (FIG. 7B). Samples from mouse taken 4 days, 8 days, and 14 days after administration of vaccine.

FIG. 8. Schematic showing receptor binding inhibition assay.

FIGS. 9A-9C. Cellular Receptor binding inhibition Assay. RBD mRNA-CARTs+CpG vaccine induce RBD-specific antibody response that block SARS-CoV-2 RBD binding to the ACE-2 receptor (FIG. 9A). Results show RBD is still blocked Day 12 post-vaccination (FIG. 9B) and Day 26 post-vaccination (FIG. 9C).

FIGS. 10A-10B. Assay for Antibodies that block ACE2-Spike RBD interaction (FIG. 10A). Results show that the serum of mice vaccinated with RBD mRNA-CARTs+CpG inhibit Spike RBD from binding to the ACE2 receptor (FIG. 10B).

FIGS. 11A-11B. Pseudoviral neutralization Assay. RBD mRNA-CARTs+CpG vaccine induce RBD-specific antibody response that completely block SARS-CoV-2 spike protein expressing pseudoviral entry into ACE-2 expressing cells (FIG. 11A). Results show that serum of mice vaccinated with RBD mRNA-CARTs+CpG inhibits pseudoviral enters into ACE-2 expressing cells (FIG. 11B).

FIGS. 12A-12B. CART-RBD mRNA-CpG Vaccine in Humanized mice. RBD mRNA-CARTs+CpG vaccine induce RBD-specific antibody responses in mice reconstituted with human immune cells (FIG. 12A). Results show detection of RBD-specific IgG in human PBMC administered mice (FIG. 12B).

FIGS. 13A-13B. RBD mRNA-CARTs+CpG vaccine induce RBD-specific antibody response independently of TLR9 (CpG) source and route of administration (e.g. the vaccine induces an immune response with both subcutaneous and intramuscular administration routes). Results show that mRNA vaccine is superior to protein vaccine. Results further indicate that administration of components in the same syringe is better than administration of the components through separate injections. RBD specific antibody detection was completed on Day 14 (FIG. 13A) and Day 21 (FIG. 13B) after vaccination.

FIG. 14. Isotype switching as early as D+14 in all CpG groups is observed. CpG classes including CpG 1826 and CpG SD101 were tested.

FIGS. 15A-15B. Partial Receptor blocking is observed as early as D+14. Results confirm that various CpG classes induce inhibition of RBD-spike protein binding to ACE-2 as early as D+14 (FIG. 15A). The results are consistent with mRNA vaccine inducing higher RBD-specific antibody levels than protein vaccine. Further, various administration methods (e.g. subcutaneous, intravenous, and intramuscular) induce receptor blocking at D+14 (FIG. 15B).

FIG. 16. Schematic showing that Charge-Altering Releasable Transporters (CARTs) effectively deliver co-formulated SARS-CoV-2 RBD mRNA and adjuvant. Prime-Boost immunization leads to robust antigen-specific B and T cell responses in mice.

FIGS. 17A-17E. CART delivery platform methodology effectively complexes, delivers, and releases mRNA via both systemic and local administration. (FIG. 17A) CART electrostatic formulation, cellular uptake, endosomal escape, and translation of SARS-CoV-2 RBD mRNA. (FIG. 17B) CART synthesis via ring-opening polymerization (DBU=1,8-diazabicyclo[5.4.0]undec-7-ene, TU=1-(3,5-bis(trifluoromethyl)phenyl)-3-cyclohexylthiourea). (FIG. 17C) CART chemical structure, degradation products, and charge-altering mechanism. (FIG. 17D) In vivo luciferase reporter gene expression via systemic IV administration (left, 5 μg of fLuc mRNA), and local IM administration (2.5 μLg of fLuc mRNA each flank). (FIG. 17E) Quantification of in vivo mRNA expression at 4 h postadministration.

FIGS. 18A-18D. Addition of CpG to RBD mRNA-CART elicits a stronger anti-RBD immunoglobulin response and leads to earlier isotype switching. (FIG. 18A) BALB/c mice were immunized intravenously with either 3 μg of RBD mRNA-CART (n=5), 3 μg of RBD mRNA-CART plus 3 μg of CpG (n=5), 3 μg of CpG CART (n=5), or Naïve untreated (n=5) and boosted on day 4 and day 8 after priming. (FIG. 18B) Serum levels of RBD-specific IgGs from RBD mRNA-CART (white circle), RBD mRNA+CpG-CART (square), CpG CART (black circle), and Naïve (diamond) mice were monitored over the course of 60 days postpriming by ELISA. (FIG. 18C) On day 14 and day 60 after priming, the distribution of IgG isotypes specific to RBD was analyzed using antimouse IgG1 (circle marker), IgG2a (black), IgG2b (square marker), and IgG3 (white) monoclonal antibodies by ELISA. (FIG. 18D) On day 14 and day 60 after priming, the absolute concentration of the anti-RBD IgG was evaluated. *=P<0.05 unpaired Student's t test (two-tailed). Data representative of 2 individual experiments.

FIGS. 19A-19E. RBD mRNA+CpG-CART generates early high levels of RBD neutralizing antibodies. BALB/c mice (n=5) were immunized as described in FIG. 18A. (FIG. 19A) Sera from mice immunized with RBD mRNA-CART (circle), RBD mRNA+CpG-CART (square), and CpG CART (diamond) were collected on D28 and D60 and tested in a commercially available RBD-ACE-2 inhibition assay. The same set of serum samples was tested in a pseudotyped virus neutralization assay. RBD-expressing pseudovirus particles containing a zsGreen and firefly luciferase vectors were coincubated with titrated concentrations of heat-inactivated mouse serum. (FIG. 19B) The pseudovirus particle—serum mix was then added to wells containing ACE-2-overexpressing 293F cells. Firefly luciferase expression was measured at 48 and 72 h after the start of the experiment. On D60, BAL was harvested from RBD mRNA-CART (circle), RBD mRNA+CpG-CART (square), and CpG-CART (diamond) immunized mice. (FIG. 19C) RBD-specific total IgG was assayed by ELISA. (FIG. 19D) BAL containing immunoglobulins was tested for their ability to inhibit binding of RBD to hACE-2 using a commercial ACE-2 inhibition kit. (FIG. 19E) Lung single-cell suspensions from naive mice (gray, n=3) or mice vaccinated on DO and D21 IV with 3 μg of RBD-mRNA+3 μg of CpG (black, n=3) were collected on D28 and incubated with media alone, RBD protein, or an irrelevant protein (CD81-His) [5 μg/mL] for 48 h and stained for T cell activation markers CD134, CD137, and intracellular TNFα on CD4+ T cells. Data are shown as mean±SD. Data representative of 3 independent experiments (FIGS. 19B-19D) and 1 experiment (FIG. 19E). *=P<0.05, **=P<0.01 ***=P<0.001, ****=P<0.0001 one-way ANOVA (Tukey's multiple comparison test) (FIG. 19D) or two-way ANOVA (Tukey's multiple comparison test) (FIG. 19E).

FIGS. 20A-20D. RBD mRNA+CpG-CART elicits neutralizing anti-RBD immunoglobulin responses after IV and IM vaccination. (FIG. 20A) BALB/c mice (n=5 per group) were immunized intravenously (IV) or intramuscularly (IM) with 3 μg of RBD mRNA plus 3 μg of CpG and boosted on day 21 after priming. (FIG. 20B, FIG. 20C) RBD-specific immunoglobulin titers in serum were measured and quantified on day 21 and day 28. Measurements are shown as (FIG. 20B) A450 and in (FIG. 20C) ng/mL of anti-RBD. (FIG. 20D) On day 28, BAL was harvested from both IV and IM treated mice, and anti-RBD immunoglobulins were assayed by ELISA. Data are shown as mean±SD. Data representative of 2 independent experiments. Statistical significance was assessed by a Student's t test (two-tailed, unpaired) ns=P>0.05.

FIGS. 21A-21D. RBD mRNA plus CpG vaccination induces long-lasting memory T_{H}1 CD4⁺ and CD8⁺ T cell responses. (FIG. 21A, FIG. 21B) BALB/c mice (n=5 per group) were immunized intravenously (IV) or intramuscularly (IM) with 3 μg of ctrl mRNA+3 μg of CpG or 3 μg of RBD mRNA+3 μg of CpG on D1 and boosted on D21 after priming. On day 10⁵, pooled splenocytes were harvested, enriched for CD4⁺ and CD8⁺ T cells, and stimulated separately for 16 h with an RBD peptide mix for a direct ex vivo IFNγ ELISpot assay (FIG. 21A). For ELISpot analysis, splenocytes from the respective groups were measured in triplicates. Additionally, whole splenocytes of individual mice (n=5 per group) were incubated with media or an RBD peptide pool for 18 h (FIG. 21B). (FIG. 21C, FIG. 21D) After incubation, cells were collected and stained for T cell memory marker CD44 and IFNγ (FIG. 21C) or CD44 as well as intracellular cytokines IFNγ, TNFα, and IL-4 (FIG. 21D). Each dot represents the measurement of an individual mouse. **=P<0.01, ***=P<0.001, ****=P<0.0001 two-way ANOVA (FIG. 21B) or one-way ANOVA (FIG. 21D) (Tukey's multiple comparisons test).

FIGS. 22A-22B. Neutralizing antibody levels of immunized mice are comparable to those achieved in vaccinated humans. Serum from immunized mice (IM—triangle, IV-square, n=5) was harvested on day 28. Serum from blood donors (n=13) who were vaccinated with the Pfizer/BioNTech mRNA vaccine was collected either within 7 days before (preboost, circle) or 15±4 days after the boost (postboost, diamond) (FIG. 22A) and was tested for the ability to inhibit RBD/ACE-2 binding using a commercially available surrogate Virus Neutralization Test (FIG. 22B). *=P<0.05, ****=P<0.0001 one-way ANOVA (Tukey's multiple comparison test).

FIGS. 23A-23B. CART oligomer and CART/mRNA NP characterization. (FIG. 23A) ¹H NMR spectrum (CD₃OH, 500 MHz) of CART O₆-stat-N₆: A₉. Relative ratios in CD₃OD: BnOH end group 7.35 ppm, 5H(C₅H₅); nonenyl segment 0.9 ppm, 18H(CH3); oleyl segment 0.85 ppm, 18H(CH3), aminoester segment 3.5 ppm, 18H(CH2). Number avg. molecular weight (gel permeation chromatography vs. polystyrene standards on N-Boc protected oligomer): Mn=4.3 kDa, D=1.19. Comparing these ratios indicates a statistical triblock CART with DP 1:6:6:9. (FIG. 23B) CART-nucleotide nanoparticle formulation and physical characterization. ^{a}Nanoparticle sizes were measured using dynamic light scattering (DLS). Each value is the average from 3 trials using independently prepared formulations with the corresponding standard deviation in the same units. The plot of intensity of scattered light as a function of nanoparticle size from analysis of these samples exhibits one, monomodal distribution of particle sizes. ^{b}Zeta potential measurements were conducted using electrophoretic light scattering (ELS). Each value is the average of 6 measurements from 3 trials using independently prepared formulations (for each trial, 2 measurements were acquired consecutively) with the corresponding standard deviation in the same units. ^{c}Encapsulation efficiency was measured by the fluorescence of the RNA-specific Qubit RNA BR dye (Q10210; Invitrogen). RBD-mRNA or RBD-mRNA+CpG were complexed using a total 840 ng of nucleotide cargo and enough CART for a net 10:1 (cation:anion) ratio. This was added to 0.75 mL of RNase-free deionized water containing 5 μL of Qubit reagent. The fluorescence of the solution was immediately measuring using an excitation wavelength of 630 (4-nm slit width) and emission wavelength of 680 nm (4 nm slit width). Percent encapsulation was determined by subtracting the fluorescence of the Qubit dye alone (no nucleotide) and normalizing to the fluorescence of Qubit with uncomplexed mRNA.

FIGS. 24A-24B. 293F cells were transfected with CART-RBD mRNA. 16 h later the supernatant was analyzed by western blot for RBD-His expression. Negative controls are media from non-transfected cells. 100 ng of purified RBD protein was used as positive control (FIG. 24A). CD69 upregulation on circulating immune cell subsets 24 h after IV injection of mRNA-CART complex using indicated mRNA. Non-immunostimulatory mRNA EGFP mRNA (5moU) (L-7201 Trilink), Immunostimulatory mRNA: CleanCap™ FLuc mRNA (L-7602 Trilink) (FIG. 24B). Pooled data from multiple independent experiments. Statistical significance was assessed by One-Way ANOVA: P>0.05 (ns), P£0.001(***).

FIGS. 25A-25B. RBD mRNA+CpG-CART elicits anti-RBD immunoglobulin responses as early as 4 days post priming. BALB/c mice were immunized intravenously with either 3 ug RBD-CARTS (n=5), 3 ug RBD-CART mRNA plus 3 ug CpG (n=5), CpG CART (n=5), or Naïve untreated (n=5) and boosted on Day +4 and Day +8. Serum levels of RBD-specific IgGs from RBD-CART mRNA (circle), RBD-CART mRNA plus CpG (square), CpG CART (diamond), and Naïve (triangle) mice were tested 4 days post priming by ELISA (FIG. 25A). The same serum samples were tested for cross reactivity with an irrelevant His-tagged GFP (GFP-His) protein using ELISA plate coated with 5 ug/ml GFP-His protein and compared to an anti His antibody positive plate control (diamond) (FIG. 25B).

FIGS. 26A-26B. RBD mRNA+CpG-CART induces an early immunoglobulin isotype switch and generates high levels of ACE-2-RBD neutralizing antibodies. BALB/c mice were immunized intravenously with either 3 ug RBD-CARTS (n=5), 3 ug RBD-CART mRNA plus 3 ug CpG (n=5), CpG CART (n=5), or Naïve untreated (n=5) and boosted on D4 and D8 after priming. Serum from RBD-CART mRNA (circle), RBD-CART mRNA plus CpG (square), immunized mice was collected on Day +14 (FIG. 26A) and Day +60 (FIG. 26B) and the distribution of IgG isotypes specific to RBD was analyzed using anti-mouse IgG1, IgG2a, IgG2b and IgG3 monoclonal antibodies in ELISA.

FIGS. 27A-27C. Subcutaneous and intramuscular RBD mRNA+CpG-CART vaccine administration induces robust isotype switched anti-RBD immunoglobulin responses. 5 BALB/c mice per group were immunized intravenously (IV), intramuscular (IM), or sub cutaneous (SC) with 3 ug RBD-CART mRNA plus 3 ug CpG and boosted on D8 after priming (FIG. 27A). RBD-specific Immunoglobulin titers in serum were measured on D14 and D28 (FIG. 27B). RBD-specific IgG1, IgG2a, IgG2b, and IgG3 was measured on Day +14 (FIG. 27C). Data representative of two individual experiments.

FIGS. 28A-28C. Induction of neutralizing antibodies is independent of CpG source. 5 BALB/c mice per group were immunized intravenously (IV) or Intramuscular (IM) with 3 ug RBD-CART mRNA plus 3 ug CpG using 3 different sources of CpG (IMO/2055, SD101 or ODN2395) and boosted on D21 after priming (FIG. 28A). Serum from immunized mice harvested on D21 and D28 was tested for ability to inhibit RBD-ACE-2 binding using a commercially available RBD-ACE-2 inhibition assay (FIG. 28B). Serum from immunized mice harvested on D21 and D28 was tested for ability to bind to the full Spike protein by ELISA (FIG. 28C).

FIGS. 29A-29C. Mice were immunized as described in FIGS. 28A-28C. RBD-specific Immunoglobulin titers in serum were measured on D28 (FIG. 29A) and D21 (FIG. 29B). Quantification of absolute concentration of RBD-specific antibodies in serum on D21 (FIG. 29C).

FIG. 30. Splenocytes from mice vaccinated with 3.ig RBD-mRNA+3.ig CpG-CART or 3.ig ctrl mRNA+3.ig CpG-CART either IV or IM on D1 and D21 were harvested on D105 after vaccination and incubated for 18 h with RBD peptide pool or media. After stimulation cells were analyzed for IFN, TNF or IL-4 production by intracellular cytokine staining flow cytometry.

FIG. 31A-31N. Safety evaluation of the RBD mRNA-CpG-CART Sars-Cov-2 vaccine. Mice (6 mice per group) were injected with PBS, 3 ug GFP mRNA formulated with CART or 3 ug GFP mRNA+3 ug CpG formulated with CART or 3 ug RBD mRNA+3 ug CpG formulated with CART. Treatments were injected in the tail vain (IV) or in the muscle (IM) on day 0 (prime) and day 21 (boost). (FIG. 31A) Schematic representing the schedule of injection and safety measurements. Body weight of the mice was measured on Day 0 (before first treatment), Day 1, Day 2, Day 4 and Day? for IM (FIG. 31B) and IV (FIG. 31C) treated mice. White Blood Cell (WBC) count was assessed on Day 1 (FIG. 31D), Day 2 (FIG. 31E) and Day 4 (FIG. 31F). Data displayed percentage relative to the control group. Sera were harvested on day 1 and day 7 for cytokines measurement. On Day 22 and Day 26, 3 mice per group were sacrificed and necropsy assessment and complete blood count were assessed, and serum was collected for cytokines and liver enzymes measurements. (FIG. 31G-31J) IP10 was measured in the sera harvested on Day 1 (D1 post prime) (FIG. 31G) and Day 7 (1 week post prime) (FIG. 31H), and IFα was measured in the sera harvested on Day 1 (D1 post prime) (FIG. 31I) and Day 7 (1 week post prime) (FIG. 31J). (FIG. 31K-31L) Alanine transferase (ALT) was measured in serum on Day 22 (D1 post boost) (FIG. 31K) and Day 26 (D5 post boost) (FIG. 31L), and (FIG. 31M-31N) aspartate transferase (AST) was measured in serum on Day 22 (D1 post boost) (FIG. 31M) and Day 26 (D5 post boost) (FIG. 31N). The experiment was performed once. Statistical significance was assessed by One-Way ANOVA: P>0.05 (ns), P≤0.05 (*), P≤0.01 (**), P≤0.001(***), P≤0.0001(****).

FIG. 32. CART storage stability assay. CARTs were formulated with Fluc mRNA at a 10:1+/−ratio. These formulations were stored at either RT, 4C, or −20C. After storing CART formulations for 11 days the samples were thawed and injected into tail vein (50 uL total volume, 5 ug mRNA per mouse). At the same time, a fresh formulation of CART/Fluc mRNA was prepared and injected into the tail vein. The levels of luciferase expression were determined by BLI (units: p/s), comparing efficacy to the freshly formulated CART/mRNA. The experiment was performed once. Statistical significance was assessed by One Way Anova: P>0.05 (ns), P≤0.05 (*), P≤0.01 (**), P≤0.001(***), P≤0.0001(****).

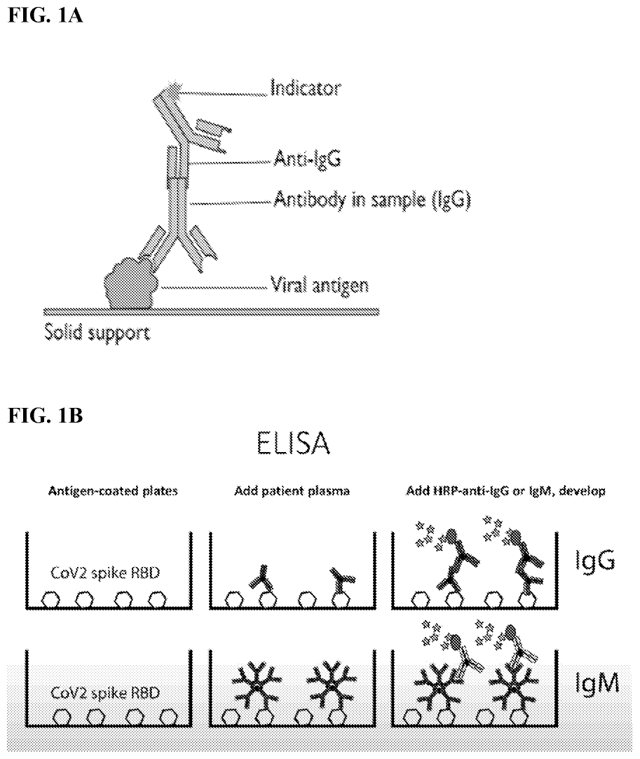

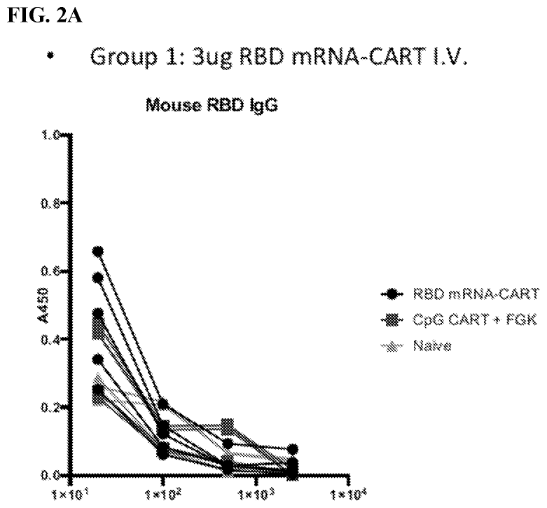

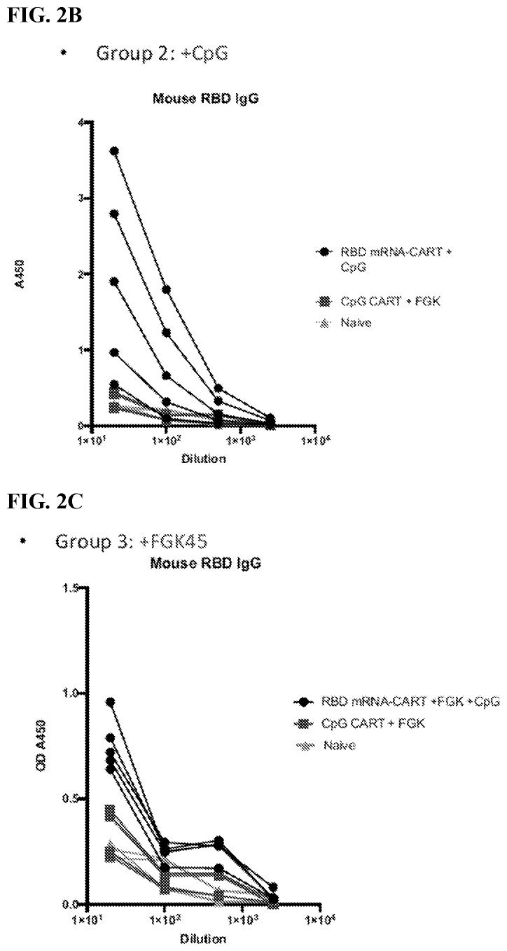

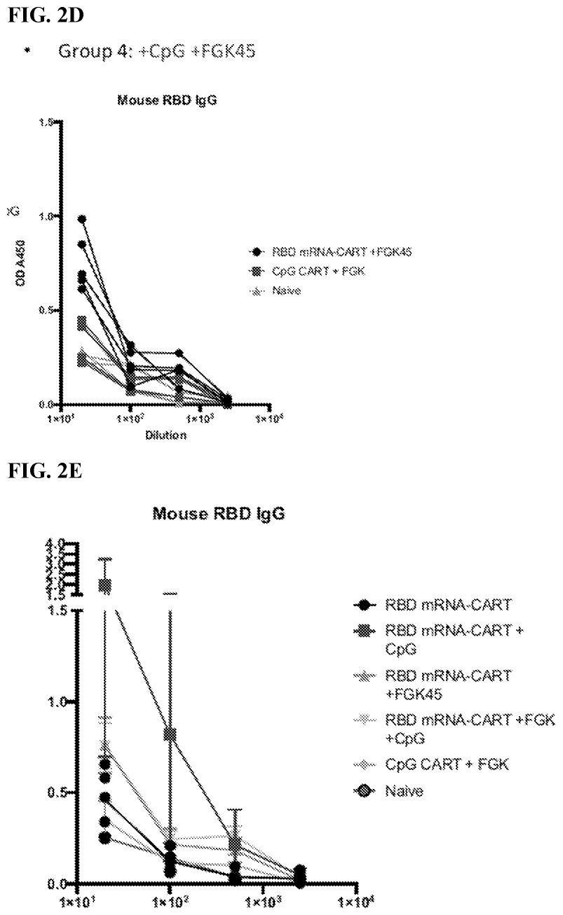

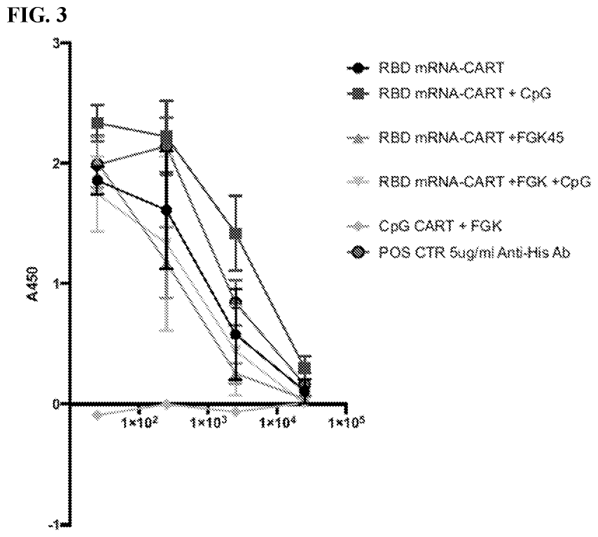

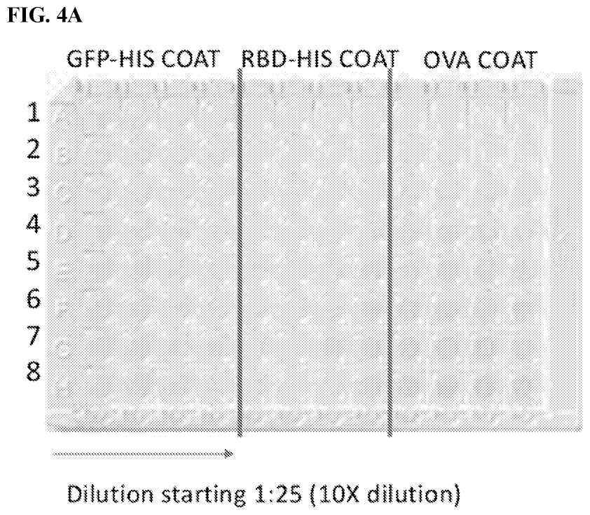

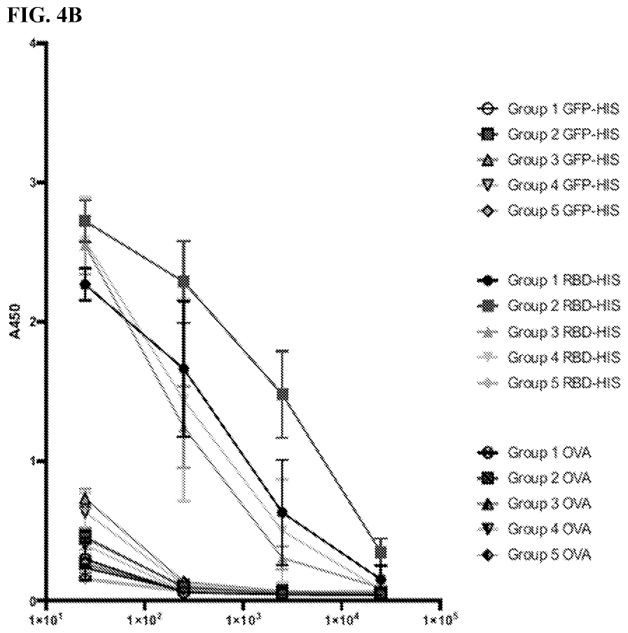

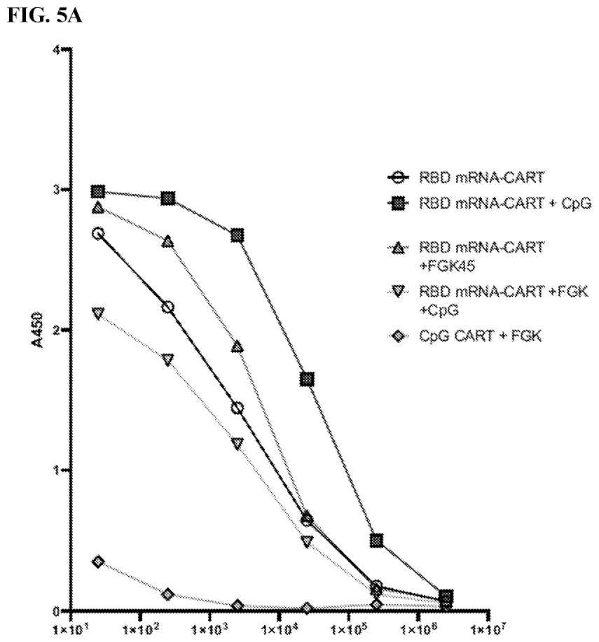

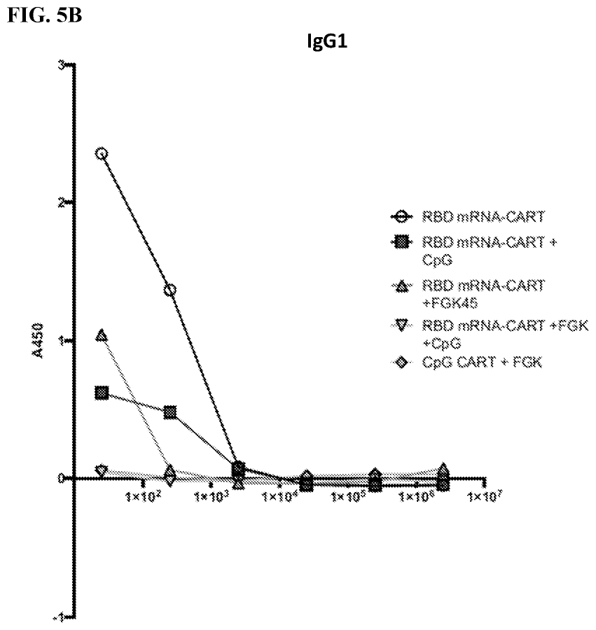

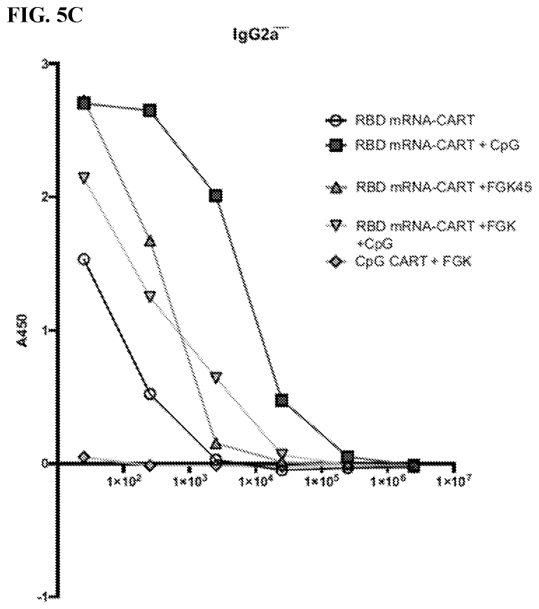

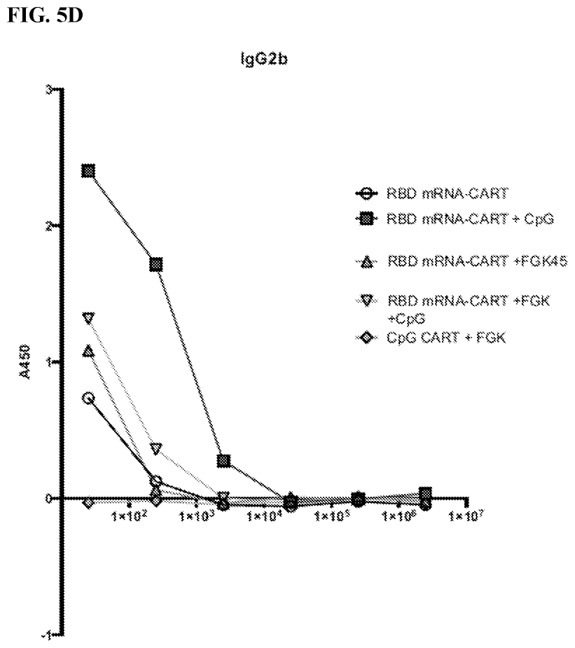

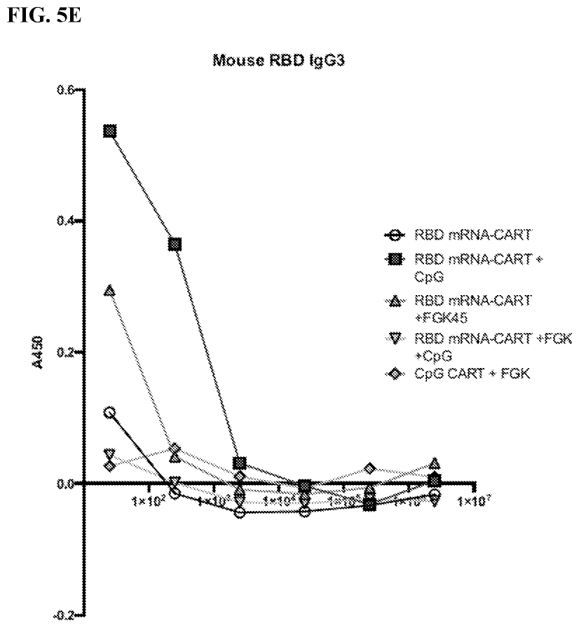

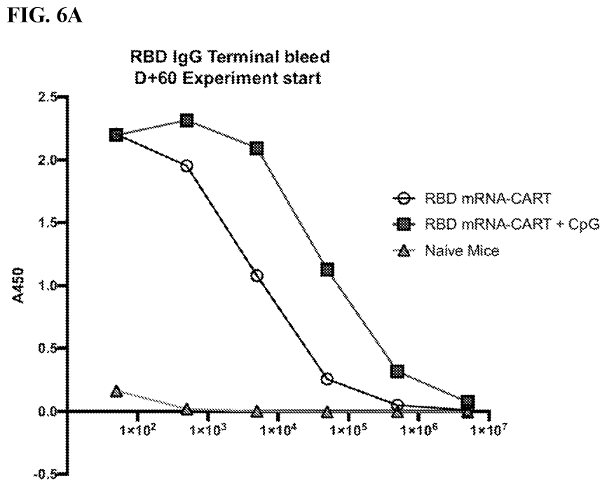

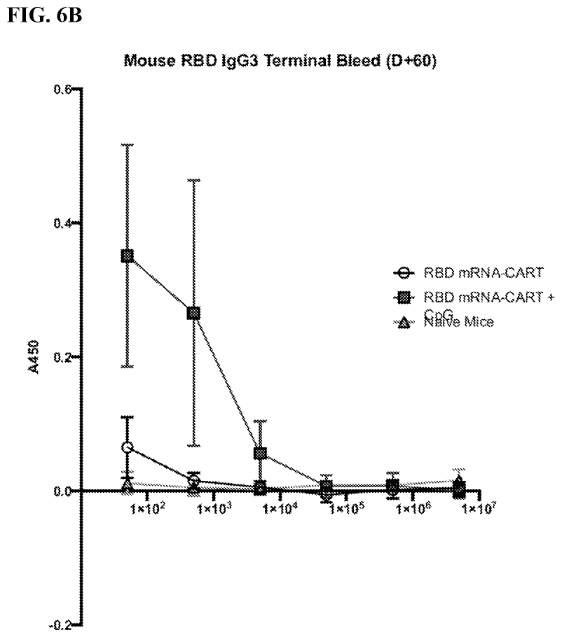

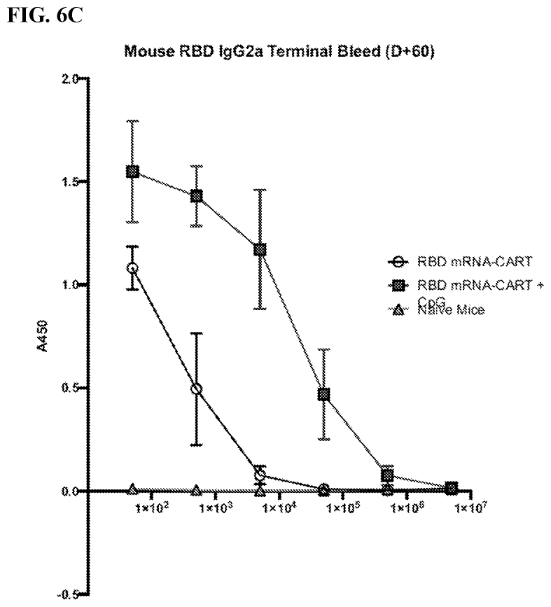

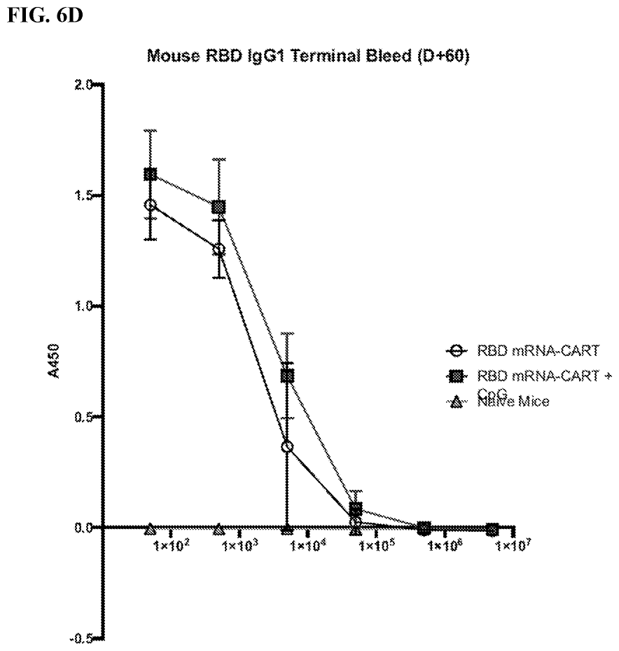

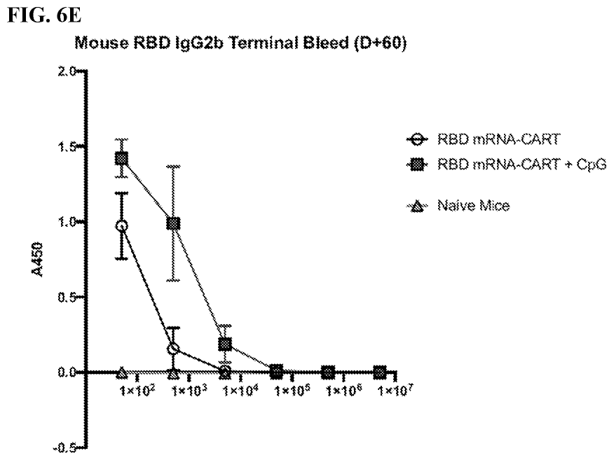

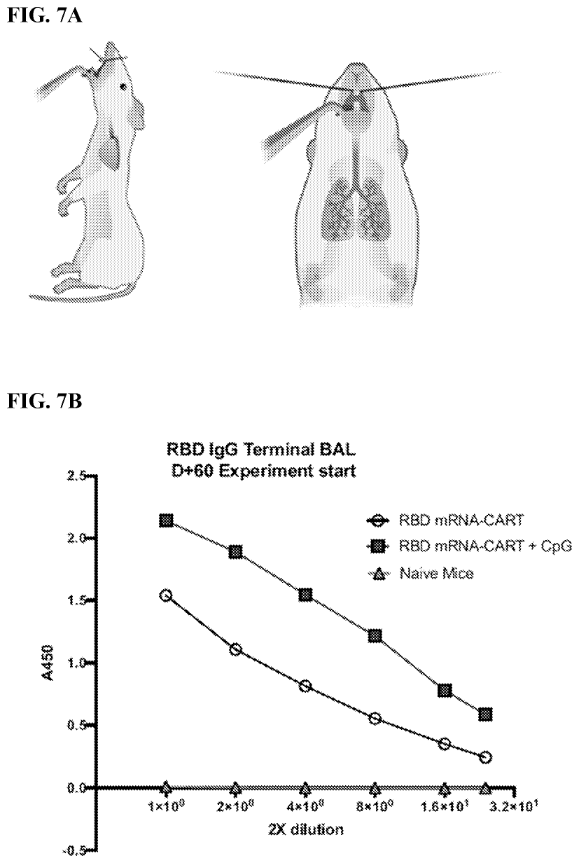

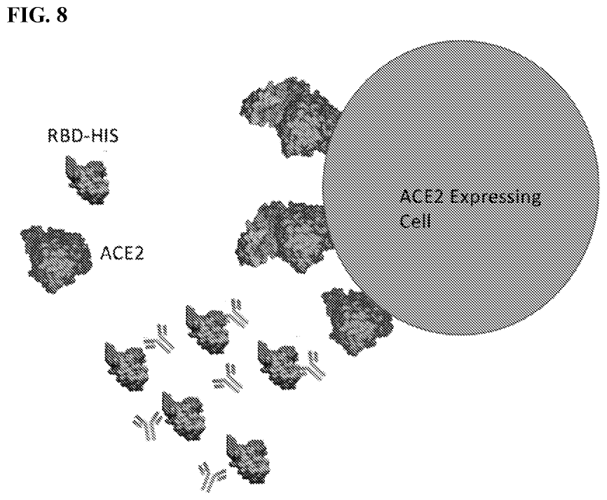

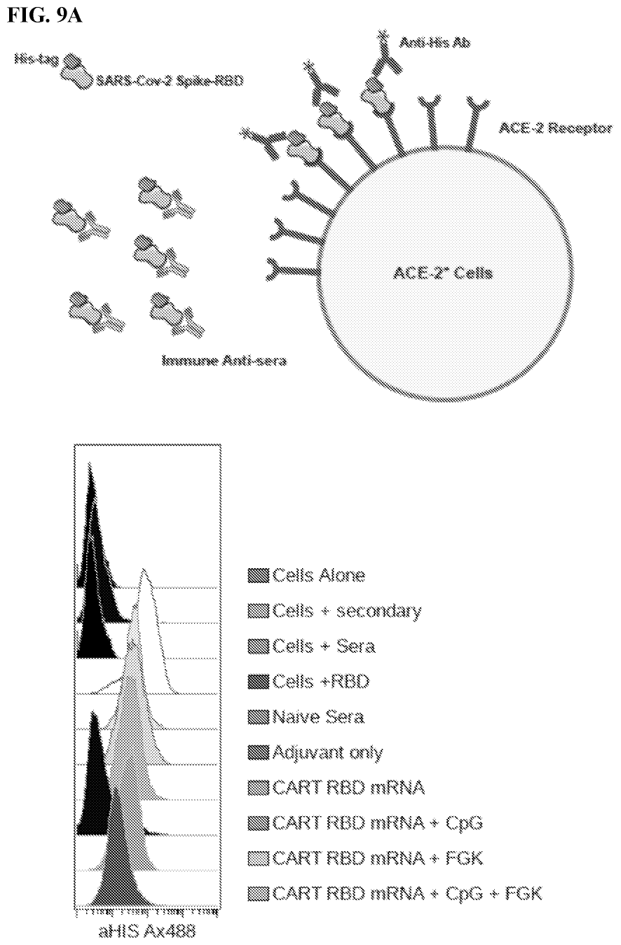

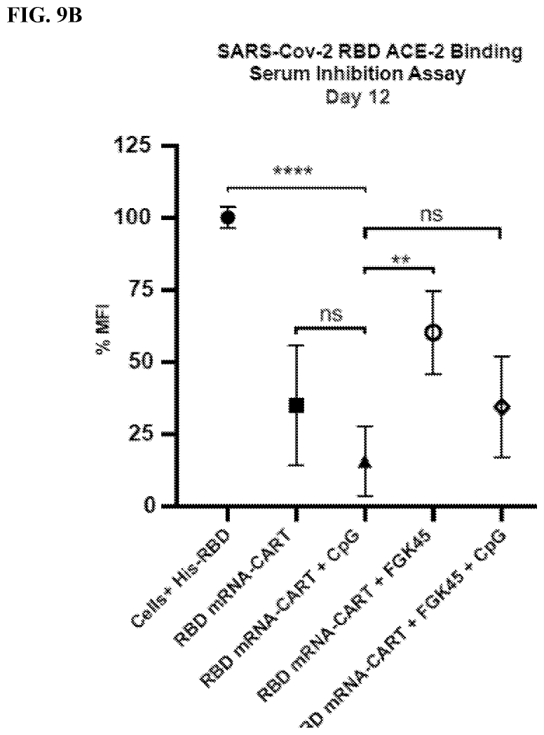

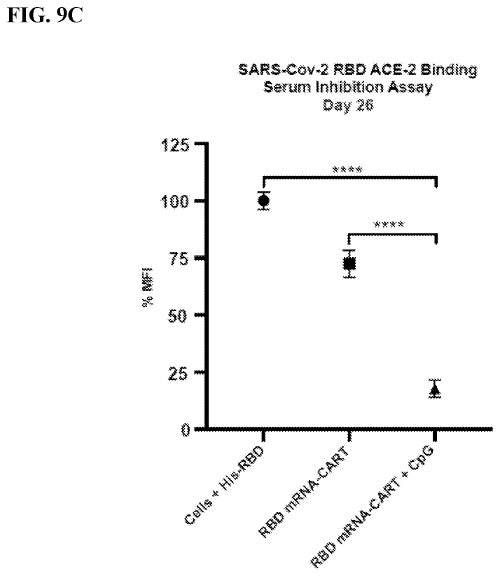

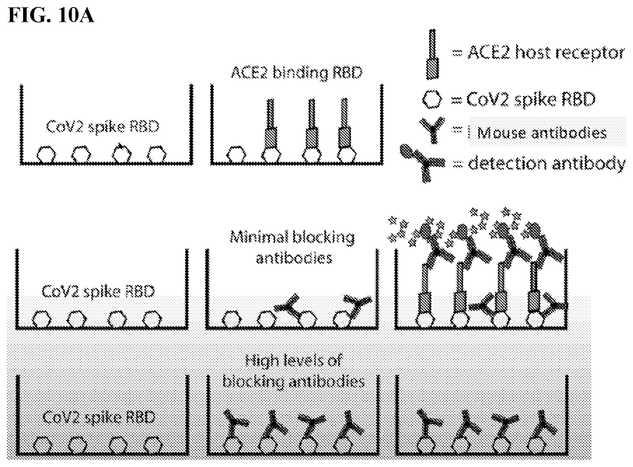

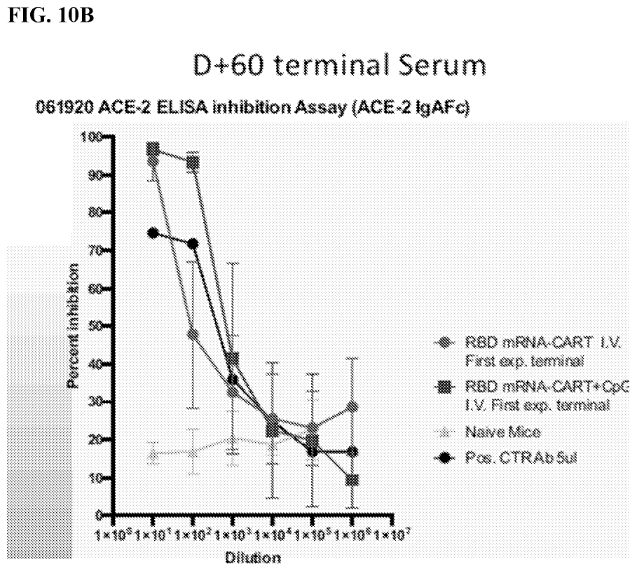

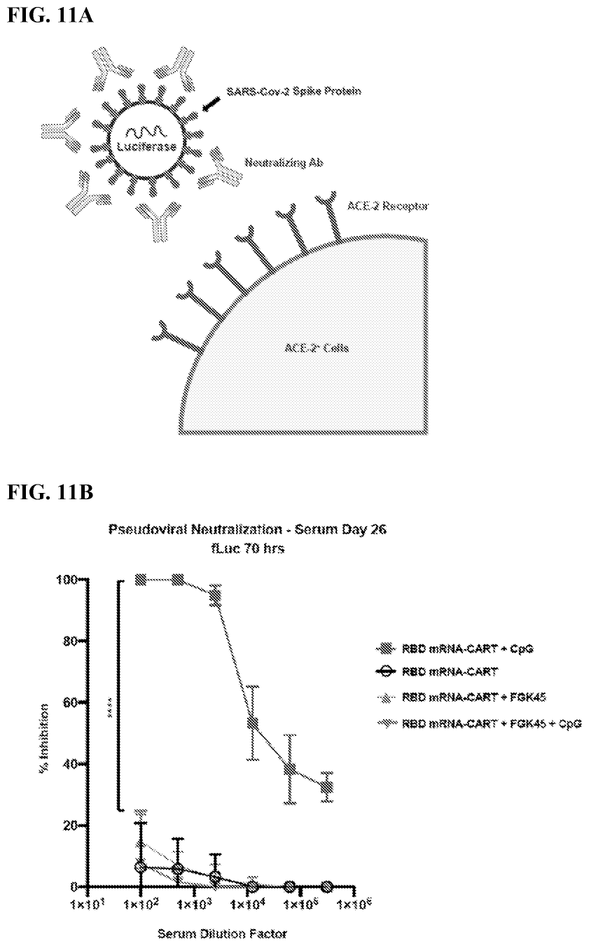

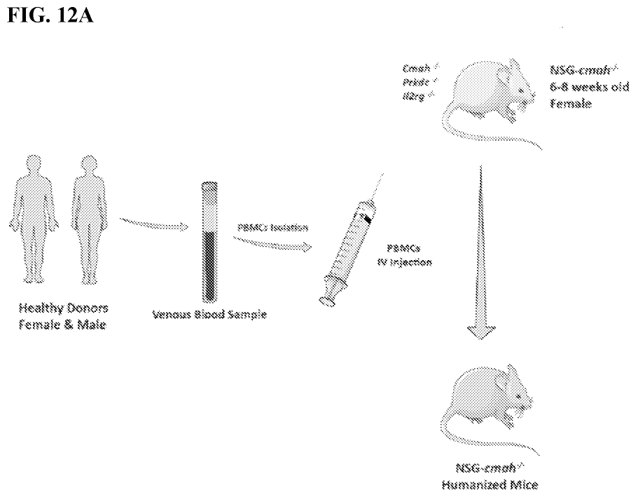

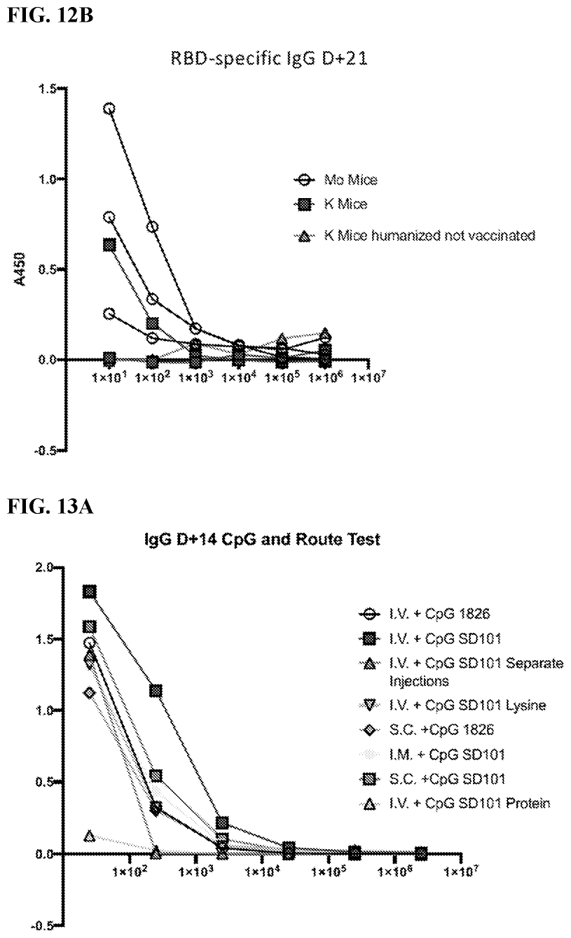

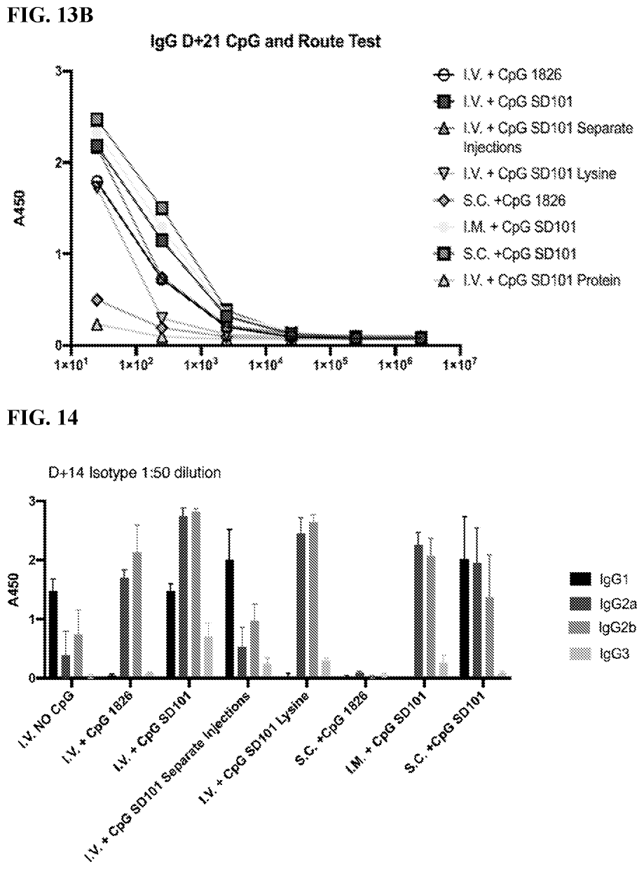

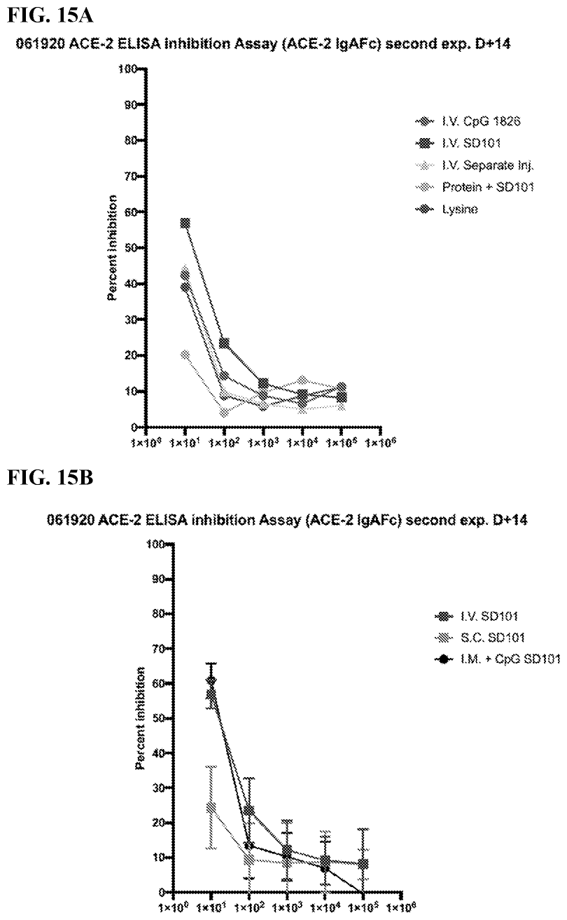

### DETAILED DESCRIPTION

While various embodiments and aspects of the present disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be employed in practicing the disclosure.

Unless the context indicates otherwise, it is specifically intended that the various features of the disclosure described herein can be used in any combination. Moreover, the disclosure also contemplates that in some embodiments any feature or combination of features set forth herein can be excluded or omitted. To illustrate, if the specification states that a complex has components A, B and C, it is specifically intended that any of A, B or C, or a combination thereof, can be omitted and disclaimed singularly or in any combination.

It must be noted that as used herein and in the appended claims, the singular forms “a”, “an”, and “the” include plural referents unless the context clearly dictates otherwise. Thus, for example, reference to “a cancer cell” includes a plurality of cancer cells. In other examples, reference to “a nucleic acid” or “nucleic acid” includes a plurality of nucleic acid molecules, i.e. nucleic acids.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The abbreviations used herein have their conventional meaning within the chemical and biological arts. The chemical structures and formulae set forth herein are constructed according to the standard rules of chemical valency known in the chemical sciences.

Where substituent groups are specified by their conventional chemical formulae, written from left to right, they equally encompass the chemically identical substituents that would result from writing the structure from right to left, e.g., —CH₂O— is equivalent to —OCH₂—.

As used herein the terms “oligomer” and “polymer” refer to a compound that has a plurality of repeating subunits, (e.g., polymerized monomers). The terms “co-oligomer” or “co-polymer” refers to an oligomer or polymer that includes 2 or more different residues (monomer units or monomers, which are interchangeably used herein). The number of monomers in oligomers is generally less than the number of monomers in polymers. Therefore, in some examples, oligomers can have 1 to about 10 monomers, 1 to about 20 monomers, 1 to about 30 monomers, 1 to about 40 monomers, 1 to about 50 monomers, 1 to about 100 monomers, 1 to about 150 monomers, 1 to about 200 monomers, 1 to about 250 monomers, 1 to about 300 monomers, 1 to about 350 monomers, 1 to about 400 monomers, 1 to about 450 monomers or 1 to about 500 monomers is in length. In some examples, oligomers can have less than about 500 monomers, less than about 450 monomers, less than about 400 monomers, less than about 350 monomers, less than about 300 monomers, less than about 250 monomers, less than about 200 monomers, less than about 150 monomers, less than about 100 monomers, less than about 50 monomers, less than about 40 monomers, less than about 30 monomers, less than about 20 monomers or less than about 10 monomers in length. In the context of polymers, the number of monomers in polymers is generally more than the number of monomers in oligomers. Therefore, in some examples, polymers can have about 500 to about 1000 monomers, about 500 to about 2000 monomers, about 500 to about 3000 monomers, about 500 to about 4000 monomers, about 500 to about 5000 monomers, about 500 to about 6000 monomers, about 500 to about 7000 monomers, about 500 to about 8000 monomers, about 500 to about 9000 monomers, about 500 to about 10000 monomers, or more than 10000 monomers in length.

The term “polymerizable monomer” is used in accordance with its meaning in the art of polymer chemistry and refers to a compound that may covalently bind chemically to other monomer molecules (such as other polymerizable monomers that are the same or different) to form a polymer.

The term “block copolymer” is used in accordance with its ordinary meaning and refers to two or more portions (e.g., blocks) of polymerized monomers linked by a covalent bond. In embodiments, a block copolymer is a repeating pattern of polymers. In embodiments, the block copolymer includes two or more monomers in a periodic (e.g., repeating pattern) sequence. For example, a diblock copolymer has the formula: -B-B-B-B-B-B-A-A-A-A-A-, where ‘B’ is a first subunit and ‘A’ is a second subunit covalently bound together. A triblock copolymer therefore is a copolymer with three distinct blocks, two of which may be the same (e.g., -A-A-A-A-A-B-B-B-B-B-B-A-A-A-A-A-) or all three are different (e.g., -A-A-A-A-A-B-B-B-B-B-B-C-C-C-C-C-) where ‘A’ is a first subunit, ‘B’ is a second subunit, and ‘C’ is a third subunit, covalently bound together.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SW, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heteroalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

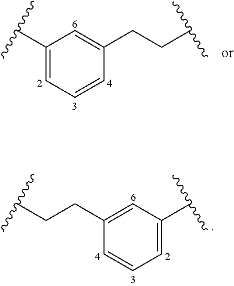 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″″, —S(O)R′, —S(O)₂R′, —S(O)₂NR′R″, —NRSO₂R′, —NR′NR″R″″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO ₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCH Br₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —C₁₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO ₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (a) oxo, halogen, —CCl₃, —CBr₃, —CF₃, —C₁₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OC Br₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO ₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth herein.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C. , 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.;
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g., phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13B}, etc., wherein each of R^{13A}, R^{13B}, R^{13C}, R^{13B}, etc. is defined within the scope of the definition of R¹³ and optionally differently.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R²⁰¹ substituents are present, each R²⁰¹ substituent may be distinguished as R^{201A}, R^{201B}, R^{201C}, R^{201B}, etc., wherein each of R^{201A}, R^{201B}, R^{201C}, R^{201B}, etc. is defined within the scope of the definition of R²⁰¹ and optionally differently.

The term “nucleophilic moiety” refers to a chemical species or functional group that is capable of donating one or more electrons (e.g., 2) to an electrophile. In embodiments, a nucleophilic moiety refers to a chemical species or functional group that can donate an electron to an electrophile in a chemical reaction to form a bond.

The term “electrophilic moiety” refers to a chemical species or functional group that is capable of receiving one or more electrons (e.g., 2). In embodiments, an electrophilic moiety refers to a chemical species or functional group that has a vacant orbital and can thus accept an electron to form a bond in a chemical reaction.

The term “oligoglycol moiety” refers to a chemical entity with the general formula: R⁴⁰⁰—O—(CH2-CH2-O)n₃₀₀- [symbol] where R⁴⁰⁰ is H, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl and n300 is an integer of 1 or more. In some examples, R⁴⁰⁰ is H or alkyl.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

“Nucleic acid” refers to deoxyribonucleotides or ribonucleotides and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA), and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids has one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g. mRNA) or altering transcript splicing (e.g. single stranded morpholino oligo). In embodiments, the nucleic acid is RNA (e.g. mRNA). In embodiments the nucleic acid is 10 to 100,000 bases in length. In embodiments the nucleic acid is 50 and 10,000 bases in length. In embodiments the nucleic acid is 50 and 5,000 bases in length. In embodiments the nucleic acid is 50 and 1,000 bases in length.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The terms “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. In embodiments, contacting includes, for example, allowing a nucleic acid to interact with an endonuclease.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

A “vector” is a nucleic acid that is capable of transporting another nucleic acid into a cell. A vector is capable of directing expression of a protein or proteins encoded by one or more genes carried by the vector when it is present in the appropriate environment.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/. By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, having the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “specific binding” or “specifically binds” refer to two molecules forming a complex (e.g., viral protein and a receptor for said viral protein) that is relatively stable under physiologic conditions.

Methods for determining whether a ligand binds another species (e.g., a protein or nucleic acid) and/or the affinity of such ligand-species interaction are known in the art. For example, the binding of a ligand to a protein can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA).

Immunoassays which can be used to analyze immunospecific binding and cross-reactivity of the ligand include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

An exemplary immunoglobulin (antibody) structural unit can have a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

As used herein, the terms “immolation,” “self-immolation,” “self-immolation mechanism,” “immolation moiety,” “immolation domain” and the like refer herein to the ability of a chemical group to undergo an intramolecular reaction thereby resulting in a chemical rearrangement of the chemical group and release of the rearranged chemical group from the remainder of the compound to which it was attached. A “pH-sensitive” immolation domain refers to a chemical group that undergoes an immolation reaction within a discreet pH range and does not substantially undergo the immolation reaction outside of the discreet pH range (e.g., pH about 1-5, pH about 5-7 or pH about 7-10). In embodiments, the discreet pH range is: pH 1-3, pH 2-4, pH 3-5, pH 4-6, pH 5-7, pH 6-8, pH 7-9, or pH 8-10. In embodiments, the pH-sensitive immolation region includes a cationic alpha amino ester (oligo(α-aminoester)). In embodiments, the cationic component of the cationic alpha amino ester is a positively charged nitrogen atom (e.g. a cationic amine). In embodiments, the cationic component of the cationic alpha amino ester is not a guanidinium group. In embodiments, the cationic component of the cationic alpha amino ester is not a piperidinium group.

The term “cell-penetrating complex” or the like refer, in the usual and customary sense, to a chemical complex (e.g., a complex or composition disclosed herein and embodiments thereof), capable of penetrating into a cell (a biological cell, such as a eukaryotic cell or prokaryotic cell). In embodiments, the cell-penetrating complex includes a nucleic acid ionically bound to a cationic amphipathic polymer. In embodiments, the nucleic acid is unable to substantially penetrate the cell in the absence of the cationic amphipathic polymer. Thus, in embodiments, the cationic amphipathic polymer facilitates the transport of the nucleic acid into the cell. As used herein, the terms “cationic charge altering releasable transporter,” “CART” and the like refer to the cell-penetrating complexes disclosed herein. The CART compounds are able to release the nucleic acid component within the cell through the action of a pH-sensitive immolation domain within the cationic amphipathic polymer component, which reacts in response to an intracellular pH thereby releasing the nucleic acid with in the cell. In embodiments, the cationic amphipathic polymer degrades rapidly within the cell (e.g. a T1/2 of less than 6 hours at pH 7.4). At least in some embodiments, a polyplex, a complex, an electrostatic complex, a CART/mRNA complex, a CART/oligonucleotide complex and nanoparticle can interchangeably be used to refer to a cell-penetrating complex.

The term “amphipathic polymer” as used herein refers to a polymer containing both hydrophilic and hydrophobic portions. In embodiments, the hydrophilic to hydrophobic portions are present in a 1 to 1 mass ratio. In embodiments, the hydrophilic to hydrophobic portions are present in a 1 to 2 mass ratio. In embodiments, the hydrophilic to hydrophobic portions are present in a 1 to 5 mass ratio. In embodiments, the hydrophilic to hydrophobic portions are present in a 2 to 1 mass ratio. In embodiments, the hydrophilic to hydrophobic portions are present in a 5 to 1 mass ratio. An amphipathic polymer may be a diblock or triblock copolymer. In embodiments, the amphiphilic polymer may include two hydrophilic portions (e.g., blocks) and one hydrophobic portion (e.g., block).

The term “lipophilic polymer domain” or the like, often referred to as “lipid block” refers to a region of the cationic amphipathic polymer that is not hydrophilic (e.g. is insoluble in water alone). In embodiments, the lipophilic polymer domain has low solubility in water. For example, low solubility in water refers to the solubility of a lipophilic polymer domain which is about 0.0005 mg/mL to about 10 mg/mL soluble in water.

The term “initiator” refers to a compound that is involved in a reaction synthesizing a cationic amphipathic polymer having the purpose of initiating the polymerization reaction. Thus, the initiator is typically incorporated at the end of a synthesized polymer. For example, a plurality of molecules of one type (or formula) of monomer or more than one type of monomers (e.g. two different types of monomers) can be reacted with an initiator to provide a cationic amphipathic polymer. The initiator can be present on at least one end of the resulting polymer and not constitute a repeating (or polymerized) unit(s) present in the polymer.

The terms “disease” or “condition” refer to a state of being or health status of a subject capable of being treated with a compound, pharmaceutical composition, or method provided herein. The disease can be an autoimmune, inflammatory, cancer, infectious, metabolic, developmental, cardiovascular, liver, intestinal, endocrine, neurological, or other disease. In some examples, the disease is cancer (e.g. breast cancer, ovarian cancer, sarcoma, osteosarcoma, lung cancer, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, neuroblastoma).

The term “infection” or “infectious disease” refers to a disease or condition that can be caused by organisms such as a bacterium, virus, fungi or any other pathogenic microbial agents.

The terms “virus” or “virus particle” are used according to their plain ordinary meaning in the biological arts and refer to a particle including a viral genome (e.g. DNA, RNA, single strand, double strand), a protective coat of proteins (e.g. capsid) and associated proteins, and in the case of enveloped viruses (e.g. herpesvirus), an envelope including lipids and optionally components of host cell membranes, and/or viral proteins.

The term “viral infection” or “viral disease” refers to a disease or condition that is caused by a virus. Non-limiting examples of viral infections include hepatic viral diseases (e.g., hepatitis A, B, C, D, E), herpes virus infection (e.g., HSV-1, HSV-2, herpes zoster), flavivirus infection, Zika virus infection, cytomegalovirus infection, a respiratory viral infetion (e.g., adenovirus infection, influenza, severe acute respiratory syndrome, coronavirus infection (e.g., SARS-CoV-1, SARS-CoV-2, MERS-CoV, COVID-19, MERS)), a gastrointestinal viral infection (e.g., norovirus infection, rotavirus infection, astrovirus infection), an exanthematous viral infection (e.g., measles, shingles, smallpox, rubella), viral hemorrhagic disease (e.g., Ebola, Lassa fever, dengue fever, yellow fever), a neurologic viral infection (e.g., West Nile viral infection, polio, viral meningitis, viral encephalitis, Japanese enchephalitis, rabies), and human papilloma viral infection.

The term “infection” or “infectious disease” refers to a disease or condition that can be caused by organisms such as a bacterium, virus, fungi or any other pathogenic microbial agents. In embodiments, the infectious disease is caused by a pathogenic virus. Pathogenic viruses are viruses that can infect and replicate within cells (e.g. human cells) and cause diseases. In embodiments, the infectious disease is a virus associated disease. Non-limiting virus associated diseases include hepatic viral diseases (e.g., hepatitis A, B, C, D, E), herpes virus infection (e.g., HSV-1, HSV-2, herpes zoster), flavivirus infection, Zika virus infection, cytomegalovirus infection, a respiratory viral infetion (e.g., adenovirus infection, influenza, severe acute respiratory syndrome, coronavirus infection (e.g., SARS-CoV-1, SARS-CoV-2, MERS-CoV, COVID-19, MERS)), a gastrointestinal viral infection (e.g., norovirus infection, rotavirus infection, astrovirus infection), an exanthematous viral infection (e.g., measles, shingles, smallpox, rubella), viral hemorrhagic disease (e.g., Ebola, Lassa fever, dengue fever, yellow fever), a neurologic viral infection (e.g., West Nile viral infection, polio, viral meningitis, viral encephalitis, Japanese enchephalitis, rabies), and human papilloma viral infection.

As defined herein, the term “inhibition”, “inhibit”, “inhibiting” and the like in reference to an activity and/or functionality of a molecule (e.g. polynucleotide or protein) means negatively affecting (e.g., decreasing or reducing) the activity or function of the molecule relative to the activity or function of the protein in the absence of the inhibition. Thus, inhibition includes, at least in part, partially or totally blocking stimulation, decreasing, preventing, or delaying activation, or inactivating, desensitizing, or down-regulating signal transduction or enzymatic activity or the amount of a protein or polynucleotide. Similarly an “inhibitor” is a compound that inhibits a target bio-molecule (i.e. nucleic acid, peptide, carbohydrate, lipid or any other molecules that can be found from nature), e.g., by binding, partially or totally blocking, decreasing, preventing, delaying, inactivating, desensitizing, or down-regulating activity of the target bio-molecule. In the context of disease prevention treatment, inhibition refers to reduction of a disease or symptoms of disease.

SARS-CoV-2 belongs to the family of betacoronaviruses, whose members include two other zoonotic viruses that have caused severe disease outbreaks in the new millennium: severe acute respiratory syndrome coronavirus (SARS-CoV) and Middle East respiratory syndrome coronavirus (MERS-CoV). SARS-CoV-2 shows nearly 80 percent genetic similarity to SARS-CoV, which triggered the severe acute respiratory syndrome (SARS) epidemic in 2002-2003. SARS-CoV-2 is more distantly related to MERS-CoV, which is responsible for the Middle East respiratory syndrome (MERS) epidemic that began in 2012 and still persists. See, e.g., Yuki et al., 2020, Clin. Immun. 215, 108427; Chen et al. 2020, J. Med. Virol. 92, 418-423. The term “SARS-CoV” refers to SARS coronavirus. The term “SARS-CoV” includes any coronovirus, such as SARS-CoV-2, SARS-CoV-1, and MERS-CoV.

“COVID-19” refers to the disease caused by SARS-CoV-2. COVID-19 has an incubation period of 2-14 days, and symptoms include, e.g., fever, tiredness, cough, and shortness of breath (e.g., difficulty breathing).

“MERS-CoV” refers to Middle Eastern respiratory syndrome-associated coronavirus. See, e.g., Chung et al, Genetic Characterization of Middle East Respiratory Syndrome Coronavirus, South Korea, 2018. Emerging Infectious Diseases, 25(5):958-962 (2019).

“Middle Eastern respiratory syndrome” or “MERS” refers to the disease caused by MERS-coronavirus.

“Treatment,” “treating,” and “treat” are defined as acting upon a disease, disorder, or condition with an agent to reduce or ameliorate harmful or any other undesired effects of the disease, disorder, or condition and/or its symptoms. “Treating” or “treatment of” a condition or subject in need thereof refers to (1) taking steps to obtain beneficial or desired results, including clinical results such as the reduction of symptoms; (2) inhibiting the disease, for example, arresting or reducing the development of the disease or its clinical symptoms; (3) relieving the disease, for example, causing regression of the disease or its clinical symptoms; or (4) delaying the disease. For example, beneficial or desired clinical results include, but are not limited to, reduction and/or elimination of cancer cells and prevention and/or reduction of metastasis of cancer cells.

The term “prevent,” “preventing” or “prevention”, in the context of a disease, refers to causing the clinical symptoms of the disease not to develop in a subject that does not yet experience or display symptoms of the disease. In some examples, such prevention can be applied to a subject who can be considered predisposed of the disease, whereas in some other examples, the subject may not be necessarily considered predisposed to the disease.

As used herein, “administering” refers to the physical introduction of a composition to a subject, using any of the various methods and delivery systems known to those skilled in the art. Preferred routes of administration for the composition described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, the composition described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “associated” or “associated with” in the context of a substance or substance activity or function associated with a disease means that the disease can be caused by (in whole or in part), or a symptom of the disease can be caused by (in whole or in part) the substance or substance activity or function. When the term is used in the context of a symptom, e.g. a symptom being associated with a disease or condition, it means that a symptom can be indicative of the disease or condition present in the subject who shows the symptom.

The term “subject,” “individual,” “host” or “subject in need thereof” refers to a living organism suffering from a disease or condition or having a possibility to have a disease or condition in the future. A term “patient” refers to a living organism that already has a disease or condition, e.g. a patient who has been diagnosed with a disease or condition or has one or more symptoms associated with a disease or condition. Non-limiting examples include humans, other mammals, bovines, rats, mice, dogs, monkeys, goat, sheep, cows, deer, and other non-mammalian animals. In some embodiments, a patient is human.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infection disease, the vaccine composition can provide mRNA encoding antigenic molecules that are associated with a certain pathogen, e.g. one or more peptides that are known to be expressed in the pathogen (e.g. pathogenic bacterium or virus). In the context of a viral vaccine, the vaccine composition can provide mRNA encoding certain viral peptides that are characteristic for the virus that immunity is sought for, e.g. peptides that are substantially exclusively or highly expressed on the viral surface (e.g., capsid). The subject, after vaccination with the viral vaccine composition, can have immunity against the viral peptide t kill the cells expressing it with specificity.

The term “adjuvant” is used in accordance with its plain ordinary meaning within Immunology and refers to a substance that is commonly used as a component of a vaccine. Adjuvants may increase an antigen specific immune response in a subject when administered to the subject with one or more specific antigens as part of a vaccine. In embodiments, an adjuvant accelerates an immune response to an antigen. In embodiments, an adjuvant prolongs an immune response to an antigen. In embodiments, an adjuvant enhances an immune response to an antigen.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

According to the methods provided herein, the subject can be administered an effective amount of one or more of agents, compositions or complexes, all of which are interchangeably used herein, (e.g. cell-penetrating complex or vaccine composition including the same) provided herein. The terms “effective amount” and “effective dosage” are used interchangeably. The term “effective amount” is defined as any amount necessary to produce a desired effect (e.g., transfection of nucleic acid into cells and exhibiting intended outcome of the transfected nucleic acid). Effective amounts and schedules for administering the agent can be determined empirically by one skilled in the art. The dosage ranges for administration are those large enough to produce the desired effects, e.g. transfection of nucleic acid, modulation in gene expression, gene-edition, induction of stem cells, induction of immune response and more. The dosage should not be so large as to cause substantial adverse side effects, such as unwanted cross-reactions, anaphylactic reactions, and the like. Generally, the dosage can vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount can show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation can depend on the purpose of the treatment, and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

### Cell-Penetrating Complexes—Section 1

According to the embodiments of Formula (I), (II), (III), and (IV), all substituents are defined as listed below.

Further to the cell-penetration complex disclosed herein and embodiments thereof, in embodiments, the counter-anion to the above cationic sequences can include common counterions known in the art, such as for example acetate, trifluoroacetate, triflate, chloride, bromide, sulfate, phosphate, succinate, or citrate. In embodiments, the counter-anion is acetate, trifluoroacetate, triflate, chloride, bromide, sulfate, phosphate, succinate, or citrate. Further, it is noted that any of the cell-penetrating complexes described in the following sections 2-4, may be used for the compositions and methods provided under section 1. For example, any of the nucleic acid, cationic amphipathic polymer and cationic amphipathic polymer described in this section may form part of a cell-penetrating complex including a ribonucleic acid including a sequence encoding a viral protein, a nucleic acid adjuvant and a cationic amphipathic polymer as provided herein including embodiments thereof.

In an aspect is provided a cell-penetrating complex including a ribonucleic acid including a sequence encoding a viral protein, a nucleic acid adjuvant and a cationic amphipathic polymer.

In embodiments, the ribonucleic acid is non-covalently bound to the cationic amphipathic polymer.

In embodiments, the viral protein is a respiratory syncytial virus (RSV) protein, human metapneumovirus (hMPV) protein, parainfluenza virus type 3 (PIV3) protein, influenza H10N8 virus protein, influenza H7N9 virus protein, cytomegalovirus (CMV) protein, Zika virus protein, chikungunya virus protein, or a severe acute respiratory syndrome (SARS) associated coronavirus (CoV) protein. In embodiments, the viral protein is a respiratory syncytial virus (RSV) protein. In embodiments, the viral protein is a human metapneumovirus (hMPV) protein. In embodiments, the viral protein is a parainfluenza virus type 3 (PIV3) protein. In embodiments, the viral protein is an influenza H10N8 virus protein. In embodiments, the viral protein is an influenza H7N9 virus protein. In embodiments, the viral protein is a cytomegalovirus (CMV) protein. In embodiments, the viral protein is a Zika virus protein. In embodiments, the viral protein is a chikungunya virus protein. In embodiments, the viral protein is a severe acute respiratory syndrome (SARS) associated coronavirus (CoV) protein.

In embodiments, the viral protein is a SARS-CoV-1 protein or a SARS-CoV-2 protein. In embodiments, the viral protein is a SARS-CoV-2 spike protein or fragment thereof. In embodiments, the viral protein is the soluble receptor binding domain (RBD) of a SARS-CoV-2 spike protein.

In embodiments, the ribonucleic acid includes the sequence of SEQ ID NO:3. In embodiments, the ribonucleic acid is the sequence of SEQ ID NO:3.

In embodiments, the ribonucleic acid includes a portion or fragment of a SARS-CoV-2 spike protein. In embodiments, the ribonucleic acid includes a portion or fragment of the sequence of SEQ ID NO:4. In further embodiments, the portion or fragment is at least 100, 200, 300, 400, 500, 600, or 700 nucleotides in length. In further embodiments, the portion or fragment is at least 100, 200, 300, 400, 500, 600, or 700 consecutive nucleotides of SEQ ID NO:4. In further embodiments, the portion or fragment is about 100, 200, 300, 400, 500, 600, or 700 nucleotides in length. In further embodiments, the portion or fragment is about 100, 200, 300, 400, 500, 600, or 700 consecutive nucleotides of SEQ ID NO:4. In further embodiments, the portion or fragment is from about 50 to about 900 nucleotides in length. In further embodiments, the portion or fragment is from about 100 to about 900 nucleotides in length. In further embodiments, the portion or fragment is from about 150 to about 900 nucleotides in length. In further embodiments, the portion or fragment is from about 200 to about 900 nucleotides in length. In further embodiments, the portion or fragment is from about 250 to about 900 nucleotides in length. In further embodiments, the portion or fragment is from about 300 to about 900 nucleotides in length. In further embodiments, the portion or fragment is from about 350 to about 900 nucleotides in length. In further embodiments, the portion or fragment is from about 400 to about 900 nucleotides in length. In further embodiments, the portion or fragment is from about 450 to about 900 nucleotides in length. In further embodiments, the portion or fragment is from about 500 to about 900 nucleotides in length. In further embodiments, the portion or fragment is from about 550 to about 900 nucleotides in length. In further embodiments, the portion or fragment is from about 600 to about 900 nucleotides in length. In further embodiments, the portion or fragment is from about 650 to about 900 nucleotides in length. In further embodiments, the portion or fragment is from about 700 to about 900 nucleotides in length. In further embodiments, the portion or fragment is from about 750 to about 900 nucleotides in length. In further embodiments, the portion or fragment is from about 800 to about 900 nucleotides in length. The protion or fragment provided herein may include 50 to about 900 consecutive nucleotides of SEQ ID NO:4.

In embodiments, the nucleic acid adjuvant is a DNA adjuvant.

In embodiments, the nucleic acid adjuvant is a toll-like receptor (TLR) agonist.

In embodiments, the nucleic acid adjuvant includes one or more unmethylated CpG oligonucleotides.

In embodiments, the nucleic acid adjuvant is a TLR-9 agonist.

In embodiments, the nucleic acid adjuvant has the sequence of SEQ ID NO:1 (tccatgacgttcctgacgtt) or SEQ ID NO:2 (tcgaacgttcgaacgttcgaacgttcgaat). In embodiments, the nucleic acid adjuvant has the sequence of SEQ ID NO:1. In embodiments, the nucleic acid adjuvant has the sequence of SEQ ID NO:2.

In embodiments, the nucleic acid adjuvant is referred to as ODN 1826 and has the sequence of SEQ ID NO:1 (5′-tccatgacgttcctgacgtt-3′). The nucleic acid adjuvant may include a full phosphorothioate backbone. In embodiments, the nucleic acid adjuvant is nuclease resistant.

In embodiments, the nucleic acid adjuvant is referred to as ODN SD-101 and has the sequence of SEQ ID NO:2 (5′-tcgaacgttcgaacgttcgaacgttcgaat 3′). The nucleic acid adjuvant may include a full phosphorothioate backbone. In embodiments, the nucleic acid adjuvant is nuclease resistant.

In embodiments, the cationic amphipathic polymer includes a pH-sensitive immolation domain and a lipophilic polymer domain.

In embodiments, the cationic amphipathic polymer has the formula:

R^{1A}-[L¹-[(LP¹)_{z1}-(LP²)_{z3}-(IM)_{z2}]_{z4}-L²-R^{2A}]_{z5} (I),

wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —C₁₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain; IM is the pH-sensitive immolation domain;
z5 is an integer from 1 to 10;
z1 and z3 are independently integers from 0 to 100, wherein at least one of z1 or z3 is not 0;
z4 is an integer from 1 to 100; and
z2 is an integer from 2 to 100.

In embodiments, the lipophilic polymer domain has the formula:

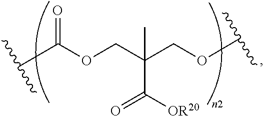

wherein n2 is an integer from 1 to 100; R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, LP¹ has the formula:

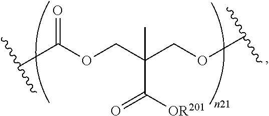

wherein n21 is an integer from 1 to 100; R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n21 is 5 and R²⁰¹ is unsubstituted C₁₈ alkenyl. In embodiments, n21 is 6 and R²⁰¹ is unsubstituted C₁₈ alkenyl.

In embodiments, the unsubstituted C₁₈ alkenyl is oleyl.

In embodiments, LP² has the formula:

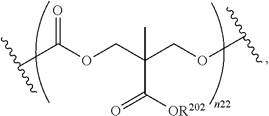

wherein n22 is an integer from 1 to 100; R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n22 is 5 and R²⁰² is unsubstituted C₉ alkenyl. In embodiments, n22 is 6 and R²⁰² is unsubstituted C₉ alkenyl.

In embodiments, the unsubstituted C₉ alkenyl is nonenyl.

In embodiments, the cationic amphipathic polymer has the formula:

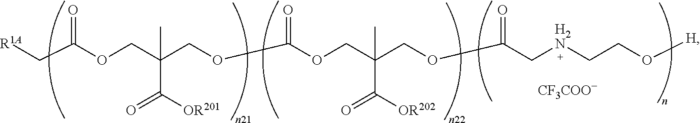

wherein n21 is 5, R²⁰¹ is oleyl, n22 is 5, R²⁰² is nonenyl and n is 7.

In embodiments, the cationic amphipathic polymer has the formula:

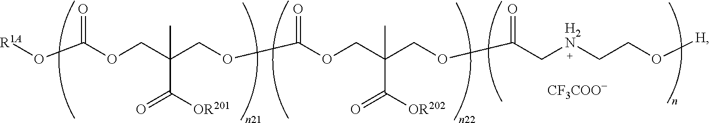

wherein n21 is 6, R²⁰¹ is oleyl, n22 is 6, R²⁰² is nonenyl and n is 9.

In embodiments, the cationic amphipathic polymer has the formula:

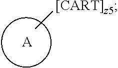

wherein
Ring A is a substituted or unsubstituted cycloalkyl, substituted or unsubstituted heterocycloalkyl, substituted or unsubstituted aryl, or substituted or unsubstituted heteroaryl;
CART has the formula: -L¹-[(LP¹)_{z1}-(LP²)_{z3}-(IM)_{z2}]_{z4}-L²-R^{2A}
   wherein,
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain;
IM is the pH-sensitive immolation domain;
z5 is an integer from 1 to 10;
z1 and z3 are independently integers from 0 to 100, wherein at least one of z1 or z3 is not 0;
z4 is an integer from 1 to 100, and
z2 is an integer from 2 to 100.

In embodiments, Ring A is a substituted or unsubstituted aryl. In embodiments, Ring A is a substituted or unsubstituted phenyl. In embodiments, Ring A is a substituted or unsubstituted phenyl or naphthalenyl. In embodiments, Ring A is a substituted or unsubstituted naphthalenyl.

In embodiments, the cationic amphipathic polymer has the formula:

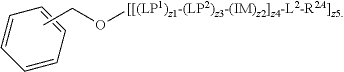

In embodiments, the cationic amphipathic polymer has the formula:

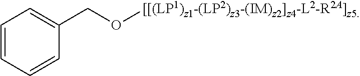

In embodiments, the cationic amphipathic polymer has the formula:

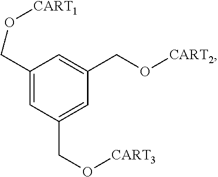

wherein CART₁, CART₂ and CART₃ are independently CART as defined herein.

In embodiments, z5 is an integer from 1 to 3. In embodiments, z5 is 1 or 3. In embodiments, z5 is 1. In embodiments, z5 is 3.

In embodiments, R^{2A} is hydrogen.

In embodiments, L² is a bond.

In embodiments, the pH-sensitive immolation domain has the formula:

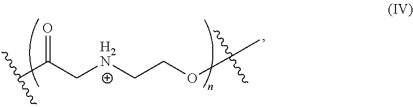

wherein n is an integer of 2 or more.

In embodiments, n is an integer in the range of 2-50. In embodiments, n is an integer from 2 to 50. In embodiments, n is 7. In embodiments, n is 9.

In embodiments, the cationic amphipathic polymer has the formula:

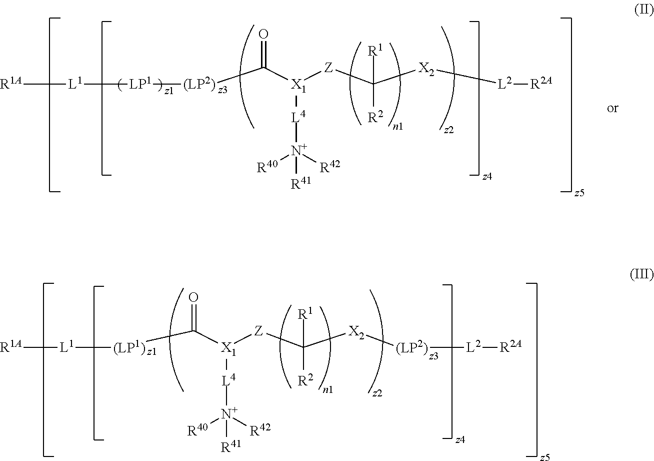

wherein
R^{1A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, independently —COOH, —CONH₂, —NO₂, —SH, —SO₃H, —SO₄H, —SO₂NH₂, —NHNH₂, —ONH₂, independently —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain;
X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—;
X² is —O— or —S—;
R¹, R², R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
L⁴ is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene;
R⁴⁰, R⁴¹, and R⁴² are independently hydrogen, substituted or unsubstituted alkyl or susbstituted or unsubstituted heteroalkyl;
Z is —S—, —S⁺R¹³—, —NR¹³ —, or —N⁺(R¹³)(H)—;
R¹³ is hydrogen, —CCl₃, —CBr₃, —CF₃, —CI₃, —CN, —OH, ═O, —NH₂, —COOH, —CONH₂, —SH, —SO₃H, SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
n1 is an integer from 0 to 50; z1 and z3 are independently integers from 0 to 100, wherein at least one of z1 or z3 is not 0; z2 is an integer from 2 to 100; z4 is an integer from 1 to 100;
and z5 is an integer from 1 to 10.

In embodiments, LP¹ has the formula:

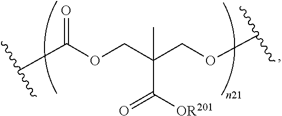

wherein n21 is an integer from 1 to 100; R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n21 is 10-40.

In embodiments, R²⁰¹ is unsubstituted C₁₂ alkyl.

In embodiments, LP² has the formula:

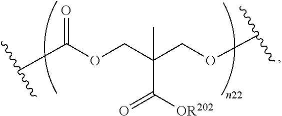

wherein n22 is an integer from 1 to 100; R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n22 is 10-35.

In embodiments, R²⁰² is unsubstituted C₁₂ alkenyl.

In embodiments, the cationic amphipathic polymer has the formula:

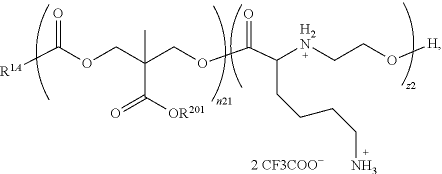

wherein n21 is an integer from 10 to 20; R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and z2 is independently an integer from 3-10.

In embodiments, n21 is 14, R²⁰¹ is dodecyl and z2 is 8.

In embodiments, the cationic amphipathic polymer has the formula:

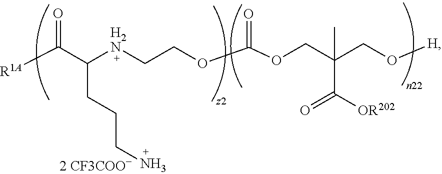

wherein n22 is an integer from 10 to 35; R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and z2 is independently an integer from 5-20.

In embodiments, n22 is 14, R²⁰² is dodecyl and z2 is 7.

In embodiments, the cell penetrating complex as described herein, further includes a second cationic amphipathic polymer, wherein the second cationic amphipathic polymer is different from the cationic amphipathic polymer.

In embodiments, the second cationic amphipathic polymer has the formula:

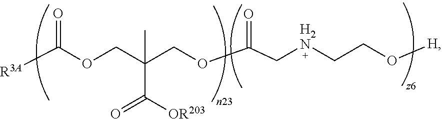

n23 is an integer from 1 to 100; z6 is an integer from 5-15; R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n23 is 13, z6 is 11 and R²⁰³ is dodecyl.

In embodiments, X₁ is CH₂.

In embodiments, L⁴ is independently substituted or unsubstituted C₂-C₈ alkylene. In embodiments, L⁴ is independently unsubstituted C₂-C₈ alkylene. In embodiments, L⁴ is independently unsubstituted C₂ alkylene, unsubstituted C₃ alkylene or unsubstituted C₄ alkylene. In embodiments, L⁴ is independently unsubstituted C₃ alkylene or unsubstituted C₄ alkylene. In embodiments, L⁴ is independently unsubstituted C₂ alkylene. In embodiments, L⁴ is independently unsubstituted C₃ alkylene. In embodiments, L⁴ is independently unsubstituted C₄ alkylene.

In embodiments, R⁴⁰, R⁴¹, and R⁴² are independently hydrogen or substituted heteroalkyl. In embodiments, R⁴⁰, R⁴¹, and R⁴² are independently hydrogen or —C(NH)NH₂.

In embodiments, at least two of R⁴⁰, R⁴¹ and R⁴² are hydrogen and one is —C(NH)NH₂.

In embodiments, Z is —N⁺(R¹³)(H)— and R¹³ is hydrogen.

In embodiments, R¹ and R² are independently hydrogen or substituted or unsubstituted alkyl.

In embodiments, n1 is 2.

In embodiments, X₂ is —O—.

In embodiments, z1 or z3 are independently integers from 10-40.

In embodiments, z2 is independently an integer from 3-20.

### Cell-Penetrating Complexes—Section 2

According to the embodiments of Formula (I-A), (II-A), (III-A), (IV), (IV-A), (V-A), (Va-A), (Ia-A), (VI-A), (Ib-A), (Ic-A), (Id-A), (VII-A), (VIII-A), (IX-A), (X-A), and (XI-A), all substituents are defined as listed below. Any of the cell-penetrating complexes described in this section may be used for the compositions and methods provided herein. For example, any of the nucleic acid, cationic amphipathic polymer and cationic amphipathic polymer described in this section may form part of a cell-penetrating complex including a ribonucleic acid including a sequence encoding a viral protein, a nucleic acid adjuvant and a cationic amphipathic polymer as provided herein including embodiments thereof.

The cell-penetrating complex provided herein may include a nucleic acid non-covalently bound to a cationic amphipathic polymer, the cationic amphipathic polymer including a pH-sensitive immolation domain. In embodiments, one or more counter ions (e.g., anions) may also be present as countercharges to the positive charges in the cationic amphipathic polymer. In embodiments, the nucleic acid is non-covalently bound to the cationic amphipathic polymer. In embodiments, the nucleic acid is ionically bound to the cationic amphipathic polymer. In embodiments, the cell penetrating complex includes a plurality of optionally different nucleic acids (e.g. 1 to 10 additional nucleic acids, 1 to 5 additional nucleic acids, 1 to 5 additional nucleic acids, 2 additional nucleic acids or 1 additional nucleic acid). In embodiments, the nucleic acid is RNA. In embodiments, the nucleic acid is mRNA.

In embodiments, a ratio between the number of cations in the cationic amphipathic polymer molecules and the number of anions on the nucleic acid molecules present in a cell-penetrating complex can be about 0.5:1, about 1:1, about 5:1, about 10:1, about 20:1, about 30:1, about 40:1, about 50:1, about 60:1, about 70:1, about 80:1, about 90:1, about 10²:1, about 10³:1, about 10⁴:1, about 10⁵:1, about 10⁶:1, about 10⁷:1, about 10⁸:1, about 10⁹:1 about 10¹⁰:1, or more or any intervening ranges of the foregoing. In other embodiments, a ratio between the number of anions on the nucleic acid molecules and the number of cations on the cationic amphipathic polymer molecules present in a cell-penetrating complex can be about 0.5:1, about 1:1, about 5:1, about 10:1, about 20:1, about 30:1, about 40:1, about 50:1, about 60:1, about 70:1, about 80:1, about 90:1, about 10:1, about 10²:1, about 10³:1, about 10⁴:1, about 10⁵:1, about 10⁶:1, about 10⁷:1, about 10⁸:1, about 10⁹:1 about 10¹⁰:1, or more or any intervening ranges of the foregoing. In some preferred embodiments, this ratio is approximately 10 cationic charges on the amphipathic polymer molecule to 1 negative charge on the nucleic acid. Other embodiments can have 5 cationic charges on the amphipathic polymer molecule to 1 negative charge on the nucleic acid or 20 cationic charges on the amphipathic polymer molecule to 1 negative charge on the nucleic acid.

In embodiments, a ratio between the number of nucleic acid molecules and the number of cationic amphipathic polymer molecules present in a cell-penetrating complex can be about 0.5:1, about 1:1, about 10:1, about 10²:1, about 10³:1, about 10⁴:1, about 10⁵:1, about 10⁶:1, about 10⁷:1, about 10⁸:1, about 10⁹:1 about 10¹⁰:1, or more or any intervening ranges of the foregoing. In other embodiments, a ratio between the number of cationic amphipathic polymer molecules and the number of nucleic acid molecules present in a cell-penetrating complex can be about 0.5:1, about 1:1, about 10:1, about 10²:1, about 10³:1, about 10⁴:1, about 10⁵:1, about 10⁶:1, about 10⁷:1, about 10⁸:1, about 10⁹:1 about 10¹⁰:1, or more or any intervening ranges of the foregoing.

In embodiments, the cationic amphipathic polymer may be a cationic charge altering releasable transporter (CART). In embodiments, the CART may include an oligomeric chain containing a series of cationic sequences that undergo a pH-sensitive change in charge from cationic to neutral or cationic to anionic.

In embodiments, the cationic amphipathic polymer has a pH-sensitive immolation domain and a lipophilic polymer domain. In embodiments, the lipophilic polymer domain may facilitate cell permeation, cell delivery and/or transport across cell membrane. In embodiments, the lipophilic polymer domain may be substantially insoluble in water (e.g., less than about 0.0005 mg/mL to about 10 mg/mL soluble in water). In embodiments, the lipophilic polymer domain may facilitate aggregation of the cationic amphipathic polymers into nanoparticles. In embodiments, such nanoparticles may have an average longest dimension of about 50 nm to about 500 nm. In embodiments, the lipophilic polymer domain may facilitate endosome fusion of the remnants of the cationic amphipathic polymer subsequent to entry and immolation within the endosome. In embodiments, the cell-penetrating complexes of the present disclosure protect the nucleic acid cargo from degradation. The term “nucleic acid cargo” or the like refers, in the usual and customary sense, to a species desired for transport into a cell by the cell-penetrating complex disclosed herein, and embodiments thereof.

In embodiments, the cationic amphipathic polymer has the formula (I-A): H-L¹-[(LP¹)_{z1}-(IM)_{z2}-(LP²)_{z3}]_{z4}-L²-H (I-A), wherein L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a bond or a lipophilic polymer domain, wherein at least one of LP¹ or LP² is a lipophilic polymer domain; IM is the pH-sensitive immolation domain; z1, z3 and z4 are independently integers from 0 to 100, wherein at least one of z1 or z3 is not 0; and z2 is an integer from 2 to 100.

In embodiments, L¹ is substituted or unsubstituted C₁-C₃ alkylene. In embodiments, L¹ is substituted or unsubstituted methylene. In embodiments, L¹ is substituted or unsubstituted C₁-C₆ alkylene, or substituted or unsubstituted 2 to 6 membered heteroalkylene. In embodiments, L¹ is substituted or unsubstituted C₁-C₃ alkylene, or substituted or unsubstituted 2 to 3 membered heteroalkylene.

In embodiments, L¹ is substituted or unsubstituted alkylene (e.g., C₁-C₃, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹ is unsubstituted alkylene, unsubstituted heteroalkylene, unsubstituted cycloalkylene, unsubstituted heterocycloalkylene, unsubstituted arylene, or unsubstituted heteroarylene. In embodiments, L¹ is unsubstituted alkylene (e.g., C₁-C₆ alkylene). In embodiments, L¹ is a bond.

In embodiments, L² is substituted or unsubstituted C₁-C₃ alkylene. In embodiments, L² is substituted or unsubstituted methylene. In embodiments, L² is substituted or unsubstituted C₁-C₆ alkylene, or substituted or unsubstituted 2 to 6 membered heteroalkylene. In embodiments, L² is substituted or unsubstituted C₁-C₃ alkylene, or substituted or unsubstituted 2 to 3 membered heteroalkylene.

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene. In embodiments, L² is unsubstituted alkylene (e.g., C₁-C₆ alkylene). In embodiments, L² is a bond.

In embodiments z2 is an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25. In embodiments, z1, z3 and z4 are independently integers from 0 to 90 (e.g. 5 to 90, 10 to 90 or 20 to 90), 0 to 80 (e.g. 5 to 80, 10 to 80 or 20 to 80), 0 to 70 (e.g. 5 to 70, 10 to 70 or 20 to 70), 0 to 50 (e.g. 5 to 50, 10 to 50 or 20 to 50) or 2 to 25. In embodiments, z1, z3 and z4 are independently integers from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25.

In embodiments of the cell-penetrating complex, the pH-sensitive immolation domain includes a first nucleophilic moiety and a first electrophilic moiety, wherein the first nucleophilic moiety is reactive with the first electrophilic moiety within a pH range and is not substantially reactive with the electrophilic moiety outside that pH range (e.g., pH about 1-5, pH about 5-7 or pH about 7-10). In embodiments, the pH range within which the first nucleophilic moiety is most reactive with the first electrophilic moiety is: pH 1-3, pH 2-4, pH 3-5, pH 4-6, pH 5-7, pH 6-8, pH 7-9, or pH 8-10. A nucleophilic moiety is used in accordance with its plain ordinary meaning in chemistry and refers to a moiety (e.g., functional group) capable of donating electrons.

In embodiments, the pH range within which the first nucleophilic moiety is most reactive with the first electrophilic moiety is pH 1-3. In embodiments, the pH range within which the first nucleophilic moiety is most reactive with the first electrophilic moiety is pH 2-4. In embodiments, the pH range within which the first nucleophilic moiety is most reactive with the first electrophilic moiety is pH 3-5. In embodiments, the pH range within which the first nucleophilic moiety is most reactive with the first electrophilic moiety is pH 4-6. In embodiments, the pH range within which the first nucleophilic moiety is most reactive with the first electrophilic moiety is pH 5-7. In embodiments, the pH range within which the first nucleophilic moiety is most reactive with the first electrophilic moiety is pH 6-8. In embodiments, the pH range within which the first nucleophilic moiety is most reactive with the first electrophilic moiety is pH 7-9. In embodiments, the pH range within which the first nucleophilic moiety is most reactive with the first electrophilic moiety is pH 8-10. In embodiments, the pH is 1. In embodiments, the pH is 2. In embodiments, the pH is 3. In embodiments, the pH is 4. In embodiments, the pH is 5. In embodiments, the pH is 6. In embodiments, the pH is 7. In embodiments, the pH is 8. In embodiments, the pH is 9. In embodiments, the pH is 10. In embodiments, the pH is about 1. In embodiments, the pH is about 2. In embodiments, the pH is about 3. In embodiments, the pH is about 4. In embodiments, the pH is about 5. In embodiments, the pH is about 6. In embodiments, the pH is about 7. In embodiments, the pH is about 8. In embodiments, the pH is about 9. In embodiments, the pH is about 10.

In embodiments, the first nucleophilic moiety is substantially protonated at low pH (e.g., pH about 1 to about 5). In embodiments, the first nucleophilic moiety is substantially protonated in the range pH 5-7. In embodiments, the first nucleophilic moiety is cationic. In embodiments, the first nucleophilic moiety includes a cationic nitrogen (e.g. a cationic amine).

In embodiments, the first nucleophilic moiety can be attached to a pH-labile protecting group. The term “pH-labile protecting group” or the like refers, in the usual and customary sense, to a chemical moiety capable of protecting another functionality to which it is attached, and which protecting group can be cleaved or otherwise inactivated as a protecting group under certain pH conditions (e.g., such as decreasing the pH). In one embodiment, the pH-labile protecting group is —CO₂-t-Bu, a group removed under acidic conditions (e.g., pH below 7). Additional nucleophile protecting groups could also include those that are cleaved by light, heat, nucleophile, and bases.

In embodiments of the cell-penetrating complex disclosed above and embodiments thereof, the pH-sensitive immolation domain has the structure of Formula (II-A) following:

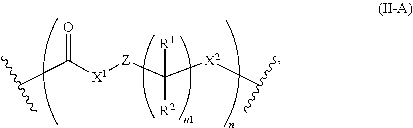

wherein n is an integer of 2 or more; n1 is an integer from 0 to 50; Z is a nucleophilic moiety; X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—; X² is —O— or —S—, and R¹, R², R³, R⁴, R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, n is an integer in the range 2-100, 2-90, 2-80, 2-70, 2-60, 2-50, 2-40, 2-30, 2-2, or 2-10. In embodiments, n1 is an integer in the range 0-25, 0-10, 0-5. In embodiments, n1 is 0, 1, 2, 3, 4 or 5. In embodiments, n1 is 1 or 2.

In embodiments, R¹, R², R³, R⁴, R⁵, R⁶, R⁷, and R⁸ are independently substituted or unsubstituted alkyl (e.g., C₁-C₃, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R³, R⁴, R⁵, R⁶, R⁷, and R⁸ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R³, R⁴, R⁵, R⁶, R⁷, and R⁸ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R³, R⁴, R⁵, R⁶, R⁷, and R⁸ are independently hydrogen or unsubstituted alkyl (e.g., C₁-C₆ alkyl). In embodiments, R¹, R², R³, R⁴, R⁵, R⁶, R⁷, and R⁸ are hydrogen.

In embodiments of the cell-penetrating complex disclosed above and embodiments thereof, the pH-sensitive immolation domain may be have the structure following

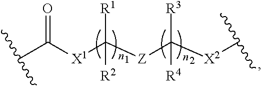

wherein R¹, R², R³, R⁴, X¹, Z and X² are as defined herein, and n1 and n2 are integers greater than 1.

In embodiments of the cell-penetrating complex disclosed herein and embodiments thereof, the pH-sensitive immolation domain has the structure of Formula (III-A) following:

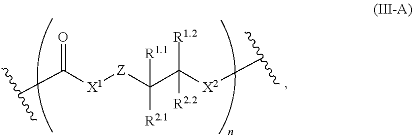

wherein n is an integer of 2 or more; Z is a nucleophilic moiety; X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—; X² is —O— or —S—, and R^{1.1}, R^{1.2}, R^{2.1}, R^{2.2}, R³, R⁴, R⁵, R⁶, R⁷ and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In embodiments, R^{1.1}, R^{1.2}, R^{2.1}, R^{2.2}, R³, R⁴, R⁵, R⁶, R⁷, and R⁸ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{1.1}, R^{1.2}, R^{2.1}, R^{2.2}, R³, R⁴, R⁵, R⁶, R⁷ and R⁸ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1.1}, R^{1.2}, R^{2.1}, R^{2.2}, R³, R⁴, R⁵, R⁶, R⁷ and R⁸ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R^{1.1}, R^{1.2}, R^{2.1}, R^{2.2}, R³, R⁴, R⁵, R⁶, R⁷ and R⁸ are independently hydrogen or unsubstituted alkyl (e.g., C₁-C₆ alkyl). In embodiments, R^{1.1}, R^{1.2}, R^{2.1}, R^{2.2}, R³, R⁴, R⁵, R⁶, R⁷, and R⁸ are hydrogen.

In embodiments of the cell-penetrating complex disclosed herein and embodiments thereof, the pH-sensitive immolation domain has the structure of Formula (IV) following:

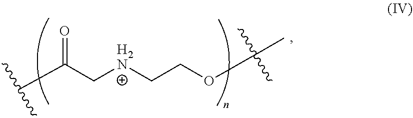

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50. In embodiments, n is 2 to 15.

In embodiments of the cell-penetrating complex disclosed herein and embodiments thereof, the pH-sensitive immolation domain has the structure of Formula (IV-A) following:

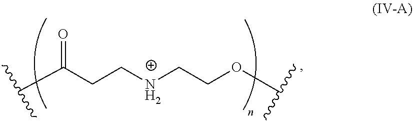

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In embodiments of the cell-penetrating complex disclosed herein and embodiments thereof, the pH-sensitive immolation domain has the structure of Formula (V-A) following:

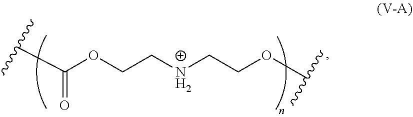

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In embodiments of the cell-penetrating complex disclosed herein and embodiments thereof, the pH-sensitive immolation domain has the structure of Formula (Va-A) following:

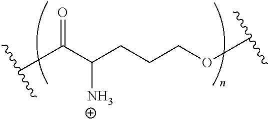

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In embodiments, the pH-sensitive immolation domain has the structure following:

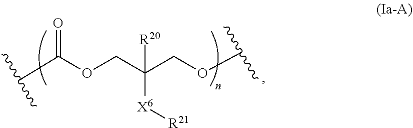

wherein X⁶ is —O—, —NH—, —CONH—, —COO—, —OCO—, —NHCO—, R²⁰ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl, and R²¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, R²⁰ is an oligoglycol moiety.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl). In embodiments, R²⁰ is hydrogen.

In embodiments, R²¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²¹ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²¹ is unsubstituted alkyl, unsubstituted heteroalkyl, unsubstituted cycloalkyl, unsubstituted heterocycloalkyl, unsubstituted aryl, or unsubstituted heteroaryl. In embodiments, R²¹ is hydrogen or unsubstituted alkyl (e.g., C₁-C₆ alkyl). In embodiments, R²¹ is hydrogen.

In embodiments of the cell-penetrating complex disclosed herein and embodiments thereof, the pH-sensitive immolation domain has the structure following:

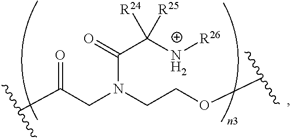

wherein R²⁴, R²⁵ and R²⁶ are hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and n3 is an integer from 0 to 50.

In embodiments, R²⁴, R²⁵ and R²⁶ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁴, R²⁵ and R²⁶ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1.1}, R²⁴, R²⁵ and R²⁶ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R²⁴, R²⁵ and R²⁶ are independently hydrogen or unsubstituted alkyl (e.g., C₁-C₆ alkyl). In embodiments, R²⁴, R²⁵ and R²⁶ are independently hydrogen.

In embodiments of the cell-penetrating complex disclosed herein and embodiments thereof, the pH-sensitive immolation domain has the structure of Formula (VI-A) following:

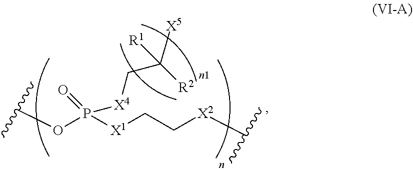

wherein n is an integer of 2 or more; n1 is an integer from 0 to 50; X¹ is a bond, —O—, —NR⁵—, —C(R⁵)(R⁶)— or —C(R⁵)(R⁶)—C(R⁷)(R⁸)—; X² is a bond, —O—, —C(R⁹)(R¹⁰)— or —C(R⁹)(R¹⁰)—C(R¹¹)(R¹²)—; X⁴ is a bond, —NR¹⁶—, —O—, —C(R¹⁶)(R¹⁷)— or —C(R¹⁶)(R¹⁷)—C(R¹⁸)(R¹⁹)—; X⁵ is a nucleophilic moiety; and R¹, R², R⁵, R⁶, R⁷, R⁸, R⁹, R¹⁰, R¹¹, R¹², R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R¹, R², R⁵, R⁶, R⁷, R⁸, R⁹, R¹⁰, R¹¹, R¹², R¹⁵, R¹⁶, R¹⁷, R¹⁸ and R¹⁹ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R⁵, R⁶, R⁷, R⁸, R⁹, R¹⁰, R¹¹, R¹², R¹⁵, R¹⁶, R¹⁷, R¹⁸ and R¹⁹ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R⁵, R⁶, R⁷, R⁸, R⁹, R¹⁰, R¹¹, R¹², R¹⁵, R¹⁶, R¹⁷, R¹⁸ and R¹⁹ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R⁵, R⁶, R⁷, R⁸, R⁹, R¹⁰, R¹¹, R¹², R¹⁵, R¹⁶, R¹⁷, R¹⁸ and R¹⁹ are independently hydrogen or unsubstituted alkyl (e.g., C₁-C₆ alkyl). In embodiments, R¹, R², R⁵, R⁶, R⁷, R⁸, R⁹, R¹⁰, R¹¹, R¹², R¹⁵, R¹⁶, R¹⁷, R¹⁸, and R¹⁹ are hydrogen.

In embodiments, Z is a nucleophilic moiety. In embodiments, Z is —S—, —OR¹³—, —S⁺R¹³—, —NR¹³ —, or —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments Z is —S—. In embodiments, Z is —S⁺R¹³—. In embodiments, Z is —NR¹³—. In embodiments, Z is —N⁺(R¹³)(H)—. In embodiments, Z is —S⁺H—. In embodiments, Z is —NH—. In embodiments, Z is —N⁺H₂—. In embodiments, Z is —OH—.

In embodiments, R¹³ is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³ is independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹³ is independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³ is independently hydrogen or unsubstituted alkyl (e.g., C₁-C₆ alkyl). In embodiments, R¹³ is hydrogen.

In embodiments, Z is

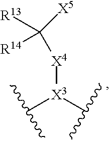

wherein X³ is —C(R¹⁵)— or —N—; X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—; X⁵ is a nucleophilic moiety; and R¹³, R¹⁴, R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, X³ is —CH.

In embodiments, R¹³, R¹⁴, R¹⁵, R¹⁶, R¹⁷, R¹⁸ and R¹⁹ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³, R¹⁴, R¹⁵, R¹⁶, R¹⁷, R¹⁸ and R¹⁹ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments R¹³, R¹⁴, R¹⁵, R¹⁶, R¹⁷, R¹⁸ and R¹⁹ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³, R¹⁴, R¹⁵, R¹⁶, R¹⁷, R¹⁸ and R¹⁹ are independently hydrogen or unsubstituted alkyl (e.g., C₁-C₆ alkyl).

In embodiments, X⁵ is —N⁺(R¹³)(H), wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

Further to the cell-penetrating complex disclosed herein and embodiments thereof, in embodiments the lipophilic polymer domain has the formula:

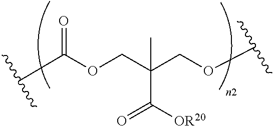

wherein, n2 is an integer from 1 to 100; R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl).

In embodiments, R²⁰ is an unsubstituted C₁-C₃₀ alkyl. In embodiments, R²⁰ is an unsubstituted C₁-C₂₀ alkyl. In embodiments, R²⁰ is an unsubstituted C₈-C₃₀ alkyl. In embodiments, R²⁰ is an unsubstituted C₈-C₂₀ alkyl. In embodiments, R²⁰ is an unsubstituted C₉-C₂₀ alkyl. In embodiments, R²⁰ is an unsubstituted C₉-C₁₈ alkyl. In embodiments, R²⁰ is an unsubstituted C₁₈ alkyl. In embodiments, R²⁰ is an unsubstituted C₁₇ alkyl. In embodiments, R²⁰ is an unsubstituted C₁₆ alkyl. In embodiments, R²⁰ is an unsubstituted C₁₅ alkyl. In embodiments, R²⁰ is an unsubstituted C₁₄ alkyl. In embodiments, R²⁰ is an unsubstituted C₁₃ alkyl. In embodiments, R²⁰ is an unsubstituted C₁₂ alkyl. In embodiments, R²⁰ is an unsubstituted C₁₁ alkyl. In embodiments, R²⁰ is an unsubstituted C₁₀ alkyl. In embodiments, R²⁰ is an unsubstituted C₉ alkyl. In embodiments, R²⁰ is an unsubstituted C₈ alkyl. In embodiments, R²⁰ is an unsubstituted C₇ alkyl. In embodiments, R²⁰ is an unsubstituted C₆ alkyl. In embodiments, R²⁰ is an unsubstituted C₅ alkyl. In embodiments, R²⁰ is an unsubstituted C₄ alkyl. In embodiments, R²⁰ is an unsubstituted C₃ alkyl. In embodiments, R²⁰ is an unsubstituted C₂ alkyl.

In embodiments, R²⁰ is an unsubstituted C₁-C₃₀ alkenyl. In embodiments, R²⁰ is an unsubstituted C₁-C₂₀ alkenyl. In embodiments, R²⁰ is an unsubstituted C₈-C₃₀ alkenyl. In embodiments, R²⁰ is an unsubstituted C₈-C₂₀ alkenyl. In embodiments, R²⁰ is an unsubstituted C₉-C₂₀ alkenyl. In embodiments, R²⁰ is an unsubstituted C₉-C₁₈ alkenyl. In embodiments, R²⁰ is an unsubstituted C₁₈ alkenyl. In embodiments, R²⁰ is an unsubstituted C₁₇ alkenyl. In embodiments, R²⁰ is an unsubstituted C₁₆ alkenyl. In embodiments, R²⁰ is an unsubstituted C₁₅ alkenyl. In embodiments, R²⁰ is an unsubstituted C₁₄ alkenyl. In embodiments, R²⁰ is an unsubstituted C₁₃ alkenyl. In embodiments, R²⁰ is an unsubstituted C₁₂ alkenyl. In embodiments, R²⁰ is an unsubstituted C₁₁ alkenyl. In embodiments, R²⁰ is an unsubstituted C₁₀ alkenyl. In embodiments, R²⁰ is an unsubstituted C₉ alkenyl. In embodiments, R²⁰ is an unsubstituted C₈ alkenyl. In embodiments, R²⁰ is an unsubstituted C₇ alkenyl. In embodiments, R²⁰ is an unsubstituted C₆ alkenyl. In embodiments, R²⁰ is an unsubstituted C₅ alkenyl. In embodiments, R²⁰ is an unsubstituted C₄ alkenyl. In embodiments, R²⁰ is an unsubstituted C₃ alkenyl. In embodiments, R²⁰ is an unsubstituted C₂ alkenyl.

In embodiments, R²⁰ is a stearyl moiety (e.g., an unsubstituted C₁₈ alkyl). In embodiments, R²⁰ is an oleyl moiety (e.g., an unsubstituted C₁₀ alkenyl). In embodiments, R²⁰ is an linoleyl moiety (e.g., an unsubstituted C₁₀ alkenyl). In embodiments, R²⁰ is an dodecyl moiety (e.g., an unsubstituted C₁₂ alkyl). In embodiments, R²⁰ is an nonenyl moiety (e.g., an unsubstituted C₉ alkenyl). In embodiments, R²⁰ is

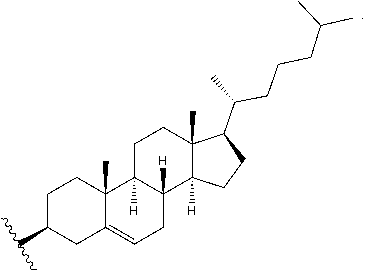

In embodiments, the lipophilic polymer domain is a compound of Formula (Ia-A) following:

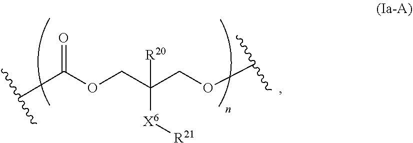

wherein X⁶ may be —O—, —NH—, —CO₂—, —CONH—, —O₂C—, or —NHCO—, R²⁰ is hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, R²¹ is hydrogen, substituted or unsubstituted alkyl, and n is as defined herein. In embodiments, R²⁰ is an oligoglycol moiety.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl).

In embodiments, R²¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²¹ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²¹ is unsubstituted alkyl, unsubstituted heteroalkyl, unsubstituted cycloalkyl, unsubstituted heterocycloalkyl, unsubstituted aryl, or unsubstituted heteroaryl. In embodiments, R²¹ is hydrogen or unsubstituted alkyl (e.g., C₁-C₆ alkyl).

In embodiments of the cell-penetrating complex disclosed herein and embodiments thereof, the pH-sensitive immolation domain has the structure:

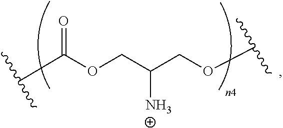

wherein n4 is an integer from 0 to 50. In embodiments, n4 is 0 to 10. In embodiments, n4 is an integer from 1 to 15.

In embodiments of the cell-penetrating complex disclosed herein and embodiments thereof, the lipophilic polymer has the structure:

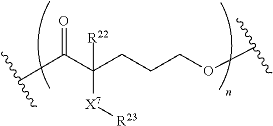

wherein X⁷ is —O—, —NH—, —CO₂—, —CONH—, —O₂C—, or —NHCO—; R²² is hydrogen, or substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl, and R²³ is substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, R²² is an oligoglycol moiety.

In embodiments, R²² is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²² is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²² is unsubstituted alkyl, unsubstituted heteroalkyl, unsubstituted cycloalkyl, unsubstituted heterocycloalkyl, unsubstituted aryl, or unsubstituted heteroaryl. In embodiments, R²² is hydrogen or unsubstituted alkyl (e.g., C₁-C₆ alkyl).

In embodiments, the lipophilic polymer domain may be a compound of Formula (Ib-A) following:

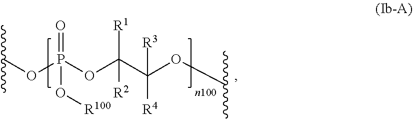

wherein R¹⁰⁰ is hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl. R¹, R², R³, R⁴ are hydrogen, substituted or unsubstituted heteroalkyl, substituted or unsubstituted heterocycloalkyl, substituted or unsubstituted aryl, or substituted or unsubstituted heteroaryl, and n100 is an integer of 2 or more is as defined herein.

In embodiments, R¹, R², R³, R⁴ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R³, R⁴ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R³, R⁴ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R³, R⁴ are independently hydrogen or unsubstituted alkyl (e.g., C₁-C₆ alkyl). In embodiments, R¹, R², R³, R⁴ are hydrogen.

In embodiments, the lipophilic polymer domain may be a compound of Formula (Ic-A) following:

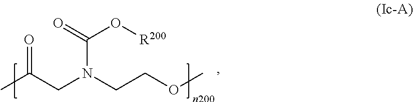

wherein R²⁰⁰ is substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and n200 is an integer of 2 or more. In embodiments R²⁰⁰ is an oligoglycol moiety. In embodiments, R²⁰⁰ is an amine-terminated oligoglycol moiety. The term “oligoglycol moiety” refers to

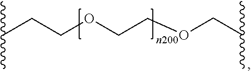

and “amine-terminated oligoglycol moiety” refers to

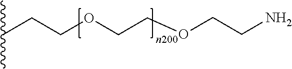

wherein n200 is an integer of 2 or more.

In embodiments, R²⁰⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰⁰ is hydrogen or unsubstituted alkyl (e.g., C₁-C₆ alkyl). In embodiments, R²⁰⁰ is hydrogen.

In embodiments, the lipophilic polymer domain may be a compound of Formula (Id-A) following:

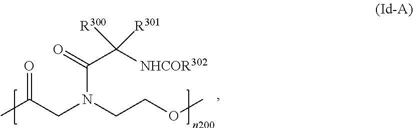

wherein R is substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, R³⁰⁰ and R³⁰¹ are independently hydrogen or substituted or unsubstituted alkyl, and n300 is as defined herein. In embodiments R³⁰² is an oligoglycol moiety. In embodiments, R is an amine-terminated an oligoglycol moiety. In embodiments R³⁰⁰, R³⁰¹, and R³⁰² are hydrogen.

In embodiments, R³⁰⁰, R³⁰¹, and R³⁰² are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R³⁰⁰, R³⁰¹, and R³⁰² are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R³⁰⁰, R³⁰¹, and R³⁰² are independently unsubstituted alkyl, unsubstituted heteroalkyl, unsubstituted cycloalkyl, unsubstituted heterocycloalkyl, unsubstituted aryl, or unsubstituted heteroaryl. In embodiments, R³⁰⁰, R³⁰¹, and R³⁰² are independently hydrogen or unsubstituted alkyl (e.g., C₁-C₆ alkyl).

The cationic amphipathic polymer can have a pH-sensitive immolation domain. In some embodiments, the cationic amphipathic polymer has a pH-sensitive immolation domain and a lipophilic polymer domain. In some embodiments, the cell-penetrating complex has a cationic amphipathic polymer of the following formula (VII-A):

R^{1A}-[L¹-[(LP¹)_{z1}-(IM)_{z2}-(LP²)_{z3}]_{z4}-L²-R^{2A}]_{z5}

wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —O CHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a bond or a lipophilic polymer domain, wherein at least one of LP¹ or LP² is a lipophilic polymer domain;
IM is a pH-sensitive immolation domain;
z5 is an integer from 1 to 10;
z1, z3 and z4 are independently integers from 0 to 100, wherein at least one of z1 or z3 is not 0; and z2 is an integer from 2 to 100.

In embodiments, R^{1A} is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{1A} is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1A} is unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1A} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{1A} is substituted or unsubstituted alkyl (e.g., C₁-C₈ alkyl, C₁-C₆ alkyl, or C₁-C₄ alkyl). In embodiments, R^{1A} is substituted alkyl (e.g., C₁-C₈ alkyl, C₁-C₆ alkyl, or C₁-C₄ alkyl). In embodiments, R^{1A} is an unsubstituted alkyl (e.g., C₁-C₈ alkyl, C₁-C₆ alkyl, or C₁-C₄ alkyl). In embodiments, R^{1A} is substituted or unsubstituted heteroalkyl (e.g., 2 to 8 membered heteroalkyl, 2 to 6 membered heteroalkyl, or 2 to 4 membered heteroalkyl). In embodiments, R^{1A} is substituted heteroalkyl (e.g., 2 to 8 membered heteroalkyl, 2 to 6 membered heteroalkyl, or 2 to 4 membered heteroalkyl). In embodiments, R^{1A} is an unsubstituted heteroalkyl (e.g., 2 to 8 membered heteroalkyl, 2 to 6 membered heteroalkyl, or 2 to 4 membered heteroalkyl). In embodiments, R^{1A} is substituted or unsubstituted cycloalkyl (e.g., C₃-C₈ cycloalkyl, C₃-C₆ cycloalkyl, or C₅-C₆ cycloalkyl). In embodiments, R^{1A} is substituted cycloalkyl (e.g., C₃-C₈ cycloalkyl, C₃-C₆ cycloalkyl, or C₅-C₆ cycloalkyl). In embodiments, R^{1A} is an unsubstituted cycloalkyl (e.g., C₃-C₈ cycloalkyl, C₃-C₆ cycloalkyl, or C₅-C₆ cycloalkyl). In embodiments, R^{1A} is substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is substituted heterocycloalkyl (e.g., 3 to 8 membered heterocycloalkyl, 3 to 6 membered heterocycloalkyl, or 5 to 6 membered heterocycloalkyl). In embodiments, R^{1A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is substituted or unsubstituted aryl (e.g., C₆-C₁₀ aryl, C₁₀ aryl, or phenyl). In embodiments, R^{1A} is substituted aryl (e.g., C₆-C₁₀ aryl, C₁₀ aryl, or phenyl). In embodiments, R^{1A} is an unsubstituted aryl (e.g., C₆-C₁₀ aryl, C₁₀ aryl, or phenyl). In embodiments, R^{1A} is substituted or unsubstituted heteroaryl (e.g., 5 to 10 membered heteroaryl, 5 to 9 membered heteroaryl, or 5 to 6 membered heteroaryl). In embodiments, R^{1A} is substituted heteroaryl (e.g., 5 to 10 membered heteroaryl, 5 to 9 membered heteroaryl, or 5 to 6 membered heteroaryl). In embodiments, R^{1A} is an unsubstituted heteroaryl (e.g., 5 to 10 membered heteroaryl, 5 to 9 membered heteroaryl, or 5 to 6 membered heteroaryl).

In embodiments, R^{1A} is a substituted or unsubstituted aryl. In some other embodiments, R^{1A} is a substituted or unsubstituted phenyl. In still some other embodiments, R^{1A} is a substituted or unsubstituted aryl. In still some other embodiments, R^{1A} is a substituted or unsubstituted phenyl or naphthalenyl.

In some embodiments, the cell-penetrating complex can have a cationic amphipathic polymer having the following formula (VIII-A):

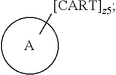

wherein
Ring A is a substituted or unsubstituted cycloalkyl, substituted or unsubstituted heterocycloalkyl, substituted or unsubstituted aryl, or substituted or unsubstituted heteroaryl;
CART can have the formula: -L¹-[(LP¹)_{z1}-(IM)_{z2}-(LP²)_{z3}]_{z4}-L²-R^{2A}
   wherein,
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a bond or a lipophilic polymer domain, wherein at least one of LP¹ or LP² is a lipophilic polymer domain; IM is a pH-sensitive immolation domain; z5 are an integer from 1 to 10; z1, z3 and z4 are independently integers from 0 to 100, wherein at least one of z1 or z3 is not 0; and z2 is an integer from 2 to 100.

In some embodiments, in the above-formula (VIII-A), Ring A is a substituted or unsubstituted aryl. In some other embodiments, Ring A is a substituted or unsubstituted phenyl. In still some other embodiments, Ring A is a substituted or unsubstituted aryl. In still some other embodiments, Ring A is a substituted or unsubstituted phenyl or naphthalenyl.

In embodiments, Ring A is an unsubstituted aryl (i.e. unsubstituted beyond the CART moiety). In embodiments, Ring A is an unsubstituted phenyl (i.e. unsubstituted beyond the CART moiety). In embodiments, Ring A is an unsubstituted phenyl or naphthalenyl (i.e. unsubstituted beyond the CART moiety). In embodiments, Ring A is a substituted aryl (i.e. substituted in addition to the CART moiety). In embodiments, Ring A is a substituted phenyl (i.e. substituted in addition to the CART moiety). In embodiments, Ring A is a substituted phenyl or naphthalenyl (i.e. substituted in addition to the CART moiety).

In embodiments, the cell-penetrating complex has a detectable agent (e.g., fluorophore).

In embodiments, R^{1A} is an aryl substituted with a methoxy linker. In embodiments, R^{1A} is an aryl substituted with a linker (e.g., —CH₂—O—). A non-limiting example wherein R^{1A} is an aryl substituted with a methoxy linker has the formula:

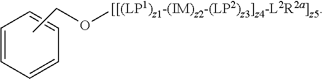

In some embodiments, a cationic amphipathic polymer has the formula (IX-A):

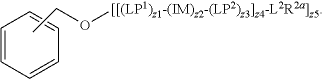

In some embodiments, a cationic amphipathic polymer has the formula (X-A):

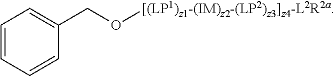

In some embodiments, a cationic amphipathic polymer can have the formula (XI-A):

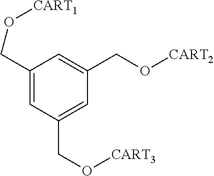

wherein CART₁, CART₂ and CART₃ are independently a CART moiety as defined in formula (VIII-A) (e.g., -L¹-[(LP¹)_{z1}-(IM)_{z2}-(LP²)_{z3}]_{z4}-L²-R^{2A}). In embodiments each CART moiety is optionally different.

In embodiments, the cationic amphipathic polymer has the formula:

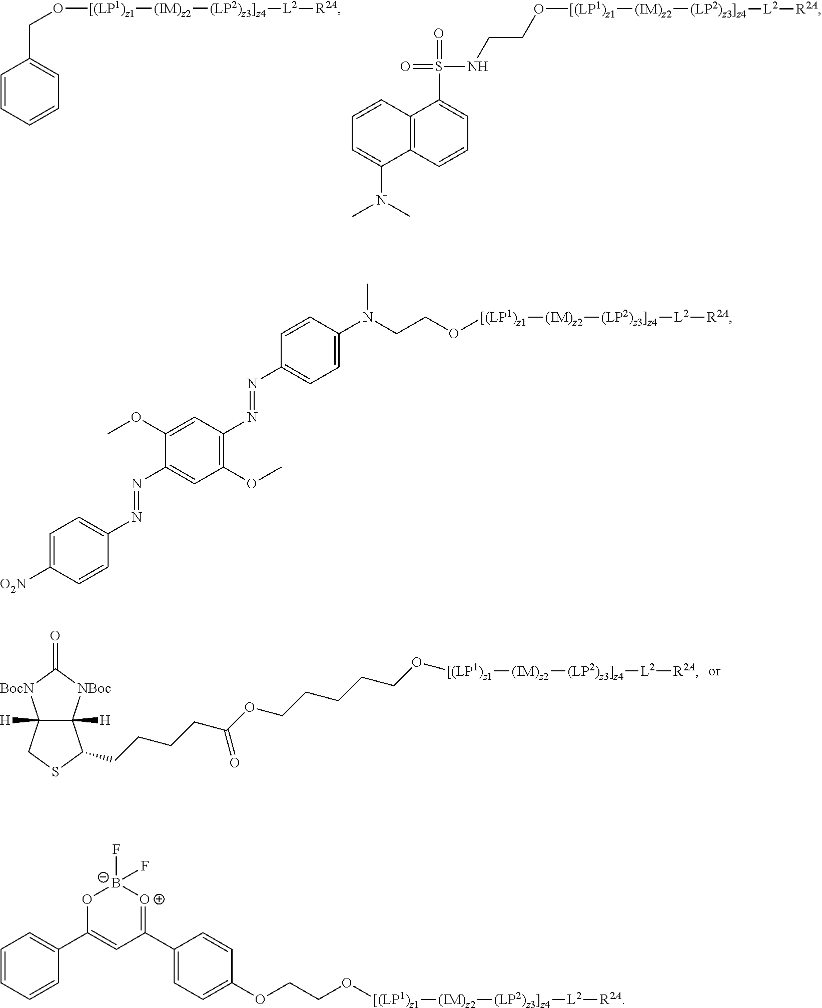

In embodiments, the cationic amphipathic polymer has the formula:

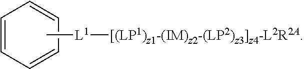

In embodiments, the cationic amphipathic polymer has the formula:

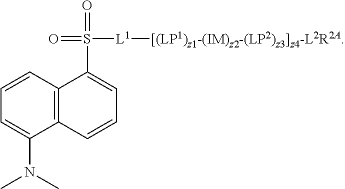

In embodiments, the cationic amphipathic polymer has the formula:

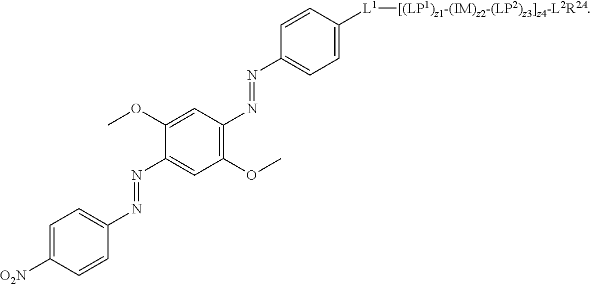

In embodiments, the cationic amphipathic polymer has the formula:

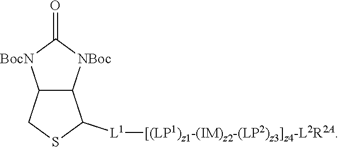

In embodiments, the cationic amphipathic polymer has the formula:

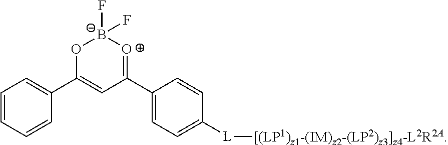

In embodiments, Ring A is substituted with a detectable agent through a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene).

In some embodiments, the cell-penetrating complex has a cationic amphipathic polymer having any of the foregoing formula in which L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is —CH₂—O—.

In some embodiments, the cationic amphipathic polymer can have any of the foregoing formula in which L¹ is —CH₂—O—,

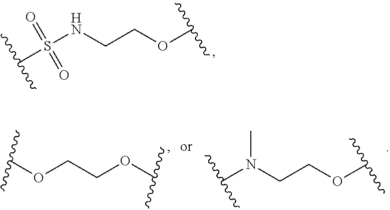

In embodiments, L¹ is —CH₂—O—. In embodiments, L¹ is

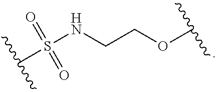 [SMILES: *OCCNS(*)(=O)=O]

In embodiments, L¹ is

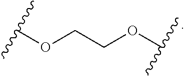 [SMILES: *OCCO*]

In embodiments, L¹ is

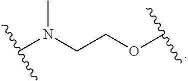 [SMILES: *OCCN(*)C]

In some embodiments, a cationic amphipathic polymer can have any of the foregoing formula in which z1, z3 and z4 can be independently integers from 0 to 100, wherein at least one of z1 or z3 is not 0. In some embodiments, z1, z3 and z4 can be independently integers in the range 2-100, 2-90, 2-80, 2-70, 2-60, 2-50, 2-40, 2-30, 2-2, or 2-10, wherein at least one of z1 or z3 is not 0. In embodiments, z1, z3 and z4 can be independently integers in the range 2-100 or 2-50, wherein at least one of z1 or z3 is not 0.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7. In embodiments, z1 is 8. In embodiments, z1 is 9. In embodiments, z1 is 10. In embodiments, z1 is 11. In embodiments, z1 is 12. In embodiments, z1 is 13. In embodiments, z1 is 14. In embodiments, z1 is 15. In embodiments, z1 is 16. In embodiments, z1 is 17. In embodiments, z1 is 18. In embodiments, z1 is 19. In embodiments, z1 is 20. In embodiments, z1 is 21. In embodiments, z1 is 22. In embodiments, z1 is 23. In embodiments, z1 is 24. In embodiments, z1 is 25. In embodiments, z1 is 26. In embodiments, z1 is 27. In embodiments, z1 is 28. In embodiments, z1 is 29. In embodiments, z1 is 30. In embodiments, z1 is 31. In embodiments, z1 is 32. In embodiments, z1 is 33. In embodiments, z1 is 34. In embodiments, z1 is 35. In embodiments, z1 is 36. In embodiments, z1 is 37. In embodiments, z1 is 38. In embodiments, z1 is 39. In embodiments, z1 is 40. In embodiments, z1 is 41. In embodiments, z1 is 42. In embodiments, z1 is 43. In embodiments, z1 is 44. In embodiments, z1 is 45. In embodiments, z1 is 46. In embodiments, z1 is 47. In embodiments, z1 is 48. In embodiments, z1 is 49. In embodiments, z1 is 50. In embodiments, z1 is 51. In embodiments, z1 is 52. In embodiments, z1 is 53. In embodiments, z1 is 54. In embodiments, z1 is 55. In embodiments, z1 is 56. In embodiments, z1 is 57. In embodiments, z1 is 58. In embodiments, z1 is 59. In embodiments, z1 is 60. In embodiments, z1 is 61. In embodiments, z1 is 62. In embodiments, z1 is 63. In embodiments, z1 is 64. In embodiments, z1 is 65. In embodiments, z1 is 66. In embodiments, z1 is 67. In embodiments, z1 is 68. In embodiments, z1 is 69. In embodiments, z1 is 70. In embodiments, z1 is 71. In embodiments, z1 is 72. In embodiments, z1 is 73. In embodiments, z1 is 74. In embodiments, z1 is 75. In embodiments, z1 is 76. In embodiments, z1 is 77. In embodiments, z1 is 78. In embodiments, z1 is 79. In embodiments, z1 is 80. In embodiments, z1 is 81. In embodiments, z1 is 82. In embodiments, z1 is 83. In embodiments, z1 is 84. In embodiments, z1 is 85. In embodiments, z1 is 86. In embodiments, z1 is 87. In embodiments, z1 is 88. In embodiments, z1 is 89. In embodiments, z1 is 90. In embodiments, z1 is 91. In embodiments, z1 is 92. In embodiments, z1 is 93. In embodiments, z1 is 94. In embodiments, z1 is 95. In embodiments, z1 is 96. In embodiments, z1 is 97. In embodiments, z1 is 98. In embodiments, z1 is 99. In embodiments, z1 is 100.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11. In embodiments, z3 is 12. In embodiments, z3 is 13. In embodiments, z3 is 14. In embodiments, z3 is 15. In embodiments, z3 is 16. In embodiments, z3 is 17. In embodiments, z3 is 18. In embodiments, z3 is 19. In embodiments, z3 is 20. In embodiments, z3 is 21. In embodiments, z3 is 22. In embodiments, z3 is 23. In embodiments, z3 is 24. In embodiments, z3 is 25. In embodiments, z3 is 26. In embodiments, z3 is 27. In embodiments, z3 is 28. In embodiments, z3 is 29. In embodiments, z3 is 30. In embodiments, z3 is 31. In embodiments, z3 is 32. In embodiments, z3 is 33. In embodiments, z3 is 34. In embodiments, z3 is 35. In embodiments, z3 is 36. In embodiments, z3 is 37. In embodiments, z3 is 38. In embodiments, z3 is 39. In embodiments, z3 is 40. In embodiments, z3 is 41. In embodiments, z3 is 42. In embodiments, z3 is 43. In embodiments, z3 is 44. In embodiments, z3 is 45. In embodiments, z3 is 46. In embodiments, z3 is 47. In embodiments, z3 is 48. In embodiments, z3 is 49. In embodiments, z3 is 50. In embodiments, z3 is 51. In embodiments, z3 is 52. In embodiments, z3 is 53. In embodiments, z3 is 54. In embodiments, z3 is 55. In embodiments, z3 is 56. In embodiments, z3 is 57. In embodiments, z3 is 58. In embodiments, z3 is 59. In embodiments, z3 is 60. In embodiments, z3 is 61. In embodiments, z3 is 62. In embodiments, z3 is 63. In embodiments, z3 is 64. In embodiments, z3 is 65. In embodiments, z3 is 66. In embodiments, z3 is 67. In embodiments, z3 is 68. In embodiments, z3 is 69. In embodiments, z3 is 70. In embodiments, z3 is 71. In embodiments, z3 is 72. In embodiments, z3 is 73. In embodiments, z3 is 74. In embodiments, z3 is 75. In embodiments, z3 is 76. In embodiments, z3 is 77. In embodiments, z3 is 78. In embodiments, z3 is 79. In embodiments, z3 is 80. In embodiments, z3 is 81. In embodiments, z3 is 82. In embodiments, z3 is 83. In embodiments, z3 is 84. In embodiments, z3 is 85. In embodiments, z3 is 86. In embodiments, z3 is 87. In embodiments, z3 is 88. In embodiments, z3 is 89. In embodiments, z3 is 90. In embodiments, z3 is 91. In embodiments, z3 is 92. In embodiments, z3 is 93. In embodiments, z3 is 94. In embodiments, z3 is 95. In embodiments, z3 is 96. In embodiments, z3 is 97. In embodiments, z3 is 98. In embodiments, z3 is 99. In embodiments, z3 is 100.

In embodiments, z4 is 0. In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11. In embodiments, z4 is 12. In embodiments, z4 is 13. In embodiments, z4 is 14. In embodiments, z4 is 15. In embodiments, z4 is 16. In embodiments, z4 is 17. In embodiments, z4 is 18. In embodiments, z4 is 19. In embodiments, z4 is 20. In embodiments, z4 is 21. In embodiments, z4 is 22. In embodiments, z4 is 23. In embodiments, z4 is 24. In embodiments, z4 is 25. In embodiments, z4 is 26. In embodiments, z4 is 27. In embodiments, z4 is 28. In embodiments, z4 is 29. In embodiments, z4 is 30. In embodiments, z4 is 31. In embodiments, z4 is 32. In embodiments, z4 is 33. In embodiments, z4 is 34. In embodiments, z4 is 35. In embodiments, z4 is 36. In embodiments, z4 is 37. In embodiments, z4 is 38. In embodiments, z4 is 39. In embodiments, z4 is 40. In embodiments, z4 is 41. In embodiments, z4 is 42. In embodiments, z4 is 43. In embodiments, z4 is 44. In embodiments, z4 is 45. In embodiments, z4 is 46. In embodiments, z4 is 47. In embodiments, z4 is 48. In embodiments, z4 is 49. In embodiments, z4 is 50. In embodiments, z4 is 51. In embodiments, z4 is 52. In embodiments, z4 is 53. In embodiments, z4 is 54. In embodiments, z4 is 55. In embodiments, z4 is 56. In embodiments, z4 is 57. In embodiments, z4 is 58. In embodiments, z4 is 59. In embodiments, z4 is 60. In embodiments, z4 is 61. In embodiments, z4 is 62. In embodiments, z4 is 63. In embodiments, z4 is 64. In embodiments, z4 is 65. In embodiments, z4 is 66. In embodiments, z4 is 67. In embodiments, z4 is 68. In embodiments, z4 is 69. In embodiments, z4 is 70. In embodiments, z4 is 71. In embodiments, z4 is 72. In embodiments, z4 is 73. In embodiments, z4 is 74. In embodiments, z4 is 75. In embodiments, z4 is 76. In embodiments, z4 is 77. In embodiments, z4 is 78. In embodiments, z4 is 79. In embodiments, z4 is 80. In embodiments, z4 is 81. In embodiments, z4 is 82. In embodiments, z4 is 83. In embodiments, z4 is 84. In embodiments, z4 is 85. In embodiments, z4 is 86. In embodiments, z4 is 87. In embodiments, z4 is 88. In embodiments, z4 is 89. In embodiments, z4 is 90. In embodiments, z4 is 91. In embodiments, z4 is 92. In embodiments, z4 is 93. In embodiments, z4 is 94. In embodiments, z4 is 95. In embodiments, z4 is 96. In embodiments, z4 is 97. In embodiments, z4 is 98. In embodiments, z4 is 99. In embodiments, z4 is 100.

In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24. In embodiments, n is 25. In embodiments, n is 26. In embodiments, n is 27. In embodiments, n is 28. In embodiments, n is 29. In embodiments, n is 30. In embodiments, n is 31. In embodiments, n is 32. In embodiments, n is 33. In embodiments, n is 34. In embodiments, n is 35. In embodiments, n is 36. In embodiments, n is 37. In embodiments, n is 38. In embodiments, n is 39. In embodiments, n is 40. In embodiments, n is 41. In embodiments, n is 42. In embodiments, n is 43. In embodiments, n is 44. In embodiments, n is 45. In embodiments, n is 46. In embodiments, n is 47. In embodiments, n is 48. In embodiments, n is 49. In embodiments, n is 50. In embodiments, n is 51. In embodiments, n is 52. In embodiments, n is 53. In embodiments, n is 54. In embodiments, n is 55. In embodiments, n is 56. In embodiments, n is 57. In embodiments, n is 58. In embodiments, n is 59. In embodiments, n is 60. In embodiments, n is 61. In embodiments, n is 62. In embodiments, n is 63. In embodiments, n is 64. In embodiments, n is 65. In embodiments, n is 66. In embodiments, n is 67. In embodiments, n is 68. In embodiments, n is 69. In embodiments, n is 70. In embodiments, n is 71. In embodiments, n is 72. In embodiments, n is 73. In embodiments, n is 74. In embodiments, n is 75. In embodiments, n is 76. In embodiments, n is 77. In embodiments, n is 78. In embodiments, n is 79. In embodiments, n is 80. In embodiments, n is 81. In embodiments, n is 82. In embodiments, n is 83. In embodiments, n is 84. In embodiments, n is 85. In embodiments, n is 86. In embodiments, n is 87. In embodiments, n is 88. In embodiments, n is 89. In embodiments, n is 90. In embodiments, n is 91. In embodiments, n is 92. In embodiments, n is 93. In embodiments, n is 94. In embodiments, n is 95. In embodiments, n is 96. In embodiments, n is 97. In embodiments, n is 98. In embodiments, n is 99. In embodiments, n is 100.

In embodiments, n1 is 0. In embodiments, n1 is 1. In embodiments, n1 is 2. In embodiments, n1 is 3. In embodiments, n1 is 4. In embodiments, n1 is 5. In embodiments, n1 is 6. In embodiments, n1 is 7. In embodiments, n1 is 8. In embodiments, n1 is 9. In embodiments, n1 is 10. In embodiments, n1 is 11. In embodiments, n1 is 12. In embodiments, n1 is 13. In embodiments, n1 is 14. In embodiments, n1 is 15. In embodiments, n1 is 16. In embodiments, n1 is 17. In embodiments, n1 is 18. In embodiments, n1 is 19. In embodiments, n1 is 20. In embodiments, n1 is 21. In embodiments, n1 is 22. In embodiments, n1 is 23. In embodiments, n1 is 24. In embodiments, n1 is 25. In embodiments, n1 is 26. In embodiments, n1 is 27. In embodiments, n1 is 28. In embodiments, n1 is 29. In embodiments, n1 is 30. In embodiments, n1 is 31. In embodiments, n1 is 32. In embodiments, n1 is 33. In embodiments, n1 is 34. In embodiments, n1 is 35. In embodiments, n1 is 36. In embodiments, n1 is 37. In embodiments, n1 is 38. In embodiments, n1 is 39. In embodiments, n1 is 40. In embodiments, n1 is 41. In embodiments, n1 is 42. In embodiments, n1 is 43. In embodiments, n1 is 44. In embodiments, n1 is 45. In embodiments, n1 is 46. In embodiments, n1 is 47. In embodiments, n1 is 48. In embodiments, n1 is 49. In embodiments, n1 is 50.

In embodiments, n2 is 1. In embodiments, n2 is 2. In embodiments, n2 is 3. In embodiments, n2 is 4. In embodiments, n2 is 5. In embodiments, n2 is 6. In embodiments, n2 is 7. In embodiments, n2 is 8. In embodiments, n2 is 9. In embodiments, n2 is 10. In embodiments, n2 is 11. In embodiments, n2 is 12. In embodiments, n2 is 13. In embodiments, n2 is 14. In embodiments, n2 is 15. In embodiments, n2 is 16. In embodiments, n2 is 17. In embodiments, n2 is 18. In embodiments, n2 is 19. In embodiments, n2 is 20. In embodiments, n2 is 21. In embodiments, n2 is 22. In embodiments, n2 is 23. In embodiments, n2 is 24. In embodiments, n2 is 25. In embodiments, n2 is 26. In embodiments, n2 is 27. In embodiments, n2 is 28. In embodiments, n2 is 29. In embodiments, n2 is 30. In embodiments, n2 is 31. In embodiments, n2 is 32. In embodiments, n2 is 33. In embodiments, n2 is 34. In embodiments, n2 is 35. In embodiments, n2 is 36. In embodiments, n2 is 37. In embodiments, n2 is 38. In embodiments, n2 is 39. In embodiments, n2 is 40. In embodiments, n2 is 41. In embodiments, n2 is 42. In embodiments, n2 is 43. In embodiments, n2 is 44. In embodiments, n2 is 45. In embodiments, n2 is 46. In embodiments, n2 is 47. In embodiments, n2 is 48. In embodiments, n2 is 49. In embodiments, n2 is 50. In embodiments, n2 is 51. In embodiments, n2 is 52. In embodiments, n2 is 53. In embodiments, n2 is 54. In embodiments, n2 is 55. In embodiments, n2 is 56. In embodiments, n2 is 57. In embodiments, n2 is 58. In embodiments, n2 is 59. In embodiments, n2 is 60. In embodiments, n2 is 61. In embodiments, n2 is 62. In embodiments, n2 is 63. In embodiments, n2 is 64. In embodiments, n2 is 65. In embodiments, n2 is 66. In embodiments, n2 is 67. In embodiments, n2 is 68. In embodiments, n2 is 69. In embodiments, n2 is 70. In embodiments, n2 is 71. In embodiments, n2 is 72. In embodiments, n2 is 73. In embodiments, n2 is 74. In embodiments, n2 is 75. In embodiments, n2 is 76. In embodiments, n2 is 77. In embodiments, n2 is 78. In embodiments, n2 is 79. In embodiments, n2 is 80. In embodiments, n2 is 81. In embodiments, n2 is 82. In embodiments, n2 is 83. In embodiments, n2 is 84. In embodiments, n2 is 85. In embodiments, n2 is 86. In embodiments, n2 is 87. In embodiments, n2 is 88. In embodiments, n2 is 89. In embodiments, n2 is 90. In embodiments, n2 is 91. In embodiments, n2 is 92. In embodiments, n2 is 93. In embodiments, n2 is 94. In embodiments, n2 is 95. In embodiments, n2 is 96. In embodiments, n2 is 97. In embodiments, n2 is 98. In embodiments, n2 is 99. In embodiments, n2 is 100.

In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z2 is 9. In embodiments, z2 is 10. In embodiments, z2 is 11. In embodiments, z2 is 12. In embodiments, z2 is 13. In embodiments, z2 is 14. In embodiments, z2 is 15. In embodiments, z2 is 16. In embodiments, z2 is 17. In embodiments, z2 is 18. In embodiments, z2 is 19. In embodiments, z2 is 20. In embodiments, z2 is 21. In embodiments, z2 is 22. In embodiments, z2 is 23. In embodiments, z2 is 24. In embodiments, z2 is 25. In embodiments, z2 is 26. In embodiments, z2 is 27. In embodiments, z2 is 28. In embodiments, z2 is 29. In embodiments, z2 is 30. In embodiments, z2 is 31. In embodiments, z2 is 32. In embodiments, z2 is 33. In embodiments, z2 is 34. In embodiments, z2 is 35. In embodiments, z2 is 36. In embodiments, z2 is 37. In embodiments, z2 is 38. In embodiments, z2 is 39. In embodiments, z2 is 40. In embodiments, z2 is 41. In embodiments, z2 is 42. In embodiments, z2 is 43. In embodiments, z2 is 44. In embodiments, z2 is 45. In embodiments, z2 is 46. In embodiments, z2 is 47. In embodiments, z2 is 48. In embodiments, z2 is 49. In embodiments, z2 is 50. In embodiments, z2 is 51. In embodiments, z2 is 52. In embodiments, z2 is 53. In embodiments, z2 is 54. In embodiments, z2 is 55. In embodiments, z2 is 56. In embodiments, z2 is 57. In embodiments, z2 is 58. In embodiments, z2 is 59. In embodiments, z2 is 60. In embodiments, z2 is 61. In embodiments, z2 is 62. In embodiments, z2 is 63. In embodiments, z2 is 64. In embodiments, z2 is 65. In embodiments, z2 is 66. In embodiments, z2 is 67. In embodiments, z2 is 68. In embodiments, z2 is 69. In embodiments, z2 is 70. In embodiments, z2 is 71. In embodiments, z2 is 72. In embodiments, z2 is 73. In embodiments, z2 is 74. In embodiments, z2 is 75. In embodiments, z2 is 76. In embodiments, z2 is 77. In embodiments, z2 is 78. In embodiments, z2 is 79. In embodiments, z2 is 80. In embodiments, z2 is 81. In embodiments, z2 is 82. In embodiments, z2 is 83. In embodiments, z2 is 84. In embodiments, z2 is 85. In embodiments, z2 is 86. In embodiments, z2 is 87. In embodiments, z2 is 88. In embodiments, z2 is 89. In embodiments, z2 is 90. In embodiments, z2 is 91. In embodiments, z2 is 92. In embodiments, z2 is 93. In embodiments, z2 is 94. In embodiments, z2 is 95. In embodiments, z2 is 96. In embodiments, z2 is 97. In embodiments, z2 is 98. In embodiments, z2 is 99. In embodiments, z2 is 100.

In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7. In embodiments, z5 is 8. In embodiments, z5 is 9. In embodiments, z5 is 10.

In some embodiments, a cationic amphipathic polymer can have any of the foregoing formula in which z2 is an integer from 2 to 100. In some embodiments, z2 can be an integer in the range 2-100, 2-90, 2-80, 2-70, 2-60, 2-50, 2-40, 2-30, 2-2, or 2-10. In embodiments, z2 can be an integer in the range 2-100 or 2-50.

In some embodiments, a cationic amphipathic polymer can have any of the foregoing formula in which z5 is an integer from 1 to 3. In some other embodiments, z5 is 1 or 3. In still some other embodiments, z5 is 1. In some still other embodiments, z5 is 3.

In some embodiments, a cationic amphipathic polymer can have any of the foregoing formula in which R² is hydrogen.

In some embodiments, a cationic amphipathic polymer can have any of the foregoing formula in which L² is a bond.

In embodiments, CART has the formula:

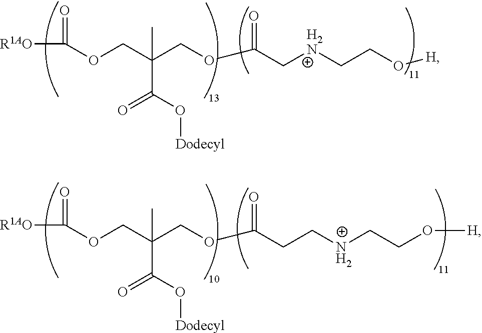

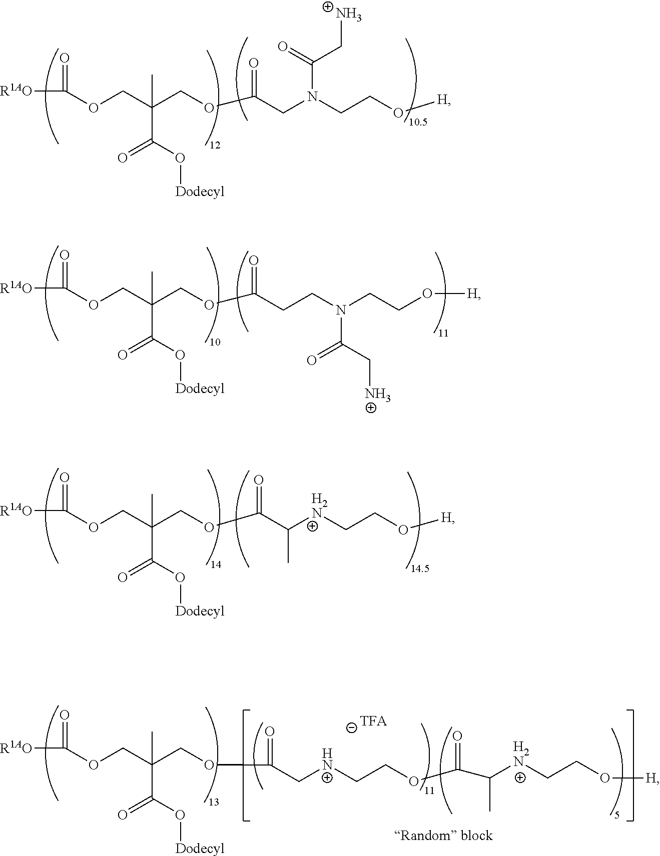

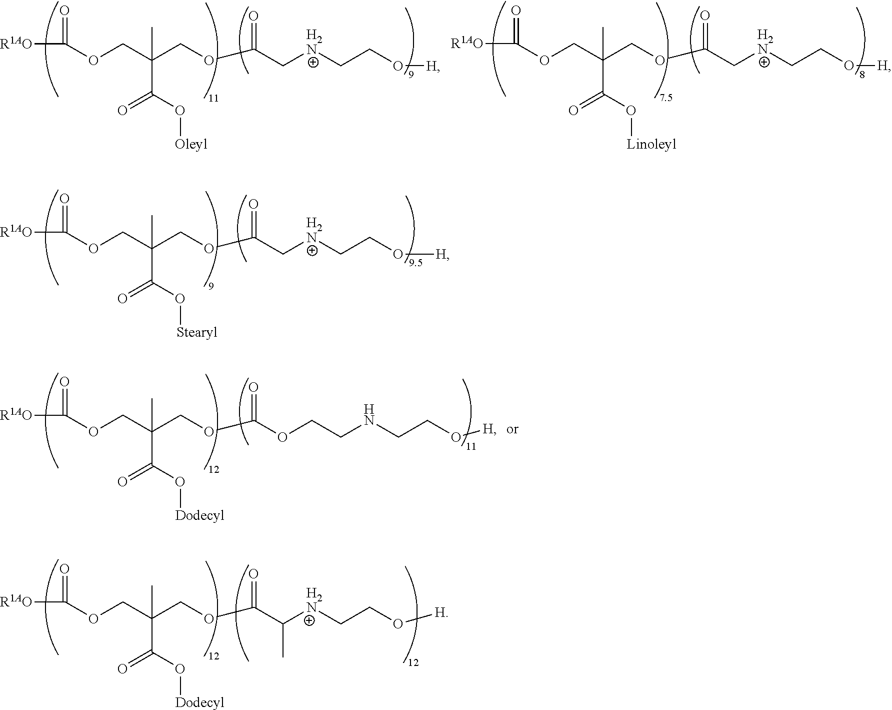

In some embodiments, a pH-sensitive immolation domain can have the formula:

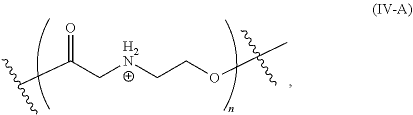

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In some embodiments, in the foregoing formula (IV-A), n is an integer in the range of 2-50.

In some embodiments, a pH-sensitive immolation domain can have the formula:

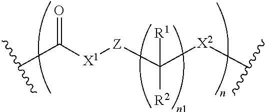

wherein
n is an integer of 2 or more;
n1 is an integer from 0 to 50;
Z is a nucleophilic moiety;
X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—;
X² is —O—or —S—; and
R¹, R², R³, R⁴, R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, a pH-sensitive immolation domain can have the formula:

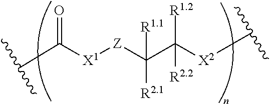

wherein
n is an integer of 2 or more;
Z is a nucleophilic moiety;
X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—;
X² is —O—or —S—; and
R^{1.1}, R^{1.2}, R²¹, R^{2.2}, R³, R⁴, R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, a pH-sensitive immolation domain can have the formula:

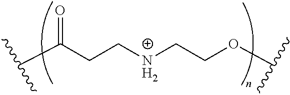

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In some embodiments, a pH-sensitive immolation domain can have the formula:

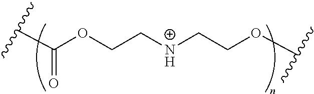

wherein n is an integer of 2 or more. In embodiments, n is an integer in the range 2-100, 2-90, 2-80, 2-70, 2-60, 2-50, 2-40, 2-30, 2-2, or 2-10. In embodiments, n is an integer in the range 2-100 or 2-50.

In some embodiments, a pH-sensitive immolation domain can have the formula:

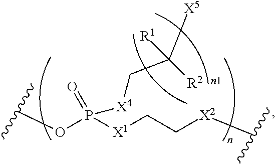

wherein n is an integer of 2 or more; n1 is an integer from 0 to 50; X¹ is a bond, —O—, —NR⁵—, —C(R⁵)(R⁶)— or —C(R⁵)(R⁶)—C(R⁷)(R⁸)—; X² is a bond, —O—, —C(R⁹)(R¹⁹)— or —C(R⁹)(R¹⁰)—C(R¹¹)(R¹²)—; X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—; X⁵ is a nucleophilic moiety; and R¹, R², R⁵, R⁶, R⁷, R⁸, R⁹, R¹⁰, R¹¹, R¹², R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, in a pH-sensitive immolation domain having any of the designated foregoing formula, Z is —S—, —S⁺R¹³—, —NR¹³ —, or —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, in a pH-sensitive immolation domain having any of the designated foregoing formula, Z is —S—, —S⁺R¹³—, —NR¹³ —, or —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, in a pH-sensitive immolation domain having any of the designated foregoing formula, Z is

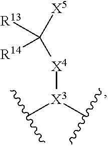

wherein
X³ is C(R¹⁵) or N;
X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—;
X⁵ is a nucleophilic moiety; and
R¹³, R¹⁴, R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, in a pH-sensitive immolation domain having any of the designated foregoing formula, Z is

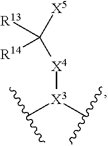

wherein
X³ is C(R¹⁵) or N;
X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—;
X⁵ is a nucleophilic moiety; and
R¹³, R¹⁴, R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, in a pH-sensitive immolation domain having any of the designated foregoing formula, X⁵ is —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, a pH-sensitive immolation domain can have one of the following formula: In embodiments of the cell-penetrating complex disclosed herein and embodiments thereof, the pH-sensitive immolation domain has the structure of Formula (IV) following:

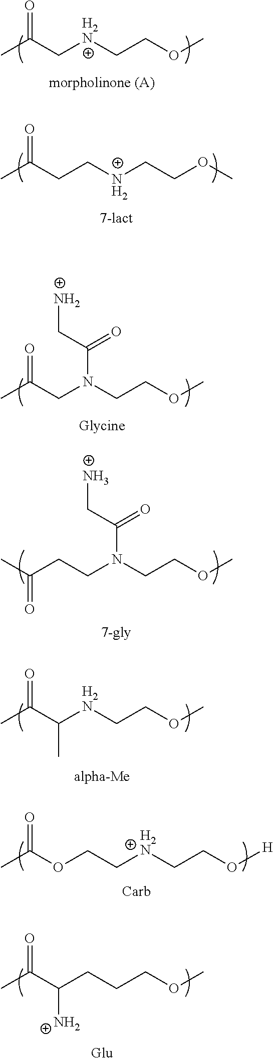

In some embodiments, a lipophilic polymer domain, or interchangeably referred to a lipophilic polymer domain, can have one of the following variations of R group:

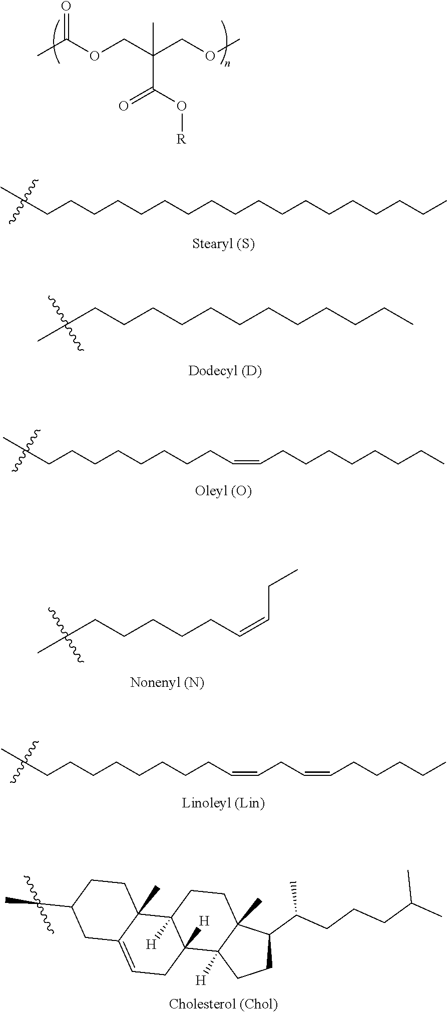

Further to the cell-penetration complex disclosed herein and embodiments thereof, in embodiments, the nucleic acid may be DNA or RNA, such as messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). The cell-penetration complex may further include a protein or peptide.

Further to the cell-penetration complex disclosed herein and embodiments thereof, in embodiments the cell-penetrating complex further includes a plurality of lipophilic moieties.

Further to the cell-penetration complex disclosed herein and embodiments thereof, in embodiments the cell-penetrating complex further includes a plurality of immolation domains.

Further to the cell-penetration complex disclosed herein and embodiments thereof, in embodiments, the counter-anion to the above cationic sequences can include common counterions known in the art, such as for example acetate, trifluoroacetate, triflate, chloride, bromide, sulfate, phosphate, succinate, or citrate. In embodiments, the counter-anion is acetate, trifluoroacetate, triflate, chloride, bromide, sulfate, phosphate, succinate, or citrate.

### Cell-Penetrating Complexes—Section 3

According to the embodiments of Formula (I) and (IV), all substituents are defined as listed below. Any of the cell-penetrating complexes described in this section may be used for the compositions and methods provided herein. For example, any of the nucleic acid, cationic amphipathic polymer and cationic amphipathic polymer described in this section may form part of a cell-penetrating complex including a ribonucleic acid including a sequence encoding a viral protein, a nucleic acid adjuvant and a cationic amphipathic polymer as provided herein including embodiments thereof.

As described above the cationic amphipathic polymer provided herein including embodiments thereof, may have the formula:

R^{1A}-[L¹-[(LP¹)_{z1}-(LP²)_{z3}-(IM)_{z2}]_{z4}-L²-R^{2A}]_{z5}

wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain; IM is the pH-sensitive immolation domain; z5 is an integer from 1 to 10; z1 and z3 are independently integers from 0 to 100, wherein at least one of z1 or z3 is not 0; z4 is an integer from 1 to 100, and z2 is an integer from 2 to 100.

The substituents of the above formula are defined as described throughout the entire application. Thus, in other embodiments of the compounds herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene. In some embodiments, the compound is a chemical species set forth in the Examples section, figures, or tables below.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, or —OCH₂I, or —OCH₂F. In embodiments, R^{1A} is independently R^{1A1}-substituted or unsubstituted (e.g., C₁-C₂₀ or C₁-C₆) alkyl, R^{1A1}-substituted or unsubstituted (e.g., 2 to 20 membered or 2 to 6 membered) heteroalkyl, R^{1A1}-substituted or unsubstituted (e.g., C₃-C₈ or C₅-C₇) cycloalkyl, R^{1A1}-substituted or unsubstituted (e.g., 3 to 8 membered or 3 to 6 membered) heterocycloalkyl, R^{1A1}-substituted or unsubstituted (e.g., C₅-C₁₀ or C₅-C₆) aryl, or R^{1A1}-substituted or unsubstituted (e.g., 5 to 10 membered or 5 to 6 membered) heteroaryl.

R^{1A1} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or. —OCH₂F. In embodiments, R^{1A1} is independently R^{1A2}-substituted or unsubstituted (e.g., C₁-C₂₀ or C₁-C₆) alkyl, R^{1A2}-substituted or unsubstituted (e.g., 2 to 20 membered or 2 to 6 membered) heteroalkyl, R^{1A2}-substituted or unsubstituted (e.g., C₃-C₈ or C₅-C₇) cycloalkyl, R^{1A2}-substituted or unsubstituted (e.g., 3 to 8 membered or 3 to 6 membered) heterocycloalkyl, R^{1A2}-substituted or unsubstituted (e.g., C₅-C₁₀ or C₅-C₆) aryl, or R^{1A2}-substituted or unsubstituted (e.g., 5 to 10 membered or 5 to 6 membered) heteroaryl.

R^{1A2} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or —OCH₂F. In embodiments, R^{1A2} is independently R^{1A3}-substituted or unsubstituted (e.g., C₁-C₂₀ or C₁-C₆) alkyl, R^{1A3}-substituted or unsubstituted (e.g., 2 to 20 membered or 2 to 6 membered) heteroalkyl, R^{1A3}-substituted or unsubstituted (e.g., C₃-C₈ or C₅-C₇) cycloalkyl, R^{1A3}-substituted or unsubstituted (e.g., 3 to 8 membered or 3 to 6 membered) heterocycloalkyl, R^{1A3}-substituted or unsubstituted (e.g., C₅-C₁₀ or C₅-C₆) aryl, or R^{1A3}-substituted or unsubstituted (e.g., 5 to 10 membered or 5 to 6 membered) heteroaryl.

R^{1A3} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or —OCH₂F. In embodiments, R^{1A3} is independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

R^{2A} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, or —OCH₂I, or —OCH₂F. In embodiments, R^{2A} is independently R^{2A1}-substituted or unsubstituted (e.g., C₁-C₂₀ or C₁-C₆) alkyl, R^{2A1}-substituted or unsubstituted (e.g., 2 to 20 membered or 2 to 6 membered) heteroalkyl, R^{2A1}-substituted or unsubstituted (e.g., C₃-C₈ or C₅-C₇) cycloalkyl, R^{2A1}-substituted or unsubstituted (e.g., 3 to 8 membered or 3 to 6 membered) heterocycloalkyl, R^{2A1}-substituted or unsubstituted (e.g., C₅-C₁₀ or C₅-C₆) aryl, or R^{2A1}-substituted or unsubstituted (e.g., 5 to 10 membered or 5 to 6 membered) heteroaryl.

R^{2A1} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or. —OCH₂F. In embodiments, R^{2A1} is independently R^{2A2}-substituted or unsubstituted (e.g., C₁-C₂₀ or C₁-C₆) alkyl, R^{2A2}-substituted or unsubstituted (e.g., 2 to 20 membered or 2 to 6 membered) heteroalkyl, R^{2A2}-substituted or unsubstituted (e.g., C₃-C₈ or C₅-C₇) cycloalkyl, R^{2A2}-substituted or unsubstituted (e.g., 3 to 8 membered or 3 to 6 membered) heterocycloalkyl, R^{2A2}-substituted or unsubstituted (e.g., C₅-C₁₀ or C₅-C₆) aryl, or R^{2A2}-substituted or unsubstituted (e.g., 5 to 10 membered or 5 to 6 membered) heteroaryl.

R^{2A2} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or —OCH₂F. In embodiments, R^{2A2} is independently R^{2A3}-substituted or unsubstituted (e.g., C₁-C₂₀ or C₁-C₆) alkyl, R^{2A3}-substituted or unsubstituted (e.g., 2 to 20 membered or 2 to 6 membered) heteroalkyl, R^{2A3}-substituted or unsubstituted (e.g., C₃-C₈ or C₅-C₇) cycloalkyl, R^{2A3}-substituted or unsubstituted (e.g., 3 to 8 membered or 3 to 6 membered) heterocycloalkyl, R^{2A3}-substituted or unsubstituted (e.g., C₅-C₁₀ or C₅-C₆) aryl, or R^{2A3}-substituted or unsubstituted (e.g., 5 to 10 membered or 5 to 6 membered) heteroaryl.

R^{2A3} may be independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, or —OCH₂F. In embodiments, R^{2A3} is independently unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

L¹ is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is unsubstituted (e.g., C₁-C₂₀ or C₁-C₆) alkylene, unsubstituted (e.g., 2 to 20 membered or 2 to 6 membered) heteroalkylene, unsubstituted (e.g., C₃-C₈ or C₅-C₇) cycloalkylene, unsubstituted (e.g., 3 to 8 membered or 3 to 6 membered) heterocycloalkylene, unsubstituted (e.g., C₅-C₁₀ or C₅-C₆) arylene, or unsubstituted (e.g., 5 to 10 membered or 5 to 6 membered) heteroarylene. In embodiments, L¹ is R^{L1}-substituted (e.g., C₁-C₂₀ or C₁-C₆) alkylene, R^{L1}-substituted (e.g., 2 to 20 membered or 2 to 6 membered) heteroalkylene, R^{L1}-substituted (e.g., C₃-C₈ or C₅-C₇) cycloalkylene, R^{L1}-substituted (e.g., 3 to 8 membered or 3 to 6 membered) heterocycloalkylene, R^{L1}-substituted (e.g., C₅-C₁₀ or C₅-C₆) arylene, or R^{L1}-substituted (e.g., 5 to 10 membered or 5 to 6 membered) heteroarylene. R^{L1} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, SO₂NH₂, —NO₂, —NH₂, —NHNH₂, —ONH₂, —NHC═(O)NHNH₂, unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

L² is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is unsubstituted (e.g., C₁-C₂₀ or C₁-C₆) alkylene, unsubstituted (e.g., 2 to 20 membered or 2 to 6 membered) heteroalkylene, unsubstituted (e.g., C₃-C₈ or C₅-C₇) cycloalkylene, unsubstituted (e.g., 3 to 8 membered or 3 to 6 membered) heterocycloalkylene, unsubstituted (e.g., C₅-C₁₀ or C₅-C₆) arylene, or unsubstituted (e.g., 5 to 10 membered or 5 to 6 membered) heteroarylene. In embodiments, L² is R^{L2}-substituted (e.g., C₁-C₂₀ or C₁-C₆) alkylene, R^{L2}-substituted (e.g., 2 to 20 membered or 2 to 6 membered) heteroalkylene, R^{L2}-substituted (e.g., C₃-C₈ or C₅-C₇) cycloalkylene, R^{L2}-substituted (e.g., 3 to 8 membered or 3 to 6 membered) heterocycloalkylene, R^{L2}-substituted (e.g., C₅-C₁₀ or C₅-C₆) arylene, or R^{L2}-substituted (e.g., 5 to 10 membered or 5 to 6 membered) heteroarylene. R^{L2} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, SO₂NH₂, —NO₂, —NH₂, —NHNH₂, —ONH₂, —NHC═(O)NHNH₂, unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, the cationic amphipathic polymer has the formula:

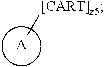

wherein
Ring A is a substituted or unsubstituted cycloalkyl, substituted or unsubstituted heterocycloalkyl, substituted or unsubstituted aryl, or substituted or unsubstituted heteroaryl; CART has the formula: -L¹-[(LP¹)_{z1}-(LP²)_{z3}-(IM)_{z2}]_{z4}-L²-R^{2A} wherein, R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a lipophilic polymer domain; IM is the pH-sensitive immolation domain; z5 is an integer from 1 to 10; z1 and z3 are independently integers from 0 to 100, wherein at least one of z1 or z3 is not 0; z4 is an integer from 1 to 100, and z2 is an integer from 2 to 100.

In embodiments, Ring A is a substituted or unsubstituted aryl. In embodiments, Ring A is a substituted or unsubstituted phenyl. In embodiments, Ring A is a substituted or unsubstituted aryl. In embodiments, Ring A is a substituted or unsubstituted phenyl or naphthalenyl.

In embodiments, the cationic amphipathic polymer has the formula:

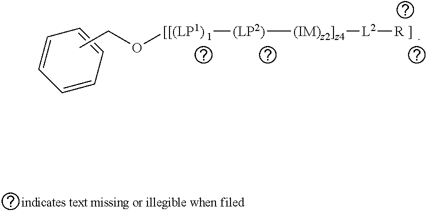

In embodiments, the cationic amphipathic polymer has the formula:

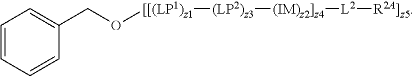

In embodiments, the cationic amphipathic polymer has the formula:

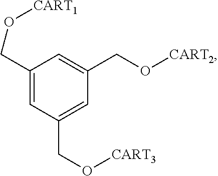

wherein CART₁, CART₂ and CART₃ are independently CART as defined herein.

In embodiments, z5 is an integer from 1 to 3. In embodiments, z5 is 1 or 3. In embodiments, z5 is 1. In embodiments, z5 is 3. In embodiments, R^{2A} is hydrogen. In embodiments, L² is a bond.

In embodiments, the pH-sensitive immolation domain has the formula:

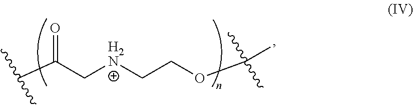

wherein n is an integer of 2 or more. In embodiments, n is an integer in the range of 2-50. In embodiments, n is 7. In embodiments, n is 9.

In embodiments, the lipophilic polymer domain has the formula:

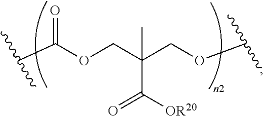

wherein n2 is an integer from 1 to 100; R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, LP¹ has the formula:

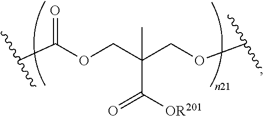

wherein n21 is an integer from 1 to 100; R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R²⁰¹ is independently R^{201A}-substituted or unsubstituted (e.g., C₁-C₃₀ or C₁-C₆) alkyl, R^{201A}-substituted or unsubstituted (e.g., 2 to 20 membered or 2 to 6 membered) heteroalkyl, R^{201A}-substituted or unsubstituted (e.g., C₃-C₈ or C₅-C₇) cycloalkyl, R^{201A}-substituted or unsubstituted (e.g., 3 to 8 membered or 3 to 6 membered) heterocycloalkyl, R^{201A}-substituted or unsubstituted (e.g., C₅-C₁₀ or C₅-C₆) aryl, or R^{201A}-substituted or unsubstituted (e.g., 5 to 10 membered or 5 to 6 membered) heteroaryl.

In embodiments, R^{201A} is independently R^{201B}-substituted or unsubstituted (e.g., C₁-C₃₀ or C₁-C₆) alkyl, R^{201B}-substituted or unsubstituted (e.g., 2 to 20 membered or 2 to 6 membered) heteroalkyl, R^{201B}-substituted or unsubstituted (e.g., C₃-C₈ or C₅-C₇) cycloalkyl, R^{201B}-substituted or unsubstituted (e.g., 3 to 8 membered or 3 to 6 membered) heterocycloalkyl, R^{201B}-substituted or unsubstituted (e.g., C₅-C₁₀ or C₅-C₆) aryl, or R^{201B}-substituted or unsubstituted (e.g., 5 to 10 membered or 5 to 6 membered) heteroaryl.

In embodiments, R^{201B} is independently unsubstituted (e.g., C₁-C₃₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, n21 is 5 and R²⁰¹ is unsubstituted C₁₈ alkenyl. In embodiments, n21 is 6 and R²⁰¹ is unsubstituted C₁₈ alkenyl. In embodiments, the unsubstituted C₁₈ alkenyl is oleyl.

In embodiments, n21 is an integer from 0 to 100. In embodiments, n21 is an integer from 0 to 90. In embodiments, n21 is an integer from 0 to 80. In embodiments, n21 is an integer from 0 to 70. In embodiments, n21 is an integer from 0 to 60. In embodiments, n21 is an integer from 0 to 50. In embodiments, n21 is an integer from 0 to 40. In embodiments, n21 is an integer from 0 to 30. In embodiments, n21 is an integer from 0 to 20. In embodiments, n21 is an integer from or 0 to 10. In embodiments, n21 is an integer from 5 to 100. In embodiments, n21 is an integer from 15 to 100. In embodiments, n21 is an integer from 25 to 100. In embodiments, n21 is an integer from 35 to 100. In embodiments, n21 is an integer from 45 to 100. In embodiments, n21 is an integer from 55 to 100. In embodiments, n21 is an integer from 65 to 100. In embodiments, n21 is an integer from 75 to 100. In embodiments, n21 is an integer from 85 to 100. In embodiments, n21 is an integer from 95 to 100. In embodiments, n21 is 0. In embodiments, n21 is 1. In embodiments, n21 is 2. In embodiments, n21 is 3. In embodiments, n21 is 4. In embodiments, n21 is 5. In embodiments, n21 is 6. In embodiments, n21 is 7. In embodiments, n21 is 8. In embodiments, n21 is 9. In embodiments, n21 is 10. In embodiments, n21 is 11. In embodiments, n21 is 12. In embodiments, n21 is 13. In embodiments, n21 is 14. In embodiments, n21 is 15. In embodiments, n21 is 16. In embodiments, n21 is 17. In embodiments, n21 is 18. In embodiments, n21 is 19. In embodiments, n21 is 20. In embodiments, n21 is 21. In embodiments, n21 is 22. In embodiments, n21 is 23. In embodiments, n21 is 24. In embodiments, n21 is 25. In embodiments, n21 is 26. In embodiments, n21 is 27. In embodiments, n21 is 28. In embodiments, n21 is 29. In embodiments, n21 is 30. In embodiments, n21 is 31. In embodiments, n21 is 32. In embodiments, n21 is 33. In embodiments, n21 is 34. In embodiments, n21 is 35. In embodiments, n21 is 36. In embodiments, n21 is 37. In embodiments, n21 is 38. In embodiments, n21 is 39. In embodiments, n21 is 40. In embodiments, n21 is 41. In embodiments, n21 is 42. In embodiments, n21 is 43. In embodiments, n21 is 44. In embodiments, n21 is 45. In embodiments, n21 is 46. In embodiments, n21 is 47. In embodiments, n21 is 48. In embodiments, n21 is 49. In embodiments, n21 is 50. In embodiments, n21 is 51. In embodiments, n21 is 52. In embodiments, n21 is 53. In embodiments, n21 is 54. In embodiments, n21 is 55. In embodiments, n21 is 56. In embodiments, n21 is 57. In embodiments, n21 is 58. In embodiments, n21 is 59. In embodiments, n21 is 60. In embodiments, n21 is 61. In embodiments, n21 is 62. In embodiments, n21 is 63. In embodiments, n21 is 64. In embodiments, n21 is 65. In embodiments, n21 is 66. In embodiments, n21 is 67. In embodiments, n21 is 68. In embodiments, n21 is 69. In embodiments, n21 is 70. In embodiments, n21 is 71. In embodiments, n21 is 72. In embodiments, n21 is 73. In embodiments, n21 is 74. In embodiments, n21 is 75. In embodiments, n21 is 76. In embodiments, n21 is 77. In embodiments, n21 is 78. In embodiments, n21 is 79. In embodiments, n21 is 80. In embodiments, n21 is 81. In embodiments, n21 is 82. In embodiments, n21 is 83. In embodiments, n21 is 84. In embodiments, n21 is 85. In embodiments, n21 is 86. In embodiments, n21 is 87. In embodiments, n21 is 88. In embodiments, n21 is 89. In embodiments, n21 is 90. In embodiments, n21 is 91. In embodiments, n21 is 92. In embodiments, n21 is 93. In embodiments, n21 is 94. In embodiments, n21 is 95. In embodiments, n21 is 96. In embodiments, n21 is 97. In embodiments, n21 is 98. In embodiments, n21 is 99. In embodiments, n21 is 100.

In embodiments, LP² has the formula:

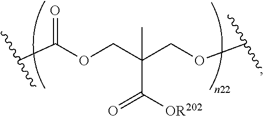

wherein n22 is an integer from 1 to 100; R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, n22 is 5 and R²⁰² is unsubstituted C₉ alkenyl. In embodiments, n22 is 6 and R²⁰² is unsubstituted C₉ alkenyl. In embodiments, the unsubstituted C₉ alkenyl is nonenyl.

In embodiments, R²⁰² is independently R^{202A}-substituted or unsubstituted (e.g., C₁-C₃₀ or C₁-C₆) alkyl, R^{202A}-substituted or unsubstituted (e.g., 2 to 20 membered or 2 to 6 membered) heteroalkyl, R^{202A}-substituted or unsubstituted (e.g., C₃-C₈ or C₅-C₇) cycloalkyl, R^{202A}-substituted or unsubstituted (e.g., 3 to 8 membered or 3 to 6 membered) heterocycloalkyl, R^{202A}-substituted or unsubstituted (e.g., C₅-C₁₀ or C₅-C₆) aryl, or R^{202A}-substituted or unsubstituted (e.g., 5 to 10 membered or 5 to 6 membered) heteroaryl.

In embodiments, R^{202A} is independently R^{202B}-substituted or unsubstituted (e.g., C₁-C₃₀ or C₁-C₆) alkyl, R^{202B}-substituted or unsubstituted (e.g., 2 to 20 membered or 2 to 6 membered) heteroalkyl, R^{202B}-substituted or unsubstituted (e.g., C₃-C₈ or C₅-C₇) cycloalkyl, R^{202B}-substituted or unsubstituted (e.g., 3 to 8 membered or 3 to 6 membered) heterocycloalkyl, R^{202B}-substituted or unsubstituted (e.g., C₅-C₁₀ or C₅-C₆) aryl, or R^{202B}-substituted or unsubstituted (e.g., 5 to 10 membered or 5 to 6 membered) heteroaryl.

In embodiments, R^{202B} is independently unsubstituted (e.g., C₁-C₃₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, n22 is an integer from 0 to 100. In embodiments, n22 is an integer from 0 to 90. In embodiments, n22 is an integer from 0 to 80. In embodiments, n22 is an integer from 0 to 70. In embodiments, n22 is an integer from 0 to 60. In embodiments, n22 is an integer from 0 to 50. In embodiments, n22 is an integer from 0 to 40. In embodiments, n22 is an integer from 0 to 30. In embodiments, n22 is an integer from 0 to 20. In embodiments, n22 is an integer from or 0 to 10. In embodiments, n22 is an integer from 5 to 100. In embodiments, n22 is an integer from 15 to 100. In embodiments, n22 is an integer from 25 to 100. In embodiments, n22 is an integer from 35 to 100. In embodiments, n22 is an integer from 45 to 100. In embodiments, n22 is an integer from 55 to 100. In embodiments, n22 is an integer from 65 to 100. In embodiments, n22 is an integer from 75 to 100. In embodiments, n22 is an integer from 85 to 100. In embodiments, n22 is an integer from 95 to 100. In embodiments, n22 is 0. In embodiments, n22 is 1. In embodiments, n22 is 2. In embodiments, n22 is 3. In embodiments, n22 is 4. In embodiments, n22 is 5. In embodiments, n22 is 6. In embodiments, n22 is 7. In embodiments, n22 is 8. In embodiments, n22 is 9. In embodiments, n22 is 10. In embodiments, n22 is 11. In embodiments, n22 is 12. In embodiments, n22 is 13. In embodiments, n22 is 14. In embodiments, n22 is 15. In embodiments, n22 is 16. In embodiments, n22 is 17. In embodiments, n22 is 18. In embodiments, n22 is 19. In embodiments, n22 is 20. In embodiments, n22 is 21. In embodiments, n22 is 22. In embodiments, n22 is 23. In embodiments, n22 is 24. In embodiments, n22 is 25. In embodiments, n22 is 26. In embodiments, n22 is 27. In embodiments, n22 is 28. In embodiments, n22 is 29. In embodiments, n22 is 30. In embodiments, n22 is 31. In embodiments, n22 is 32. In embodiments, n22 is 33. In embodiments, n22 is 34. In embodiments, n22 is 35. In embodiments, n22 is 36. In embodiments, n22 is 37. In embodiments, n22 is 38. In embodiments, n22 is 39. In embodiments, n22 is 40. In embodiments, n22 is 41. In embodiments, n22 is 42. In embodiments, n22 is 43. In embodiments, n22 is 44. In embodiments, n22 is 45. In embodiments, n22 is 46. In embodiments, n22 is 47. In embodiments, n22 is 48. In embodiments, n22 is 49. In embodiments, n22 is 50. In embodiments, n22 is 51. In embodiments, n22 is 52. In embodiments, n22 is 53. In embodiments, n22 is 54. In embodiments, n22 is 55. In embodiments, n22 is 56. In embodiments, n22 is 57. In embodiments, n22 is 58. In embodiments, n22 is 59. In embodiments, n22 is 60. In embodiments, n22 is 61. In embodiments, n22 is 62. In embodiments, n22 is 63. In embodiments, n22 is 64. In embodiments, n22 is 65. In embodiments, n22 is 66. In embodiments, n22 is 67. In embodiments, n22 is 68. In embodiments, n22 is 69. In embodiments, n22 is 70. In embodiments, n22 is 71. In embodiments, n22 is 72. In embodiments, n22 is 73. In embodiments, n22 is 74. In embodiments, n22 is 75. In embodiments, n22 is 76. In embodiments, n22 is 77. In embodiments, n22 is 78. In embodiments, n22 is 79. In embodiments, n22 is 80. In embodiments, n22 is 81. In embodiments, n22 is 82. In embodiments, n22 is 83. In embodiments, n22 is 84. In embodiments, n22 is 85. In embodiments, n22 is 86. In embodiments, n22 is 87. In embodiments, n22 is 88. In embodiments, n22 is 89. In embodiments, n22 is 90. In embodiments, n22 is 91. In embodiments, n22 is 92. In embodiments, n22 is 93. In embodiments, n22 is 94. In embodiments, n22 is 95. In embodiments, n22 is 96. In embodiments, n22 is 97. In embodiments, n22 is 98. In embodiments, n22 is 99. In embodiments, n22 is 100.

In embodiments, the cationic amphipathic polymer has the formula:

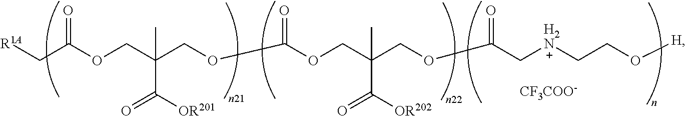

wherein n21 is 5, R²⁰¹ is oleyl, n22 is 5, R²⁰² is nonenyl and n is 7.

In embodiments, the cationic amphipathic polymer has the formula:

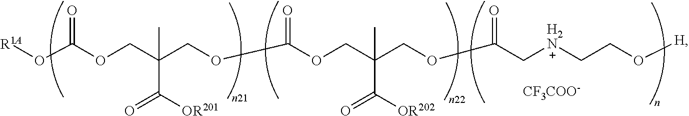

wherein n21 is 6, R²⁰¹ is oleyl, n22 is 6, R²⁰² is nonenyl and n is 9.

In embodiments, the nucleic acid is an messenger RNA (mRNA), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). In embodiments, the nucleic acid includes a sequence encoding a chimeric antigen receptor (CAR).

In an aspect, a nanoparticle composition including a plurality of cell-penetrating complexes as provided herein including embodiments thereof is provided.

In an aspect, a pharmaceutical composition including the complex as provided herein including embodiments thereof and a pharmaceutically acceptable carrier is provided.

In an aspect, method of transfecting a nucleic acid into a cell is provided, the method including contacting a cell with the complex as provided herein including embodiments thereof.

In embodiments, the cationic amphipathic polymer is allowed to degrade within the cell thereby forming a degradation product. In embodiments, the degradation product is a substituted or unsubstituted diketopiperazine.

In embodiments, the nucleic acid includes a CAR encoding messenger RNA (mRNA).

The mRNA provided herein may be expressed in a cell. In embodiments, the cell is an eukaryotic cell. In embodiments, the cell is a mammalian or human cell. In embodiments, the cell forms part of an organism. In embodiments, the organism is a human. In embodiments, the cell is a lymphoid cell or a myeloid cell. In embodiments, the cell is a T cell. In embodiments, the cell is a myeloid cell.

In embodiments, the cationic amphipathic polymer has the formula:

R^{1A}-[L¹[(LP¹)_{z1}-(IM)_{z2}-(LP²)_{z3}]_{z4}-L²-R^{2A}]_{z5}

wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —O CHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a bond or a lipophilic polymer domain, wherein at least one of LP¹ or LP² is a lipophilic polymer domain; IM is the pH-sensitive immolation domain; z5 is an integer from 1 to 10; z1 and z3 are independently integers from 0 to 100, wherein at least one of z1 or z3 is not 0; z4 is an integer from 1 to 100, and z2 is an integer from 2 to 100.

In embodiments, the cationic amphipathic polymer has the formula:

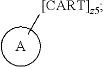

wherein Ring A is a substituted or unsubstituted cycloalkyl, substituted or unsubstituted heterocycloalkyl, substituted or unsubstituted aryl, or substituted or unsubstituted heteroaryl; CART has the formula: -L¹-[(LP¹)_{z1}-(IM)_{z2}-(LP²)_{z3}]_{z4}-L²-R^{2A}
wherein,
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a bond or a lipophilic polymer domain, wherein at least one of LP¹ or LP² is a lipophilic polymer domain;
IM is the pH-sensitive immolation domain; z5 is an integer from 1 to 10; z1 and z3 are independently integers from 0 to 100, wherein at least one of z1 or z3 is not 0; z4 is an integer from 1 to 100, and z2 is an integer from 2 to 100.

In embodiments, Ring A is a substituted or unsubstituted aryl. In embodiments, Ring A is a substituted or unsubstituted phenyl. In embodiments, Ring A is a substituted or unsubstituted aryl. In embodiments, Ring A is a substituted or unsubstituted phenyl or naphthalenyl. In embodiments, the cationic amphipathic polymer has the formula:

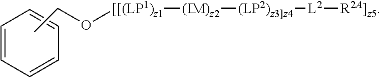

In embodiments, the cationic amphipathic polymer has the formula:

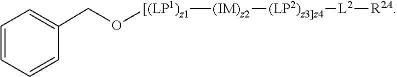

In embodiments, the cationic amphipathic polymer has the formula:

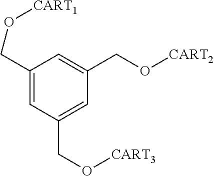

wherein CART₁, CART₂ and CART₃ are independently CART as defined herein.

In embodiments, the L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the pH-sensitive immolation domain has the formula:

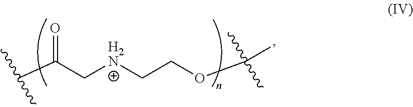

wherein n is an integer of 2 or more.

### Cell-Penetrating Complexes—Section 4

According to the embodiments of Formula (Ia-C), (Ib-C), (Ic-C), (Id-C), (VIII-C), (IX-C), (X-C), (XI-C), (II), (III), (XIV-C), (XV-C), (XVI-C), (XVII-C), (XVIII-C), (XIX-C), (XX-C), (XXb-C), (XXI-C), and (XXIa-C), all substituents are defined as listed below. Any of the cell-penetrating complexes described in this section may be used for the compositions and methods provided herein. For example, any of the nucleic acid, cationic amphipathic polymer and cationic amphipathic polymer described in this section may form part of a cell-penetrating complex including a ribonucleic acid including a sequence encoding a viral protein, a nucleic acid adjuvant and a cationic amphipathic polymer as provided herein including embodiments thereof.

The cell-penetrating complexes provided herein including embodiments thereof, include a nucleic acid non-covalently bound to a cationic amphipathic polymer (e.g., having the formula (II), (III), (XIV-C), and (XV-C)). The cell-penetrating complexes provided herein, including embodiments thereof, may further include a plurality (more than one, e.g., two) of cationic amphipathic polymer types (e.g., a mixture of a first cationic amphipathic polymer and a second amphipathic polymer) wherein each of the cationic amphipathic polymer types is chemically different.

The cell-penetrating complex provided herein may include a nucleic acid non-covalently bound to a cationic amphipathic polymer (e.g., having the formula (II), (III), (XIV-C), and (XV-C)), the cationic amphipathic polymer including a pH-sensitive immolation domain (e.g., having Formula (XVI-C), (XVII-C), (XVIII-C), (XIX-C), (XX-C), and (XXI-C)). In embodiments, one or more counter ions (e.g., anions) may also be present as countercharges to the positive charges in the cationic amphipathic polymer. In embodiments, the nucleic acid is non-covalently bound to the cationic amphipathic polymer. In embodiments, the nucleic acid is ionically bound to the cationic amphipathic polymer. In embodiments, the cell penetrating complex includes a plurality of optionally different nucleic acids (e.g. 1 to 10 additional nucleic acids, 1 to 5 additional nucleic acids, 1 to 5 additional nucleic acids, 2 additional nucleic acids or 1 additional nucleic acid). In embodiments, the nucleic acid is DNA. In embodiments, the nucleic acid is RNA. In embodiments, the nucleic acid is mRNA.

In embodiments, the cationic amphipathic polymer has the formula:

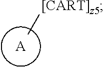

wherein Ring A is a substituted or unsubstituted cycloalkyl, substituted or unsubstituted heterocycloalkyl, substituted or unsubstituted aryl, or substituted or unsubstituted heteroaryl;
CART has the formula: -L¹-[(LP¹)_{z1}-(LP²)_{z3}-(IM)_{z2}]_{z4}-L²-R^{2A};
   wherein, R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a lipophilic polymer domain, wherein at least one of LP¹ or LP² is a lipophilic polymer domain; IM has the formula:

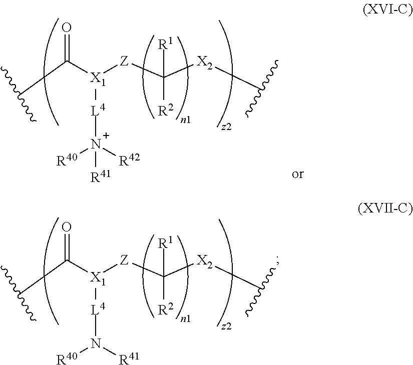

z5 are an integer from 1 to 10; z1 and z3 are independently integers from 0 to 100, wherein at least one of z1 or z3 is not 0; z4 is an integer from 1 to 100; and z2 is an integer from 2 to 100.

In formula (XVI-C) and (XVII-C)×1 may be a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—. X² is —O— or —S—. R¹ and R² are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. L⁴ is a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene. R⁴⁰, R⁴¹, and R⁴² are independently hydrogen, substituted or unsubstituted alkyl or substituted or unsubstituted heteroalkyl. Z is —S—, —S⁺R¹³—, —NR¹³ —, or —N⁺(R¹³)(H)—. R¹³ is hydrogen, —CCl₃, —CBr₃, —CF₃, —CI₃, —CN, —OH, ═O, —NH₂, —COOH, —CONH₂, —SH, —SO₃H, —SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. n1 is an integer from 0 to 50. z2 is an integer from 2 to 100; and z5 is an integer from 1 to 10.

In embodiments, Ring A is a substituted or unsubstituted aryl. In embodiments, Ring A is a substituted or unsubstituted phenyl. In embodiments, Ring A is a substituted or unsubstituted aryl. In embodiments, Ring A is a substituted or unsubstituted phenyl or naphthalenyl.

In embodiments, the cationic amphipathic polymer has the formula:

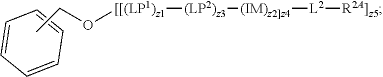

wherein IM has the formula:

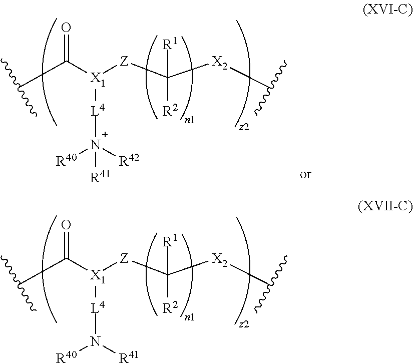

and wherein the substituents and variables are defined as described herein.

In embodiments, the cationic amphipathic polymer has the formula:

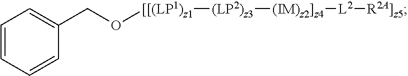

wherein IM has the formula:

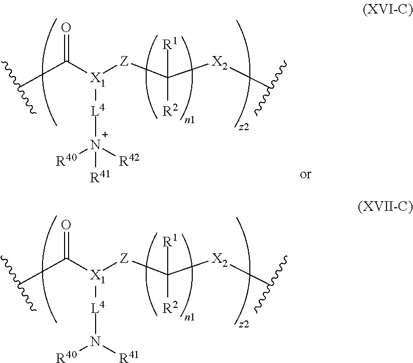

wherein the substituents and variables are defined as described herein.

In embodiments, the cationic amphipathic polymer has the formula:

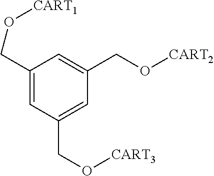

wherein CART₁, CART₂ and CART₃ are independently CART as defined herein.

In embodiments, z5 is an integer from 1 to 3. In embodiments, z5 is 1 or 3. In embodiments, z5 is 1. In embodiments, z5 is 3. In embodiments, R^{2A} is hydrogen. In embodiments, L² is a bond.

In embodiments, the cationic amphipathic polymer has the formula:

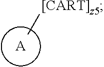

wherein Ring A is a substituted or unsubstituted cycloalkyl, substituted or unsubstituted heterocycloalkyl, substituted or unsubstituted aryl, or substituted or unsubstituted heteroaryl; CART has the formula: -L¹-[(LP¹)_{z1}-(IM)_{z2}-(LP²)_{z3}]_{z4}-L²-R^{2A};
wherein, R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; LP¹ and LP² are independently a bond or a lipophilic polymer domain, wherein at least one of LP¹ or LP² is a lipophilic polymer domain; IM has the formula:

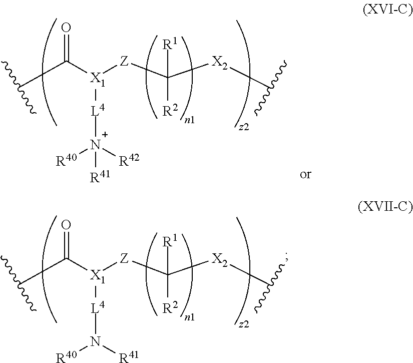

z5 are an integer from 1 to 10; z1 and z3 are independently integers from 0 to 100, wherein at least one of z1 or z3 is not 0; z4 is an integer from 1 to 100; and z2 is an integer from 2 to 100.

In formula (XVI-C) and (XVII-C) X₁ may be a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—. X² is —O— or —S—. R¹ and R² are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. L⁴ is a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene. R⁴⁰, R⁴¹ and R⁴² are independently hydrogen, substituted or unsubstituted alkyl or substituted or unsubstituted heteroalkyl. Z is —S—, —S⁺R¹³—, —NR¹³—, or —N⁺(R¹³)(H)—. R¹³ is hydrogen, —CCl₃, —CBr₃, —CF₃, —CI₃, —CN, —OH, ═O, —NH₂, —COOH, —CONH₂, —SH, —SO₃H, —SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. n1 is an integer from 0 to 50. z2 is an integer from 2 to 100; and z5 is an integer from 1 to 10.

In embodiments, Ring A is a substituted or unsubstituted aryl. In embodiments, Ring A is a substituted or unsubstituted phenyl. In embodiments, Ring A is a substituted or unsubstituted aryl. In embodiments, Ring A is a substituted or unsubstituted phenyl or naphthalenyl.

In embodiments, the cationic amphipathic polymer has the formula:

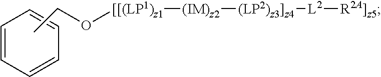

wherein IM has the formula:

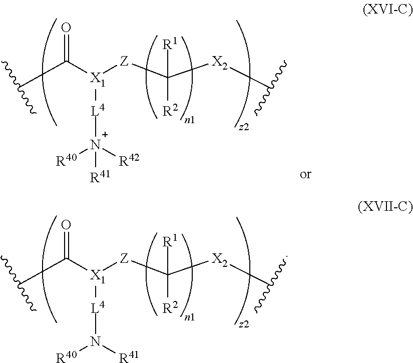

In embodiments, the cationic amphipathic polymer has the formula:

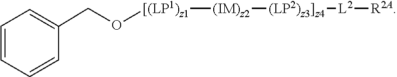

wherein IM has the formula:

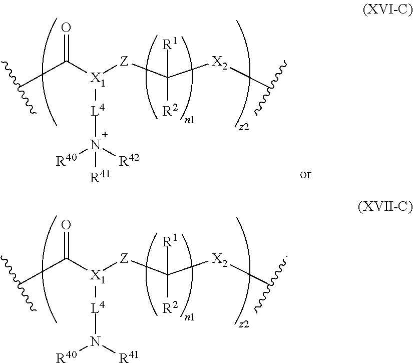

In embodiments, the cationic amphipathic polymer has the formula:

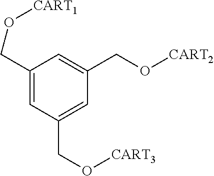

wherein CART₁, CART₂ and CART₃ are independently CART as defined herein.

In embodiments, Ring A is a substituted or unsubstituted aryl. In some other embodiments, Ring A is a substituted or unsubstituted phenyl. In still some other embodiments, Ring A is a substituted or unsubstituted aryl. In still some other embodiments, Ring A is a substituted or unsubstituted phenyl or naphthalenyl.

In embodiments, Ring A is an unsubstituted aryl (i.e. unsubstituted beyond the CART moiety). In embodiments, Ring A is an unsubstituted phenyl (i.e. unsubstituted beyond the CART moiety). In embodiments, Ring A is an unsubstituted phenyl or naphthalenyl (i.e. unsubstituted beyond the CART moiety). In embodiments, Ring A is a substituted aryl (i.e. substituted in addition to the CART moiety). In embodiments, Ring A is a substituted phenyl (i.e. substituted in addition to the CART moiety). In embodiments, Ring A is a substituted phenyl or naphthalenyl (i.e. substituted in addition to the CART moiety).

In embodiments, the cell-penetrating complex has a detectable agent (e.g., fluorophore).

In embodiments, R^{1A} is an aryl substituted with a methoxy linker. In embodiments, R^{1A} is an aryl substituted with a linker (e.g., —CH₂—O—). A non-limiting example wherein R^{1A} is an aryl substituted with a methoxy linker has the formula:

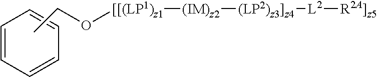

wherein LP¹, LP², IM, L², R^{2A}, z1, z2, z3, z4, and z5 are defined as herein.

In embodiments, the cationic amphipathic polymer has the formula (IX-C):

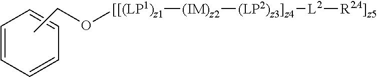

wherein LP¹, LP², IM, L², R^{2A}, z1, z2, z3, z4, and z5 are defined as herein.

In embodiments, the cationic amphipathic polymer has the formula (X-C):

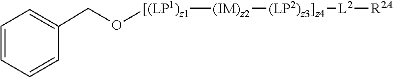

wherein LP¹, LP², IM, L², R^{2A}, z1, z2, z3, z4, and z5 are defined as herein.

In some embodiments, the cationic amphipathic polymer has the formula (XI-C):

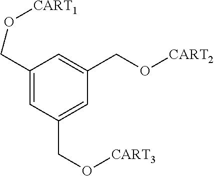

wherein CART₁, CART₂ and CART₃ are independently a CART moiety as defined in formula (VIII-C) (e.g., -L₁-[(LP¹)_{z1}-(IM)_{z2}-(LP²)_{z3}]_{z4}-L²-R^{2A}). In embodiments each CART moiety is optionally different.

In embodiments, the L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, the cationic amphipathic polymer has the formula:

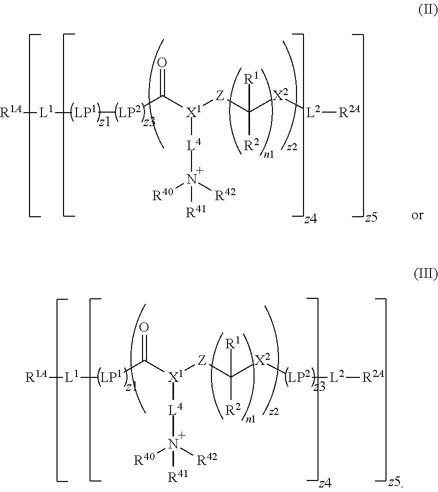

In Formula (II) and (III), R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
LP¹ and LP² are independently a lipophilic polymer domain.
X¹ is a bond, —C(R⁵)(R⁶)—, —C(R⁵)(R⁶)—C(R⁷)(R⁸)—, —O—C(R⁵)(R⁶)—, or —O-C(R⁵)(R⁶)—C(R⁷)(R⁸)—.
X² is —O— or —S—.
R¹, R², R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.
L⁴ is a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene.
R⁴⁰, R⁴¹ and R⁴² are independently hydrogen, substituted or unsubstituted alkyl or substituted or unsubstituted heteroalkyl.
Z is —S—, —S⁺R¹³—, —NR¹³ —, or —N⁺(R¹³)(H)—.
R¹³ is hydrogen, —CCl₃, —CBr₃, —CF₃, —CI₃, —CN, —OH, ═O, —NH₂, —COOH, —CONH₂, —SH, —SO₃H, —SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.
n1 is an integer from 0 to 50.
z1 and z3 are independently integers from 0 to 100, wherein at least one of z1 or z3 is not 0.
z4 is an integer from 1 to 100.
z2 is an integer from 2 to 100; and z5 is an integer from 1 to 10.

In embodiments, the cationic amphipathic polymer has the formula:

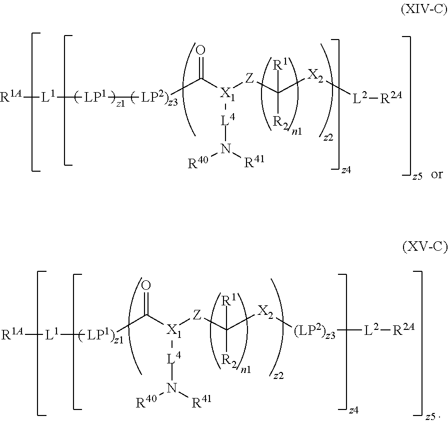

In Formula (XIV-C) and (XV-C), R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

LP¹ and LP² are independently a lipophilic polymer domain;

X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—.

X² is —O— or —S—.

R¹, R², R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

L⁴ is a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene.

R⁴⁰ and R⁴¹ are independently hydrogen, substituted or unsubstituted alkyl or substituted or unsubstituted heteroalkyl.

Z is —S—, —S⁺R¹³—, —NR¹³ —, or —N⁺(R¹³)(H)—.

R¹³ is hydrogen, —CCl₃, —CBr₃, —CF₃, —CI₃, —CN, —OH, ═O, —NH₂, —COOH, —CONH₂, —SH, —SO₃H, —SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

n1 is an integer from 0 to 50.

z1 and z3 are independently integers from 0 to 100, wherein at least one of z1 or z3 is not 0. z4 is an integer from 1 to 100. z2 is an integer from 2 to 100; and z5 is an integer from 1 to 10.

In embodiments, L⁴ is substituted or unsubstituted C₂-C₈ alkylene. In embodiments, L⁴ is substituted or unsubstituted C₈ alkylene. In embodiments, L⁴ is substituted or unsubstituted C₇ alkylene. In embodiments, L⁴ is substituted or unsubstituted C₆ alkylene. In embodiments, L⁴ is substituted or unsubstituted C₅ alkylene. In embodiments, L⁴ is substituted or unsubstituted C₄ alkylene. In embodiments, L⁴ is substituted or unsubstituted C₃ alkylene. In embodiments, L⁴ is substituted or unsubstituted C₂ alkylene. In embodiments, L⁴ is unsubstituted C₂-C₈ alkylene. In embodiments, L⁴ is unsubstituted C₈ alkylene. In embodiments, L⁴ is unsubstituted C₇ alkylene. In embodiments, L⁴ is unsubstituted C₆ alkylene. In embodiments, L⁴ is unsubstituted C₅ alkylene. In embodiments, L⁴ is unsubstituted C₄ alkylene. In embodiments, L⁴ is unsubstituted C₃ alkylene. In embodiments, L⁴ is unsubstituted C₂ alkylene. In embodiments, L⁴ is unsubstituted C₂ alkylene, unsubstituted C₃ alkylene or unsubstituted C₄ alkylene.

In embodiments, L⁴ is substituted (e.g., substituted with at least one substituent group, size-limited substituent group, or lower substituent group) or unsubstituted C₂-C₈ alkylene (e.g., C₂-C₈, C₂-C₆, C₂-C₄, or C₂). In embodiments, L⁴ is unsubstituted C₂-C₈ alkylene (e.g., C₂-C₈, C₂-C₆, C₂-C₄, or C₂). In embodiments, L⁴ is unsubstituted C₂ alkylene, unsubstituted C₃ alkylene or unsubstituted C₄ alkylene.

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, R⁴⁰ is independently hydrogen, substituted or unsubstituted alkyl or substituted or unsubstituted heteroalkyl. In embodiments, R⁴⁰ is independently hydrogen, substituted (e.g., substituted with at least one substituent group, size-limited substituent group, or lower substituent group) or unsubstituted alkyl (e.g., C₁-C₈, C₁-C₆, C₁-C₄, or C₁-C₂) or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, a substituted R⁴⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴⁰ is substituted with a plurality of groups selected from substituent groups, size-limited substituent groups, and lower substituent groups; each substituent group, size-limited substituent group, and/or lower substituent group may optionally be different. In embodiments, when R⁴⁰ is substituted, it is substituted with at least one substituent group. In embodiments, when R⁴⁰ is substituted, it is substituted with at least one size-limited substituent group. In embodiments, when R⁴⁰ is substituted, it is substituted with at least one lower substituent group.

In embodiments, R⁴¹ is independently hydrogen, substituted or unsubstituted alkyl or substituted or unsubstituted heteroalkyl. In embodiments, R⁴¹ is independently hydrogen, substituted (e.g., substituted with at least one substituent group, size-limited substituent group, or lower substituent group) or unsubstituted alkyl (e.g., C₁-C₈, C₁-C₆, C₁-C₄, or C₁-C₂) or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, a substituted R⁴¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴¹ is substituted with a plurality of groups selected from substituent groups, size-limited substituent groups, and lower substituent groups; each substituent group, size-limited substituent group, and/or lower substituent group may optionally be different. In embodiments, when R⁴¹ is substituted, it is substituted with at least one substituent group. In embodiments, when R⁴¹ is substituted, it is substituted with at least one size-limited substituent group. In embodiments, when R⁴¹ is substituted, it is substituted with at least one lower substituent group.

In embodiments, R⁴² is independently hydrogen, substituted or unsubstituted alkyl or substituted or unsubstituted heteroalkyl. In embodiments, R⁴² is independently hydrogen, substituted (e.g., substituted with at least one substituent group, size-limited substituent group, or lower substituent group) or unsubstituted alkyl (e.g., C₁-C₈, C₁-C₆, C₁-C₄, or C₁-C₂) or substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered).

In embodiments, a substituted R⁴² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴² is substituted with a plurality of groups selected from substituent groups, size-limited substituent groups, and lower substituent groups; each substituent group, size-limited substituent group, and/or lower substituent group may optionally be different. In embodiments, when R⁴² is substituted, it is substituted with at least one substituent group. In embodiments, when R⁴² is substituted, it is substituted with at least one size-limited substituent group. In embodiments, when R⁴² is substituted, it is substituted with at least one lower substituent group.

In embodiments, R⁴⁰, R⁴¹, and R⁴² are independently hydrogen or substituted heteroalkyl. In embodiments, R⁴⁰, R⁴¹, and R⁴² are independently hydrogen or —C(NH)NH₂. In embodiments, at least two of R⁴⁰, R⁴¹, and R⁴² are hydrogen and one is —C(NH)NH₂.

In embodiments, R^{1A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{1A} is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1A} is unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1A} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{1A} is substituted or unsubstituted alkyl (e.g., C₁-C₈ alkyl, C₁-C₆ alkyl, or C₁-C₄ alkyl). In embodiments, R^{1A} is substituted alkyl (e.g., C₁-C₈ alkyl, C₁-C₆ alkyl, or C₁-C₄ alkyl). In embodiments, R^{1A} is an unsubstituted alkyl (e.g., C₁-C₈ alkyl, C₁-C₆ alkyl, or C₁-C₄ alkyl). In embodiments, R^{1A} is substituted or unsubstituted heteroalkyl (e.g., 2 to 8 membered heteroalkyl, 2 to 6 membered heteroalkyl, or 2 to 4 membered heteroalkyl). In embodiments, R^{1A} is substituted heteroalkyl (e.g., 2 to 8 membered heteroalkyl, 2 to 6 membered heteroalkyl, or 2 to 4 membered heteroalkyl). In embodiments, R^{1A} is an unsubstituted heteroalkyl (e.g., 2 to 8 membered heteroalkyl, 2 to 6 membered heteroalkyl, or 2 to 4 membered heteroalkyl). In embodiments, R^{1A} is substituted or unsubstituted cycloalkyl (e.g., C₃-C₈ cycloalkyl, C₃-C₆ cycloalkyl, or C₅-C₆ cycloalkyl). In embodiments, R^{1A} is substituted cycloalkyl (e.g., C₃-C₈ cycloalkyl, C₃-C₆ cycloalkyl, or C₅-C₆ cycloalkyl). In embodiments, R^{1A} is an unsubstituted cycloalkyl (e.g., C₃-C₈ cycloalkyl, C₃-C₆ cycloalkyl, or C₅-C₆ cycloalkyl). In embodiments, R^{1A} is substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is substituted heterocycloalkyl (e.g., 3 to 8 membered heterocycloalkyl, 3 to 6 membered heterocycloalkyl, or 5 to 6 membered heterocycloalkyl). In embodiments, R^{1A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is substituted or unsubstituted aryl (e.g., C₆-C₁₀ aryl, C₁₀ aryl, or phenyl). In embodiments, R^{1A} is substituted aryl (e.g., C₆-C₁₀ aryl, C₁₀ aryl, or phenyl). In embodiments, R^{1A} is an unsubstituted aryl (e.g., C₆-C₁₀ aryl, C₁₀ aryl, or phenyl). In embodiments, R^{1A} is substituted or unsubstituted heteroaryl (e.g., 5 to 10 membered heteroaryl, 5 to 9 membered heteroaryl, or 5 to 6 membered heteroaryl). In embodiments, R^{1A} is substituted heteroaryl (e.g., 5 to 10 membered heteroaryl, 5 to 9 membered heteroaryl, or 5 to 6 membered heteroaryl). In embodiments, R^{1A} is an unsubstituted heteroaryl (e.g., 5 to 10 membered heteroaryl, 5 to 9 membered heteroaryl, or 5 to 6 membered heteroaryl).

In embodiments, R^{1A} is a substituted or unsubstituted aryl. In some other embodiments, R^{1A} is a substituted or unsubstituted phenyl. In still some other embodiments, R^{1A} is a substituted or unsubstituted aryl. In still some other embodiments, R^{1A} is a substituted or unsubstituted phenyl or naphthalenyl.

In embodiments, R^{2A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{2A} is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{2A} is unsubstituted alkyl, unsubstituted heteroalkyl, unsubstituted cycloalkyl, unsubstituted heterocycloalkyl, unsubstituted aryl, or unsubstituted heteroaryl.

In embodiments, R^{2A} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{2A} is substituted or unsubstituted alkyl (e.g., C₁-C₈ alkyl, C₁-C₆ alkyl, or C₁-C₄ alkyl). In embodiments, R^{2A} is substituted alkyl (e.g., C₁-C₈ alkyl, C₁-C₆ alkyl, or C₁-C₄ alkyl). In embodiments, R^{2A} is an unsubstituted alkyl (e.g., C₁-C₈ alkyl, C₁-C₆ alkyl, or C₁-C₄ alkyl). In embodiments, R^{2A} is substituted or unsubstituted heteroalkyl (e.g., 2 to 8 membered heteroalkyl, 2 to 6 membered heteroalkyl, or 2 to 4 membered heteroalkyl). In embodiments, R^{2A} is substituted heteroalkyl (e.g., 2 to 8 membered heteroalkyl, 2 to 6 membered heteroalkyl, or 2 to 4 membered heteroalkyl). In embodiments, R^{2A} is an unsubstituted heteroalkyl (e.g., 2 to 8 membered heteroalkyl, 2 to 6 membered heteroalkyl, or 2 to 4 membered heteroalkyl). In embodiments, R^{2A} is substituted or unsubstituted cycloalkyl (e.g., C₃-C₈ cycloalkyl, C₃-C₆ cycloalkyl, or C₅-C₆ cycloalkyl). In embodiments, R^{2A} is substituted cycloalkyl (e.g., C₃-C₈ cycloalkyl, C₃-C₆ cycloalkyl, or C₅-C₆ cycloalkyl). In embodiments, R^{2A} is an unsubstituted cycloalkyl (e.g., C₃-C₈ cycloalkyl, C₃-C₆ cycloalkyl, or C₅-C₆ cycloalkyl). In embodiments, R^{2A} is substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is substituted heterocycloalkyl (e.g., 3 to 8 membered heterocycloalkyl, 3 to 6 membered heterocycloalkyl, or 5 to 6 membered heterocycloalkyl). In embodiments, R^{2A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{2A} is substituted or unsubstituted aryl (e.g., C₆-C₁₀ aryl, C₁₀ aryl, or phenyl). In embodiments, R^{2A} is substituted aryl (e.g., C₆-C₁₀ aryl, C₁₀ aryl, or phenyl). In embodiments, R^{2A} is an unsubstituted aryl (e.g., C₆-C₁₀ aryl, C₁₀ aryl, or phenyl). In embodiments, R^{2A} is substituted or unsubstituted heteroaryl (e.g., 5 to 10 membered heteroaryl, 5 to 9 membered heteroaryl, or 5 to 6 membered heteroaryl). In embodiments, R^{2A} is substituted heteroaryl (e.g., 5 to 10 membered heteroaryl, 5 to 9 membered heteroaryl, or 5 to 6 membered heteroaryl). In embodiments, R^{2A} is an unsubstituted heteroaryl (e.g., 5 to 10 membered heteroaryl, 5 to 9 membered heteroaryl, or 5 to 6 membered heteroaryl).

In embodiments, R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{3A} is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{3A} is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{3A} is unsubstituted alkyl, unsubstituted heteroalkyl, unsubstituted cycloalkyl, unsubstituted heterocycloalkyl, unsubstituted aryl, or unsubstituted heteroaryl.

In embodiments, R^{3A} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R^{3A} is a substituted or unsubstituted aryl. In some other embodiments, R^{3A} is a substituted or unsubstituted phenyl. In still some other embodiments, R^{3A} is a substituted or unsubstituted aryl. In still some other embodiments, R^{3A} is a substituted or unsubstituted phenyl or naphthalenyl.

In embodiments, R^{3A} is substituted or unsubstituted alkyl (e.g., C₁-C₈ alkyl, C₁-C₆ alkyl, or C₁-C₄ alkyl). In embodiments, R^{3A} is substituted alkyl (e.g., C₁-C₈ alkyl, C₁-C₆ alkyl, or C₁-C₄ alkyl). In embodiments, R^{3A} is an unsubstituted alkyl (e.g., C₁-C₈ alkyl, C₁-C₆ alkyl, or C₁-C₄ alkyl). In embodiments, R^{3A} is substituted or unsubstituted heteroalkyl (e.g., 2 to 8 membered heteroalkyl, 2 to 6 membered heteroalkyl, or 2 to 4 membered heteroalkyl). In embodiments, R^{3A} is substituted heteroalkyl (e.g., 2 to 8 membered heteroalkyl, 2 to 6 membered heteroalkyl, or 2 to 4 membered heteroalkyl). In embodiments, R^{3A} is an unsubstituted heteroalkyl (e.g., 2 to 8 membered heteroalkyl, 2 to 6 membered heteroalkyl, or 2 to 4 membered heteroalkyl). In embodiments, R^{3A} is substituted or unsubstituted cycloalkyl (e.g., C₃-C₈ cycloalkyl, C₃-C₆ cycloalkyl, or C₅-C₆ cycloalkyl). In embodiments, R^{3A} is substituted cycloalkyl (e.g., C₃-C₈ cycloalkyl, C₃-C₆ cycloalkyl, or C₅-C₆ cycloalkyl). In embodiments, R^{3A} is an unsubstituted cycloalkyl (e.g., C₃-C₈ cycloalkyl, C₃-C₆ cycloalkyl, or C₅-C₆ cycloalkyl). In embodiments, R^{3A} is substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{3A} is substituted heterocycloalkyl (e.g., 3 to 8 membered heterocycloalkyl, 3 to 6 membered heterocycloalkyl, or 5 to 6 membered heterocycloalkyl). In embodiments, R^{3A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{3A} is substituted or unsubstituted aryl (e.g., C₆-C₁₀ aryl, C₁₀ aryl, or phenyl). In embodiments, R^{3A} is substituted aryl (e.g., C₆-C₁₀ aryl, C₁₀ aryl, or phenyl). In embodiments, R^{3A} is an unsubstituted aryl (e.g., C₆-C₁₀ aryl, C₁₀ aryl, or phenyl). In embodiments, R^{3A} is substituted or unsubstituted heteroaryl (e.g., 5 to 10 membered heteroaryl, 5 to 9 membered heteroaryl, or 5 to 6 membered heteroaryl). In embodiments, R^{3A} is substituted heteroaryl (e.g., 5 to 10 membered heteroaryl, 5 to 9 membered heteroaryl, or 5 to 6 membered heteroaryl). In embodiments, R^{3A} is an unsubstituted heteroaryl (e.g., 5 to 10 membered heteroaryl, 5 to 9 membered heteroaryl, or 5 to 6 membered heteroaryl).

In Formula II, III, XIV-C, and XV-C as provided herein, including embodiments thereof, L¹ may be substituted or unsubstituted C₁-C₃ alkylene. In embodiments, L¹ is substituted or unsubstituted methylene. In embodiments, L¹ is substituted or unsubstituted C₁-C₆ alkylene, or substituted or unsubstituted 2 to 6 membered heteroalkylene. In embodiments, L¹ is substituted or unsubstituted C₁-C₃ alkylene, or substituted or unsubstituted 2 to 3 membered heteroalkylene.

In embodiments, L¹ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹ is unsubstituted alkylene, unsubstituted heteroalkylene, unsubstituted cycloalkylene, unsubstituted heterocycloalkylene, unsubstituted arylene, or unsubstituted heteroarylene. In embodiments, L¹ is unsubstituted alkylene (e.g., C₁-C₆ alkylene). In embodiments, L¹ is a bond.

In embodiments, the L¹ is —CH₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is —CH₂—O—.

In embodiments, L¹ is —CH₂—O—,

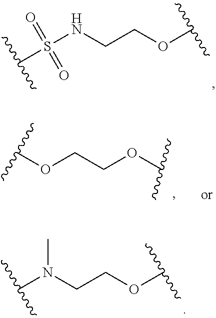

In embodiments, L¹ is —CH₂—O—. In embodiments, L¹ is

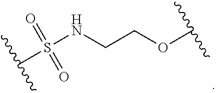 [SMILES: *OCCNS(*)(=O)=O]

In embodiments, L¹ is

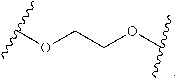 [SMILES: *OCCO*]

In embodiments, L¹ is

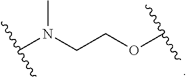 [SMILES: *OCCN(*)C]

In Formula (II), (III), (XIV-C), and (XV-C), as provided herein, including embodiments thereof, L² may be substituted or unsubstituted C₁-C₃ alkylene. In embodiments, L² is substituted or unsubstituted methylene. In embodiments, L² is substituted or unsubstituted C₁-C₆ alkylene, or substituted or unsubstituted 2 to 6 membered heteroalkylene. In embodiments, L² is substituted or unsubstituted C₁-C₃ alkylene, or substituted or unsubstituted 2 to 3 membered heteroalkylene.

In embodiments, L² is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L² is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene. In embodiments, L² is unsubstituted alkylene (e.g., C₁-C₆ alkylene). In embodiments, L² is a bond.

In Formula (II), (III), (XIV-C), and (XV-C), as provided herein, including embodiments thereof, L⁴ may be substituted or unsubstituted C₁-C₃ alkylene. In embodiments, L⁴ is substituted or unsubstituted methylene. In embodiments, L⁴ is substituted or unsubstituted C₁-C₆ alkylene, or substituted or unsubstituted 2 to 6 membered heteroalkylene. In embodiments, L⁴ is substituted or unsubstituted C₁-C₃ alkylene, or substituted or unsubstituted 2 to 3 membered heteroalkylene.

L⁴ as provided herein may be an aliphatic linker, a peptide linker or a pegylated linker. In embodiments, L⁴ is substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L⁴ is substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L⁴ is unsubstituted alkylene, unsubstituted heteroalkylene, unsubstituted cycloalkylene, unsubstituted heterocycloalkylene, unsubstituted arylene, or unsubstituted heteroarylene. In embodiments, L⁴ is unsubstituted alkylene (e.g., C₁-C₆ alkylene). In embodiments, L⁴ is a bond.

In Formula (II), (III), (XIV-C), and (XV-C), as provided herein, including embodiments thereof, z2 may be an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25. In embodiments, z1 and z3 are independently integers from 0 to 90 (e.g. 5 to 90, 10 to 90 or 20 to 90), 0 to 80 (e.g. 5 to 80, 10 to 80 or 20 to 80), 0 to 70 (e.g. 5 to 70, 10 to 70 or 20 to 70), 0 to 50 (e.g. 5 to 50, 10 to 50 or 20 to 50) or 2 to 25. In embodiments, z1 and z3 are independently integers from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25. In embodiments, z4 is independently an integer from 1 to 90 (e.g. 5 to 90, 10 to 90 or 20 to 90), 1 to 80 (e.g. 5 to 80, 10 to 80 or 20 to 80), 1 to 70 (e.g. 5 to 70, 10 to 70 or 20 to 70), 1 to 50 (e.g. 5 to 50, 10 to 50 or 20 to 50) or 2 to 25. In embodiments, z4 is independently an integer from 2 to 90 (e.g. 5 to 90, 10 to 90 or 20 to 90), 2 to 80 (e.g. 5 to 80, 10 to 80 or 20 to 80), 2 to 70 (e.g. 5 to 70, 10 to 70 or 20 to 70), 2 to 50 (e.g. 5 to 50, 10 to 50 or 20 to 50) or 2 to 25.

In embodiments the pH-sensitive immolation domain has the structure of Formula:

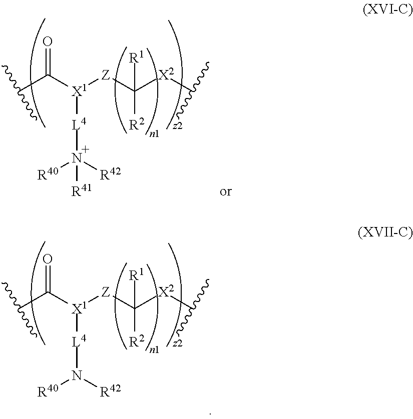

In formula (XVI-C) or (XVII-C) X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—. X² is —O— or —S—; R¹ and R² are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; L⁴ is a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene. R⁴⁰, R⁴¹, and R⁴² are independently hydrogen, substituted or unsubstituted alkyl or substituted or unsubstituted heteroalkyl; Z is —S—, —S⁺R¹³—, —NR¹³ —, or —N⁺(R¹³)(H)—; R¹³ is hydrogen, —CCl₃, —CBr₃, —CF₃, —CI₃, —CN, —OH, ═O, —NH₂, —COOH, —CONH₂, —SH, —SO₃H, —SO₂NH₂, —OCCl₃, —OCF₃, —OCBr₃, —OCl₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; n1 is an integer from 0 to 50; z2 is an integer from 2 to 100; and z5 is an integer from 1 to 10.

In Formula (II), (III), (XIV-C), (XV-C), (XVI-C) and (XVII-C), R¹, R², R⁵, R⁶, R⁷, and R⁸ may independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R⁵, R⁶, R⁷, and R⁸ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R⁵, R⁶, R⁷, and R⁸ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R⁵, R⁶, R⁷, and R⁸ are independently hydrogen or unsubstituted alkyl (e.g., C₁-C₆ alkyl). In embodiments, R¹, R², R⁵, R⁶, R⁷, and R⁸ are hydrogen.

In embodiments, the pH-sensitive immolation domain has the formula:

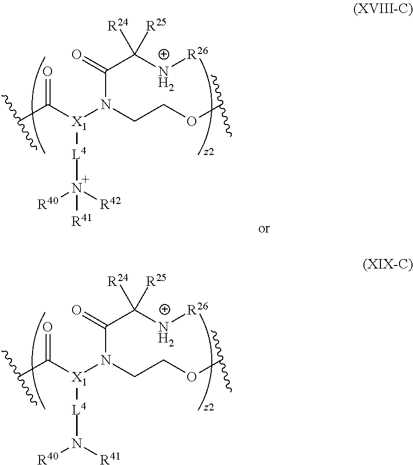

wherein R²⁴, R²⁵ and R²⁶ are hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and z2 is an integer from 1 to 50.

In embodiments, R²⁴, R²⁵ and R²⁶ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁴, R²⁵ and R²⁶ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{1.1}, R²⁴, R²⁵ and R²⁶ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R²⁴, R²⁵ and R²⁶ are independently hydrogen or unsubstituted alkyl (e.g., C₁-C₆ alkyl). In embodiments, R²⁴, R²⁵ and R²⁶ are independently hydrogen.

In Formula (II), (III), (XIV-C), (XV-C), (XVI-C), (XVII-C), (XVIII-C), and (XIX-C) provided herein, including embodiments thereof, R¹, R^{1A}, R^{2A}, R^{3A}, R⁵, R⁶, R⁷, R⁸, R¹³, R¹⁴, R¹⁵, R¹⁶, R¹⁷, R¹⁸, R¹⁹, R²⁴, R²⁵, R²⁶, R⁴⁰, R⁴¹, R⁴², R²⁰¹, R²⁰² and R²⁰³ may be independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R^{2A}, R^{3A}, R⁵, R⁶, R⁷, R⁸, R¹³, R¹⁴, R¹⁵, R¹⁶, R¹⁷, R¹⁸, R¹⁹, R²⁴, R²⁵, R²⁶, R⁴⁰, R⁴¹, R⁴², R²⁰¹, R²⁰² and R²⁰³ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R^{1A}, R^{2A}, R^{3A}, R⁵, R⁶, R⁷, R⁸, R¹³, R¹⁴, R¹⁵, R¹⁶, R¹⁷, R¹⁸, R¹⁹, R²⁴, R²⁵, R²⁶, R⁴⁰, R⁴¹, R⁴², R²⁰¹, R²⁰² and R²⁰³ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R^{1A}, R^{2A}, R^{3A}, R⁵, R⁶, R⁷, R⁸, R¹³, R¹⁴, R¹⁵, R¹⁶, R¹⁷, R¹⁸, R¹⁹, R²⁴, R²⁵, R²⁶, R⁴⁰, R⁴¹, R⁴², R²⁰¹, R²⁰² and R²⁰³ are independently hydrogen or unsubstituted alkyl (e.g., C₁-C₆ alkyl). In embodiments, R¹, R^{1A}, R^{2A}, R^{3A}, R⁵, R⁶, R⁷, R⁸, R¹³, R¹⁴, R¹⁵, R¹⁶, R¹⁷, R¹⁸, R¹⁹, R⁴⁰, R⁴¹, R⁴², R²⁰¹, R²⁰² and R²⁰³ are hydrogen.

In Formula (II), (III), (XIV-C), (XV-C), (XVI-C), (XVII-C), (XVIII-C), and (XIX-C) provided herein, including embodiments thereof, R⁴⁰, R⁴¹, and R⁴² may be independently substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R⁴⁰, R⁴¹, and R⁴², are independently substituted (e.g., substituted with a substituent group, a size-limited substituent group, or lower substituent group) or unsubstituted alkyl or substituted (e.g., substituted with a substituent group, a size-limited substituent group, or lower substituent group) or unsubstituted heteroalkyl. In embodiments, R⁴⁰, R⁴¹, and R⁴² are independently unsubstituted alkyl or unsubstituted heteroalkyl. In embodiments, R⁴⁰, R⁴¹, and R⁴² are independently hydrogen or unsubstituted alkyl (e.g., C₁-C₆ alkyl). In embodiments, R⁴⁰, R⁴¹, and R⁴² are hydrogen.

In embodiments, Z is a nucleophilic moiety. In embodiments, Z is —S—, —OR¹³—, —S⁺R¹³—, —NR¹³ —, or —N⁺(R¹³)(H)—, wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments Z is —S—. In embodiments, Z is —S⁺R¹³—. In embodiments, Z is —NR¹³—. In embodiments, Z is —N⁺(R¹³)(H)—. In embodiments, Z is —S⁺H—. In embodiments, Z is —NH—. In embodiments, Z is —N⁺H₂—. In embodiments, Z is —OH—. In embodiments, Z is —N⁺(R¹³)(H)— and R¹³ is hydrogen.

In embodiments, R¹³ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹³ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³ are independently hydrogen or unsubstituted alkyl (e.g., C₁-C₆ alkyl). In embodiments, R¹³ is hydrogen. In embodiments, R¹³ is —NH₃+. In embodiments, R¹³ is —NH₂.

In embodiments, R^{13A} are independently substituted or unsubstituted alkyl (e.g., C₁-C₈, C₁-C₆, C₁-C₄, or C₁-C₂), substituted or unsubstituerrted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R^{13A} are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R^{13A} are independently unsubstituted alkyl, unsubstituted heteroalkyl, unsubstituted cycloalkyl, unsubstituted heterocycloalkyl, unsubstituted aryl, or unsubstituted heteroaryl. In embodiments, R^{1A3} are independently hydrogen or unsubstituted alkyl (e.g., C₁-C₆ alkyl). In embodiments, R^{13A} is hydrogen. In embodiments, R^{13A} is —NH₃⁺. In embodiments, R^{13A} is —NH₂.

In embodiments, Z is

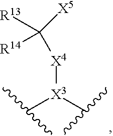

wherein X³ is —C(R¹⁵)— or —N—; X⁴ is a bond, —C(O)—, —P(O)(OR¹⁶)₂—, —S(O)(OR¹⁷)₂—, —C(R¹⁶)(R¹⁷)— or —C(R¹⁶)(R¹⁷)—C(R¹⁸)(R¹⁹)—, X⁵ is a nucleophilic moiety; R¹³, R¹⁴, R¹⁵, R¹⁶, R¹⁷, R¹⁸ and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, X³ is —CH.

In embodiments, R¹³, R¹⁴, R¹⁵, R¹⁶, R¹⁷, R¹⁸ and R¹⁹ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹³, R¹⁴, R¹⁵, R¹⁶, R¹⁷, R¹⁸ and R¹⁹ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments R¹³, R¹⁴, R¹⁵, R¹⁶, R¹⁷, R¹⁸ and R¹⁹ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹³, R¹⁴, R¹⁵, R¹⁶, R¹⁷, R¹⁸ and R¹⁹ are independently hydrogen or unsubstituted alkyl (e.g., C₁-C₆ alkyl).

In embodiments, X⁵ is —N⁺(R¹³)(H), wherein R¹³ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the pH-sensitive immolation domain has the formula (XX-C):

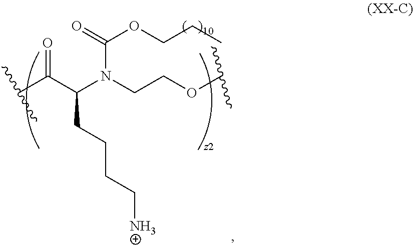

wherein z2 is as defined herein.

In embodiments, the pH-sensitive immolation domain has the formula (XXb-C):

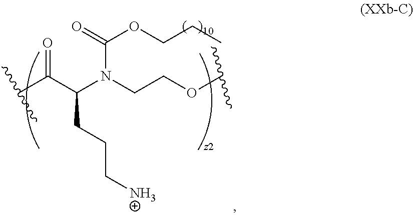

wherein z2 is as defined herein.

In embodiments, the pH-sensitive immolation domain has the formula (XXI-C):

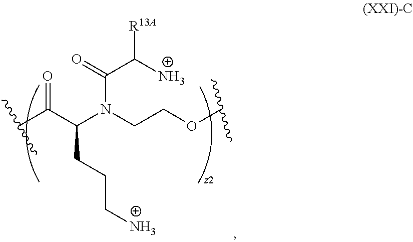

wherein z2 and R^{13A} are as defined herein.

In embodiments, the pH-sensitive immolation domain has the formula (XXIa-C):

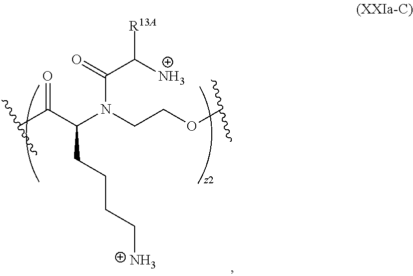

wherein z2 and R^{13A} are as defined herein.

In embodiments, the lipophilic polymer domain (LP¹ or LP²) has the formula:

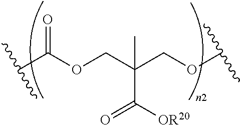

wherein, n2 is an integer from 1 to 100; R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl).

In embodiments, R²⁰ is an unsubstituted C₁-C₃₀ alkyl. In embodiments, R²⁰ is an unsubstituted C₁-C₂₀ alkyl. In embodiments, R²⁰ is an unsubstituted C₈-C₃₀ alkyl. In embodiments, R²⁰ is an unsubstituted C₈-C₂₀ alkyl. In embodiments, R²⁰ is an unsubstituted C₉-C₂₀ alkyl. In embodiments, R²⁰ is an unsubstituted C₉-C₁₈ alkyl. In embodiments, R²⁰ is an unsubstituted C₁₈ alkyl. In embodiments, R²⁰ is an unsubstituted C₁₇ alkyl. In embodiments, R²⁰ is an unsubstituted C₁₆ alkyl. In embodiments, R²⁰ is an unsubstituted C₁₅ alkyl. In embodiments, R²⁰ is an unsubstituted C₁₄ alkyl. In embodiments, R²⁰ is an unsubstituted C₁₃ alkyl. In embodiments, R²⁰ is an unsubstituted C₁₂ alkyl. In embodiments, R²⁰ is an unsubstituted C₁₁ alkyl. In embodiments, R²⁰ is an unsubstituted C₁₀ alkyl. In embodiments, R²⁰ is an unsubstituted C₉ alkyl. In embodiments, R²⁰ is an unsubstituted C₈ alkyl. In embodiments, R²⁰ is an unsubstituted C₇ alkyl. In embodiments, R²⁰ is an unsubstituted C₆ alkyl. In embodiments, R²⁰ is an unsubstituted C₅ alkyl. In embodiments, R²⁰ is an unsubstituted C₄ alkyl. In embodiments, R²⁰ is an unsubstituted C₃ alkyl. In embodiments, R²⁰ is an unsubstituted C₂ alkyl.

In embodiments, R²⁰ is an unsubstituted C₁-C₃₀ alkenyl. In embodiments, R²⁰ is an unsubstituted C₁-C₂₀ alkenyl. In embodiments, R²⁰ is an unsubstituted C₈-C₃₀ alkenyl. In embodiments, R²⁰ is an unsubstituted C₈-C₂₀ alkenyl. In embodiments, R²⁰ is an unsubstituted C₉-C₂₀ alkenyl. In embodiments, R²⁰ is an unsubstituted C₉-C₁₈ alkenyl. In embodiments, R²⁰ is an unsubstituted C₁₈ alkenyl. In embodiments, R²⁰ is an unsubstituted C₁₇ alkenyl. In embodiments, R²⁰ is an unsubstituted C₁₆ alkenyl. In embodiments, R²⁰ is an unsubstituted C₁₅ alkenyl. In embodiments, R²⁰ is an unsubstituted C₁₄ alkenyl. In embodiments, R²⁰ is an unsubstituted C₁₃ alkenyl. In embodiments, R²⁰ is an unsubstituted C₁₂ alkenyl. In embodiments, R²⁰ is an unsubstituted C₁₁ alkenyl. In embodiments, R²⁰ is an unsubstituted C₁₀ alkenyl. In embodiments, R²⁰ is an unsubstituted C₉ alkenyl. In embodiments, R²⁰ is an unsubstituted C₈ alkenyl. In embodiments, R²⁰ is an unsubstituted C₇ alkenyl. In embodiments, R²⁰ is an unsubstituted C₆ alkenyl. In embodiments, R²⁰ is an unsubstituted C₅ alkenyl. In embodiments, R²⁰ is an unsubstituted C₄ alkenyl. In embodiments, R²⁰ is an unsubstituted C₃ alkenyl. In embodiments, R²⁰ is an unsubstituted C₂ alkenyl.

In embodiments, R²⁰ is a stearyl moiety (e.g., an unsubstituted C₁₈ alkyl). In embodiments, R²⁰ is an oleyl moiety (e.g., an unsubstituted C₁₈ alkenyl). In embodiments, R²⁰ is an linoleyl moiety (e.g., an unsubstituted C₁₈ alkenyl). In embodiments, R²⁰ is an dodecyl moiety (e.g., an unsubstituted C₁₂ alkyl). In embodiments, R²⁰ is an nonenyl moiety (e.g., an unsubstituted C₉ alkenyl). In embodiments, R²⁰ is

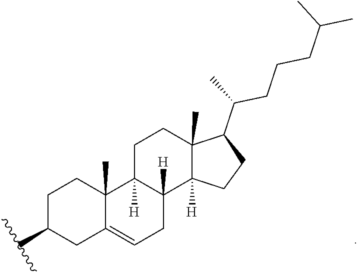

In embodiments, R¹ and R² are independently hydrogen or substituted or unsubstituted alkyl.

In embodiments, n1 is 2.

In embodiments, X² is —O—.

In embodiments, z1 or z3 are independently integers from 10-40.

In embodiments, z2 is independently an integer from 3-20.

In embodiments, LP¹ has the formula:

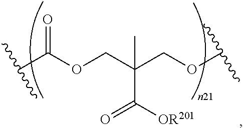

wherein n21 is an integer from 1 to 100;

R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, n21 is 10-40. In embodiments, R²⁰¹ is unsubstituted C₁₂ alkyl.

In embodiments, R²⁰¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰¹ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰¹ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰¹ is hydrogen or unsubstituted alkyl (e.g., C₁-C₆ alkyl).

In embodiments, R²⁰¹ is an unsubstituted C₁-C₃₀ alkyl. In embodiments, R²⁰¹ is an unsubstituted C₁-C₂₀ alkyl. In embodiments, R²⁰¹ is an unsubstituted C₈-C₃₀ alkyl. In embodiments, R²⁰¹ is an unsubstituted C₈-C₂₀ alkyl. In embodiments, R²⁰¹ is an unsubstituted C₉-C₂₀ alkyl. In embodiments, R²⁰¹ is an unsubstituted C₉-C₁₈ alkyl. In embodiments, R²⁰¹ is an unsubstituted C₁₈ alkyl. In embodiments, R²⁰¹ is an unsubstituted C₁₇ alkyl. In embodiments, R²⁰¹ is an unsubstituted C₁₆ alkyl. In embodiments, R²⁰¹ is an unsubstituted C₁₅ alkyl. In embodiments, R²⁰¹ is an unsubstituted C₁₄ alkyl. In embodiments, R²⁰¹ is an unsubstituted C₁₃ alkyl. In embodiments, R²⁰¹ is an unsubstituted C₁₂ alkyl. In embodiments, R²⁰¹ is an unsubstituted C₁₁ alkyl. In embodiments, R²⁰¹ is an unsubstituted C₁₀ alkyl. In embodiments, R²⁰¹ is an unsubstituted C₉ alkyl. In embodiments, R²⁰¹ is an unsubstituted C₈ alkyl. In embodiments, R²⁰¹ is an unsubstituted C₇ alkyl. In embodiments, R²⁰¹ is an unsubstituted C₆ alkyl. In embodiments, R²⁰¹ is an unsubstituted C₅ alkyl. In embodiments, R²⁰¹ is an unsubstituted C₄ alkyl. In embodiments, R²⁰¹ is an unsubstituted C₃ alkyl. In embodiments, R²⁰¹ is an unsubstituted C₂ alkyl.

In embodiments, R²⁰¹ is an unsubstituted C₁-C₃₀ alkenyl. In embodiments, R²⁰¹ is an unsubstituted C₁-C₂₀ alkenyl. In embodiments, R²⁰¹ is an unsubstituted C₈-C₃₀ alkenyl. In embodiments, R²⁰¹ is an unsubstituted C₈-C₂₀ alkenyl. In embodiments, R²⁰¹ is an unsubstituted C₉-C₂₀ alkenyl. In embodiments, R²⁰¹ is an unsubstituted C₉-C₁₈ alkenyl. In embodiments, R²⁰¹ is an unsubstituted C₁₈ alkenyl. In embodiments, R²⁰¹ is an unsubstituted C₁₇ alkenyl. In embodiments, R²⁰¹ is an unsubstituted C₁₆ alkenyl. In embodiments, R²⁰¹ is an unsubstituted C₁₅ alkenyl. In embodiments, R²⁰¹ is an unsubstituted C₁₄ alkenyl. In embodiments, R²⁰¹ is an unsubstituted C₁₃ alkenyl. In embodiments, R²⁰¹ is an unsubstituted C₁₂ alkenyl. In embodiments, R²⁰¹ is an unsubstituted C₁₁ alkenyl. In embodiments, R²⁰¹ is an unsubstituted C₁₀ alkenyl. In embodiments, R²⁰¹ is an unsubstituted C₉ alkenyl. In embodiments, R²⁰¹ is an unsubstituted C₈ alkenyl. In embodiments, R²⁰¹ is an unsubstituted C₇ alkenyl. In embodiments, R²⁰¹ is an unsubstituted C₆ alkenyl. In embodiments, R²⁰¹ is an unsubstituted C₅ alkenyl. In embodiments, R²⁰¹ is an unsubstituted C₄ alkenyl. In embodiments, R²⁰¹ is an unsubstituted C₃ alkenyl. In embodiments, R²⁰¹ is an unsubstituted C₂ alkenyl.

In embodiments, R²⁰¹ is a stearyl moiety (e.g., an unsubstituted C₁₈ alkyl). In embodiments, R²⁰¹ is an oleyl moiety (e.g., an unsubstituted C₁₈ alkenyl). In embodiments, R²⁰¹ is an linoleyl moiety (e.g., an unsubstituted C₁₈ alkenyl). In embodiments, R²⁰¹ is an dodecyl moiety (e.g., an unsubstituted C₁₂ alkyl). In embodiments, R²⁰¹ is an nonenyl moiety (e.g., an unsubstituted C₉ alkenyl). In embodiments, R²⁰¹ is

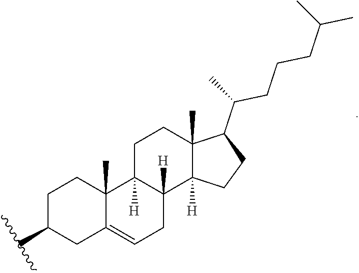

In embodiments, n21 is 5 and R²⁰¹ is unsubstituted C₁₈ alkenyl. In embodiments, n21 is 6 and R²⁰¹ is unsubstituted C₁₈ alkenyl. In embodiments, the unsubstituted C₁₈ alkenyl is oleyl.

In embodiments, LP² has the formula:

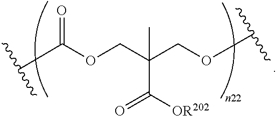

n22 is an integer from 1 to 100. R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R²⁰² is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰² is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰² is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰² is hydrogen or unsubstituted alkyl (e.g., C₁-C₆ alkyl).

In embodiments, R²⁰² is an unsubstituted C₁-C₃₀ alkyl. In embodiments, R²⁰² is an unsubstituted C₁-C₂₀ alkyl. In embodiments, R²⁰² is an unsubstituted C₈-C₃₀ alkyl. In embodiments, R²⁰² is an unsubstituted C₈-C₂₀ alkyl. In embodiments, R²⁰² is an unsubstituted C₉-C₂₀ alkyl. In embodiments, R²⁰² is an unsubstituted C₉-C₁₈ alkyl. In embodiments, R²⁰² is an unsubstituted C₁₃ alkyl. In embodiments, R²⁰² is an unsubstituted C₁₇ alkyl. In embodiments, R²⁰² is an unsubstituted C₁₆ alkyl. In embodiments, R²⁰² is an unsubstituted C₁₅ alkyl. In embodiments, R²⁰² is an unsubstituted C₁₄ alkyl. In embodiments, R²⁰² is an unsubstituted C₁₃ alkyl. In embodiments, R²⁰² is an unsubstituted C₁₂ alkyl. In embodiments, R²⁰² is an unsubstituted C₁₁ alkyl. In embodiments, R²⁰² is an unsubstituted C₁₀ alkyl. In embodiments, R²⁰² is an unsubstituted C₉ alkyl. In embodiments, R²⁰² is an unsubstituted C₈ alkyl. In embodiments, R²⁰² is an unsubstituted C₇ alkyl. In embodiments, R²⁰² is an unsubstituted C₆ alkyl. In embodiments, R²⁰² is an unsubstituted C₅ alkyl. In embodiments, R²⁰² is an unsubstituted C₄ alkyl. In embodiments, R²⁰² is an unsubstituted C₃ alkyl. In embodiments, R²⁰² is an unsubstituted C₂ alkyl.

In embodiments, R²⁰² is an unsubstituted C₁-C₃₀ alkenyl. In embodiments, R²⁰² is an unsubstituted C₁-C₂₀ alkenyl. In embodiments, R²⁰² is an unsubstituted C₈-C₃₀ alkenyl. In embodiments, R²⁰² is an unsubstituted C₈-C₂₀ alkenyl. In embodiments, R²⁰² is an unsubstituted C₉-C₂₀ alkenyl. In embodiments, R²⁰² is an unsubstituted C₉-C₁₈ alkenyl. In embodiments, R²⁰² is an unsubstituted C₁₃ alkenyl. In embodiments, R²⁰² is an unsubstituted C₁₇ alkenyl. In embodiments, R²⁰² is an unsubstituted C₁₆ alkenyl. In embodiments, R²⁰² is an unsubstituted C₁₃ alkenyl. In embodiments, R²⁰² is an unsubstituted C₁₄ alkenyl. In embodiments, R²⁰² is an unsubstituted C₁₃ alkenyl. In embodiments, R²⁰² is an unsubstituted C₁₂ alkenyl. In embodiments, R²⁰² is an unsubstituted C₁₁ alkenyl. In embodiments, R²⁰² is an unsubstituted C₁₀ alkenyl. In embodiments, R²⁰² is an unsubstituted C₉ alkenyl. In embodiments, R²⁰² is an unsubstituted C₈ alkenyl. In embodiments, R²⁰² is an unsubstituted C₇ alkenyl. In embodiments, R²⁰² is an unsubstituted C₆ alkenyl. In embodiments, R²⁰² is an unsubstituted C₅ alkenyl. In embodiments, R²⁰² is an unsubstituted C₄ alkenyl. In embodiments, R²⁰² is an unsubstituted C₃ alkenyl. In embodiments, R²⁰² is an unsubstituted C₂ alkenyl.

In embodiments, R²⁰² is a stearyl moiety (e.g., an unsubstituted C₁₈ alkyl). In embodiments, R²⁰² is an oleyl moiety (e.g., an unsubstituted C₁₃ alkenyl). In embodiments, R²⁰² is an linoleyl moiety (e.g., an unsubstituted C₁₃ alkenyl). In embodiments, R²⁰² is an dodecyl moiety (e.g., an unsubstituted C₁₂ alkyl). In embodiments, R²⁰² is an nonenyl moiety (e.g., an unsubstituted C₉ alkenyl). In embodiments, R²⁰² is

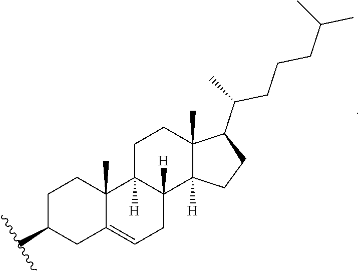

In embodiments, n22 is 10-35. In embodiments, R²⁰² is unsubstituted C₁₂ alkenyl.

In embodiments, n22 is 5 and R²⁰² is unsubstituted C₉ alkenyl. In embodiments, n22 is 6 and R²⁰² is unsubstituted C₉ alkenyl. In embodiments, the unsubstituted C₉ alkenyl is nonenyl.

In embodiments, the lipophilic polymer domain is a compound of Formula (Ia-C) following:

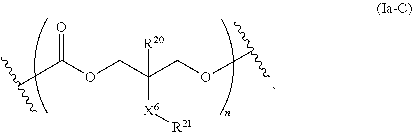

wherein X⁶ may be —O—, —NH—, —CO₂—, —CONH—, —O₂C—, or —NHCO—, R²⁰ is hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, R²¹ is hydrogen, substituted or unsubstituted alkyl, and n is an integer from 1 to 100. In embodiments, R²⁰ is an oligoglycol moiety.

In embodiments, R²⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰ is hydrogen or unsubstituted alkyl (e.g., C₁-C₆ alkyl).

In embodiments, R²¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²¹ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²¹ is unsubstituted alkyl, unsubstituted heteroalkyl, unsubstituted cycloalkyl, unsubstituted heterocycloalkyl, unsubstituted aryl, or unsubstituted heteroaryl. In embodiments, R²¹ is hydrogen or unsubstituted alkyl (e.g., C₁-C₆ alkyl).

In embodiments, the lipophilic polymer has the structure:

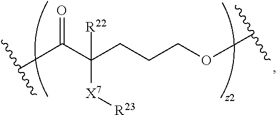

wherein X⁷ is —O—, —NH—, —CO₂—, —CONH—, —O₂C—, or —NHCO—; R²² is hydrogen, or substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl, and R²³ is substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, R²² is an oligoglycol moiety.

In embodiments, R²² is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²² is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²² is unsubstituted alkyl, unsubstituted heteroalkyl, unsubstituted cycloalkyl, unsubstituted heterocycloalkyl, unsubstituted aryl, or unsubstituted heteroaryl. In embodiments, R²² is hydrogen or unsubstituted alkyl (e.g., C₁-C₆ alkyl).

In embodiments, the lipophilic polymer domain (e.g., LP¹, LP²) has the Formula:

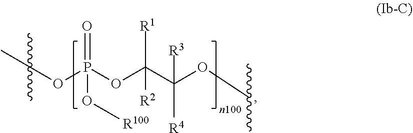

wherein R¹⁰⁰ is hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl. R¹, R², R³, R⁴ are hydrogen, substituted or unsubstituted heteroalkyl, substituted or unsubstituted heterocycloalkyl, substituted or unsubstituted aryl, or substituted or unsubstituted heteroaryl, and n100 is an integer of 2 or more is as defined herein.

In embodiments, R¹, R², R³, R⁴ are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R¹, R², R³, R⁴ are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R¹, R², R³, R⁴ are independently unsubstituted alkyl, unsubstituted heteroalkyl, unsubstituted cycloalkyl, unsubstituted heterocycloalkyl, unsubstituted aryl, or unsubstituted heteroaryl. In embodiments, R¹, R², R³, R⁴ are independently hydrogen or unsubstituted alkyl (e.g., C₁-C₆ alkyl). In embodiments, R¹, R², R³, R⁴ are hydrogen.

In embodiments, the lipophilic polymer domain has the Formula (Ic-C):

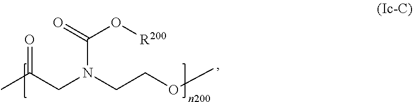

wherein R²⁰⁰ is substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, and n200 is an integer of 2 or more. In embodiments R²⁰⁰ is an oligoglycol moiety. In embodiments, R²⁰⁰ is an amine-terminated oligoglycol moiety. The term “oligoglycol moiety” refers to

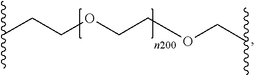

and “amine-terminated oligoglycol moiety” refers to

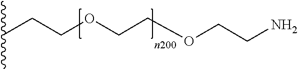

wherein n200 is an integer of 2 or more.

In embodiments, R²⁰⁰ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰⁰ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰⁰ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰⁰ is hydrogen or unsubstituted alkyl (e.g., C₁-C₆ alkyl). In embodiments, R²⁰⁰ is hydrogen.

In embodiments, the lipophilic polymer domain has the formula:

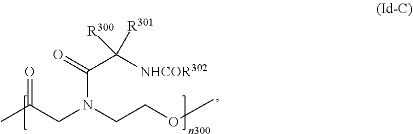

wherein R is substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl, R³⁰⁰ and R³⁰¹ are independently hydrogen or substituted or unsubstituted alkyl, and n300 is as defined herein. In embodiments R³⁰² is an oligoglycol moiety. In embodiments, R is an amine-terminated an oligoglycol moiety. In embodiments R³⁰⁰, R³⁰¹, and R³⁰² are hydrogen.

In embodiments, R³⁰⁰, R³⁰¹, and R³⁰² are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R³⁰⁰, R³⁰¹, and R³⁰² are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R³⁰⁰, R³⁰¹, and R³⁰² are independently unsubstituted alkyl, unsubstituted heteroalkyl, unsubstituted cycloalkyl, unsubstituted heterocycloalkyl, unsubstituted aryl, or unsubstituted heteroaryl. In embodiments, R³⁰⁰, R³⁰¹, and R³⁰² are independently hydrogen or unsubstituted alkyl (e.g., C₁-C₆ alkyl).

In embodiments, the lipophilic polymer domain, has the below formula, wherein R is defined therein as stearyl, oleyl, linoleyl, dodecyl, noneyl and cholesterol:

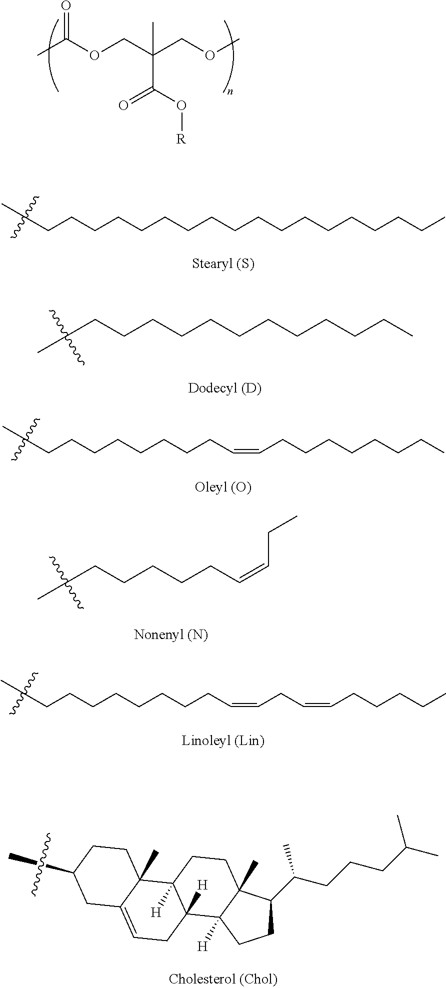

In embodiments, the cationic amphipathic polymer has the formula:

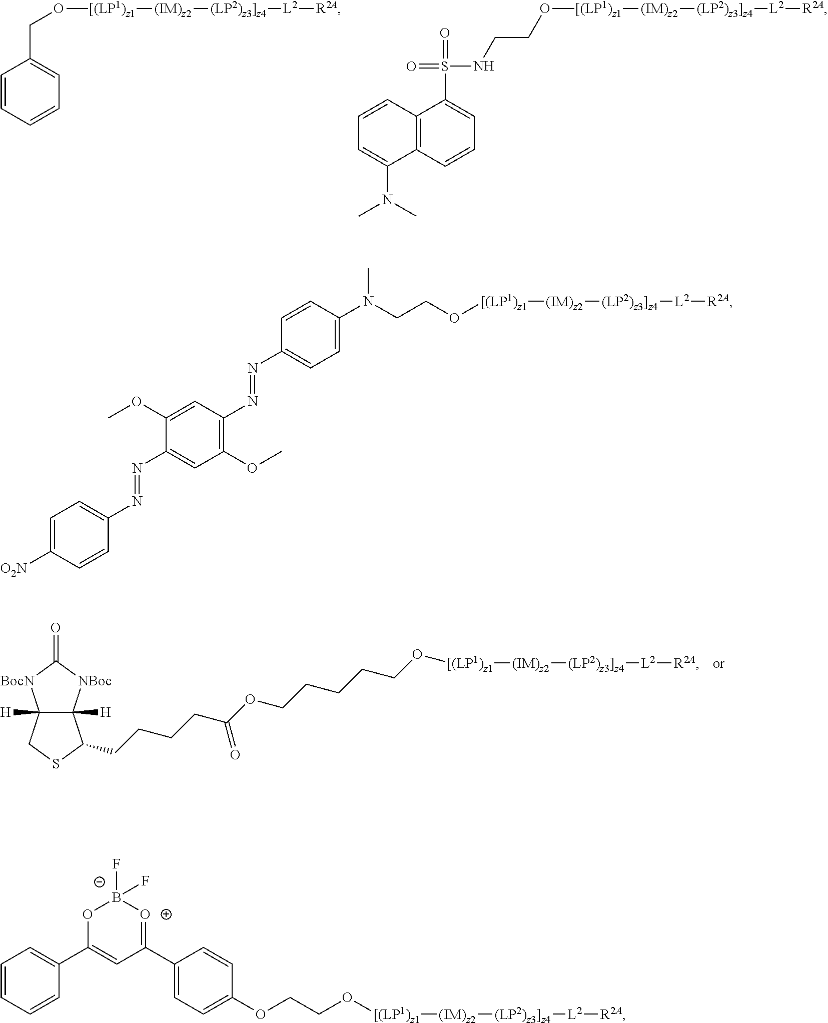

wherein LP¹, LP², IM, L², R^{2A}, z1, z2, z3, and z4 are defined as herein.

In embodiments, the cationic amphipathic polymer has the formula:

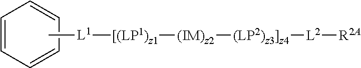

wherein LP¹, LP², IM, L¹, L², R^{2A}, z1, z2, z3, and z4 are defined as herein. In embodiments, the cationic amphipathic polymer has the formula:

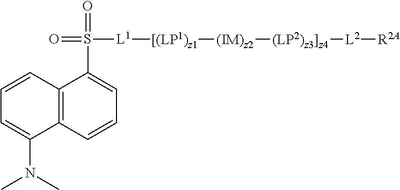

wherein LP¹, LP², IM, L¹, L², R^{2A}, z1, z2, z3, and z4 are defined as herein. In embodiments, the cationic amphipathic polymer has the formula:

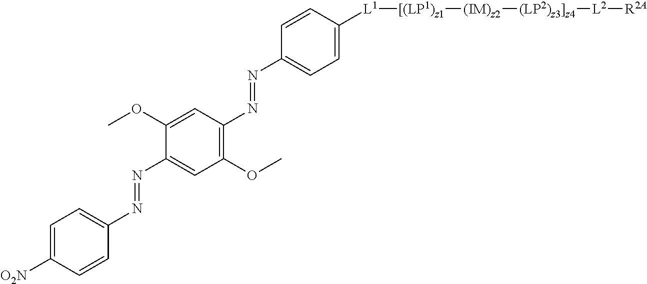

wherein LP¹, LP², IM, L¹, L², R^{2A}, z1, z2, z3, and z4 are defined as herein. In embodiments, the cationic amphipathic polymer has the formula:

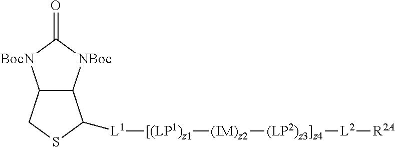

herein LP¹, LP², IM, L¹, L², R^{2A}, z1, z2, z3, and z4 are defined as herein. In embodiments, the cationic amphipathic polymer has the formula:

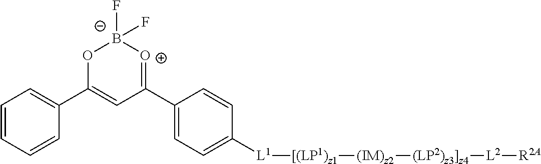

herein LP¹, LP², IM, L¹, L², R^{2A}, z1, z2, z3, and z4 are defined as herein.

In embodiments, z1, z3 and z4 can be independently integers in the range 2-100, 2-90, 2-80, 2-70, 2-60, 2-50, 2-40, 2-30, 2-2, or 2-10, wherein at least one of z1 or z3 is not 0. In embodiments, z1, z3 and z4 can be independently integers in the range 2-100 or 2-50, wherein at least one of z1 or z3 is not 0.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7. In embodiments, z1 is 8. In embodiments, z1 is 9. In embodiments, z1 is 10. In embodiments, z1 is 11. In embodiments, z1 is 12. In embodiments, z1 is 13. In embodiments, z1 is 14. In embodiments, z1 is 15. In embodiments, z1 is 16. In embodiments, z1 is 17. In embodiments, z1 is 18. In embodiments, z1 is 19. In embodiments, z1 is 20. In embodiments, z1 is 21. In embodiments, z1 is 22. In embodiments, z1 is 23. In embodiments, z1 is 24. In embodiments, z1 is 25. In embodiments, z1 is 26. In embodiments, z1 is 27. In embodiments, z1 is 28. In embodiments, z1 is 29. In embodiments, z1 is 30. In embodiments, z1 is 31. In embodiments, z1 is 32. In embodiments, z1 is 33. In embodiments, z1 is 34. In embodiments, z1 is 35. In embodiments, z1 is 36. In embodiments, z1 is 37. In embodiments, z1 is 38. In embodiments, z1 is 39. In embodiments, z1 is 40. In embodiments, z1 is 41. In embodiments, z1 is 42. In embodiments, z1 is 43. In embodiments, z1 is 44. In embodiments, z1 is 45. In embodiments, z1 is 46. In embodiments, z1 is 47. In embodiments, z1 is 48. In embodiments, z1 is 49. In embodiments, z1 is 50. In embodiments, z1 is 51. In embodiments, z1 is 52. In embodiments, z1 is 53. In embodiments, z1 is 54. In embodiments, z1 is 55. In embodiments, z1 is 56. In embodiments, z1 is 57. In embodiments, z1 is 58. In embodiments, z1 is 59. In embodiments, z1 is 60. In embodiments, z1 is 61. In embodiments, z1 is 62. In embodiments, z1 is 63. In embodiments, z1 is 64. In embodiments, z1 is 65. In embodiments, z1 is 66. In embodiments, z1 is 67. In embodiments, z1 is 68. In embodiments, z1 is 69. In embodiments, z1 is 70. In embodiments, z1 is 71. In embodiments, z1 is 72. In embodiments, z1 is 73. In embodiments, z1 is 74. In embodiments, z1 is 75. In embodiments, z1 is 76. In embodiments, z1 is 77. In embodiments, z1 is 78. In embodiments, z1 is 79. In embodiments, z1 is 80. In embodiments, z1 is 81. In embodiments, z1 is 82. In embodiments, z1 is 83. In embodiments, z1 is 84. In embodiments, z1 is 85. In embodiments, z1 is 86. In embodiments, z1 is 87. In embodiments, z1 is 88. In embodiments, z1 is 89. In embodiments, z1 is 90. In embodiments, z1 is 91. In embodiments, z1 is 92. In embodiments, z1 is 93. In embodiments, z1 is 94. In embodiments, z1 is 95. In embodiments, z1 is 96. In embodiments, z1 is 97. In embodiments, z1 is 98. In embodiments, z1 is 99. In embodiments, z1 is 100.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11. In embodiments, z3 is 12. In embodiments, z3 is 13. In embodiments, z3 is 14. In embodiments, z3 is 15. In embodiments, z3 is 16. In embodiments, z3 is 17. In embodiments, z3 is 18. In embodiments, z3 is 19. In embodiments, z3 is 20. In embodiments, z3 is 21. In embodiments, z3 is 22. In embodiments, z3 is 23. In embodiments, z3 is 24. In embodiments, z3 is 25. In embodiments, z3 is 26. In embodiments, z3 is 27. In embodiments, z3 is 28. In embodiments, z3 is 29. In embodiments, z3 is 30. In embodiments, z3 is 31. In embodiments, z3 is 32. In embodiments, z3 is 33. In embodiments, z3 is 34. In embodiments, z3 is 35. In embodiments, z3 is 36. In embodiments, z3 is 37. In embodiments, z3 is 38. In embodiments, z3 is 39. In embodiments, z3 is 40. In embodiments, z3 is 41. In embodiments, z3 is 42. In embodiments, z3 is 43. In embodiments, z3 is 44. In embodiments, z3 is 45. In embodiments, z3 is 46. In embodiments, z3 is 47. In embodiments, z3 is 48. In embodiments, z3 is 49. In embodiments, z3 is 50. In embodiments, z3 is 51. In embodiments, z3 is 52. In embodiments, z3 is 53. In embodiments, z3 is 54. In embodiments, z3 is 55. In embodiments, z3 is 56. In embodiments, z3 is 57. In embodiments, z3 is 58. In embodiments, z3 is 59. In embodiments, z3 is 60. In embodiments, z3 is 61. In embodiments, z3 is 62. In embodiments, z3 is 63. In embodiments, z3 is 64. In embodiments, z3 is 65. In embodiments, z3 is 66. In embodiments, z3 is 67. In embodiments, z3 is 68. In embodiments, z3 is 69. In embodiments, z3 is 70. In embodiments, z3 is 71. In embodiments, z3 is 72. In embodiments, z3 is 73. In embodiments, z3 is 74. In embodiments, z3 is 75. In embodiments, z3 is 76. In embodiments, z3 is 77. In embodiments, z3 is 78. In embodiments, z3 is 79. In embodiments, z3 is 80. In embodiments, z3 is 81. In embodiments, z3 is 82. In embodiments, z3 is 83. In embodiments, z3 is 84. In embodiments, z3 is 85. In embodiments, z3 is 86. In embodiments, z3 is 87. In embodiments, z3 is 88. In embodiments, z3 is 89. In embodiments, z3 is 90. In embodiments, z3 is 91. In embodiments, z3 is 92. In embodiments, z3 is 93. In embodiments, z3 is 94. In embodiments, z3 is 95. In embodiments, z3 is 96. In embodiments, z3 is 97. In embodiments, z3 is 98. In embodiments, z3 is 99. In embodiments, z3 is 100.

In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11. In embodiments, z4 is 12. In embodiments, z4 is 13. In embodiments, z4 is 14. In embodiments, z4 is 15. In embodiments, z4 is 16. In embodiments, z4 is 17. In embodiments, z4 is 18. In embodiments, z4 is 19. In embodiments, z4 is 20. In embodiments, z4 is 21. In embodiments, z4 is 22. In embodiments, z4 is 23. In embodiments, z4 is 24. In embodiments, z4 is 25. In embodiments, z4 is 26. In embodiments, z4 is 27. In embodiments, z4 is 28. In embodiments, z4 is 29. In embodiments, z4 is 30. In embodiments, z4 is 31. In embodiments, z4 is 32. In embodiments, z4 is 33. In embodiments, z4 is 34. In embodiments, z4 is 35. In embodiments, z4 is 36. In embodiments, z4 is 37. In embodiments, z4 is 38. In embodiments, z4 is 39. In embodiments, z4 is 40. In embodiments, z4 is 41. In embodiments, z4 is 42. In embodiments, z4 is 43. In embodiments, z4 is 44. In embodiments, z4 is 45. In embodiments, z4 is 46. In embodiments, z4 is 47. In embodiments, z4 is 48. In embodiments, z4 is 49. In embodiments, z4 is 50. In embodiments, z4 is 51. In embodiments, z4 is 52. In embodiments, z4 is 53. In embodiments, z4 is 54. In embodiments, z4 is 55. In embodiments, z4 is 56. In embodiments, z4 is 57. In embodiments, z4 is 58. In embodiments, z4 is 59. In embodiments, z4 is 60. In embodiments, z4 is 61. In embodiments, z4 is 62. In embodiments, z4 is 63. In embodiments, z4 is 64. In embodiments, z4 is 65. In embodiments, z4 is 66. In embodiments, z4 is 67. In embodiments, z4 is 68. In embodiments, z4 is 69. In embodiments, z4 is 70. In embodiments, z4 is 71. In embodiments, z4 is 72. In embodiments, z4 is 73. In embodiments, z4 is 74. In embodiments, z4 is 75. In embodiments, z4 is 76. In embodiments, z4 is 77. In embodiments, z4 is 78. In embodiments, z4 is 79. In embodiments, z4 is 80. In embodiments, z4 is 81. In embodiments, z4 is 82. In embodiments, z4 is 83. In embodiments, z4 is 84. In embodiments, z4 is 85. In embodiments, z4 is 86. In embodiments, z4 is 87. In embodiments, z4 is 88. In embodiments, z4 is 89. In embodiments, z4 is 90. In embodiments, z4 is 91. In embodiments, z4 is 92. In embodiments, z4 is 93. In embodiments, z4 is 94. In embodiments, z4 is 95. In embodiments, z4 is 96. In embodiments, z4 is 97. In embodiments, z4 is 98. In embodiments, z4 is 99. In embodiments, z4 is 100.

In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24. In embodiments, n is 25. In embodiments, n is 26. In embodiments, n is 27. In embodiments, n is 28. In embodiments, n is 29. In embodiments, n is 30. In embodiments, n is 31. In embodiments, n is 32. In embodiments, n is 33. In embodiments, n is 34. In embodiments, n is 35. In embodiments, n is 36. In embodiments, n is 37. In embodiments, n is 38. In embodiments, n is 39. In embodiments, n is 40. In embodiments, n is 41. In embodiments, n is 42. In embodiments, n is 43. In embodiments, n is 44. In embodiments, n is 45. In embodiments, n is 46. In embodiments, n is 47. In embodiments, n is 48. In embodiments, n is 49. In embodiments, n is 50. In embodiments, n is 51. In embodiments, n is 52. In embodiments, n is 53. In embodiments, n is 54. In embodiments, n is 55. In embodiments, n is 56. In embodiments, n is 57. In embodiments, n is 58. In embodiments, n is 59. In embodiments, n is 60. In embodiments, n is 61. In embodiments, n is 62. In embodiments, n is 63. In embodiments, n is 64. In embodiments, n is 65. In embodiments, n is 66. In embodiments, n is 67. In embodiments, n is 68. In embodiments, n is 69. In embodiments, n is 70. In embodiments, n is 71. In embodiments, n is 72. In embodiments, n is 73. In embodiments, n is 74. In embodiments, n is 75. In embodiments, n is 76. In embodiments, n is 77. In embodiments, n is 78. In embodiments, n is 79. In embodiments, n is 80. In embodiments, n is 81. In embodiments, n is 82. In embodiments, n is 83. In embodiments, n is 84. In embodiments, n is 85. In embodiments, n is 86. In embodiments, n is 87. In embodiments, n is 88. In embodiments, n is 89. In embodiments, n is 90. In embodiments, n is 91. In embodiments, n is 92. In embodiments, n is 93. In embodiments, n is 94. In embodiments, n is 95. In embodiments, n is 96. In embodiments, n is 97. In embodiments, n is 98. In embodiments, n is 99. In embodiments, n is 100.

In embodiments, n1 is 0. In embodiments, n1 is 1. In embodiments, n1 is 2. In embodiments, n1 is 3. In embodiments, n1 is 4. In embodiments, n1 is 5. In embodiments, n1 is 6. In embodiments, n1 is 7. In embodiments, n1 is 8. In embodiments, n1 is 9. In embodiments, n1 is 10. In embodiments, n1 is 11. In embodiments, n1 is 12. In embodiments, n1 is 13. In embodiments, n1 is 14. In embodiments, n1 is 15. In embodiments, n1 is 16. In embodiments, n1 is 17. In embodiments, n1 is 18. In embodiments, n1 is 19. In embodiments, n1 is 20. In embodiments, n1 is 21. In embodiments, n1 is 22. In embodiments, n1 is 23. In embodiments, n1 is 24. In embodiments, n1 is 25. In embodiments, n1 is 26. In embodiments, n1 is 27. In embodiments, n1 is 28. In embodiments, n1 is 29. In embodiments, n1 is 30. In embodiments, n1 is 31. In embodiments, n1 is 32. In embodiments, n1 is 33. In embodiments, n1 is 34. In embodiments, n1 is 35. In embodiments, n1 is 36. In embodiments, n1 is 37. In embodiments, n1 is 38. In embodiments, n1 is 39. In embodiments, n1 is 40. In embodiments, n1 is 41. In embodiments, n1 is 42. In embodiments, n1 is 43. In embodiments, n1 is 44. In embodiments, n1 is 45. In embodiments, n1 is 46. In embodiments, n1 is 47. In embodiments, n1 is 48. In embodiments, n1 is 49. In embodiments, n1 is 50.

In embodiments, n2 is 1. In embodiments, n2 is 2. In embodiments, n2 is 3. In embodiments, n2 is 4. In embodiments, n2 is 5. In embodiments, n2 is 6. In embodiments, n2 is 7. In embodiments, n2 is 8. In embodiments, n2 is 9. In embodiments, n2 is 10. In embodiments, n2 is 11. In embodiments, n2 is 12. In embodiments, n2 is 13. In embodiments, n2 is 14. In embodiments, n2 is 15. In embodiments, n2 is 16. In embodiments, n2 is 17. In embodiments, n2 is 18. In embodiments, n2 is 19. In embodiments, n2 is 20. In embodiments, n2 is 21. In embodiments, n2 is 22. In embodiments, n2 is 23. In embodiments, n2 is 24. In embodiments, n2 is 25. In embodiments, n2 is 26. In embodiments, n2 is 27. In embodiments, n2 is 28. In embodiments, n2 is 29. In embodiments, n2 is 30. In embodiments, n2 is 31. In embodiments, n2 is 32. In embodiments, n2 is 33. In embodiments, n2 is 34. In embodiments, n2 is 35. In embodiments, n2 is 36. In embodiments, n2 is 37. In embodiments, n2 is 38. In embodiments, n2 is 39. In embodiments, n2 is 40. In embodiments, n2 is 41. In embodiments, n2 is 42. In embodiments, n2 is 43. In embodiments, n2 is 44. In embodiments, n2 is 45. In embodiments, n2 is 46. In embodiments, n2 is 47. In embodiments, n2 is 48. In embodiments, n2 is 49. In embodiments, n2 is 50. In embodiments, n2 is 51. In embodiments, n2 is 52. In embodiments, n2 is 53. In embodiments, n2 is 54. In embodiments, n2 is 55. In embodiments, n2 is 56. In embodiments, n2 is 57. In embodiments, n2 is 58. In embodiments, n2 is 59. In embodiments, n2 is 60. In embodiments, n2 is 61. In embodiments, n2 is 62. In embodiments, n2 is 63. In embodiments, n2 is 64. In embodiments, n2 is 65. In embodiments, n2 is 66. In embodiments, n2 is 67. In embodiments, n2 is 68. In embodiments, n2 is 69. In embodiments, n2 is 70. In embodiments, n2 is 71. In embodiments, n2 is 72. In embodiments, n2 is 73. In embodiments, n2 is 74. In embodiments, n2 is 75. In embodiments, n2 is 76. In embodiments, n2 is 77. In embodiments, n2 is 78. In embodiments, n2 is 79. In embodiments, n2 is 80. In embodiments, n2 is 81. In embodiments, n2 is 82. In embodiments, n2 is 83. In embodiments, n2 is 84. In embodiments, n2 is 85. In embodiments, n2 is 86. In embodiments, n2 is 87. In embodiments, n2 is 88. In embodiments, n2 is 89. In embodiments, n2 is 90. In embodiments, n2 is 91. In embodiments, n2 is 92. In embodiments, n2 is 93. In embodiments, n2 is 94. In embodiments, n2 is 95. In embodiments, n2 is 96. In embodiments, n2 is 97. In embodiments, n2 is 98. In embodiments, n2 is 99. In embodiments, n2 is 100.

In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z2 is 9. In embodiments, z2 is 10. In embodiments, z2 is 11. In embodiments, z2 is 12. In embodiments, z2 is 13. In embodiments, z2 is 14. In embodiments, z2 is 15. In embodiments, z2 is 16. In embodiments, z2 is 17. In embodiments, z2 is 18. In embodiments, z2 is 19. In embodiments, z2 is 20. In embodiments, z2 is 21. In embodiments, z2 is 22. In embodiments, z2 is 23. In embodiments, z2 is 24. In embodiments, z2 is 25. In embodiments, z2 is 26. In embodiments, z2 is 27. In embodiments, z2 is 28. In embodiments, z2 is 29. In embodiments, z2 is 30. In embodiments, z2 is 31. In embodiments, z2 is 32. In embodiments, z2 is 33. In embodiments, z2 is 34. In embodiments, z2 is 35. In embodiments, z2 is 36. In embodiments, z2 is 37. In embodiments, z2 is 38. In embodiments, z2 is 39. In embodiments, z2 is 40. In embodiments, z2 is 41. In embodiments, z2 is 42. In embodiments, z2 is 43. In embodiments, z2 is 44. In embodiments, z2 is 45. In embodiments, z2 is 46. In embodiments, z2 is 47. In embodiments, z2 is 48. In embodiments, z2 is 49. In embodiments, z2 is 50. In embodiments, z2 is 51. In embodiments, z2 is 52. In embodiments, z2 is 53. In embodiments, z2 is 54. In embodiments, z2 is 55. In embodiments, z2 is 56. In embodiments, z2 is 57. In embodiments, z2 is 58. In embodiments, z2 is 59. In embodiments, z2 is 60. In embodiments, z2 is 61. In embodiments, z2 is 62. In embodiments, z2 is 63. In embodiments, z2 is 64. In embodiments, z2 is 65. In embodiments, z2 is 66. In embodiments, z2 is 67. In embodiments, z2 is 68. In embodiments, z2 is 69. In embodiments, z2 is 70. In embodiments, z2 is 71. In embodiments, z2 is 72. In embodiments, z2 is 73. In embodiments, z2 is 74. In embodiments, z2 is 75. In embodiments, z2 is 76. In embodiments, z2 is 77. In embodiments, z2 is 78. In embodiments, z2 is 79. In embodiments, z2 is 80. In embodiments, z2 is 81. In embodiments, z2 is 82. In embodiments, z2 is 83. In embodiments, z2 is 84. In embodiments, z2 is 85. In embodiments, z2 is 86. In embodiments, z2 is 87. In embodiments, z2 is 88. In embodiments, z2 is 89. In embodiments, z2 is 90. In embodiments, z2 is 91. In embodiments, z2 is 92. In embodiments, z2 is 93. In embodiments, z2 is 94. In embodiments, z2 is 95. In embodiments, z2 is 96. In embodiments, z2 is 97. In embodiments, z2 is 98. In embodiments, z2 is 99. In embodiments, z2 is 100.

In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7. In embodiments, z5 is 8. In embodiments, z5 is 9. In embodiments, z5 is 10.

In embodiments, the cationic amphipathic polymer has any of the foregoing formula wherein z2 is an integer from 2 to 100. In embodiments, z2 can be an integer in the range 2-100, 2-90, 2-80, 2-70, 2-60, 2-50, 2-40, 2-30, 2-2, or 2-10. In embodiments, z2 is an integer from 2-100 or 2-50.

In embodiments, the cationic amphipathic polymer has any of the foregoing formula wherein z5 is an integer from 1 to 3. In other embodiments, z5 is 1 or 3. In still some other embodiments, z5 is 1. In some other embodiments, z5 is 3.

In embodiments, the cationic amphipathic polymer has any of the foregoing formula wherein R² is hydrogen.

In embodiments, the cationic amphipathic polymer has any of the foregoing formula wherein L² is a bond.

In embodiments, the cationic amphipathic polymer has the formula:

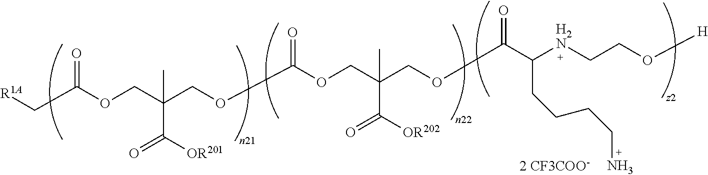

wherein n21 is 5, R²⁰¹ is oleyl, n22 is 5, R²⁰² is nonenyl and z2 is 7.

In embodiments, the cationic amphipathic polymer has the formula:

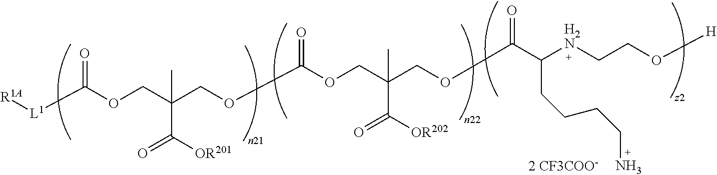

wherein n21 is 5, R²⁰¹ is oleyl, L¹ is —O—, n22 is 5, R²⁰² is nonenyl and z2 is 7.

In embodiments, the cationic amphipathic polymer has the formula:

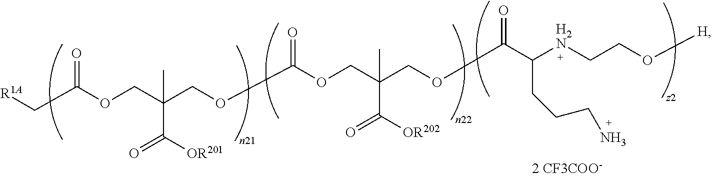

wherein n21 is 5, R²⁰¹ is oleyl, n22 is 5, R²⁰² is nonenyl and z2 is 7.

In embodiments, the cationic amphipathic polymer has the formula:

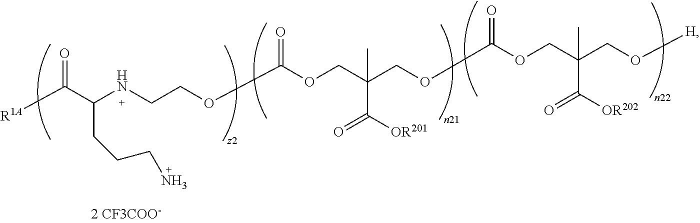

wherein n21 is 5, R²⁰¹ is oleyl, n22 is 5, R²⁰² is nonenyl and z2 is 7.

In embodiments, the cationic amphipathic polymer has the formula:

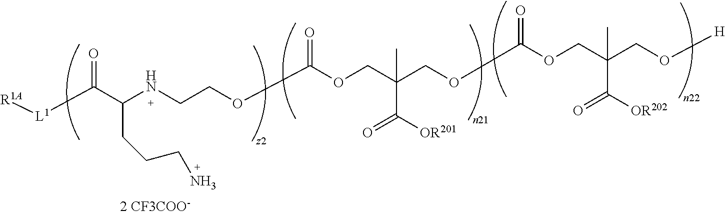

wherein n21 is 5, R²⁰¹ is oleyl, L¹ is —O—, n22 is 5, R²⁰² is nonenyl and z2 is 7.

In embodiments, the cationic amphipathic polymer has the formula:

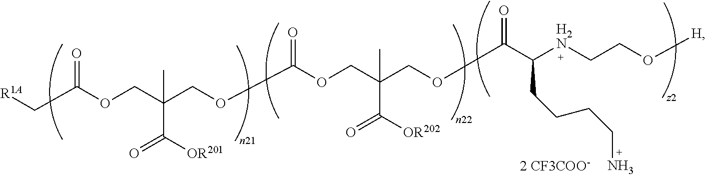

wherein n21 is 5, R²⁰¹ is oleyl, n22 is 5, R²⁰² is nonenyl and z2 is 7.

In embodiments, the cationic amphipathic polymer has the formula:

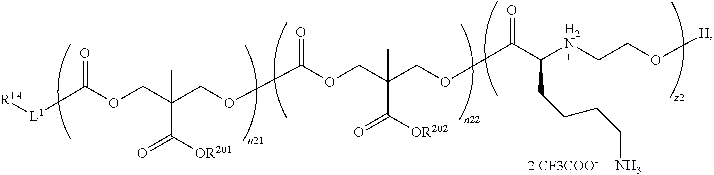

wherein n21 is 5, R²⁰¹ is oleyl, L¹ is —O—, n22 is 5, R²⁰² is nonenyl and z2 is 7.

In embodiments, the cationic amphipathic polymer has the formula:

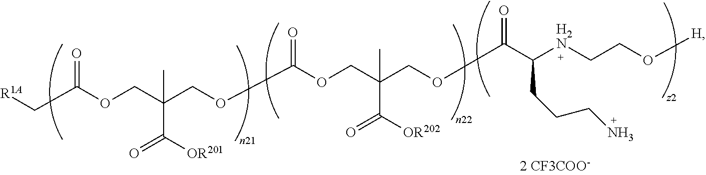

wherein n21 is 5, R²⁰¹ is oleyl, n22 is 5, R²⁰² is nonenyl and z2 is 7.

In embodiments, the cationic amphipathic polymer has the formula:

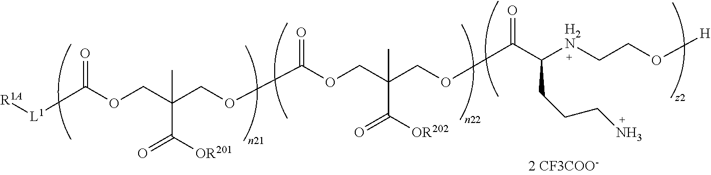

wherein n21 is 5, R²⁰¹ is oleyl, L¹ is —O—, n22 is 5, R²⁰² is nonenyl and z2 is 7.

In embodiments, the cationic amphipathic polymer has the formula:

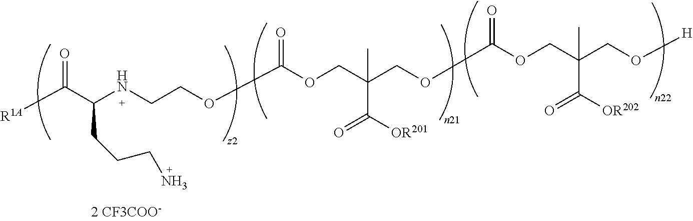

wherein n21 is 5, R²⁰¹ is oleyl, n22 is 5, R²⁰² is nonenyl and z2 is 7.

In embodiments, the cationic amphipathic polymer has the formula:

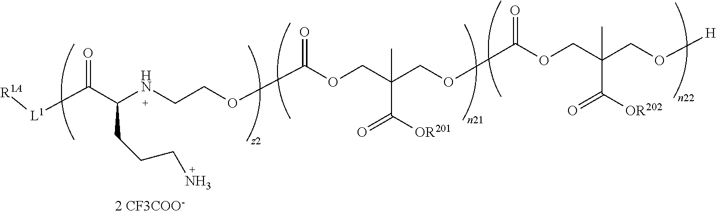

wherein n21 is 5, R²⁰¹ is oleyl, L¹ is —O—, n22 is 5, R²⁰² is nonenyl and z2 is 7.

In embodiments, the cationic amphipathic polymer has the formula:

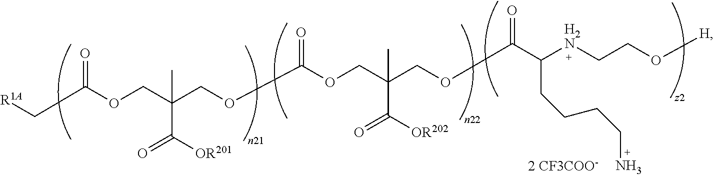

wherein n21 is 4, n22 is 4, and z2 is 8.

In embodiments, the cationic amphipathic polymer has the formula:

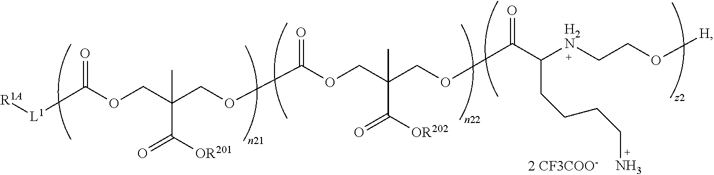

wherein n21 is 4, n22 is 4, and z2 is 8.

In embodiments, the cationic amphipathic polymer has the formula:

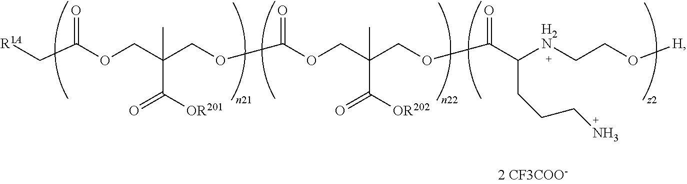

wherein n21 is 4, n22 is 4, and z2 is 8.

In embodiments, the cationic amphipathic polymer has the formula:

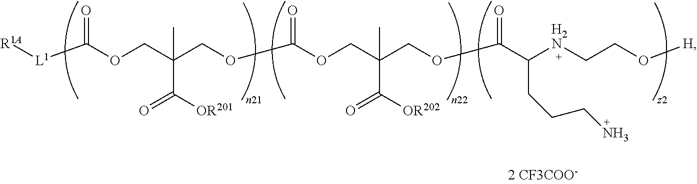

wherein n21 is 4, L¹ is —O—, n22 is 4, and z2 is 8.

In embodiments, the cationic amphipathic polymer has the formula:

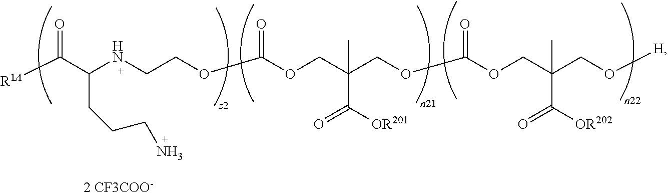

wherein n21 is 4, n22 is 4, and z2 is 8.

In embodiments, the cationic amphipathic polymer has the formula:

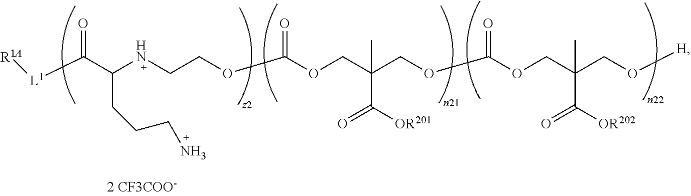

wherein n21 is 4, L¹ is —O—, n22 is 4, and z2 is 8.

In embodiments, the cationic amphipathic polymer has the formula:

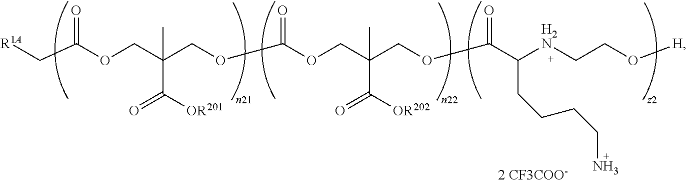

wherein n21 is 5, n22 is 6, and z2 is 10.

In embodiments, the cationic amphipathic polymer has the formula:

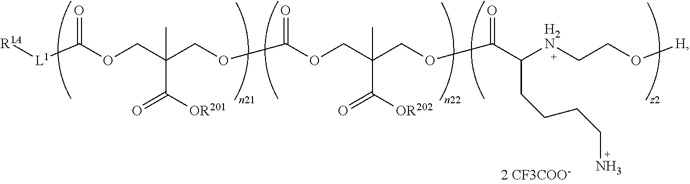

wherein n21 is 5, L¹ is —O—, n22 is 6, and z2 is 10.

In embodiments, the cationic amphipathic polymer has the formula:

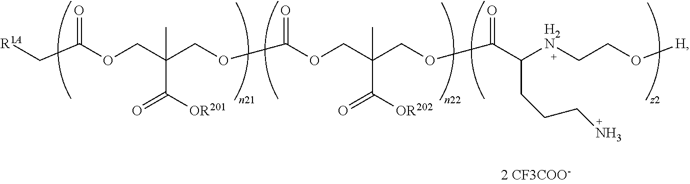

wherein n21 is 5, n22 is 6, and z2 is 10.

In embodiments, the cationic amphipathic polymer has the formula:

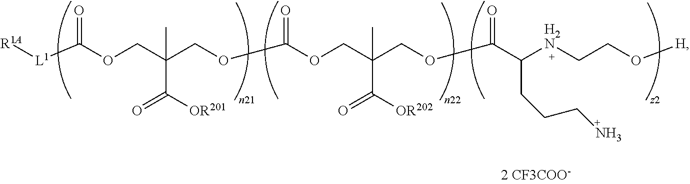

wherein n21 is 5, L¹ is —O—, n22 is 6, and z2 is 10.

In embodiments, the cationic amphipathic polymer has the formula:

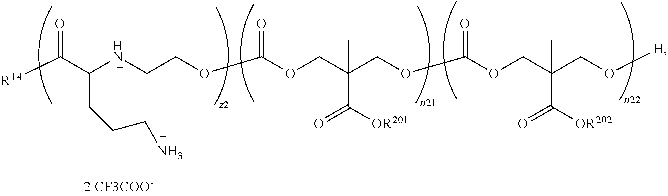

wherein n21 is 5, n22 is 6, and z2 is 10.

In embodiments, the cationic amphipathic polymer has the formula:

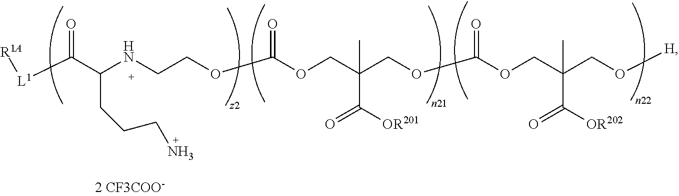

wherein n21 is 5, L¹ is —O—, n22 is 6, and z2 is 10.

In embodiments, the cationic amphipathic polymer has the formula:

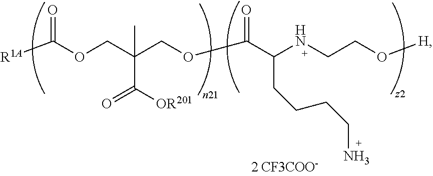

wherein n21 is 14, R²⁰¹ is dodecyl and z2 is 8.

In embodiments, the cationic amphipathic polymer has the formula:

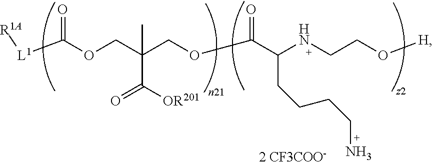

wherein n21 is 14, L¹ is —O—, R²⁰¹ is dodecyl and z2 is 8.

In embodiments, the cationic amphipathic polymer has the formula:

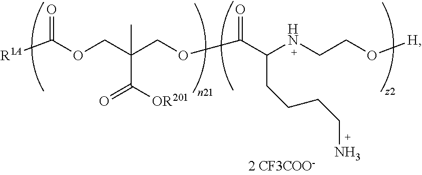

wherein n21 is 14, R²⁰¹ is dodecyl and z2 is 8.

In embodiments, the cationic amphipathic polymer has the formula:

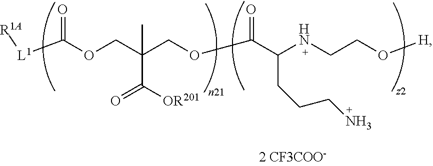

wherein n21 is 14, L¹ is —O—, R²⁰¹ is dodecyl and z2 is 8.

In embodiments, the cationic amphipathic polymer has the formula:

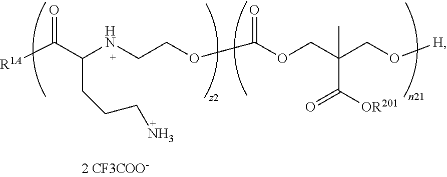

wherein n21 is 14, R²⁰¹ is dodecyl and z2 is 8.

In embodiments, the cationic amphipathic polymer has the formula:

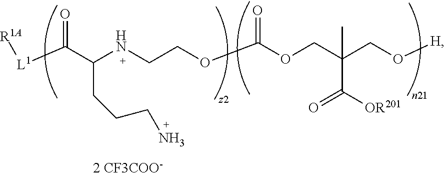

wherein n21 is 14, L¹ is —O—, R²⁰¹ is dodecyl and z2 is 8.

In embodiments, the cationic amphipathic polymer has the formula:

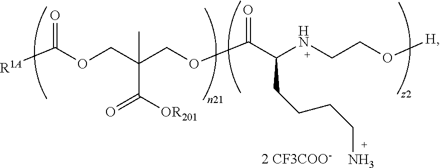

wherein n21 is 14, R²⁰¹ is dodecyl and z2 is 8.

In embodiments, the cationic amphipathic polymer has the formula:

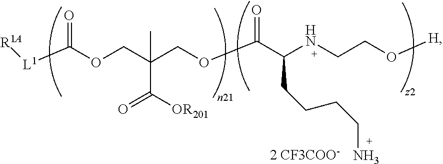

wherein n21 is 14, L¹ is —O—, R²⁰¹ is dodecyl and z2 is 8.

In embodiments, the cationic amphipathic polymer has the formula:

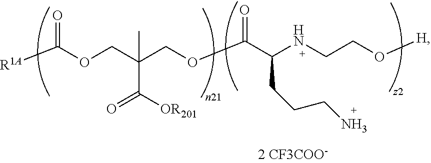

wherein n21 is 14, R²⁰¹ is dodecyl and z2 is 8.

In embodiments, the cationic amphipathic polymer has the formula:

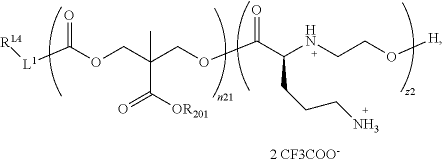

wherein n21 is 14, L¹ is —O—, R²⁰¹ is dodecyl and z2 is 8.

In embodiments, the cationic amphipathic polymer has the formula:

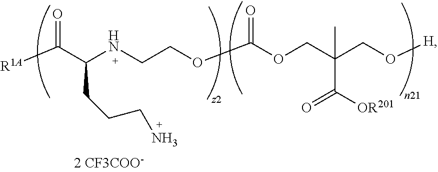

wherein n21 is 14, R²⁰¹ is dodecyl and z2 is 8.

In embodiments, the cationic amphipathic polymer has the formula:

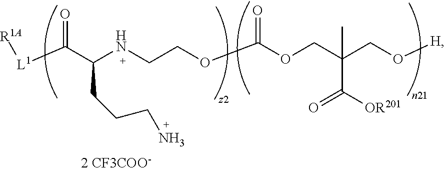

wherein n21 is 14, L¹ is —O—, R²⁰¹ is dodecyl and z2 is 8.

In embodiments, the cationic amphipathic polymer has the formula:

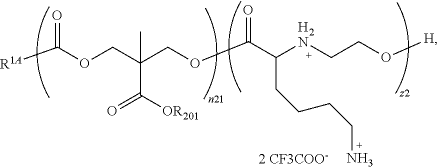

wherein n21 is an integer from 10 to 20;

R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

z2 is independently an integer from 3-10.

In embodiments, n21 is 14, R²⁰¹ is dodecyl and z2 is 8.

In embodiments, the cationic amphipathic polymer has the formula:

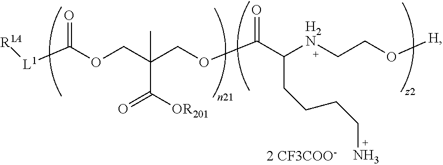

wherein R^{1A} is as described herein, n21 is an integer from 10 to 20 and L¹ is as described herein;

In embodiments, the cationic amphipathic polymer has the formula:

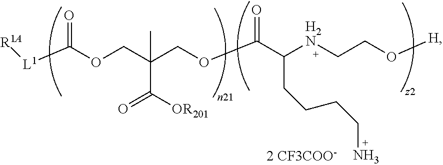

wherein R^{1A} is as described herein, n21 is 14; L¹ is —O—, R²⁰¹ is C₁₂H₂₅ and z2 is 8.

In embodiments, the cationic amphipathic polymer has the formula:

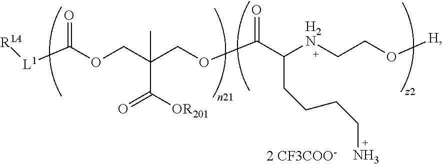

wherein R^{1A} is as described herein, n21 is 12; L¹ is —O—, R²⁰¹ is C₁₂H₂₅ and z2 is 6.

R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
z2 is independently an integer from 3-10.

In embodiments, n21 is 14, R²⁰¹ is dodecyl and z2 is 8.

In embodiments, the cationic amphipathic polymer has the formula:

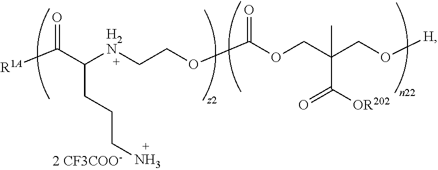

wherein n22 is an integer from 10 to 35;

R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
z2 is independently an integer from 5-20.

In embodiments, n22 is 14, R²⁰² is dodecyl and z2 is 7.

In embodiments, the cationic amphipathic polymer has the formula:

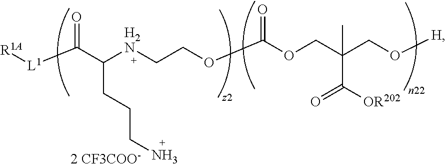

wherein n22 is an integer from 10 to 35; L¹ is defined as herein;

R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
z2 is independently an integer from 5-20.

In embodiments, n22 is 14, R²⁰² is dodecyl and z2 is 7.

In embodiments, the cationic amphipathic polymer has the formula:

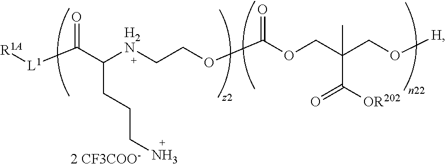

wherein R^{1A} is as described herein, n22 is 31; L¹-O—, R²⁰² is C₁₂H₂₅ and z2 is 10.

In embodiments, the cationic amphipathic polymer has the formula:

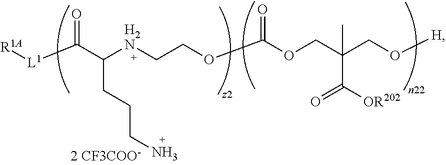

wherein R^{1A} is as described herein, n22 is 15; L¹-O—, R²⁰² is C₁₂H₂₅ and z2 is 5.

In embodiments, the cationic amphipathic polymer has the formula:

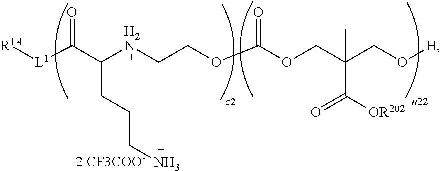

wherein R^{1A} is as described herein, n22 is 14; L¹ —O—, R²⁰² is C₁₂H₂₅ and z2 is 7.

In embodiments, the cationic amphipathic polymer has the formula:

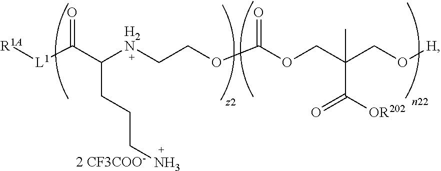

wherein R^{1A} is as described herein, n22 is 16; L¹ —O—, R²⁰² is C₁₂H₂₅ and z2 is 15.

In embodiments, the cell penetrating complex further includes a second cationic amphipathic polymer, wherein the second cationic amphipathic polymer is different from the cationic amphipathic polymer.

In embodiments, the second cationic amphipathic polymer has the formula:

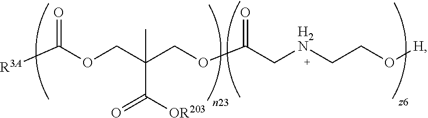

n23 is an integer from 1 to 100;
z6 is an integer from 5-15;

R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the second cationic amphipathic polymer has the formula:

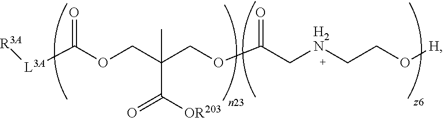

n23 is an integer from 1 to 100;
z6 is an integer from 5-15;
L^{3A} is a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, R²⁰³ is substituted or unsubstituted alkyl (e.g., C₁-C₃, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰³ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R²⁰³ is unsubstituted alkyl, unsubstituted heteroalkyl, unsubstituted cycloalkyl, unsubstituted heterocycloalkyl, unsubstituted aryl, or unsubstituted heteroaryl. In embodiments, R²⁰³ is hydrogen or unsubstituted alkyl (e.g., C₁-C₆ alkyl).

In embodiments, R²⁰³ is an unsubstituted C₁-C₃₀ alkyl. In embodiments, R²⁰³ is an unsubstituted C₁-C₂₀ alkyl. In embodiments, R²⁰³ is an unsubstituted C₈-C₃₀ alkyl. In embodiments, R²⁰³ is an unsubstituted C₈-C₂₀ alkyl. In embodiments, R²⁰³ is an unsubstituted C₉-C₂₀ alkyl. In embodiments, R²⁰³ is an unsubstituted C₉-C₁₈ alkyl. In embodiments, R²⁰³ is an unsubstituted C₁₈ alkyl. In embodiments, R²⁰³ is an unsubstituted C₁₇ alkyl. In embodiments, R²⁰³ is an unsubstituted C₁₆ alkyl. In embodiments, R²⁰³ is an unsubstituted C₁₅ alkyl. In embodiments, R²⁰³ is an unsubstituted C₁₄ alkyl. In embodiments, R²⁰³ is an unsubstituted C₁₃ alkyl. In embodiments, R²⁰³ is an unsubstituted C₁₂ alkyl. In embodiments, R²⁰³ is an unsubstituted C₁₁ alkyl. In embodiments, R²⁰³ is an unsubstituted C₁₀ alkyl. In embodiments, R²⁰³ is an unsubstituted C₉ alkyl. In embodiments, R²⁰³ is an unsubstituted C₈ alkyl. In embodiments, R²⁰³ is an unsubstituted C₇ alkyl. In embodiments, R²⁰³ is an unsubstituted C₆ alkyl. In embodiments, R²⁰³ is an unsubstituted C₅ alkyl. In embodiments, R²⁰³ is an unsubstituted C₄ alkyl. In embodiments, R²⁰³ is an unsubstituted C₃ alkyl. In embodiments, R²⁰³ is an unsubstituted C₂ alkyl.

In embodiments, R²⁰³ is an unsubstituted C₁-C₃₀ alkenyl. In embodiments, R²⁰³ is an unsubstituted C₁-C₂₀ alkenyl. In embodiments, R²⁰³ is an unsubstituted C₈-C₃₀ alkenyl. In embodiments, R²⁰³ is an unsubstituted C₈-C₂₀ alkenyl. In embodiments, R²⁰³ is an unsubstituted C₉-C₂₀ alkenyl. In embodiments, R²⁰³ is an unsubstituted C₉-C₁₈ alkenyl. In embodiments, R²⁰³ is an unsubstituted C₁₈ alkenyl. In embodiments, R²⁰³ is an unsubstituted C₁₇ alkenyl. In embodiments, R²⁰³ is an unsubstituted C₁₆ alkenyl. In embodiments, R²⁰³ is an unsubstituted C₁₅ alkenyl. In embodiments, R²⁰³ is an unsubstituted C₁₄ alkenyl. In embodiments, R²⁰³ is an unsubstituted C₁₃ alkenyl. In embodiments, R²⁰³ is an unsubstituted C₁₂ alkenyl. In embodiments, R²⁰³ is an unsubstituted C₁₁ alkenyl. In embodiments, R²⁰³ is an unsubstituted C₁₀ alkenyl. In embodiments, R²⁰³ is an unsubstituted C₉ alkenyl. In embodiments, R²⁰³ is an unsubstituted C₈ alkenyl. In embodiments, R²⁰³ is an unsubstituted C₇ alkenyl. In embodiments, R²⁰³ is an unsubstituted C₆ alkenyl. In embodiments, R²⁰³ is an unsubstituted C₅ alkenyl. In embodiments, R²⁰³ is an unsubstituted C₄ alkenyl. In embodiments, R²⁰³ is an unsubstituted C₃ alkenyl. In embodiments, R²⁰³ is an unsubstituted C₂ alkenyl.

In embodiments, the second cationic amphipathic polymer has the formula:

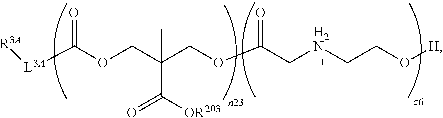

wherein R^{3A} is as described herein, L^{3A} is —O—, z6 is 16, R²⁰³ is C₁₂H₂₅ and n23 is 15.

In embodiments, the second cationic amphipathic polymer has the formula:

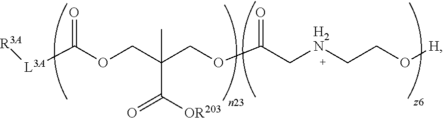

wherein R^{3A} is as described herein, L^{3A} is —O—, z6 is 11, R²⁰³ is C₁₂H₂₅ and n23 is 13.

In embodiments, R²⁰³ is a stearyl moiety (e.g., an unsubstituted C₁₈ alkyl). In embodiments, R²⁰³ is an oleyl moiety (e.g., an unsubstituted C₁₈ alkenyl). In embodiments, R²⁰³ is an linoleyl moiety (e.g., an unsubstituted C₁₈ alkenyl). In embodiments, R²⁰³ is an dodecyl moiety (e.g., an unsubstituted C₁₂ alkyl). In embodiments, R²⁰³ is an nonenyl moiety (e.g., an unsubstituted C₉ alkenyl). In embodiments, R²⁰³ is

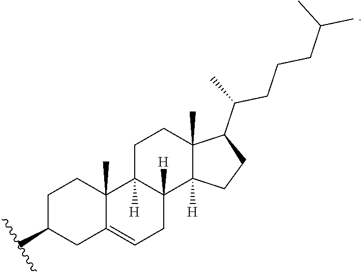

In embodiments, n23 is 13, z6 is 11 and R²⁰³ is dodecyl.

In embodiments, CART has the formula:

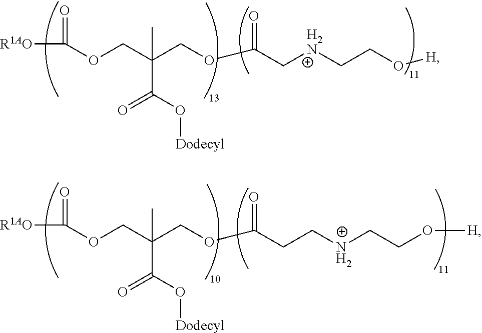

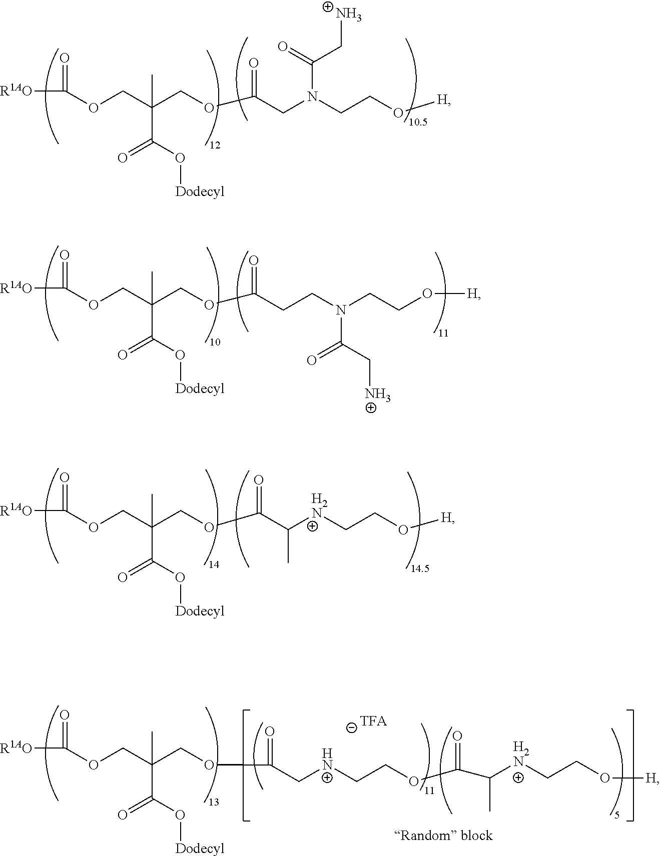

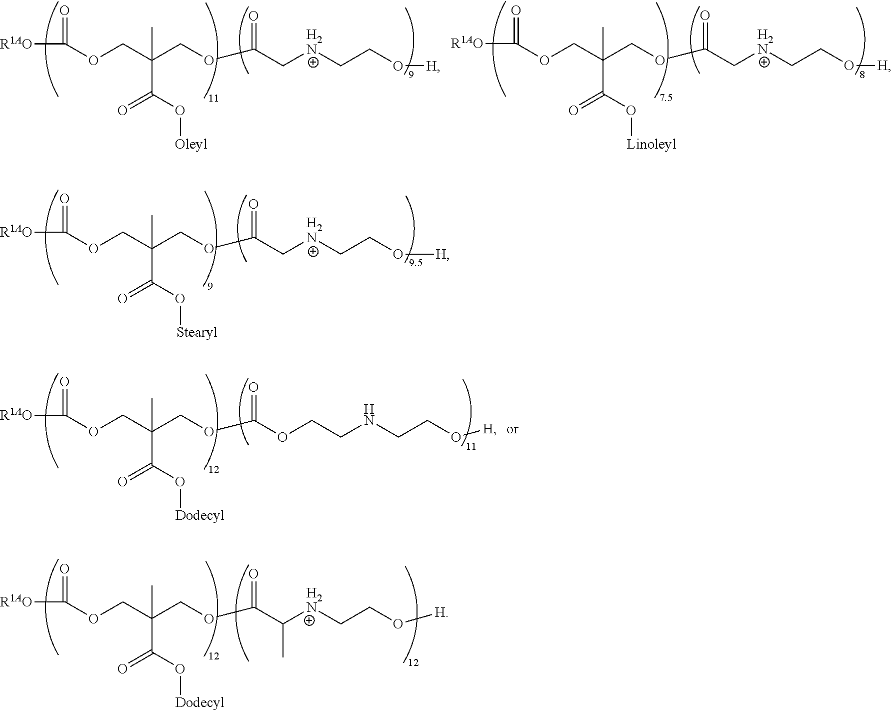

The nucleic acid (e.g., ribonucleic acid including a sequence encoding a viral protein) may be non-covalently bound to a first cationic amphipathic polymer and a second amphipathic polymer,

wherein the first cationic amphipathic polymer has the formula:

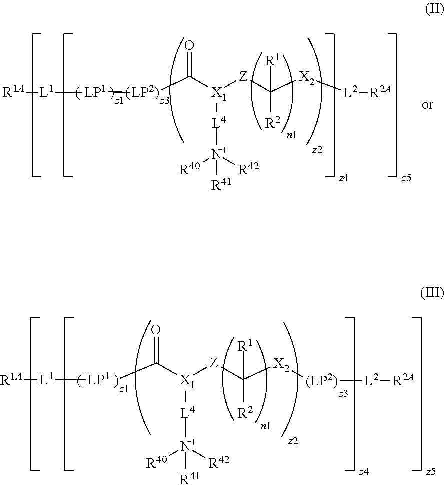

wherein

R^{1A} is independently hydrogen, halogen, —CCl₃, —CBr₃, —CF₃, —C₁₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{2A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, independently —COOH, —CONH₂, —NO₂, —SH, —SO₃H, —SO₄H, —SO₂NH₂, —NHNH₂, —ONH₂, independently —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

LP¹ and LP² are independently a lipophilic polymer domain;

X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—;

X² is —O—or —S—;

R¹, R², R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

L⁴ is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene;

R⁴⁰, R⁴¹, and R⁴² are independently hydrogen, substituted or unsubstituted alkyl or susbstituted or unsubstituted heteroalkyl;

Z is —S—, —S⁺R¹³—, —NR¹³ —, or —N⁺(R¹³)(H)—;

R¹³ is hydrogen, —CCl₃, —CBr₃, —CF₃, —CI₃, —CN, —OH, ═O, —NH₂, —COOH, —CONH₂, —SH, —SO₃H, SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

n1 is an integer from 0 to 50;

z1 and z3 are independently integers from 0 to 100, wherein at least one of z1 or z3 is not 0;

z2 is an integer from 2 to 100;

z4 is an integer from 1 to 100;

z5 is an integer from 1 to 10; and wherein the first cationic amphipathic polymer and the second amphipathic polymer are different.

The first cationic amphipathic polymer may be any of the cationic amphipathic polymers provided herein including embodiments thereof. The second cationic amphipathic polymer may be any of the cationic amphipathic polymers provided herein including embodiments thereof or it may be any other cationic amphipathic polymer useful for the complexes provided herein. In embodiments, the second cationic amphipathic polymer is any of the cationic amphipathic polymers described in PCT application serial number PCT/US17/44238 published as WO 2018/022930, which is hereby incorporated by reference in its entirety and for all purposes.

In embodiments, the first cationic amphipathic polymer has the formula:

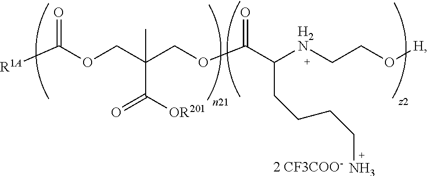

wherein n21 is an integer from 10 to 20;

R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

z2 is independently an integer from 3-10.

In embodiments, n21 is 14, R²⁰¹ is dodecyl and z2 is 8.

In embodiments, the first cationic amphipathic polymer has the formula:

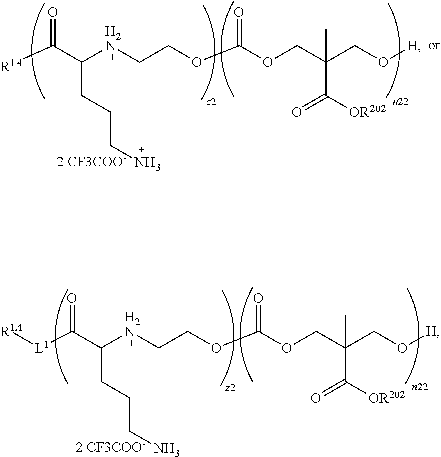

wherein n22 is an integer from 10 to 35;

R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

z2 is independently an integer from 5-20.

In embodiments, n22 is 14, R²⁰² is dodecyl and z2 is 7.

In embodiments, the second cationic amphipathic polymer has the formula:

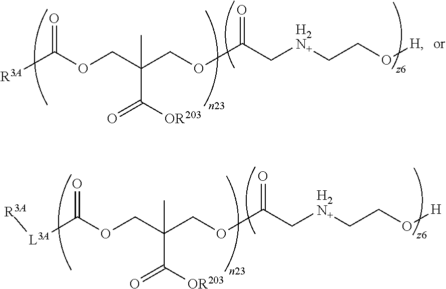

wherein n23 is an integer from 1 to 100;

z6 is an integer from 5-15; and

R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, -NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L^{3A} is a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, n23 is 13, z6 is 11 and R²⁰³ is dodecyl.

In embodiments, the first cationic amphipathic polymer has the formula:

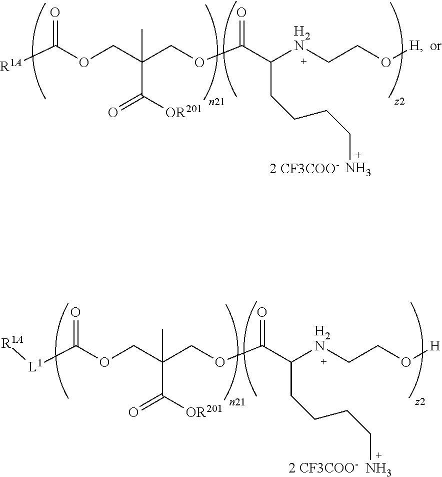

wherein n21 is 14, R²⁰¹ is dodecyl and z2 is 8; and
wherein the second cationic amphipathic polymer has the formula:

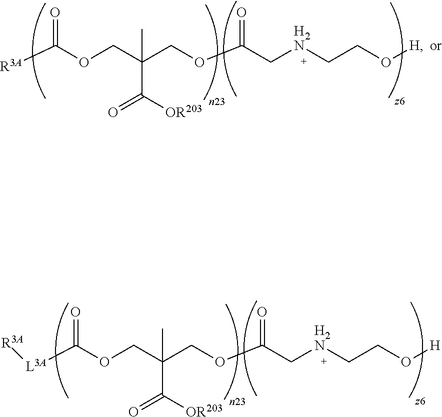

wherein n23 is 13, R²⁰³ is dodecyl and z6 is 11.

In embodiments, the first cationic amphipathic polymer has the formula:

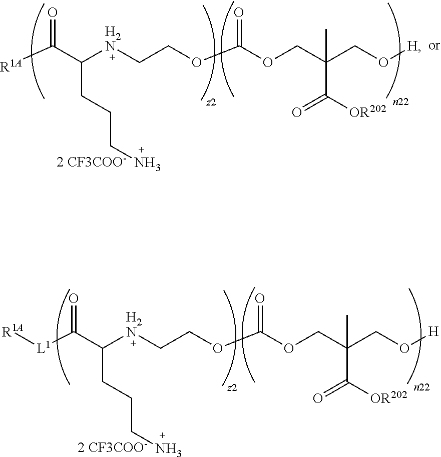

wherein n22 is an integer from 10-35, R²⁰² is dodecyl and z2 is 3-15; and
wherein the second cationic amphipathic polymer has the formula:

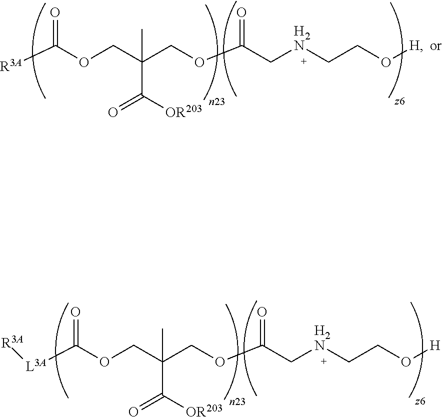

wherein n23 is 13, R²⁰³ is dodecyl and z6 is 11.

In one aspect is provided a nanoparticle composition including a plurality of cell-penetrating complexes as described herein, including embodiments.

In one aspect is provided a pharmaceutical composition including a cell-penetrating complex as described herein, including embodiments, and a pharmaceutical excipient.

In embodiments, the nucleic acid is DNA or RNA, such as messenger RNA (mRNA) (e.g., ribonucleic acid including a sequence encoding a viral protein), small interference RNA (siRNA), short hairpin RNA (shRNA), micro RNA (miRNA), guide RNA (gRNA), CRISPR RNA (crRNA), transactivating RNA (tracrRNA), plasmid DNA (pDNA), minicircle DNA, genomic DNA (gNDA). The cell-penetration complex may further include a protein or peptide.

The cell-penetration complex disclosed herein and embodiments thereof, may include a plurality of lipophilic moieties.

The cell-penetration complex disclosed herein and embodiments thereof, may include a plurality of immolation domains.

Further to the cell-penetration complexed disclosed herein and embodiments thereof, in embodiments, the counter-anion to the above cationic sequences can include common counterions known in the art, such as for example acetate, trifluoroacetate, triflate, chloride, bromide, sulfate, phosphate, succinate, or citrate. In embodiments, the counter-anion is acetate, trifluoroacetate, triflate, chloride, bromide, sulfate, phosphate, succinate, or citrate.

In another aspect, there is provided a method of transfecting a nucleic acid into a cell, the method including contacting a cell with a cell-penetrating complex as disclosed herein, or embodiment thereof.

In embodiments, the method further includes allowing the cationic amphipathic polymer to degrade within the cell thereby forming a degradation product. In embodiments, the degradation product is a substituted or unsubstituted diketopiperazine.

Further to any embodiment of the method of transfecting a nucleic acid into a cell, in embodiments the nucleic acid is an mRNA. In embodiments, the method further includes allowing the mRNA to be expressed in the cell. In embodiments, the cell forms part of an organism. In embodiments, the organism is a human.

Provided herein, inter alia, are novel materials and strategies that enable or enhance the complexation, protection, delivery and release of oligonucleotides and polyanionic cargos, e.g., messenger RNA (mRNA), into target cells, tissues, and organs both in vitro and in vivo.

For example, one strategy disclosed herein for mRNA delivery is accomplished using biodegradable poly(carbonate-co-aminoester)s oligomers and variations thereof which were discovered to electrostatically complex polyanions such as mRNA, producing noncovalent macromolecular particles that protect the mRNA cargo, readily enter cells and uniquely release oligonucleotide cargos. The mRNA released in the cell is then converted by cellular processes into peptides and proteins whose sequence and thus activity is determined by the mRNA sequence.

Thus, there is provided, for example, greatly increased improved cellular transfection efficiency over the use of nucleic acids themselves and known gene delivery vectors. The materials and strategy used for the delivery of mRNA can also be used to deliver other oligonucleotides such as siRNA, pDNA, shRNA, and gDNA. They can additionally be utilized to deliver other anionic biomolecules such as heparin, inorganic polyphosphate, and inositol polyphosphates (e.g., IP3, IP7, IP8). This delivery can be achieved with a variety of human and non-human cell lines, as well as through multiple modes of administration in vivo including but not limited to intramuscular, intravenous, intraperitoneal, intraocular, intranasal, subcutaneous, buccal and topical. The poly(carbonate-co-aminoester)s disclosed herein can be utilized, for example, as customizable, biodegradable, biocompatible materials for applications in biomedical therapies, imaging and devices. The copolymerization with biodegradable, non-toxic compounds materials such as valerolactone, caprolactone, lactide, and cyclic carbonates allows for tuning physical and biological properties including cargo release rates, hydrophobicity, incorporation of targeting ligands, biodistribution, and toxicity.

Accordingly, in some embodiments, the agents provided herein include oligomers, polymers, co-oligomers, and copolymers which may be derived from cyclic amino-ester and cyclic methyl trimethylene carbonate (MTC) monomers. Cyclic amino-esters have the base structure of morpholin-2-one and homologs thereof, with multiple substitution patterns possible including the following.
(1) N-acylation with a variety of hydrophobic groups (e.g., R=alkyl, alkenyl, aryl, polycycles including steroids, heterocycles), cationic groups (e.g., ammonium, phosphonium, sulfonium, guanidinium, including acylation with amino acids such as glycine, lysine, ornithine, arginine), anionic groups (e.g., carboxylate, sulfate, phosphate), or hydrophilic (e.g., PEG) carbamates. Protection of the morpholine nitrogen with N-Boc or N-Cbz groups followed by organocatalytic ring opening oligomerization or polymerization can afford upon deprotection cationic polymer or oligomer backbones.
(2) Alpha-alkylation or functionalization next to the ester carbonyl with the aforementioned possible functionalities selected to allow for cargo complexation and subsequent cargo release by biodegradation.
(3) Alkylation proximal to the morpholine nitrogen with the aforementioned functionalities.
(4) A combination of the above modifications.

Additionally, copolymers or co-oligomers (block or statistical) can be made by mixing two or more morpholin-2-one monomers, or by the copolymerization (or co-oligomerization) of one or multiple morpholin-2-one monomers with one or multiple cyclic carbonate monomers described herein. These carbonate monomers can incorporate a similar variety of side chain functionality, notably lipophilic groups or cationic groups to modulate oligonucleotide stability, delivery, and release properties. Furthermore, a variety of other commercially available cyclic ester monomers can be used including but not limited to lactide, glycolide, valerolactone, and/or caprolactone to incorporate lipophilic functionality.

The synthesis of polyaminoesters and poly(carbonate-co-aminoester)s is achieved through the ring-opening polymerization and/or copolymerization of morpholine-2-one and cyclic carbonate monomers. The N-Boc protected morpholinone (MBoc) polymerizes to high conversion (>85%), tunable Mn (1 kDa-20 kDa), and low molecular weight distributions (Mw/Mn-1.1-1.3) using an organocatalytic system. Post-polymerization deprotection of the Boc groups affords a cationic (diprotic, secondary amine) water-soluble polymer (−0.5M in D20, stable for >3 days). Furthermore, copolymerization of MBoc with MTC-dodecyl carbonate monomers followed by deprotection give rise to moderately charged cationic materials in high yield (>60%) with narrow polydispersity <1.4 PDI) and tunable block length. Block length is controlled by the ratio of initiator to monomer.

The polyaminoesters and poly(carbonate-co-aminoester)s are biocompatible and biodegradable. The cationic polyaminoesters rapidly degrade through a novel pH- and buffer-dependent immolation mechanism to generate in one embodiment bis-N-hydroxyethy-2,5-piperizinedionebis-hydroxyethyl glycine. This unforeseen degradation produces a product that is nontoxic at treatment concentrations, and the monomeric form (the expected product of further hydrolysis) is a natural biomarker for phospholipid modification in the Maillard reaction. The carbonate segment of the aminoester/carbonate copolymers degrades through hydrolysis and decarboxylation, and its byproducts have previously been shown to be non-toxic. The new poly- and oligo(carbonate-co-aminoester)s exhibit unanticipated performance as gene delivery agents due to their unique degradation mechanism. These new materials non-covalently complex, protect, deliver, and release mRNA at moderate theoretical charge ratios (e.g., about 10:1) resulting in exceptional transfection efficiencies (in some cases >99%) and robust induction of gene expression in vitro and in vivo. This strategy is effective for the delivery of mRNA molecules of different lengths (1000 and 2000 nucleotide transcripts tested). In one embodiment, gene delivery is achieved through formulation of cationic poly(carbonate-co-aminoester)s with anionic cargos to form self-assembled particles 200-400 nm in size. These particles are stable on the timescales necessary for intracellular gene delivery, and then they release the oligonucleotide cargo once inside the cell. While not bound to any particular theory, these materials degrade to the bis-N-hydroxyethyl-2,5-piperizinedione product bis-hydroxyethyl glycine. Treatment of a variety of human and non-human cell lines (e.g., HeLa, HaCaT, J774, HEK293) with the mRNA/amphiphile complex results in the induction of protein expression (e.g., GFP, luciferase) in vitro and in vivo through multiple modes of administration (intramuscular and intravenous tested).

Protein expression has been measured using mRNA encoding fluorescent reporter genes by flow cytometry and fluorescence microscopy (GFP), as well as bioluminescence (firefly luciferase). The poly(carbonate-co-aminoester)s have been shown to be more efficient transfection agents than the commercial standard Lipofectamine 2000, as well as many other lead compounds previously described for siRNA delivery.

In embodiments, gene delivery is achieved by formulation of the mixed amphipathic oligomer with an mRNA cargo in the presence of third components selected to tune stability and size of the resulting complex, increase cellular uptake, tune rate of mRNA release from the complex, and enhance expression of the cargo mRNA. Tertiary components include but are not limited to coordinating metal such as Zn⁺², Mg⁺², Ca⁺², etc; dynamic non-covalent cross linkers such as carbohydrates, counterions such as Cl⁻, AcO⁻, succinate, and citrate; and solubility modulators such as lipids and PEGs.

Applications of this technology may include: Clinical Applications: (1a) Nucleic acid transfection vectors: While utilizing DNA and RNA has been proposed to treat genetic disease for many years, the greatest obstacle to clinical use of gene therapy remains the effective delivery of the oligonucleotide cargos (1b) RNA Vaccination to prevent infectious diseases: mRNA-based vaccines display strong safety advantages over DNA vaccines, however they are clinically currently limited by mRNA delivery into cells. This application is currently being investigated clinically, but the most advanced technologies require removal of primary cells from patients for in vitro transfection with electroporation, followed by subsequent reintroduction of the transformed cells into the patient. This method can be significantly improved using our delivery technology to directly induce mRNA expression in vivo. (1c) Stem cell induction: pluripotency can be induced in undifferentiated stem cells by using our technology to induce the expression of 4 known transcription factors. The modular nature of the poly(carbonate-co-aminoester) delivery vehicles enables facile delivery of all four necessary mRNA transcripts simultaneously. (1) Basic research applications including, but not limited to: in vitro transfection of cultured cells, gene editing using CRISPR/Cas9, pathway validation using combination gene expression (mRNA translation) and gene knockdown (RNAi). Cancer immunotherapy, allergy tolerance, protein replacement therapy, gene editing, diagnostics,

Advantages of the presently disclosed complexes, compositions and methods may include, for example:
(1) Higher mRNA transfection efficiency in vitro than commercially available transfection agents such as Lipofectamine 2000, even in difficult-to-transfect cell lines such as J774 macrophages, thereby improving efficacy while increasingly tolerability.
(2) Robust gene expression in vivo (BALB/c mice), demonstrating the clinical applicability of this technology, thereby avoiding toxicities of cationic carriers such as lipofectamine and providing a clinical alternative to ex-vivo methods of gene delivery and expression.
(3) Differential in vivo gene expression can be achieved using distinct routes of administration, with liver and spleen expression dominating upon intravenous injection, while local expression is sustained at the site of administration with an intramuscular (for example) delivery. Nasal delivery provides a route to mucosal membrane and/or lung uptake.
(4) Rapid degradation to known metabolites (bis-hydroxyethylglycine) which enables efficient gene expression
(5) Release of mRNA in a pH-dependent manner, such as with oligonucleotide-bearing particles displaying stability in low pH environments (such as the skin or intestinal tract), but degrading in higher pH environments.
(6) Materials are easily accessed through metal-free synthesis to make oligomers, polymers, or block/statistical copolymers or co-oligomers with targeted molecular weight and a high degree of control over dispersity.
(7) Materials are amenable to targeting through addition of targeting ligands such as folate or biotin to the surface of the formed particle or through attachment to monoclonal antibodies.
(8) The specific immolation mechanism of the cationic polyaminoester domain to an isolable neutral small molecule results in the formation of the biocompatible/biodegradable product, bis-N-hydroxyethyl-2,5-piperizinedione, a cyclic dimer of hydroxyethylglycine.

Features of the complexes, compositions and methods include the following. In embodiments, the poly(carbonate-co-aminoester)s poly(aminoester)s and the cationic materials derived thereof that can exhibit at least one of the following properties and functions:
(1) A specific, pH-responsive immolation mechanism of the cationic polyaminoesters that results in bio-compatible/biodegradable hydroxyethylglycine dimers; domain of these materials that leads to the release of the oligonucleotide cargo is unique even among other responsive biomaterials in that it occurs via an unanticipated intramolecular bond-forming event that results in irreversible neutralization of the cationic ammonium to rapidly trigger the release of the anionic cargo.
(2) An isolable product of intramolecular degradation, such as bis-N-hydroxyethyl-2,5-pipericla-7-inedione, which further degrades to hydroxyethyl glycine.
(3) Providing a temporal window of activity, such that the anionic cargo is electrostatically packaged into particles for delivery, then rapidly released following cellular internalization.
(4) Enablement of copolymerization of multiple lactone monomers with control over macromolecular architecture. Functionalized monomers may be polymerized in block or statistical architectures, and this further allows the combination of multiple monomer types such as cyclic carbonates or phosphates.
(5) The use of these materials in vivo may occur without acute toxicity, even when administered locally or systemically, indicative of high tolerability at concentrations necessary for a therapeutic response.
(6) Use as gene delivery vehicles, poly(carbonate-co-aminoester)s enable the efficient delivery and release of oligonucleotides including messenger RNA. Amphipathic block co-oligomers of MTC-dodecyl carbonate and N-Boc morpholine-2-one monomers can be formulated with large anionic cargos such as mRNA, to form stable, sub-400 nm particles. These resulting particles may effectively be taken up by cells and release their mRNA cargo, resulting in robust gene expression. This concept has been demonstrated in vitro in multiple cell lines as well as in vivo in mouse studies. The efficacy of these materials has been shown to be due to the pH-responsive rearrangement of the cationic aminoester block to form the neutral small molecule bis-N-hydroxyethyl-2,5-piperidinedione bis-hydroxyethylglycine. While other cationic gene delivery vehicles have been previously reported, these oligo(carbonate-co-aminoester)s are unique and among the top performers, due to their unique ability to release the mRNA (or other oligonucleotide) cargo on a time scale appropriate for cellular uptake and their tolerability.
(7) The empirically determined optimal length for mRNA delivery teaches away from our prior art in that diblocks of average DP 12 for both MTC-dodecyl carbonate and N-Boc morpholine-2-one domains perform optimally. This length is much shorter than commercially available cationic polyamine vectors such as PEI; it is also longer than our previously discovered siRNA delivery vectors (Reference WO2013036532 A1 and PNAS 2012, 109 (33), 13171-13176).

In one aspect, provided herewith is a method of transfecting a nucleic acid into a cell (e.g., a lung cell). The method includes contacting a cell with a cell-penetrating complex described herein including embodiments thereof. In embodiments, the method causes gene-edition in the cell. In embodiments, the gene-edition can encompass genome-edition or genome editing which is a type of genetic engineering in which DNA is inserted, deleted or replaced in the genome of a living organism using an isolated or engineered nuclease system. In certain embodiments, the method disclosed herein can be used to deliver a genetic tool or system that can cause gene-edition in the transfected cells. Some non-limiting examples of a genetic tool or system for gene-edition include a CRISPR-Cas system and transposon system.

In one aspect, a nucleic acid (i.e. the cargo nucleic acid) transfected by the transfection method according to some embodiments can have one or more vectors having a first nucleotide sequence encoding a CRISPR-Cas system guide RNA that hybridizes with a target sequence in the genome of the cell and a second nucleotide sequence encoding a Cas9 protein. In certain embodiments, the first and second nucleotide sequence can be located on the same or different vectors.

In general, a system adopting CRISPR/Cas9 offers a high degree of fidelity and relatively simple construction for gene edition. The system can depend on two factors for its specificity: a target sequence and a protospacer adjacent motifs (PAM). The target sequence can be, e.g. 20 bases long as part of each CRISPR locus in a crRNA array. A crRNA array can have multiple unique target sequences. Cas9 proteins can select a correct location on the host's genome by utilizing the sequence to bind with base pairs on the host DNA. The PAM sequence on the host genome can be recognized by Cas9. Once the elements are assembled, e.g. into one or more plasmids and transfected into cells, the Cas9 protein with the help of the crRNA can find the correct sequence in the host cell's DNA and—depending on the Cas9 variant—creates a single or double strand break in the DNA. Properly spaced breaks in the host DNA can trigger homology directed repair. Providing a DNA repair template can allow for the insertion of a specific DNA sequence at an intended location within the genome. Once incorporated, the new sequence is now part of the cell's genetic material and can pass into its daughter cells. Many online tools are available in the art to aid in designing effective sgRNA sequences. According to some embodiments, the method and composition according to certain embodiments herewith can deliver or transfect a nucleotide sequence encoding CRISPR-Cas system guide RNA and a nucleotide sequence encoding a Cas9 protein to induce gene-edition in the transfected cells.

In some embodiments, a cargo nucleic acid transfected by the transfection method according to certain embodiments can have a CRISPR RNA (crRNA). In some embodiments, this crRNA can be in the same vector of the first nucleotide sequence encoding a CRISPR-Cas system guide RNA.

In some embodiments, a cargo nucleic acid transfected by the transfection method according to certain embodiments can have a transactivating RNA (tracrRNA). In some embodiments, this tracrRNA can be in the same vector of the second nucleotide sequence encoding a Cas9 protein.

In some embodiments, the Cas9 protein utilized in the transfection method according to some embodiments can be codon optimized for expression in the transfected cell.

In another aspect, a nucleic acid (i.e. the cargo nucleic acid) transfected by the transfection method according to some embodiments can have one or more vectors having a first nucleotide sequence encoding a transposase and a second nucleotide sequence having a nucleic acid sequence of a gene of interest flanked by a transposase recognition site. In some embodiments, the first and second nucleotide sequences can be located on the same or different vectors.

A transposable element (or transposon) generally refers to a DNA sequence that can change its position within a genome, sometimes creating or reversing mutations and altering the cell's genetic composition and genome size. Transposase generally refers to an enzyme that can bind to a transposon and catalyze the movement of the transposon to another part of the genome by, e.g. a cut and paste mechanism or a replicative transposition mechanism. Introduction of transposase and a gene of interest flanked by a transposase recognition site in cells can induce insertion of the gene of interest into a cellular genome. According to some embodiments, the method and composition according to certain embodiments herewith can deliver or transfect a nucleic acid encoding a transposase and a gene of interest to induce gene-edition in the transfected cells.

In embodiments, the transposase used in the transfection method according to some embodiments can recognize and excise a genomic sequence. In some other embodiments, the nucleic acid sequence of the gene of interest that is transfected via the transfection method can be integrated into a genome of the transfected cell.

In embodiments, the gene-editing done via the transfection method according to some embodiments can cause one or more of the following: a DNA deletion, a gene disruption, a DNA insertion, a DNA inversion, a point mutation, a DNA replacement, a knock-in, and a knock-down.

In one aspect a method of transfecting a nucleic acid into a cell is provided. The method includes contacting a cell with a cell-penetrating complex as provided herein, including embodiments thereof. In embodiments, the cell is a lung cell.

### Vaccine Compositions and Methods of Treating Viral Disease

In an aspect is provided a vaccine composition including a cell-penetrating complex, as described herein, including embodiments. The cell-penetrating complexes provided herein are particularily useful in vaccines, wherein the vaccine includes a ribonucleic acid including a sequence encoding a viral protein, a nucleic acid adjuvant and a cationic amphipathic polymer provided herein including embodiments thereof.

In an aspect is provided a method of treating or preventing a viral disease in a subject in need of such treatment or prevention, the method including administering a therapeutically or prophylactically effective amount of a cell-penetrating complex as described herein, including embodiments, to the subject.

In an aspect is provided a method of treating a viral disease in a subject in need thereof, including administering to the subject a therapeutically effective amount of a cell-penetrating complex as described herein, including embodiments, and a pharmaceutical carrier, thereby treating a viral disease in the subject.

The cell-penetrating complexes provided herein are particularily useful in vaccines, wherein the vaccine includes a ribonucleic acid including a sequence encoding a viral protein, a nucleic acid adjuvant and a cationic amphipathic polymer provided herein including embodiments thereof. Thus, in an aspect is provided a method for immunizing a host susceptible to a viral disease, including administering a cell-penetrating complex as described herein, including embodiments, to a host under conditions such that antibodies directed to the viral protein or a functional fragment thereof are produced. Based on the combination of adjuvant and cationic amphipathic polymer used in the vaccine a specific class of IgG isotypes is produced in the subject being administered the vaccine. In embodiments, the nucleic acid adjuvant has the sequence of SEQ ID NO:1 (tccatgacgttcctgacgtt) and the antibodies produced are mouse IgG2 antibodies (e.g., IgG2a and IgG2b). In embodiments, the nucleic acid adjuvant has the sequence of SEQ ID NO:1 (tccatgacgttcctgacgtt) and the antibodies produced are human IgG1 antibodies. In further embodiments, the cell-penetrating complex is administered intraveniously.

In embodiments, the nucleic acid adjuvant has the sequence of SEQ ID NO:2 (tcgaacgttcgaacgttcgaacgttcgaat) and the antibodies produced are IgG1, IgG2a, IgG2b and IgG3 antibodies, wherein the amount of IgG1 antibodies is lesser relative to the amount of IgG2a and IgG2b antibodies. In further embodiments, the cell-penetrating complex is administered intraveniously.

In embodiments, the antibodies are IgG2 antibodies.

In embodiments, the viral disease is SARS. In embodiments, the viral disease is COVID-19. In embodiments, the viral disease is MERS.

### Pharmaceutical Compositions

In an aspect is provided a pharmaceutical composition including a therapeutically effective amount of a cell-penetrating complex as described herein, including embodiments, and a pharmaceutically acceptable excipient.

In embodiments, the compositions provided herein are used for a therapeutic purpose. In some embodiments, a therapeutic purpose encompasses a prophylactic purpose (a purpose of preventing a disease or condition from occurring) and a treatment purpose (a purpose of treating an existing disease or condition). When the composition has a cationic amphipathic polymer but not a cargo nucleic acid, the cargo nucleic acid, which can exhibit a therapeutic effect, can be non-covalently bound to the cationic amphipathic polymer, before administration to a subject.

In some embodiments, a composition can be a vaccine or a composition thereof, i.e. a composition that contains the vaccine and optionally a pharmaceutically acceptable carrier. The vaccine or vaccine composition can be used to prevent and/or treat a n infectious disease (e.g., COVD-19) or condition or a pathogen associated with the disease or condition. In some embodiments, the vaccine or vaccine composition contains a cell-penetrating complex which has a cationic amphipathic polymer and a cargo nucleic acid. In some embodiments, the cell-penetrating complex, when administered to a subject, can induce an immune response, i.e. immunogenic. This immunogenicity can be induced, at least in part, when one or more antigenic peptides encoded by the cargo nucleic acid are expressed in the transfected cells.

In one aspect, a cationic amphipathic polymer or a cell-penetrating complex disclosed herein can be formulated in a pharmaceutical composition. The cationic amphipathic polymer can have a pH-sensitive immolation domain. In one embodiment, the pharmaceutical composition can further contain a pharmaceutically acceptable excipient and/or a pharmaceutically acceptable carrier.

In some embodiments, pharmaceutical compositions can have a cell-penetrating complex, which has a nucleic acid non-covalently bound to a cationic amphipathic polymer, as an active ingredient, a nucleic acid adjuvant and further contain pharmaceutically acceptable excipients or additives depending on the route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used can be chosen from, but not limited to, the above or combinations thereof, as appropriate, depending on the dosage form of the present disclosure.

In some embodiments, the pharmaceutically acceptable carrier is a nucleic acid adjuvant. In some examples, the nucleic acid adjuvant can include, but is not limited to, agonists of Toll-like Receptors (TLRs), agonists of the STING pathway, agonistic antibodies against CD40, OX40, CTLA4, PD1, or PD1-L, Freund's adjuvant, bryostatins and ligands for CD40, OX40, CD137, PD1, CTLA4 and any combinations thereof. In some embodiments, the nucleic acid adjuvant can increase immunogenicity that is induced when a cell-penetrating complex by co-administered with the complex to a subject. In some embodiments, the nucleic acid adjuvant can induced antibody class switching when co-administered with the complex to a subject.

Formulation of the pharmaceutical compositions of the present disclosure can vary according to the route of administration selected (e.g., solution, emulsion). Routes of administration can be, for example, intramuscular, subcutaneous, intravenous, intralymphatic, subcutaneous, intramuscular, intraocular, topical skin, topical conjunctival, oral, intravessical (bladder), intraanal and intravaginal.

In some embodiments, the composition can include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition into a matrix; enteric coatings; micro-encapsulation; gels and hydrogels; implants; and any other formulation that allows for controlled release of a composition.

In one aspect is provided a kit of parts having a cell-penetrating complex or composition thereof. In another aspect is provided a kit of parts having a cationic amphipathic polymer that is not bound to a nucleic acid or composition thereof. The kit can further contain a document or an instruction that describes a protocol for making a cell-penetrating complex using a cationic amphipathic polymer and a cargo nucleic acid. The document or instruction of the kit can also describe a protocol for administering the composition to a subject in need thereof.

Therapeutic formulations described herein can be prepared for storage by mixing the active ingredients, i.e., immunogenic agent(s) having the desired degree of purity with optional physiologically acceptable carriers, excipients or stabilizers. Acceptable carriers, excipients, or stabilizers can be nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound (e.g., a second active agent in addition to the immunogenic agent(s) that has a cell-penetrating complex), which may be selected for complementary activities that do not adversely affect each other. Such molecules can be suitably present in combination in amounts that can be effective for the purpose intended.

### Methods of Inducing an Immune Response

In another aspect, provided herein are methods of inducing an immune response in a subject. In some embodiments, the methods can treat and/or prevent a disease or condition using a cell-penetrating complex. The methods generally involve administering a subject in need thereof a therapeutically effective amount of a cell-penetrating complex or a pharmaceutical composition having the cell-penetrating complex described herein, alone (e.g., in monotherapy) or in combination (e.g., in combination therapy) with one or more additional ingredients, e.g., a pharmaceutically acceptable excipient and/or additional therapeutic agent.

In some embodiments, a cell-penetrating complex or a pharmaceutical composition having the cell-penetrating complex can be used as a vaccine that can induce an immune response in a subject who was administered with the cell-penetrating complex or a pharmaceutical composition thereof.

In some embodiments, the vaccine can have a prophylactic activity such that the vaccine can prevent or reduce a likelihood of the occurrence of a disease or condition in a subject. In embodiments, where the vaccine is used for a prophylactic purpose, a subject can be an animal who does not have the disease or condition, e.g. a human who was not diagnosed with the disease or condition or who does not have a noticeable symptom associated with the disease or condition. In some other embodiments, the vaccine has a therapeutic effect such that the vaccine can be used to treat a disease or condition (e.g., a vial disease). Some examples of therapeutic vaccines can include, but are not limited to, viral vaccines that can be administered to a patient who have or are at risk of developping a viral disease (e.g. COVID-19). The viral vaccines can exhibit one or more anti-viral activity, e.g. reduction of viral particle number, reduction and/or inhibition of viral replication and infectivity. In some other embodiments, viral vaccines are used for a prophylactic purpose, especially in a subject who is considered predisposed of infection but presently does not have the viral disease. The prophylactic vaccine can be administered to the predisposed subject and prevent or reduce a likelihood of the occurrence of the viral disease in the subject.

In one aspect, the disclosures herewith provide a method of inducing an immune response against a disease in a subject in need thereof. The method can contain administering an effective amount of a cell-penetrating complex to a subject.

In some embodiments, a cell-penetrating complex can be used as a vaccine that can induce an immune response in a subject who is administrated with the complex. The complex can contain a nucleic acid non-covalently bound to a cationic amphipathic polymer and the cationic amphipathic polymer can have a pH-sensitive immolation domain.

In embodiments, the nucleic acid that is contained in the vaccine or composition thereof can be a nucleic acid sequence encoding a viral protein or fragmetn thereof. For example, when an infectious disease is concerned, the nucleic acid contained in the vaccine can encode one or more peptides that are known to be expressed in the pathogen (e.g. pathogenic virus) of the infectious disease and can induce an immune response when administered in a subject. In embodiments, the viral protein is the soluble receptor binding domain (RBD) of a SARS-CoV-2 spike protein. When the nucleic acid encoding the viral protein is administered to a subject and delivered (i.e. transfected) into certain cells of the subject, the transfected nucleic acid can be eventually translated and expressed into the antigenic peptide(s). Since the expressed peptide(s) is antigenic or immunogenic, an immune response against the expressed peptide can be induced in the subject. The subject can have an acquired immune response via this vaccination in which adaptive immunity can elicit immunological memory after an initial response to the immunogenic peptides that is targeted by the immune response, and leads to an enhanced response to that target on subsequent encounters, exhibiting a prophylactic effect.

In some embodiments, vaccination can provide dual activities of therapeutic and prophylactic effects by delivering two separate types (or sequences) of nucleic acids in a single vaccine composition. The two separate nucleic acids can encode two different immunogenic peptides. Therefore, in some embodiments, the vaccine composition can transfect (1) a first nucleic acid encoding a first immunogenic peptide that can induce more immediate treatment effect to an existing disease or condition and (2) a second nucleic acid encoding a different, second immunogenic peptide that is aimed to induce adaptive immunity in the subject for future occurrence of a different disease or condition. In some embodiments, the vaccine can deliver two or more different nucleic acids to a subject and each nucleic acid independently exhibits a therapeutic or prophylactic effect, respectively.

In embodiments a vaccine composition can have two or more different types (or different formulas) of cationic amphipathic polymer. Alternatively, a vaccine composition can have only a single type (or single formula) of cationic amphipathic polymer. In some embodiments, a single type of cationic amphipathic polymer can be non-covalently bound to one type (sequence) of nucleic acid. Alternatively, a single type of cationic amphipathic polymer can be non-covalently bound to two or more types (sequences) of nucleic acid. Therefore in embodiments, a mixture of different types of cationic amphipathic polymers, each of which is bound to a different sequence of nucleic acid, can be administered together to a subject in order to deliver two or more sequences (or types) of nucleic acids. Alternatively, a single type (or formula) of cationic amphipathic polymer that is bound to multiple types (or sequences) of nucleic acid can be administered to a subject in order to deliver two or more sequences (or types) of nucleic acid. Still alternatively, a single type (or formula) of cationic amphipathic polymer that is bound to a single sequence (or type) of nucleic acid can be administered to a subject.

In some embodiments, the nucleic acid that is contained the vaccine or composition thereof can be mRNA. In some embodiments, nucleic acid is transfected into one or more cells in the subject via vaccination. In some embodiments, one or more than one nucleic acid sequences can be transfected via a vaccine composition. Therefore, in some embodiments, a vaccine composition contains two different nucleic acids, each of which encodes different antigenic peptides, respectively. Accordingly, when the vaccine is administered into a subject in need of the vaccination, two or more types of antigenic epitopes can be expressed and induce immune responses in the subject. In alternative embodiments, one type of nucleic acid can be transfected via vaccination such that one type of epitope can be expressed and induce an immune response in the subject.

### Administration

In aspects provided are methods for delivering the compositions provided herein including embodiments thereof to cells or a subject so as to provide a desired activity into the cells or subject. In some embodiments, the composition contains a cell-penetrating complex having a cargo nucleic acid that is non-covalently bound to a cationic amphipathic polymer. The cargo nucleic acid, when transfected into the cells or administered to the subject, can provide a variety of intended effects, depending on the nature of the nucleic acid sequence. Some non-limiting examples of intended effects include modulation on gene expression, modulation of cellular pathways, genome-edition and induction of an immune response. In some embodiments, the composition can be administered to a subject in an effective amount that is sufficient to achieve at least part of the intended effects in the subject.

“Administration,” “administering” and the like, when used in connection with a composition refer both to direct administration, which may be administration to cells in vitro, administration to cells in vivo, administration to a subject by a medical professional or by self-administration by the subject and/or to indirect administration, which may be the act of prescribing a composition of the disclosure. When used herein in reference to a cell, refers to introducing a composition to the cell. Typically, an effective amount is administered, which amount can be determined by one of skill in the art. Any method of administration may be used. Compounds (e.g., drugs and antibodies) may be administered to the cells by, for example, addition of the compounds to the cell culture media or injection in vivo. Administration to a subject can be achieved by, for example, intravascular injection, direct intratumoral delivery, and the like.

Administering may mean oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the disclosure can be administered alone or can be co-administered to the patient. Co-administration is meant to include simultaneous or sequential administration of the compounds individually or in combination (more than one compound).

The dosage and frequency (single or multiple doses) administered to a subject can vary depending upon a variety of factors, for example, whether the subject suffers from another disease, its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein including embodiments thereof. Adjustment and manipulation of established dosages (e.g., frequency and duration) are well within the ability of those skilled in the art.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

In some embodiments, the subject is a mammal, for example a human, a non-human primate, a murine (i.e., mouse and rat), a canine, a feline, or an equine. In one embodiment, the subject is a human.

In some embodiments, a composition can be administered in a dose (or an amount) of about 1 ng/kg of subject body weight, about 10 ng/kg of subject body weight, about 50 ng/kg of subject body weight, about 100 ng/kg of subject body weight, about 500 ng/kg of subject body weight, about 1 ug/kg of subject body weight, about 10 μg/kg of subject body weight, about 50 ug/kg of subject body weight, about 100 μg/kg of subject body weight, about 150 μg/kg of subject body weight, about 200 μg/kg of subject body weight, about 250 μg/kg of subject body weight, about 300 μg/kg of subject body weight, about 350 μg/kg of subject body weight, about 375 μg/kg of subject body weight, about 400 μg/kg of subject body weight, about 450 μg/kg of subject body weight, about 500 μg/kg of subject body weight, about 550 μg/kg of subject body weight, about 600 μg/kg of subject body weight, about 650 μg/kg of subject body weight, about 700 μg/kg of subject body weight, about 750 μg/kg of subject body weight, about 800 μg/kg of subject body weight, about 850 μg/kg of subject body weight, about 900 μg/kg of subject body weight, about 1 mg/kg of subject body weight, about 10 mg/kg of subject body weight, about 50 mg/kg of subject body weight, about 100 mg/kg of subject body weight, about 500 mg/kg of subject body weight, about 1 g/kg of subject body weight or more or any intervening ranges of the of the foregoing. In some embodiments, a composition can be administered in a dose (or an amount) of about 0.5 μg, about 1.0 μg, about 1.5 μg, about 2.0 μg, about 2.5 μg, about 3.0 μg, about 3.5 μg, about 4.0 μg, about 4.5 μg about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 1.0 mg, about 1.5 mg, about 2.0 mg, about 2.5 mg, about 3.0 mg, about 3.5 mg, about 4.0 mg, about 4.5 mg about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 1 g or more or any intervening ranges of the foregoing. In some embodiments, a composition can be administered in a dose (or an amount) of about 7.5 μg or about 0.375 mg/kg of subject body weight. Administration can be repeated over a desired period, e.g., repeated over a period of about 1 day to about 5 days or once every several days, for example, about five days, over about 1 month, about 2 months, etc. The weight herein can be a weight of a cell-penetrating complex or a weight of a composition or pharmaceutical formulation thereof. In some embodiments,

In one embodiment, a composition can be administered systemically or locally (e.g. intratumoral injection, intravenous injection) at intervals of 6 hours, 12 hours, daily or every other day or on a weekly or monthly basis to elicit the desired benefit or otherwise provide a therapeutic effect.

In one embodiment, a response rate to a composition, in particular a cancer vaccine, can be reduced as compared to baseline reference or control reference. The term “response rate” is used herein in its customary sense to indicate the percentage of patients who respond with cancer recession following treatment. Response rates include, for example, partial or complete recession. A partial response includes an about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, or about 99% recession of cancer cells. In some embodiments, the control reference is obtained from a healthy subject, a cancer subject (e.g., the cancer subject being treated or another cancer subject), or any population thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In embodiments, the cationic amphipathic polymer is allowed to degrade within the cell thereby forming a degradation product. In embodiments, the degradation product is a substituted or unsubstituted diketopiperazine.

In embodiments, the methods further include allowing the mRNA to be expressed in the cell. In embodiments, the cell is an eukaryotic cell. In embodiments, the cell is a mammalian or human cell. In embodiments, the cell forms part of an organism. In embodiments, the organism is a human. In embodiments, the cell is a lymphoid cell or a myeloid cell. In embodiments, the cell is a T cell. In embodiments, the cell is a myeloid cell.

In an aspect is provided, a method of inducing an immune response in a subject in need thereof, the method including administering an effective amount of the complex as provided herein including embodiments thereof.

### P EMBODIMENTS

P Embodiment 1. A cell-penetrating complex comprising a ribonucleic acid comprising a sequence encoding a viral protein, a nucleic acid adjuvant and a cationic amphipathic polymer.

P Embodiment 2. The cell-penetrating complex of P embodiment 1, wherein said ribonucleic acid is non-covalently bound to said cationic amphipathic polymer.

P Embodiment 3. The cell-penetrating complex of P embodiment 1 or 2, wherein said viral protein is a respiratory syncytial virus (RSV) protein, human metapneumovirus (hMPV) protein, parainfluenza virus type 3 (PIV3) protein, influenza H10N8 virus protein, influenza H7N9 virus protein, cytomegalovirus (CMV) protein, Zika virus protein, chikungunya virus protein, or a severe acute respiratory syndrome (SARS) associated coronavirus (CoV) protein.

P Embodiment 4. The cell-penetrating complex of one of P embodiments 1 to 3, wherein said viral protein is a SARS-CoV-1 protein or a SARS-CoV-2 protein.

P Embodiment 5. The cell-penetrating complex of one of P embodiments 1 to 3, wherein said viral protein is a SARS-CoV-2 spike protein or fragment thereof.

P Embodiment 6. The cell-penetrating complex of one of P embodiments 1 to 3, wherein said viral protein is the soluble receptor binding domain (RBD) of a SARS-CoV-2 spike protein.

P Embodiment 7. The cell-penetrating complex of one of P embodiments 1 to 6, wherein said ribonucleic acid comprises the sequence of SEQ ID NO:3.

P Embodiment 8. The cell-penetrating complex of one of P embodiments 1 to 7, wherein said nucleic acid adjuvant is a DNA adjuvant.

P Embodiment 9. The cell-penetrating complex of one of P embodiments 1 to 7, wherein said nucleic acid adjuvant is a toll-like receptor (TLR) agonist.

P Embodiment 10. The cell-penetrating complex of one of P embodiments 1 to 7, wherein said nucleic acid adjuvant comprises one or more unmethylated CpG oligonucleotides.

P Embodiment 11. The cell-penetrating complex of one of P embodiments 1 to 7, wherein said nucleic acid adjuvant is a TLR-9 agonist.

P Embodiment 12. The cell-penetrating complex of one of P embodiments 1 to 7, wherein said nucleic acid adjuvant has the sequence of SEQ ID NO:1 or SEQ ID NO:2.

P Embodiment 13. The cell-penetrating complex of one of P embodiments 1 to 12, wherein said cationic amphipathic polymer comprises a pH-sensitive immolation domain and a lipophilic polymer domain.

P Embodiment 14. The cell-penetrating complex of one of P embodiments 1 to 12, wherein said cationic amphipathic polymer has the formula:

R^{1A}-[L¹-[(LP¹)_{z1}-(LP²)_{z3}-(IM)_{z2}]_{z4}-L²-R^{2A}]_{z5} (I)
wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain;
IM is said pH-sensitive immolation domain;
z5 is an integer from 1 to 10;
z1 and z3 are independently integers from 0 to 100, wherein at least one of z1 or z3 is not 0;
z4 is an integer from 1 to 100; and z2 is an integer from 2 to 100.

P Embodiment 15. The cell-penetrating complex of P embodiment 14, wherein said lipophilic polymer domain has the formula:

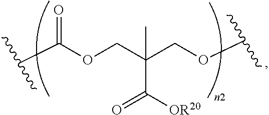

wherein
   n2 is an integer from 1 to 100;
      R²⁰ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 16. The cell-penetrating complex of P embodiment 14, wherein LP¹ has the formula:

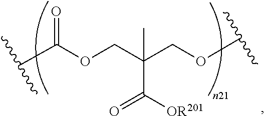

wherein
n21 is an integer from 1 to 100;
   R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 17. The cell-penetrating complex of P embodiment 16, wherein n21 is 5 and R²⁰¹ is unsubstituted C₁₃ alkenyl.

P Embodiment 18. The cell-penetrating complex of P embodiment 17, wherein said unsubstituted C₁₃ alkenyl is oleyl.

P Embodiment 19. The cell-penetrating complex of P embodiment 14, wherein LP² has the formula:

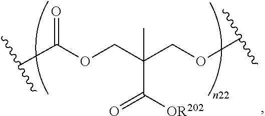

wherein
n22 is an integer from 1 to 100;
   R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 20. The cell-penetrating complex of P embodiment 19, wherein n22 is 5 and R²⁰² is unsubstituted C₉ alkenyl.

P Embodiment 21. The cell-penetrating complex of P embodiment 20, wherein said unsubstituted C₉ alkenyl is nonenyl.

P Embodiment 22. The cell-penetrating complex of P embodiment 14, wherein said cationic amphipathic polymer has the formula:

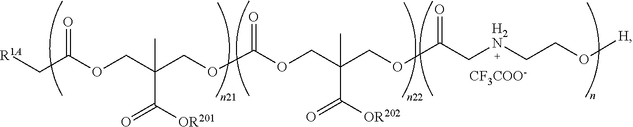

wherein n21 is 5, R²⁰¹ is oleyl, n22 is 5, R²⁰² is nonenyl and n is 7.

P Embodiment 23. The cell-penetrating complex of one of P embodiments 1 to 12, wherein said cationic amphipathic polymer has the formula:

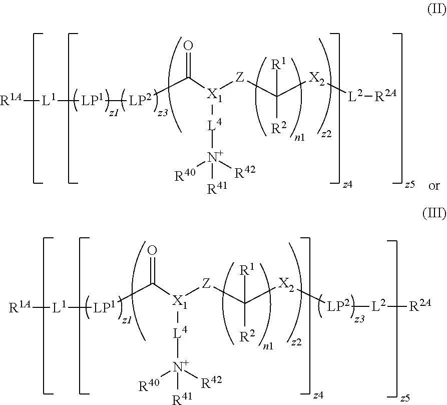

wherein
R^{1A} is independently hydrogen, halogen, —CCl₃, —CBr₃, —CF₃, —C₁₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is independently hydrogen, halogen, —CCl₃, —CBr₃, —CF₃, —C₁₃, CHCl₂, —CHBr₂, —CHF₂, —CHI₂, —CH₂Cl, —CH₂Br, —CH₂F, —CH₂I, —CN, —OH, —NH₂, independently —COOH, —CONH₂, —NO₂, —SH, —SO₃H, —SO₄H, —SO₂NH₂, —NHNH₂, —ONH₂, independently —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain;
X¹ is a bond, —C(R⁵)(R⁶)—, —C(R⁵)(R⁶)—C(R⁷)(R⁸)—, —O—C(R⁵)(R⁶)—, or —O—C(R⁵)(R⁶)—C(R⁷)(R⁸)—;
X² is —O— or —S—;
R¹, R², R⁵, R⁶, R⁷, and R⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
L⁴ is independently a bond, —C(O)O—, —O—, —S—, —NH—, —C(O)NH—, —NHC(O)—, —S(O)₂—, —S(O)NH—, —NHC(O)NH—, substituted or unsubstituted alkylene, or substituted or unsubstituted heteroalkylene;
R⁴⁰, R⁴¹, and R⁴² are independently hydrogen, substituted or unsubstituted alkyl or susbstituted or unsubstituted heteroalkyl;
Z is —S—, —S⁺R¹³—, —NR¹³ —, or —N⁺(R¹³)(H)—;
R¹³ is hydrogen, —CCl₃, —CBr₃, —CF₃, —CI₃, —CN, —OH, ═O, —NH₂, —COOH, —CONH₂, —SH, —SO₃H, SO₂NH₂, —OCCl₃, —OCF₃, —OCBr₃, —OCI₃, —OCHCl₂, —OCHBr₂, —OCHI₂, —OCHF, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
n1 is an integer from 0 to 50;
z1 and z3 are independently integers from 0 to 100, wherein at least one of z1 or z3 is not 0;
z2 is an integer from 2 to 100;
z4 is an integer from 1 to 100;
and
   z5 is an integer from 1 to 10.

P Embodiment 24. The cell penetrating complex of P embodiment 23, wherein LP¹ has the formula:

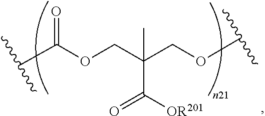

wherein
n21 is an integer from 1 to 100;
   R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 25. The cell penetrating complex of P embodiment 24, wherein n21 is 10-40.

P Embodiment 26. The cell penetrating complex of P embodiment 24, wherein R²⁰¹ is unsubstituted C₁₂ alkyl.

P Embodiment 27. The cell penetrating complex of P embodiment 24, wherein LP² has the formula:

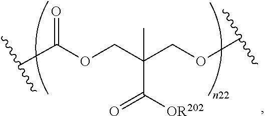

wherein
n22 is an integer from 1 to 100;
   R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 28. The cell penetrating complex of P embodiment 27, wherein n22 is 10-35.

P Embodiment 29. The cell penetrating complex of P embodiment 27, wherein R²⁰² is unsubstituted C₁₂ alkenyl.

P Embodiment 30. The cell penetrating complex of any one of P embodiments 23 to 29, wherein said cationic amphipathic polymer has the formula:

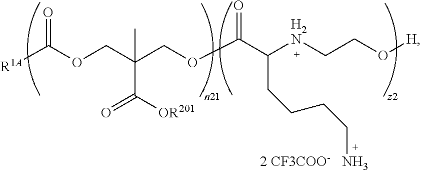

wherein n21 is an integer from 10 to 20;
R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
   z2 is independently an integer from 3-10.

P Embodiment 31. The cell penetrating complex of P embodiment 30, wherein n21 is 14, R²⁰¹ is dodecyl and z2 is 8.

P Embodiment 32. The cell penetrating complex of P embodiment 31, wherein said cationic amphipathic polymer has the formula:

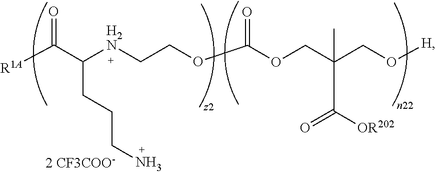

wherein n22 is an integer from 10 to 35;
R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
   z2 is independently an integer from 5-20.

P Embodiment 33. The cell penetrating complex of P embodiment 32, wherein n22 is 14, R²⁰² is dodecyl and z2 is 7.

P Embodiment 34. The cell penetrating complex of P embodiment 23, further comprising a second cationic amphipathic polymer, wherein said second cationic amphipathic polymer is different from said cationic amphipathic polymer.

P Embodiment 35. The cell penetrating complex of P embodiment 34, wherein said second cationic amphipathic polymer has the formula:

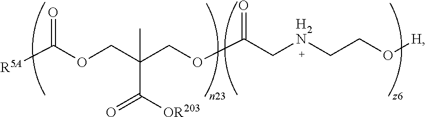

n23 is an integer from 1 to 100;
z6 is an integer from 5-15;
R^{3A} is independently hydrogen, halogen, —CCl₃, —CBr₃, —CF₃, —C₁₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
   R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

P Embodiment 36. The cell penetrating complex of P embodiment 35, wherein n23 is 13, z6 is 11 and R²⁰³ is dodecyl.

P Embodiment 37. A vaccine composition comprising a cell-penetrating complex of any one of P embodiments 1 to 36.

P Embodiment 38. A pharmaceutical composition comprising a therapeutically effective amount of a cell-penetrating complex of any one of P embodiments 1 to 36 and a pharmaceutically acceptable excipient.

P Embodiment 39. A method of treating or preventing a viral disease in a subject in need of such treatment or prevention, said method comprising administering a therapeutically or prophylactically effective amount of a cell-penetrating complex of any one of P embodiments 1 to 36 to said subject.

P Embodiment 40. A method of treating a viral disease in a subject in need thereof, comprising administering to the subject a therapeutically effective amount of a cell-penetrating complex of any one of P embodiments 1 to 36 and a pharmaceutical carrier, thereby treating a viral disease in said subject.

P Embodiment 41. A method for immunizing a host susceptible to a viral disease, comprising administering a cell-penetrating complex of any one of P embodiments 1 to 36 to a host under conditions such that antibodies directed to said viral protein or a functional fragment thereof are produced.

P Embodiment 42. The method of P embodiment 41, wherein said antibodies are IgG2 antibodies.

P Embodiment 43. The method of one of P embodiments 40 to 42, wherein the viral disease is SARS.

P Embodiment 44. The method of one of P embodiments 40 to 42, wherein the viral disease is COVID-19.

P Embodiment 45. The method of one of P embodiments 40 to 42, wherein the viral disease is MERS.

### EXAMPLES

### Example 1: mRNA Vaccine

We have used mRNA encoding a soluble Receptor Binding Domain of the SARS-CoV-2 spike protein. The immune response is focused on the essential part of the SARS-CoV-2 spike protein, which is needed for cellular entry and infectivity. Antibody mediated “disease enhancement” observed with whole spike vaccines in previous corona epidemics SARS-CoV-1 and Middle Eastern Respiratory Syndrome (MERS) is prevented by using fragments and specific domains of viral proteins (e.g., RBD). TLR9 agonist may be used as adjuvant. TLR9 agonists may be co-formulated for effective co-delivery to the same cells that received the RBD encoding mRNA. The TLR9 agonist may activate the innate immune response which in turn aids and elicits adaptive immune responses. It has further previously been shown to assist antibody isotype switching and mature B cell responses. The TLR9 agonist may further enhance T cell responses. The compositions provided herein may be used for highly efficient delivery of nucleic acids to immune cells in living animals and have a desirable toxicity and immunogenicity profile.

CART RBD-mRNA First Experiment—Adjuvant Test, ELISA to detect RBD specific Ab, T cell assays, Receptor binding inhibition/Competition and Pseudoviral neutralization assay:
Group 1: 3 ug RBD mRNA-CART I.V.
Group 2: 3 ug RBD mRNA-CART+3 ug CpG (co-formulated) I.V.
Group 3: 3 ug RBD mRNA-CART+30 ug agonistic CD40mAb (co-formulated) I.V.
Group 4: 3 ug RBD mRNA-CART+30 ug agonistic CD40mAb+3 ug CpG (co-formulated) I.V.
Group 5: 3 ug CpG-CART+30 ug agonistic CD40mAb (co-formulated) I.V CTR Group
Group 6: Naïve mice CTR. Group

We have developed a three-component mRNA vaccine that induces isotype switched neutralizing antibody responses that are detected both in circulation and the lungs of living animals. The three components are: an mRNA encoding the soluble version of the Receptor Binding Domain(RBD) of the SARS-CoV-2 Spike protein, a TLR9 agonist in the form of DNA based oligonucleotide such as CpG, and Charge Altering Releasable Transporters (CARTs). CARTs formulate the mRNA and the TLR9 agonist making a nanoparticle that efficiently delivers TLR9 agonist and the RBD mRNA to cells in living animals.

We have developed a vaccine that works in both inbred mice strains and humanized mice. The CART technology has been assessed for safety in rodent models. The vaccine gives desired responses in pre-clinical models.

### Example 2: mRNA SARS-CoV-2 Vaccine Employing Charge-Altering Releasable Transporters with TLR-9 Agonist Induces Neutralizing Antibodies and T Cell Memory

The SARS-CoV-2 pandemic has necessitated the rapid development of prophylactic vaccines. Two mRNA vaccines have been approved for emergency use by the FDA and have demonstrated extraordinary effectiveness. The success of these mRNA vaccines establishes the speed of development and therapeutic potential of mRNA. These authorized vaccines encode full-length versions of the SARS-CoV-2 spike protein. They are formulated with Lipid Nanoparticle (LNP) delivery vehicles that have inherent immunostimulatory properties. Different vaccination strategies and alternative mRNA delivery vehicles would be desirable to ensure flexibility of future generations of SARS-CoV-2 vaccines and the development of mRNA vaccines in general.

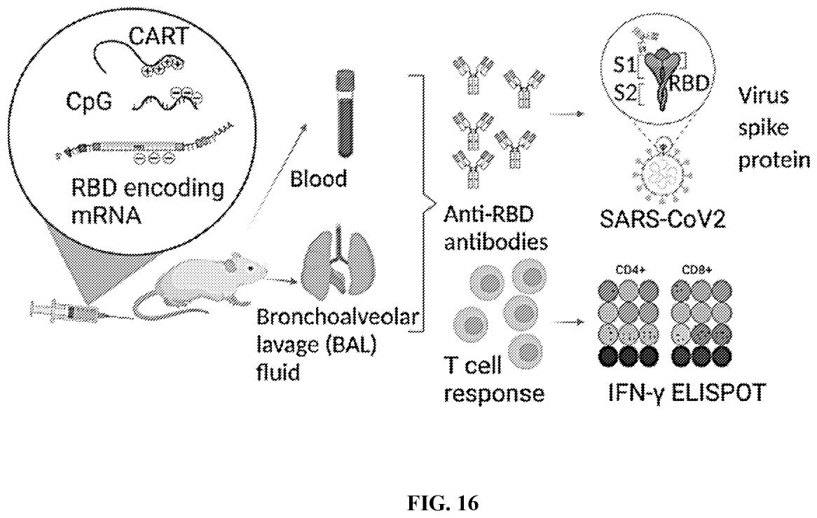

Here, we report on the development of an alternative mRNA vaccine approach using a delivery vehicle called Charge-Altering Releasable Transporters (CARTs). Using these inherently nonimmunogenic vehicles we can tailor the vaccine immunogenicity by inclusion of co-formulated adjuvants such as oligodeoxynucleotides with CpG motifs (CpG-ODN). Mice vaccinated with our mRNA-CART vaccine developed therapeutically relevant levels of RBD-specific neutralizing antibodies in both the circulation and in the lung bronchial fluids (FIG. 16). In addition, our vaccine elicited strong and long lasting RBD-specific T_{H}1 T cell responses including CD4⁺ and CD8⁺ T cell memory.

### INTRODUCTION

Coronavirus pandemics have been a growing concern for more than a decade and several attempts have been made to develop vaccines against SARS-CoV-1 and the Middle Eastern Respiratory Syndrome (MERS).¹ The global spread of the SARS-CoV-2 virus stimulated worldwide efforts to leverage previous insights from coronavirus vaccines to develop safe, effective and scalable vaccines to alleviate the COVID-19 pandemic. While a variety of vaccine candidates and approaches are being investigated worldwide,² the extraordinary pace of development and implementation of mRNA vaccines³⁻⁶ illustrates the potential of this emerging technology. The mRNA vaccines granted emergency use authorization by the FDA against SARS-Cov-2 represents a triumph of basic and applied science as these advances enabled the most rapid clinical translation from concept to clinical trial ever for a vaccine.^{5,6} mRNA is transiently expressed, does not integrate into the genome and is eliminated through natural degradation mechanisms in the body. mRNA vaccines offer flexible and fast design, that will allow for subsequent generations of products to address the emergence of new virus variants.

The currently approved mRNA vaccines^{5,6} generated by in vitro transcription use chemically-modified nucleotides incorporated in mRNAs encoding the full viral spike protein, usually containing 2 structural epitope mutations, formulated in lipid nanoparticles (LNPs) and are administered intramuscularly. Despite their extraordinary success, the underlying science that contributes to the most effective, safe, and scalable vaccine against COVID-19 continues to evolve. Modifications and sequence optimization of the mRNA and the particular components of the delivery vehicle can influence the immunological response.⁷ Previous studies of SARS-CoV and MERS have shown that the proper choice of encoded antigen is critical⁸ to avoid potential complications from antibody dependent enhancement (ADE) of disease.⁹ The chemistry of the delivery vehicle is also important as the ionizable lipids that are a component of LNPs act as adjuvants but can induce adverse events¹⁰ and the use of polyethylene glycol (PEG) in the LNP formulations can contribute to allergic reactions.^{11,12}

These continuing challenges and the degree to which the global scale of the COVID-19 pandemic has strained supply chains for the existing LNP technologies highlight the need to develop alternative approaches, both to enhance the resilience of the global response in the face of this and future pandemics, as well as to test alternative approaches that might lead to the most effective and durable immunological responses as well as to be able respond rapidly to emerging new virus variants¹³.

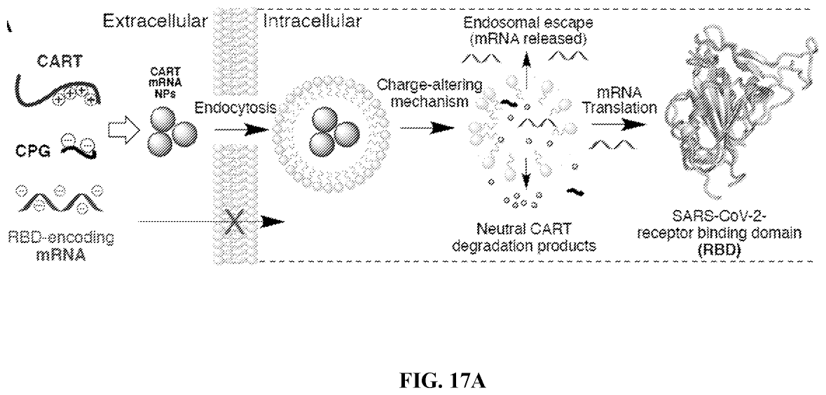

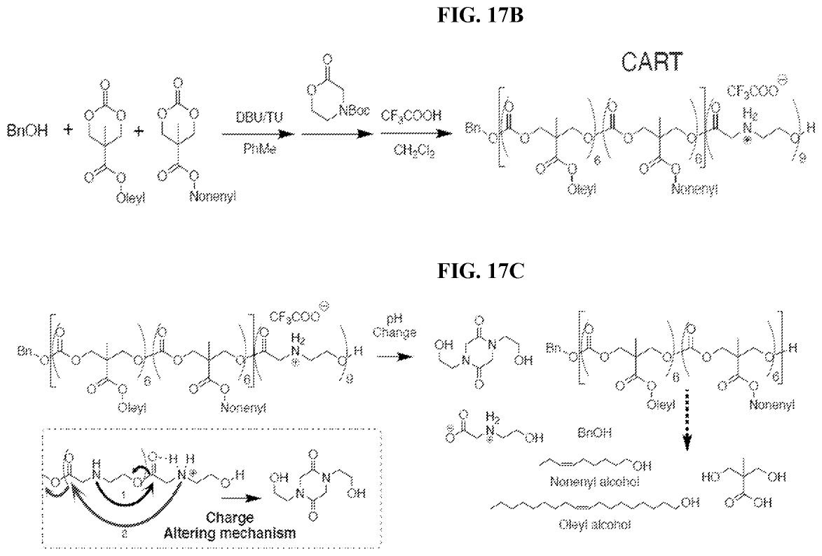

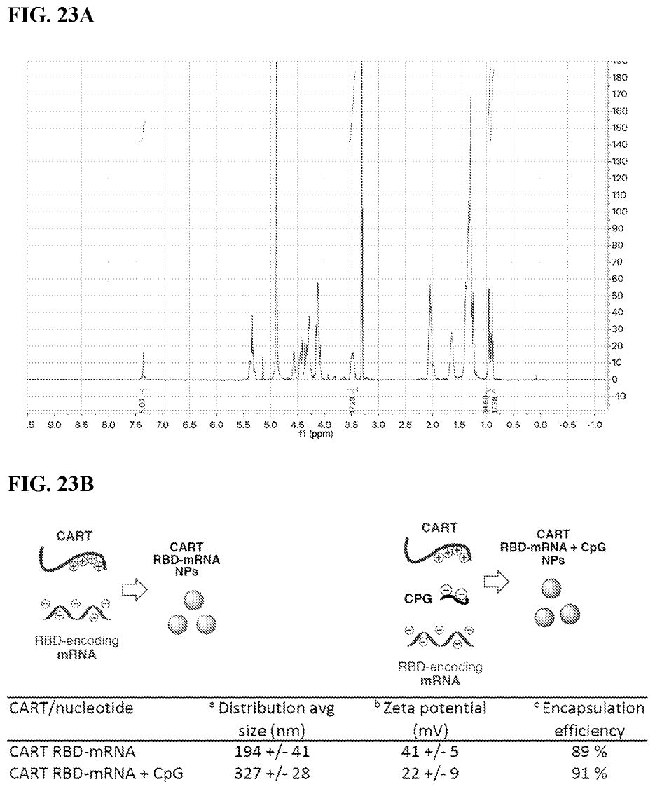

In this study we present an alternative 3-component mRNA vaccine utilizing mRNA encoding the receptor binding domain (RBD) of the SARS-CoV-2 spike protein formulated with a highly efficient, non-toxic, PEG-free mRNA delivery platform called Charge-Altering Releasable Transporters (CARTs)¹⁴⁻¹⁶ and a TLR9 agonist (CpG) as co-formulated adjuvant^{16,17} (FIG. 17A). Prior studies had indicated that the RBD is a promising antigen target¹⁸⁻²⁰ as antibodies elicited against this target are often strongly neutralizing. CARTs offer a promising new gene delivery platform and have proven to be effective deliverers of mRNA vaccines in preclinical mouse studies.^{16,21} CARTs are single component amphiphilic diblock oligomers containing a sequence of lipid monomers and a sequence of cationic monomers (FIGS. 17B, 23A-23B). They are readily produced on scale in a two-step organocatalytic oligomerization. CARTs electrostatically encapsulate mRNA (or other co-formulated nucleotides like CpG) and deliver the genetic cargo into cells. A unique feature of CARTs is their ability to undergo a charge-altering rearrangement to produce neutral diketopiperazine small molecules (DKPs). This transformation facilitates the release of mRNA (FIG. 17C) and eliminates any toxic issues associated with persistent cations.²² Previous observations had shown that upon I.V. injection, CARTs containing hydroxyethyl glycine repeating cation units selectively deliver mRNA to the spleen,¹⁴⁻¹⁶ whereas intramuscular (IM) injections of these same products result in mRNA translation locally in the muscle (FIG. 17D). Bioluminescence studies with fLuc mRNA indicate in vivo protein expression is greater in the spleen after I.V. injection than in the muscle after I.M. injection. In either site expression peaks after 4-6 hours (FIG. 17E) and decreases over a period of 3-4 days. Moreover, CARTs containing unsaturated lipid blocks exhibit enhanced transfection of antigen presenting cells¹⁵ motivating our choice of such a CART for the COVID-19 vaccine. The CART delivery vehicle does not induce nonspecific immune stimulation by itself.^{16,21} This property allows the co-formulation of oligodeoxynucleotideadjuvants such as the TLR9 agonist CpG-ODN to tune the induced immune response.

Here we show that co-formulation of a CART with mRNA encoding the SARS-CoV-2 RBD with the TLR9 agonist CpG (RBD mRNA+CpG-CART) induces robust neutralizing antibodies and RBD-specific T cell responses in mice. Moreover, we detect significant levels of these antibodies and memory T cells in the spleen and lung of vaccinated animals.

Results

RBD mRNA+CpG-CART Vaccination Elicits Anti-RBD-Specific Antibodies at Day 4 after Immunization.

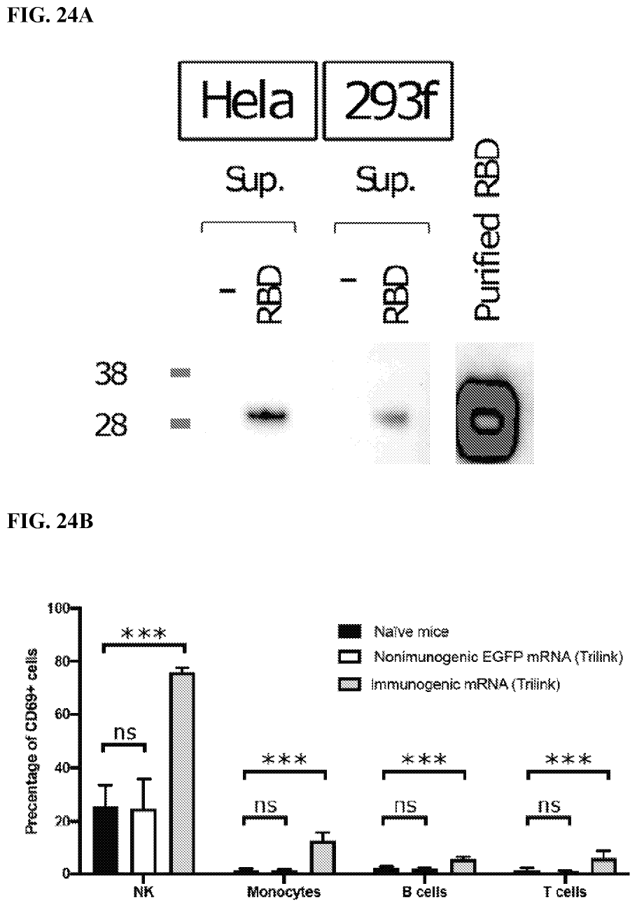

An mRNA encoding the receptor binding domain (RBD)23 of SARS-CoV-2 was made by in vitro transcription and based on the published sequence of the virus. A His tag was included to allow for protein detection of the translated mRNA product as a quality control step. The resulting mRNA contained the optimal CAP-1 structure; uridines were replaced with modified N1-methyl-pseudouridine, and cytidines were replaced with 5-methylcytidine to maximize mRNA stability and translation. Protein expression was verified by Western Blot in transfected 293F and HeLa cells (FIG. 24A). To demonstrate that the administration of mRNA-CART complexes does not lead to nonspecific immune stimulatory effects when formulated with optimally modified mRNA,²⁴ mice were injected intravenously (IV) with mRNA-CART and monitored for the activation of innate immune cells, a test that we have found to be most sensitive. mRNA-CART complexes were free of such non-specific stimulatory effects when formulated with modified mRNA. However, when formulated with unmodified mRNA, nonspecific activation of innate immune cell subsets could be observed, demonstrating that the property of nonspecific immune stimulation is dependent on the cargo and not the CARTs (FIG. 24B). We were then able to direct the immune response of our candidate vaccine by coformulating the mRNA-CART complexes with CpG oligonucleotides that trigger endosomal TLR9 receptors in antigen presenting cells. These CpG entities are known to be safe and effective vaccine adjuvants.¹⁷

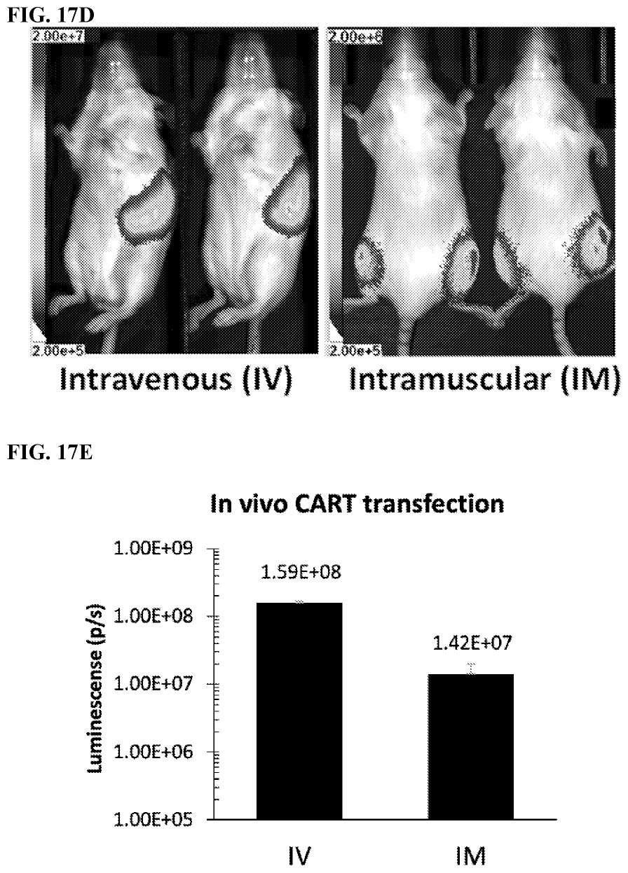

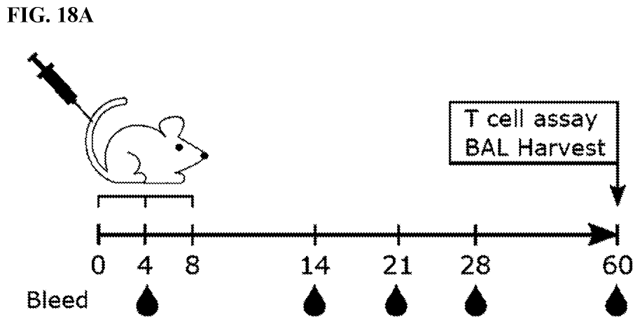

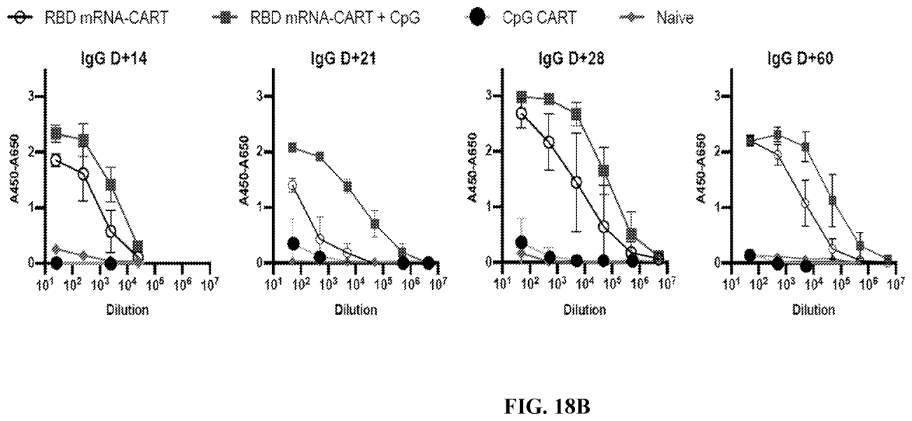

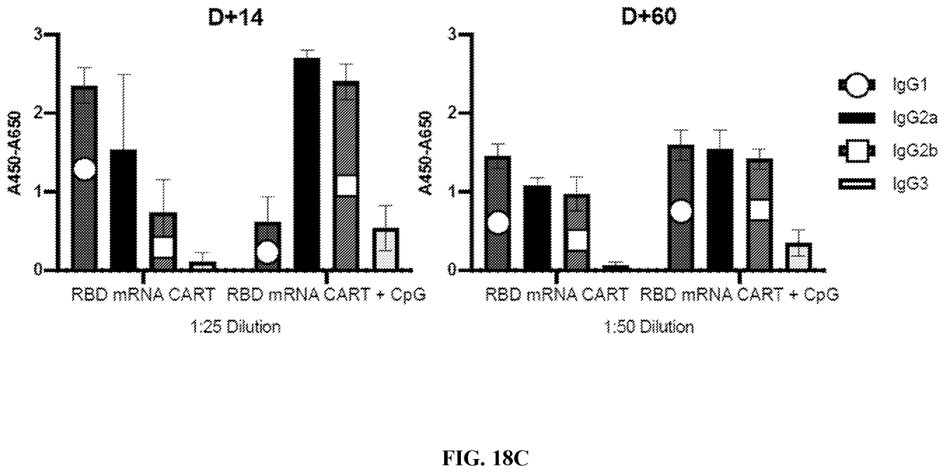

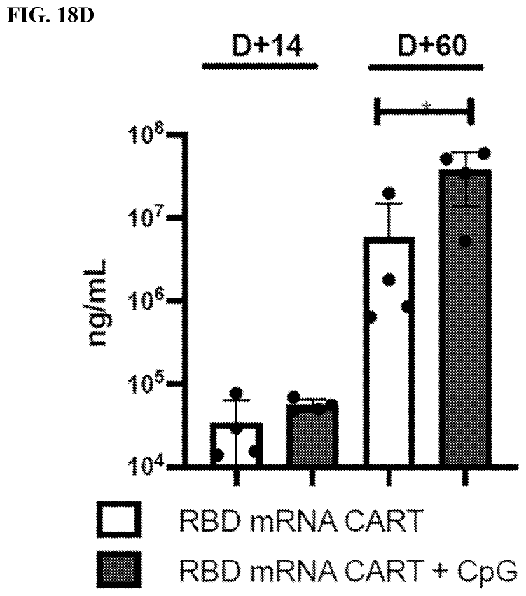

We assessed the effects of the RBD mRNA-CART vaccination with or without the addition of CpG as the adjuvant. IV injection of CARTs effectively delivers the cargo to antigen presenting cells (APCs) such as B cells, macrophages, and dendritic cells in the spleen^{15,16} (FIG. 17D). This route of administration of the mRNA-CART vaccination had proven effective in previous studies of therapeutic cancer vaccination.¹⁶ Based on this knowledge, we evaluated our vaccine first by IV administration. Mice were primed on day 0 and received two boosts on day 4 and on day 8 with 3 g of RBD mRNA-CART formulated with or without 3 g of CpG (FIG. 18A). As a control, a group that received 3 g of CpG-CART alone with no mRNA and a control group treated with phosphate buffered saline (PBS) were included. Mice vaccinated with RBD mRNA-CART plus CpG developed detectable levels of anti-RBD IgG and IgM as early as 4 days after vaccination (FIG. 25A). Over time, we observed an increase in the levels of anti-RBD antibodies in the serum of both mRNA-treated groups compared to controls (FIG. 18B). Importantly, the antibodies induced by our vaccine were specific for RBD and did not cross react to an irrelevant His tagged protein (GFP-His) (FIG. 25B). Notably, RBD-specific antibody responses in mice vaccinated with the formulation including CpG consistently exceeded those observed in groups treated without CpG (FIG. 18B, 18D).

Addition of CpG Results in Early Isotype Switching of RBD-Specific Antibody Responses.

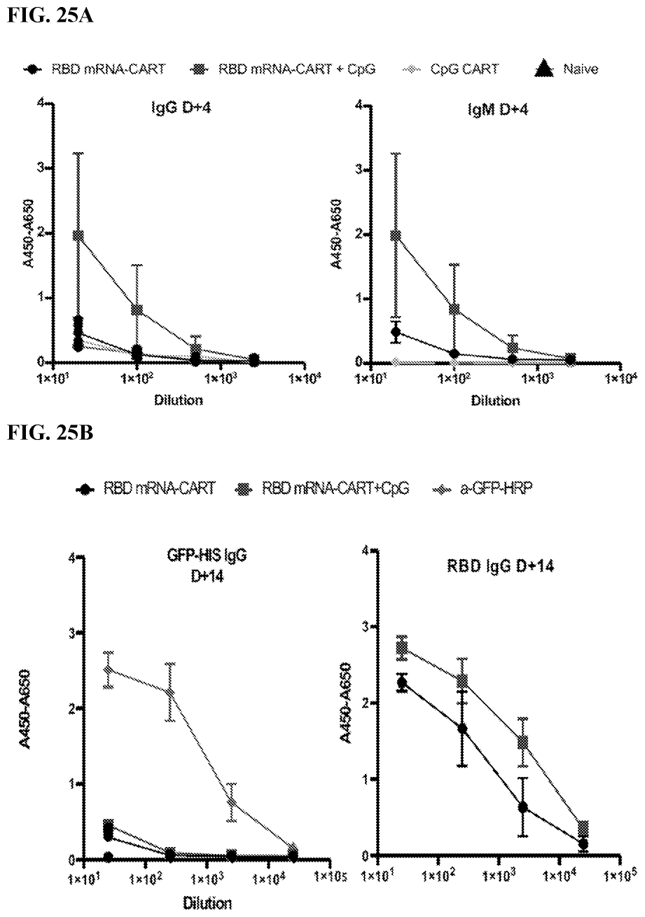

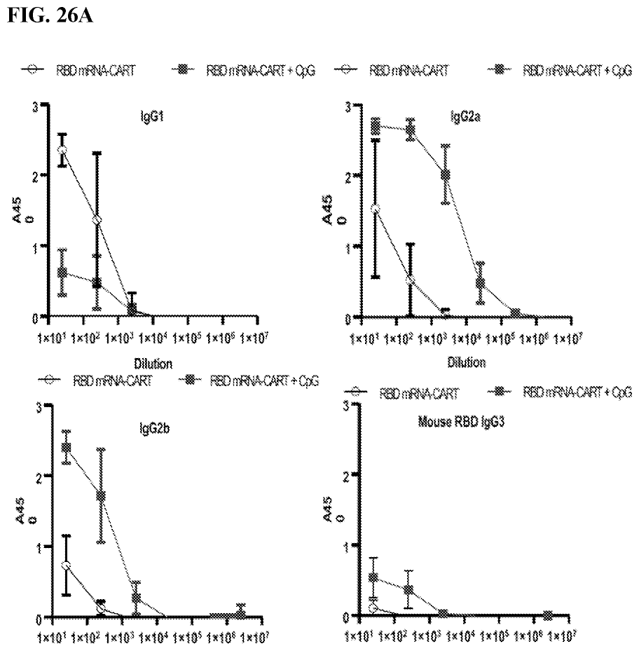

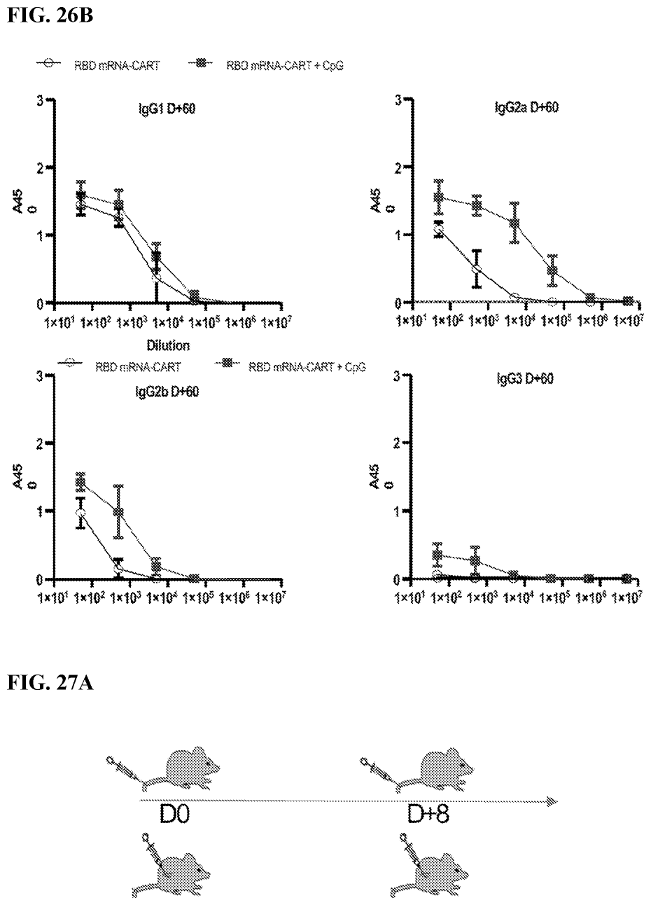

By day 14, we observed that the CpG coformulated vaccine induced an RBD-specific Ig response that had undergone isotype switching from IgG1 to IgG2a, IgG2b, and IgG3. By contrast, mice receiving the vaccine formulation without coformulated CpG produced predominantly unswitched IgG1 (FIGS. 18C, 26A and 26B). By day 60, these differences were less apparent; however, we detected higher levels of all classes of RBD-specific antibodies in the serum of mice vaccinated with the CpG-containing vaccine (FIG. 18C, 26B).

Antibodies Induced by RBD mRNA+CpG-CART Vaccination Inhibit RBD-ACE2 Binding and Neutralize Pseudotyped Viral Entry.

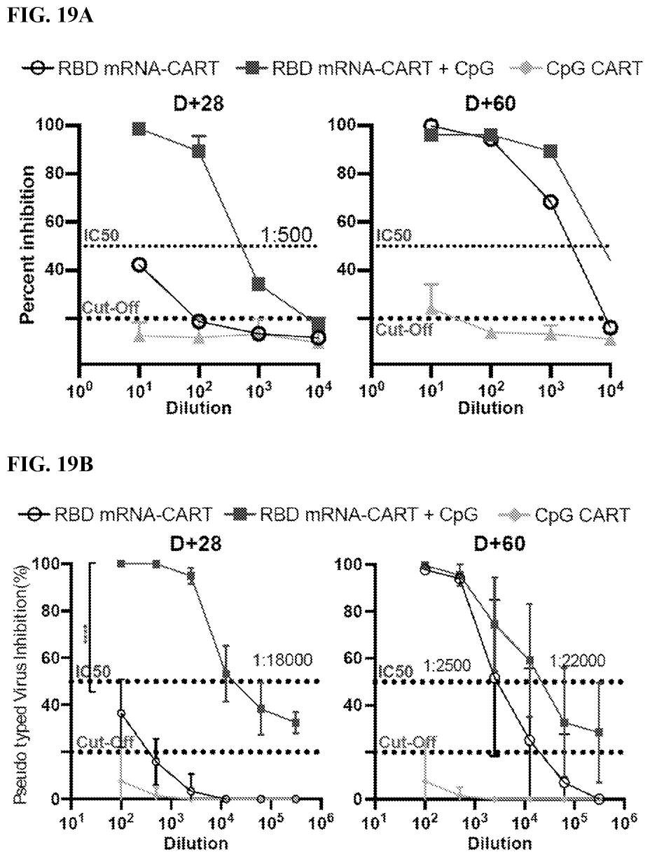

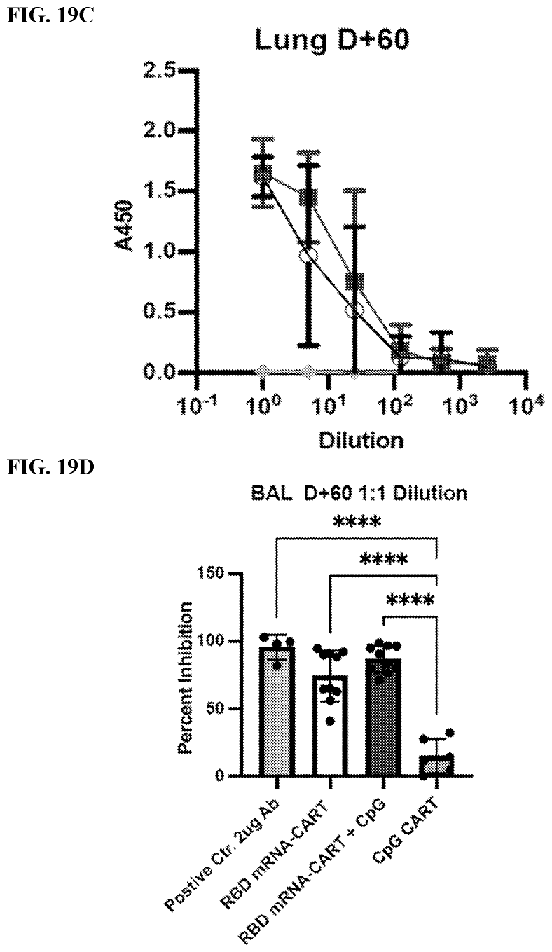

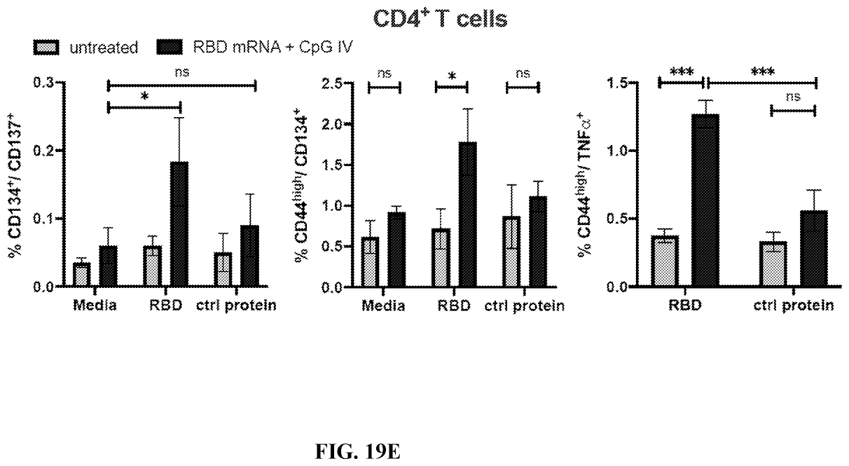

The induced antisera were next tested for their ability to inhibit the RBD-ACE2 interaction (FIG. 19A) and to block pseudotyped viral entry into ACE2-expressing target cells (FIG. 19B). Functional RBD-ACE2 receptor blocking was assayed both against the binding of RBD protein to the ACE2-expressing target cell by flow cytometry (data not shown) and by binding to the solid phase coated with ACE2 protein. On day 28, we observed a striking difference in the level of neutralizing antibodies between the group that had received the vaccine containing the CpG as compared to the group vaccinated without the adjuvant. While antisera from RBD mRNA+CpG-CART vaccinated mice displayed a high degree of receptor blocking and pseudotyped viral neutralization with IC50 of 1:500 and 1:18 000, respectively, the antisera from mice vaccinated with RBD mRNA-CART were only barely positive above the background (FIGS. 19A, 19B). However, by day 60 antisera from mice vaccinated with RBD mRNA-CART were able to block RBD-ACE2 receptor binding and neutralize pseudotyped viral entry, although to a lesser extent than antisera from mice vaccinated with RBD mRNA+CpG-CART (FIGS. 19A, 19B). The results from the receptor interaction blocking assay and the pseudovirus neutralization assay were well correlated.

RBD-Specific Antibodies in Bronchoalveolar Lavage of Vaccinated Mice.

To evaluate the presence of RBD-specific immunoglobulin in the lungs of vaccinated mice, we collected bronchoalveolar lavage (BAL) on D60 after treat-ment. RBD-specific Ig was detected in BAL from both RBD mRNA+CpG-CART and RBD mRNA-CART vaccinated mice by ELISA (FIG. 19C). Notably, as BAL is collected by flushing lungs with 2 mL of sterile PBS, the Ig titers represent highly diluted samples. Importantly, although diluted, these immunoglobulins from BAL of both vaccinated groups blocked RBD-ACE2 binding on D60 (FIG. 19D).

RBD-Specific CD4⁺ T Cells in the Lung of Vaccinated Mice.

In mice, Ig class switching is linked to T_{H}1 T cell responses.^{25,26} To evaluate the vaccine induced T cell responses, we prepared single-cell suspensions from the lungs of IV vaccinated mice on D28 and cultured the cells for 48 h in media alone or in the presence of soluble RBD-His protein or a control protein (hCD81-His). Cells were then collected and assayed by flow cytometry for T cell activation using fluorochrome conjugated monoclonal antibodies for memory and activation-specific surface proteins and intracellular cytokines. Remarkably, upon RBD protein restimulation, a defined RBD-specific CD4+/CD44^{high}/CD134⁺ and CD4⁺/CD44^{high}/TNFα⁺ activated T cell subset could be identified in lung cell suspensions from mice vaccinated with RBD mRNA+CpG-CART that could not be detected when cells were cultured with media alone or in the presence of the control protein (FIG. 19E). TNFα secretion by CD4⁺ T cells is associated with a T_{H}1 polarization.

RBD mRNA+CpG-CART Vaccine Elicits Robust Anti-RBD Ig Responses by an Intravenous, Intramuscular, and Subcutaneous Route of Administration.

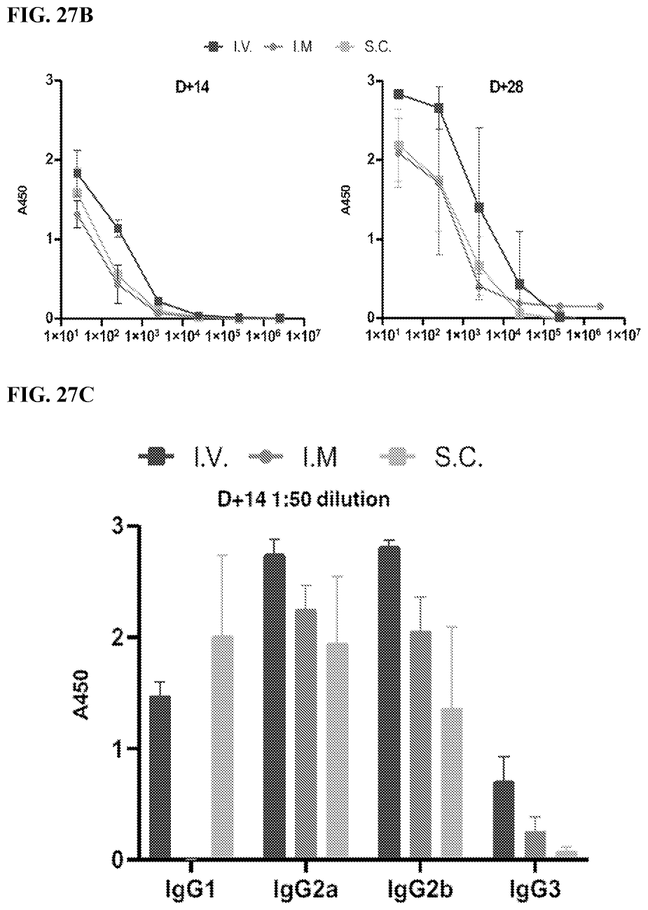

To test the efficacy of our vaccine in relation to the route of administration (ROA), we compared RBD-specific Ig responses induced by immunizations given IV, IM, or SC. Mice were primed with 3 μg of RBD mRNA and 3 μg of CpG formulated in CARTs on day 0 and received a boost on day 8 (FIG. 27A). All routes of administration led to detectable neutralizing antibody titers at the analyzed time points D14 and D28, with no significant differences between IM and SC administration (FIG. 27B). There was a tendency toward higher titers in IV vaccinated mice. Isotype switching occurred independent of the route of administration (FIG. 27C).

RBD mRNA+CpG-CART Vaccine Induces Robust RBD-Specific Ig Responses in a Clinically Relevant Prime-Boost Regimen and Is Independent of CpG Source.

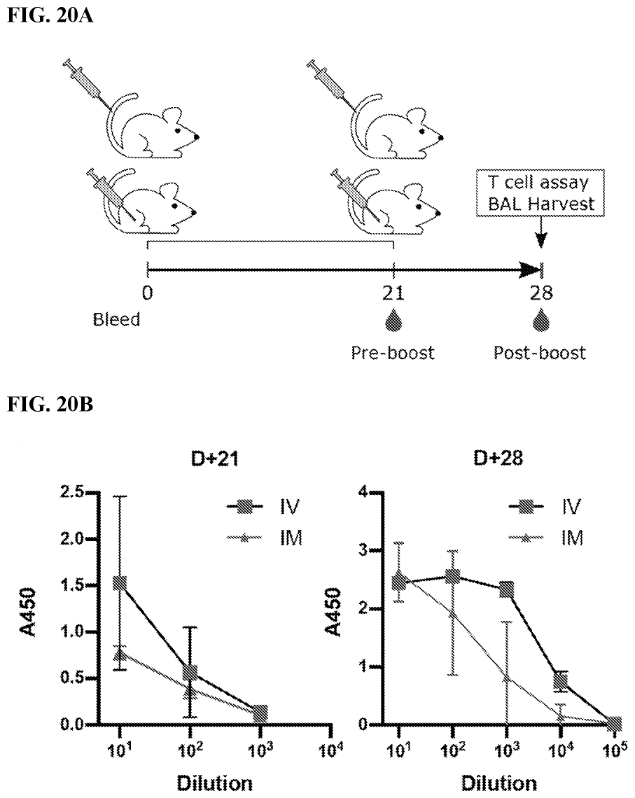

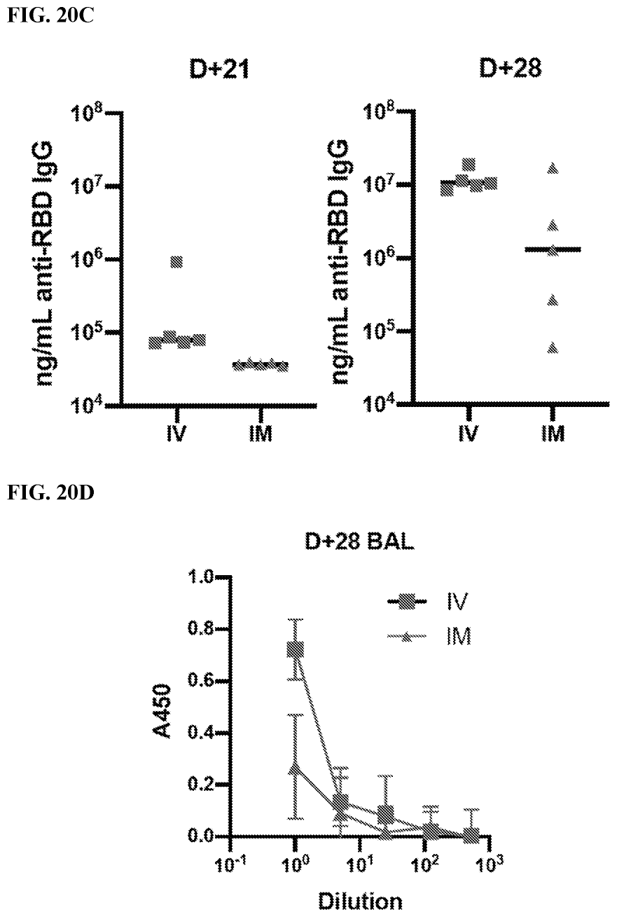

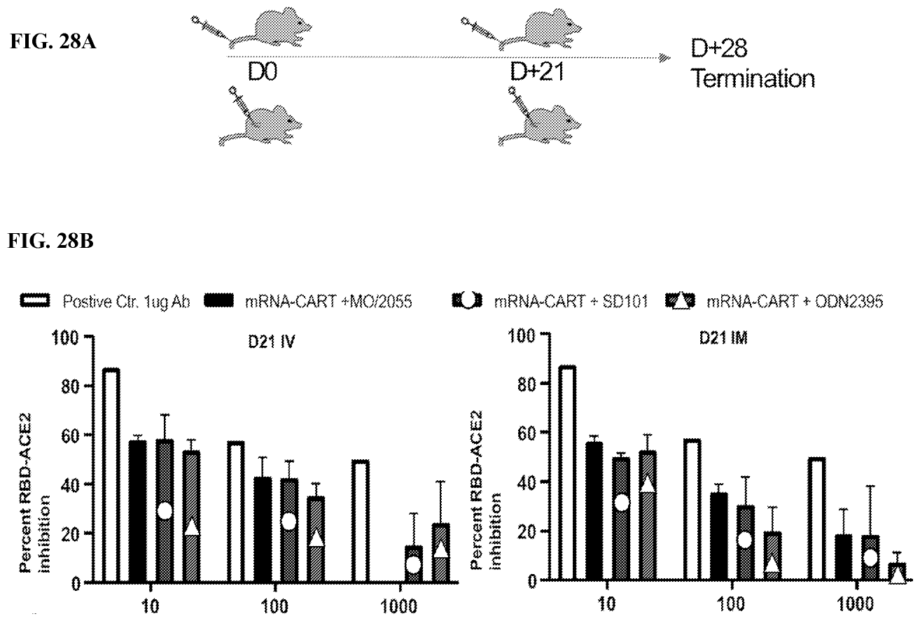

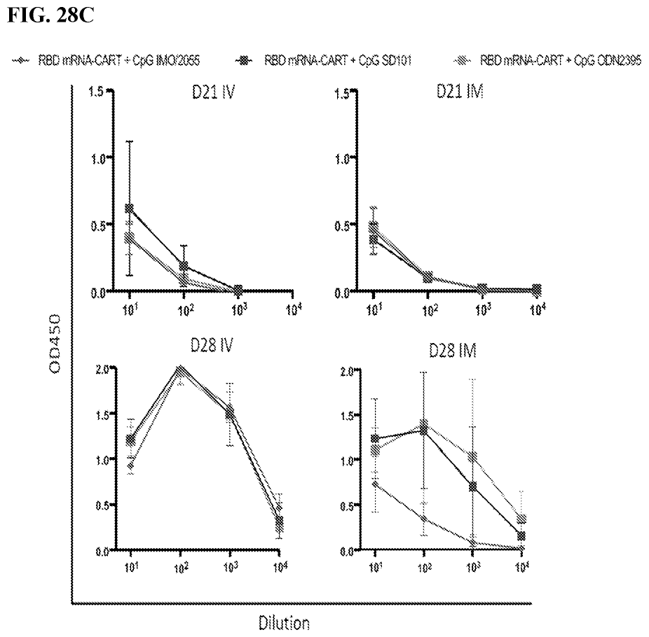

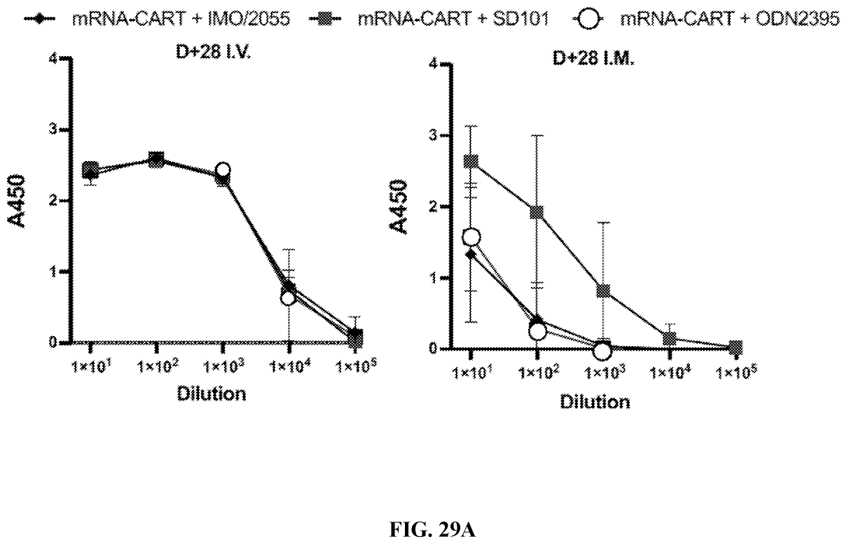

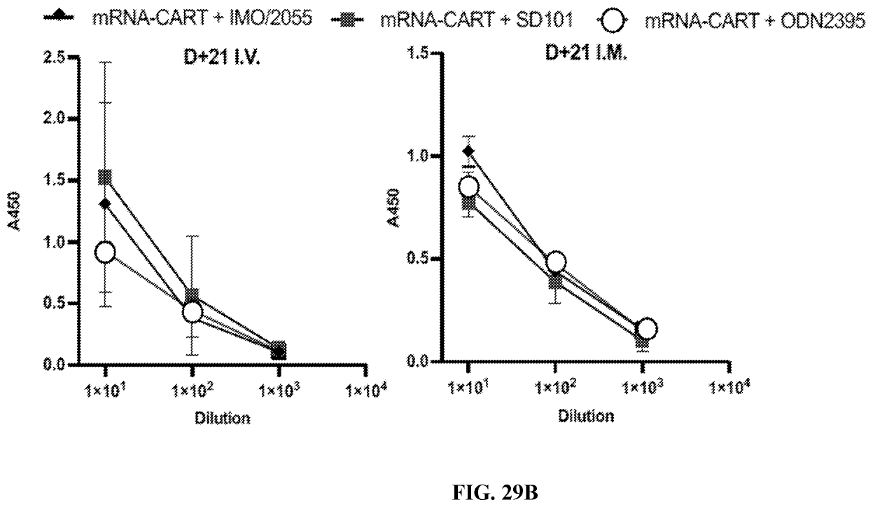

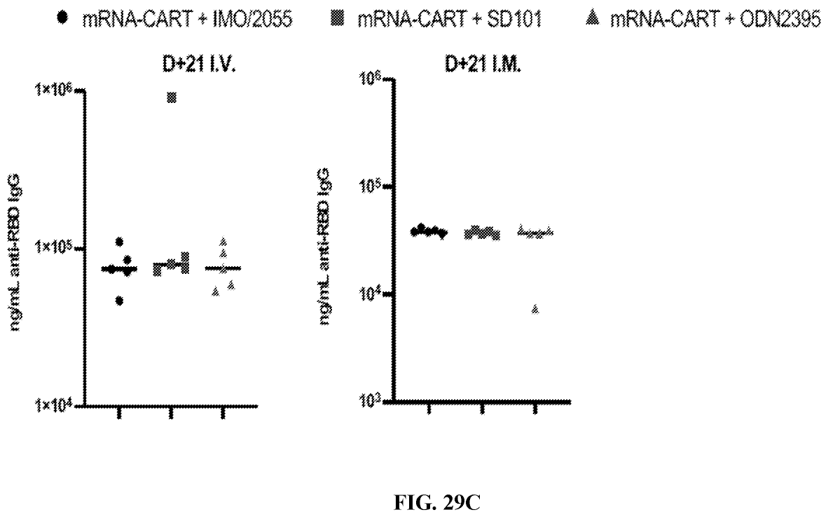

Guided by the immunization regimen chosen by the currently approved SARS-CoV-2 vaccines,²⁷ mice were primed with the vaccine on DO and boosted on D21. Mice were immunized either IV or IM with 3 μg of RBD mRNA+CpG-CART (FIG. 20A). Confirming previous observations, robust responses were observed for both groups. IV immunized mice showed higher-statistically not significant-titers of anti-RBD antibodies in serum and in the BAL on both D21 and D28 (FIGS. 20B-20D). Antisera from both groups effectively inhibited RBD-ACE2 binding on D21 (FIG. 28B), although substantially more effective on D28 reflecting the difference observed in total RBD-specific IgGs between the two groups (FIG. 20C). Moreover, robust antibody responses against the complete spike protein were observed in both groups, although higher in the intravenous group (FIG. 28C). Thus, the vaccine induced anti-RBD response is primarily directed toward exposed RBD epitopes in the complete spike protein. Importantly, although CpG is required for robust isotype switched anti-RBD immunoglobulin, the response is independent of the source of CpG. We tested 4 different sources of CpG, three of the C-subclass of CpGs (CpG-C) and one of the B-subclass (CpG-B). No significant difference was observed between the different CpG-Cs (FIGS. 28A-28C and 29A-29C), while the B-subclass CpG underperformed compared to the CpG-Cs (data not shown).

RBD mRNA+CpG-CART Vaccine Induced Long-Lasting T_{H}1 CD4+ and CD8+ T Cell Memory.

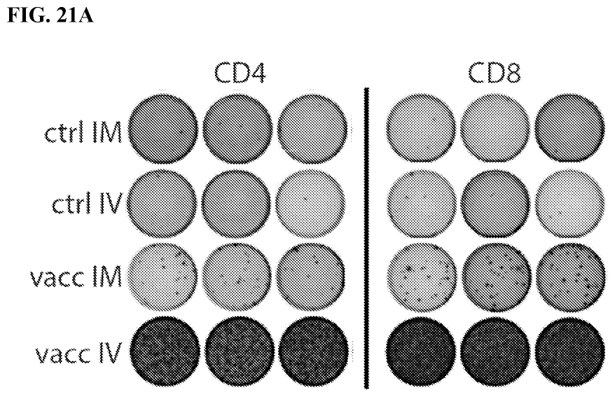

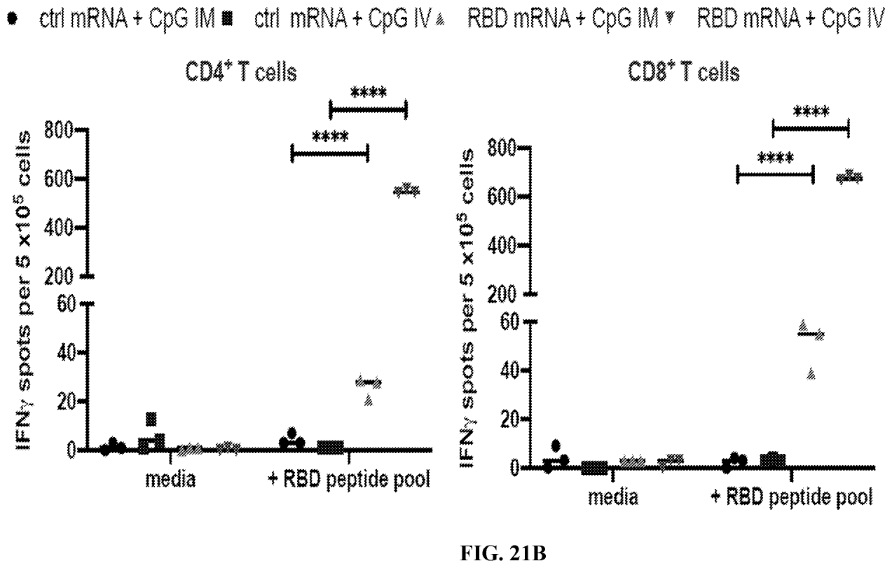

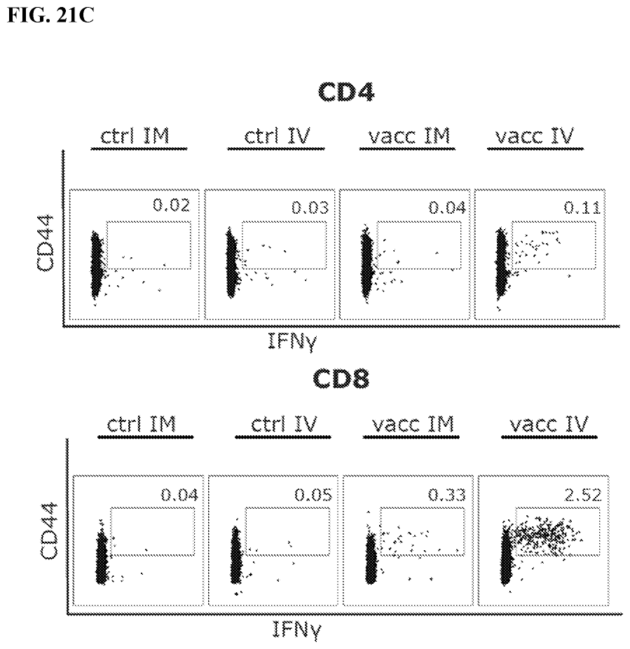

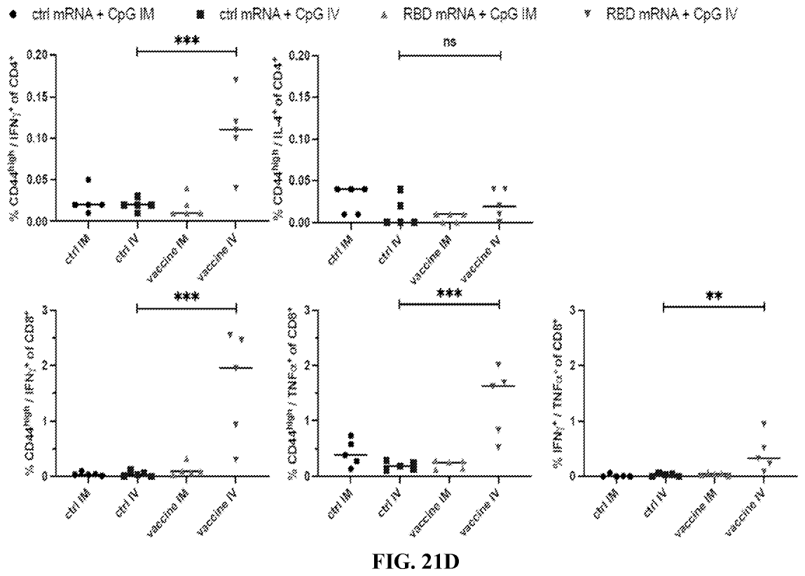

Splenocytes from mice that had received 3 μg of RBD-mRNA+3 μg of CpG-CART or 3 μg of Ctrl mRNA+3 μg of CpG-CART either IV or IM on D1 and D21 were harvested on day 105 after vaccination and characterized for T cell responses by an IFNγ enzyme-linked immunosorbent spot assay (ELISpot). In this assay, pooled splenocytes were enriched for either CD4+ or CD8+ T cells and cultured overnight with a SARS-CoV-2 RBD peptide pool or media alone. Significant IFNγ responses in CD4+ and CD8+ T cells were detected by both IV and IM vaccination. Since the route of administration of IV vaccinated mice targets the spleen, it is expected that spleen T cells would give a stronger response. On the other hand, it is remarkable to detect responding T cells 105 days after vaccination (FIGS. 21A and 21B).

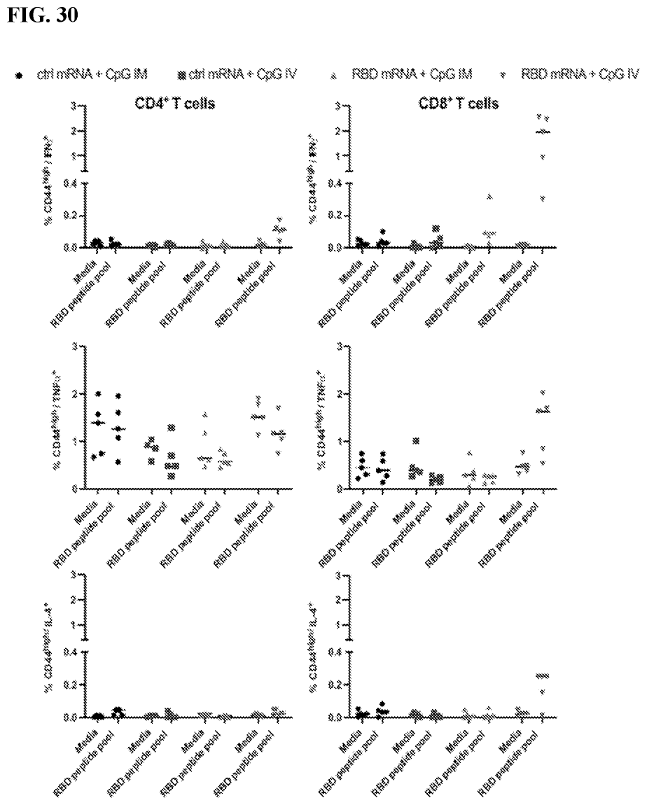

To further assess the functionality and polarization of the vaccine induced T cells, we incubated splenocytes of individual mice from the same experiment in the presence of an RBD peptide pool or media alone. After 18 h of incubation, CD4⁺ and CD8⁺ T cells were assayed separately by flow cytometry for their expression of memory markers CD44 and for the intracellular cytokines IFNγ, TNFα, and IL-4. Even at this late time point after vaccination, a significant population of RBD-specific IFNγ producing CD4⁺ and CD8⁺ T cells and TNFα producing CD8+ T cells could be identified in the RBD-mRNA+CpG-CART IV vaccinated group. There was no increase in IL-4 producing CD4⁺ T cells, indicating that T cell memory was predominately T_{H}1 (FIGS. 21C, 21D and 30). In contrast to the IFNγ ELISpot results, we were not able to identify these low-frequency populations (mean 28 and 55 IFNγ spots per 5×105 cells for CD4⁺ and CD8⁺ enriched conditions, respectively) in IM vaccinated mice by flow cytometry.

Neutralizing Antibody Levels of Immunized Mice Are Comparable to Those Achieved in Vaccinated Humans.

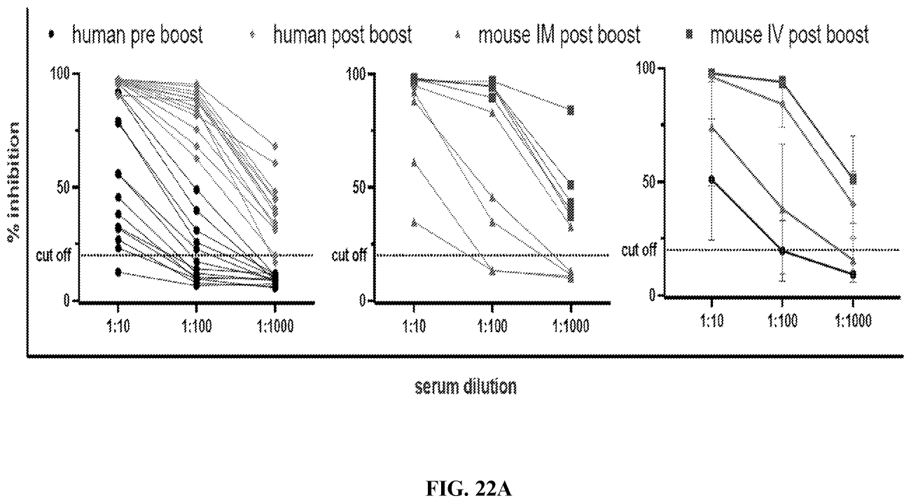

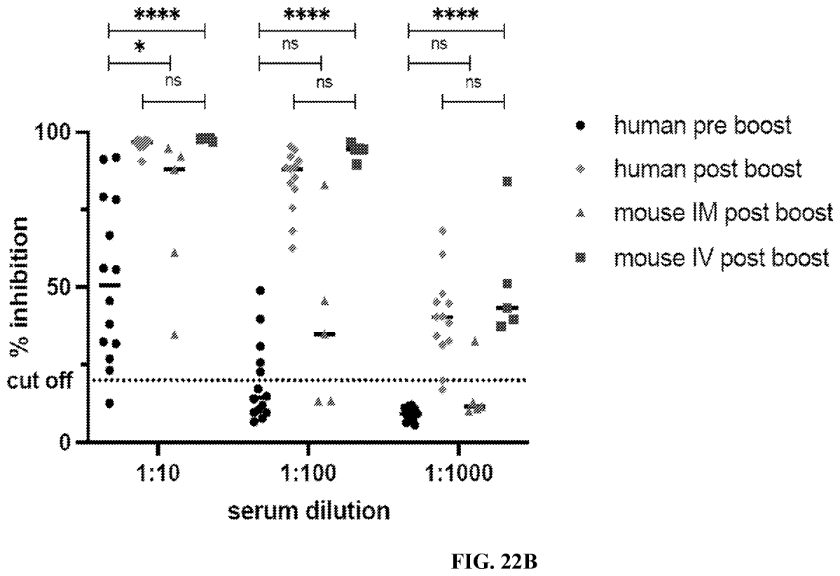

Results from clinical trials indicate that the Pfizer/BioNTech mRNA vaccine and the Moderna vaccine can both confer protection from symptomatic infection prior to administration of their second booster vaccine doses.^{28,29} This implies that the antibody levels in humans at that early preboost time point are sufficient to confer disease protection. Accordingly, we compared the levels of neutralizing antibodies achieved in our vaccinated mice to those in immunized humans both prior to and after their boosters. BALB/c mice were vaccinated with 3 μg of RBD mRNA+3 μg of CpG-CART either IM or IV on D1 and D21, and serum was collected on D28. These mice sera were then compared to sera from 13 individual Pfizer/BioNTech mRNA-LNP vaccinated humans collected 15-21 days after their priming vaccination and then again 15±4 days after their booster vaccinations. The level of RBD-ACE2 inhibition achieved with postboost sera from our IM and IV vaccinated mice was similar to or higher than that of the human preboost sera. The inhibitory antibody levels in the mice receiving the IV vaccination equaled those in humans after boosting (FIGS. 22A and 22B).

RBD mRNA+CpG-CART Vaccination Shows a Favorable Safety Profile.

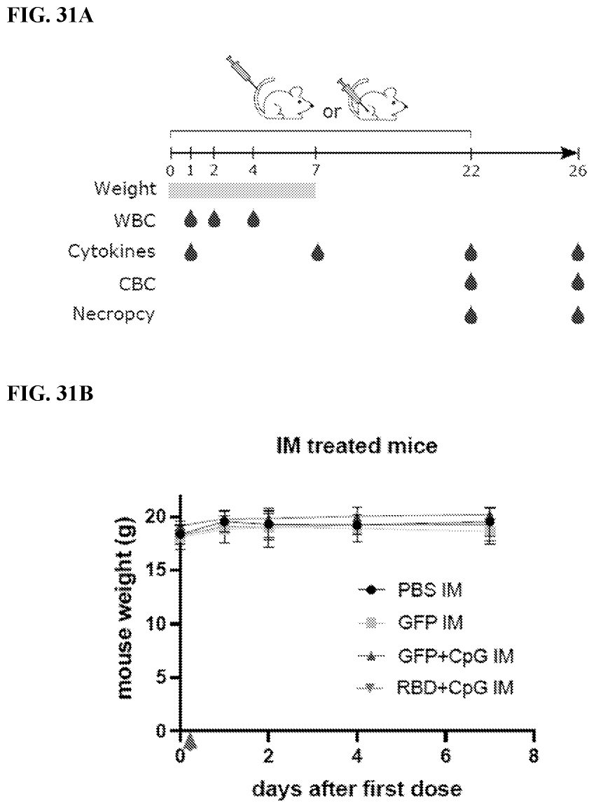

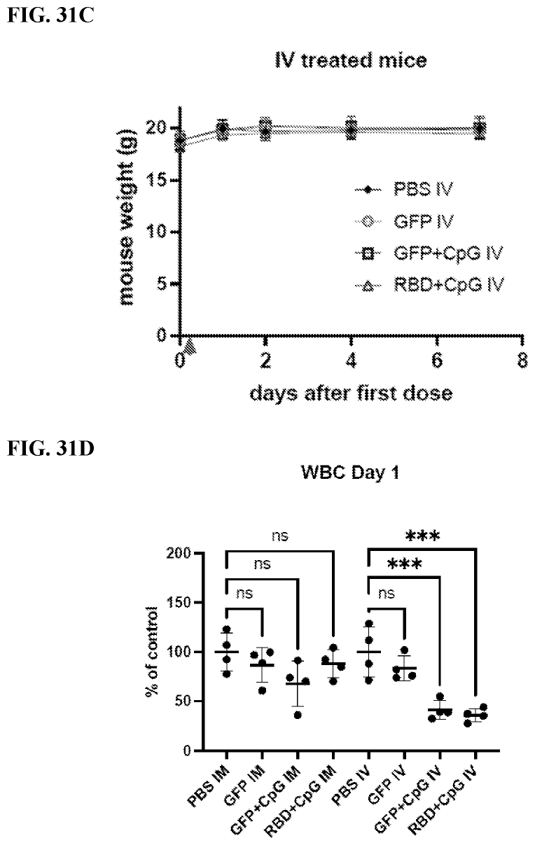

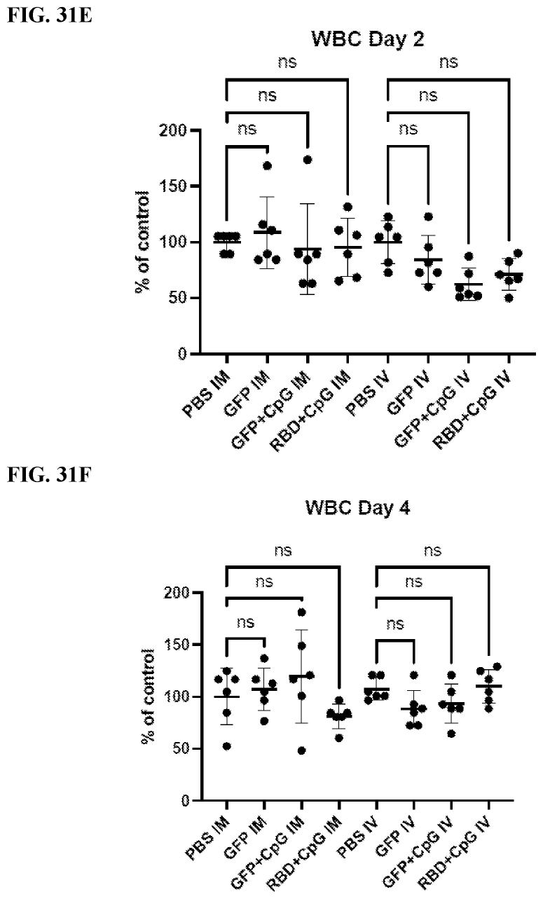

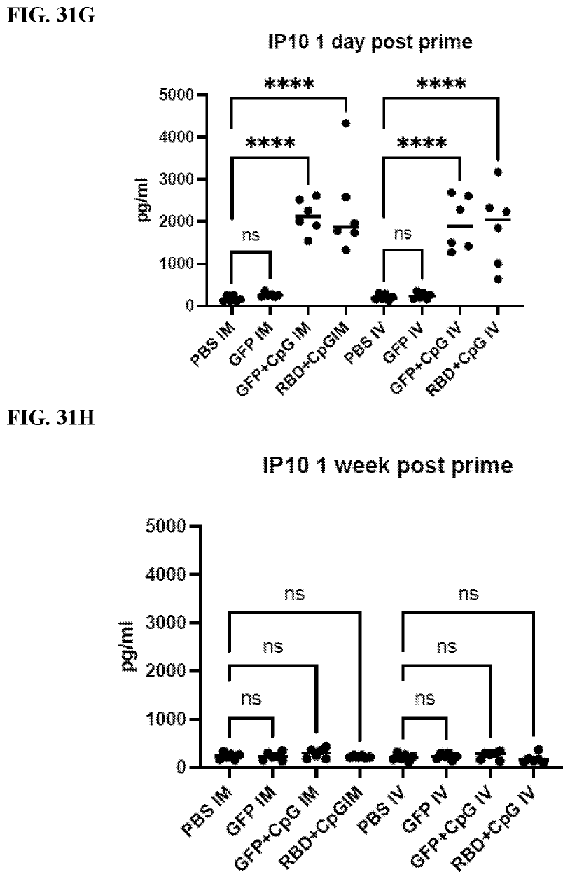

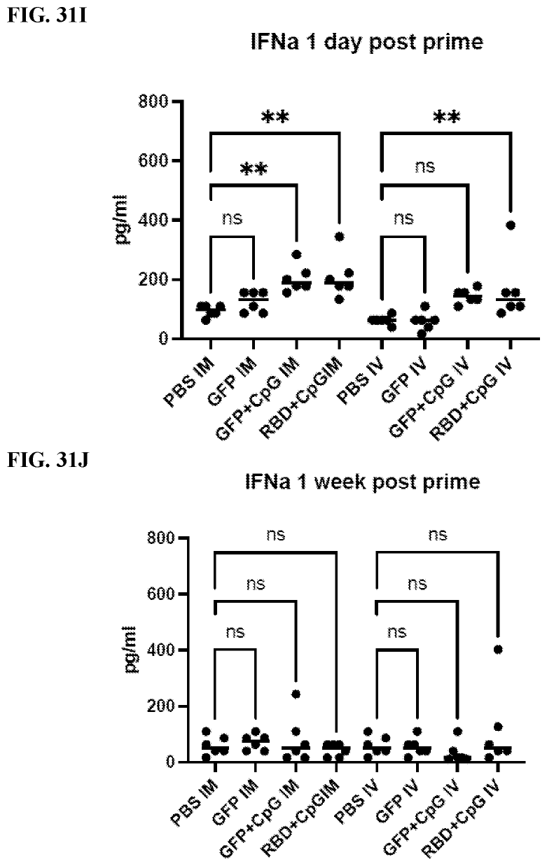

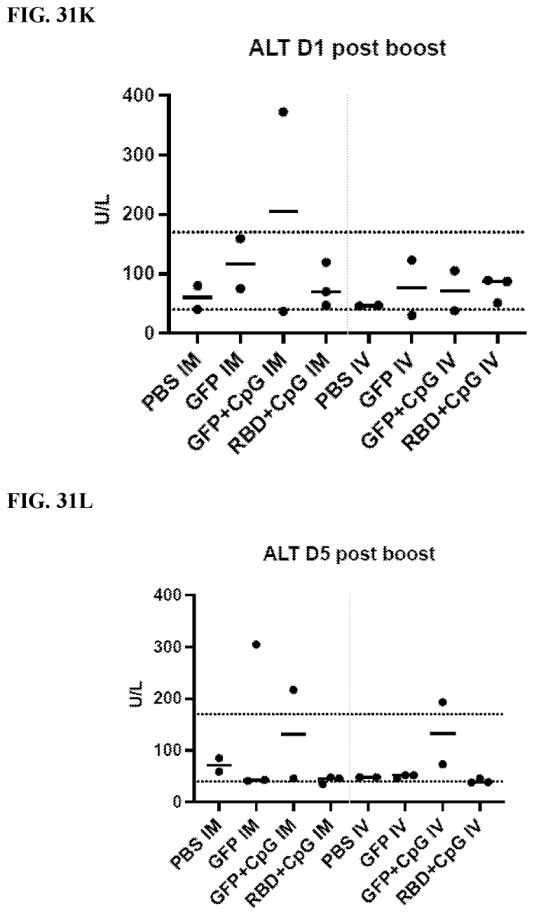

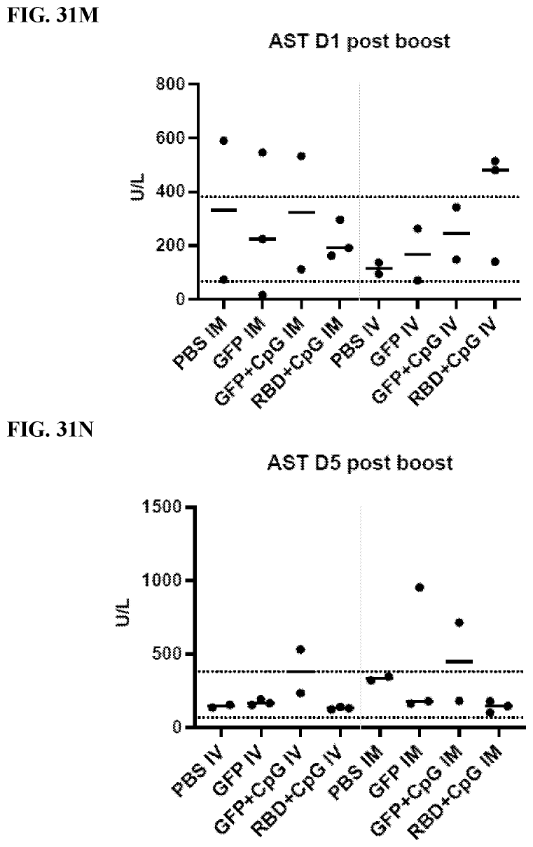

To evaluate the safety profile of the vaccine, mice were treated on DO and D21 with either PBS, 3 μg of GFP mRNA-CART, 3 μg of GFP mRNA+3 μg of CpG-CART or 3 μg of RBD mRNA+3 μg of CpG-CART (FIG. 31A). No differences in body weight were observed after treatment (FIGS. 31B and 31C). IV administration of CpG-containing formulations induced a transient decrease in white blood cell (WBC) count 24 h after treatment that recovered by D2. This was driven by CpG; injection of mRNA-CART without CpG did not alter the WBC count (FIGS. 31D-31F). Serum levels of TNFα and IL-6 measured 1 day and 1 week after treatment were not affected by vaccination. IM and IV vaccination led to a transient increase of serum IP10 and IFNα levels 1 day after prime. Serum cytokine levels were within a normal range at the second analyzed time point 7 days after prime. Again, transient effects were mediated by CpG; mice treated with mRNA-CART without CpG showed an unaltered cytokine profile after treatment compared to untreated mice (FIGS. 31G-31J). Similar dynamics for both WBC count and cytokine profile were observed after the boost treatment (data not shown). Treatment did not induce liver toxicity as assessed by serum liver enzyme levels of alanine transferase (ALT), aspartate transferase (AST) (FIGS. 31K-31N), and alkaline phosphatase (AP) (data not shown). In addition, gross histopathology performed on day 1 and 5 after booster treatment revealed no pathologic findings in the gross assessment of the main organs.

### DISCUSSION

For the first time, mRNA-based therapeutics have been approved by the FDA, and the success of both mRNA-based SARS-CoV-2 vaccines is both remarkable and mutually validating. However, challenges in production, deployment, and availability of SARS-CoV-2 vaccines remain. One important aspect for the continuous success and refinement of mRNA-based therapeutics in general will be to create access to diverse choices of safe delivery vehicles with varying chemical and biological properties. Here, we demonstrate an effective mRNA vaccination strategy against the clinically relevant RBD antigen of SARS-CoV-2 using an alternative mRNA delivery platform to the clinically used lipid nano-particles. We observed an effective SARS-CoV-2 specific immune response that was enhanced by the inclusion of the TLR9 agonist CpG as an adjuvant. We further showed that the resulting anti-RBD sera were able to neutralize pseudoviral entry into ACE2-expressing cells. Focusing on the ACE2 receptor binding domain of the SARS-CoV-2 virus, we were able to generate high amounts of isotype switched RBD-specific neutralizing antibodies in both the serum and lungs of immunized animals. In addition, we compared serum levels of neutralizing antibodies from sera of humans vaccinated with approved Pfizer/BioNTech mRNA-LNP vaccines with those of mRNA-CART vaccinated mice. The levels of neutralizing antibodies in fully immunized mice were similar (IM) or higher (IV) than the levels of neutralizing antibodies measured in preboost blood samples from humans vaccinated with the Pfizer/BioNTech mRNA vaccine. Since it has been shown that this approved mRNA vaccine confers protection as early as day 12 post prime, this is an indication that our vaccine induced neutralizing antibody levels can be sufficient to confer protection.²⁸ Of course, for further validation of these results, vaccination studies in larger animals such as nonhuman primates are needed. Finally, the RBD mRNA+CpG-CART vaccine induced RBD-specific CD4⁺ and CD8⁺ T cell responses with the induction of long-lasting T cell memory of T_{H}1 polarization. In addition, the IgG isotype profile dominated by IgG2a and IgG2b confirms a T_{H}1 polarized T cell response.

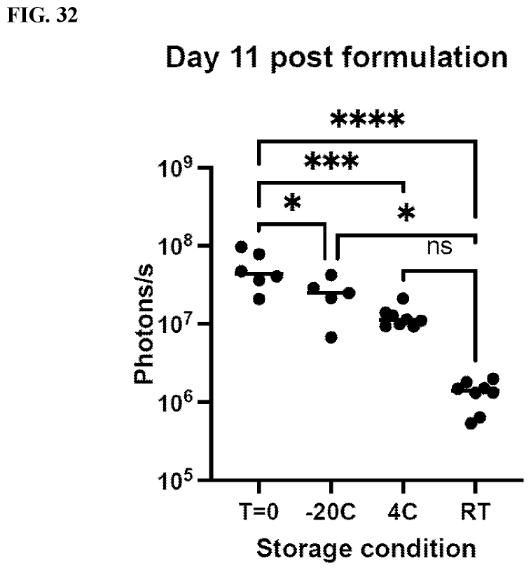

When compared to LNPs, CARTs have a unique biodistribution, selectively delivering mRNA to the spleen or other organs without the need for targeting ligands, simply through changes in the CART structure. CARTs can be readily prepared and formulated with multiple mRNAs in any desired nucleotide combination,²¹ only require a single structural component that is mixed with mRNA, and do not require specialized microfluidics instruments for their manufacture. This allows for alternative drug application strategies. Preliminary experiments show that CARTs formulated with mRNA are stable for 11 days at −20° C. (FIG. 32). However, since formulation does not require specific equipment, a mix-and-shoot administration of the vaccine could be used. Our initial experiments indicate that unformulated CARTs in DMSO are stable for more than 12 months at −20° C. CARTs and mRNA could therefore be divided into two separate chambers of a two-chamber syringe, allowing for mixing and mRNA-CART formation at the point of administration and avoiding thermostability issues of pre-formulated complexes. In contrast to LNPs, CARTs have no unspecific immunostimulatory effects, which allows more flexibility for vaccine design and the option to vary oligodeoxynucleotide adjuvant quantity when codelivered with mRNA, rather than relying on the inherent immunoge-nicity of LNPs. When CARTs are injected IM, gene expression localizes exclusively at the site of injection and does not spread to other organs. In a SARS-CoV-2 vaccine study using IM injection of LNPs, the liver showed the highest level of reporter gene and antigen expression.¹⁹ In addition, IV injection of CARTs confers mRNA expression exclusively to the spleen which could explain the higher potency of IV vaccination compared to IM. While IV administration of the vaccine induced a stronger antibody and T cell response, significant responses could be induced via both routes of administration. Interestingly, it has been shown recently that IV vaccination with a BCG vaccine against tuberculosis profoundly altered the protective outcome in nonhuman primates with an increase of antigen responsive CD4⁺ and CD8⁺ T cells in blood, spleen, BAL, and lung lymph nodes when compared to the established intradermal or aerosol administration.³⁰ Additionally, IV administered mRNA lipid nanoparticles have demonstrated potency in preclinical mouse models and a clinical phase I study of therapeutic cancer vaccination.³¹⁻³²

We chose to direct our vaccine specifically against the RBD rather than the whole spike protein sequence. Potent humoral and cellular immune responses have been observed in clinical trials with both the SARS-CoV-2 full-length spike protein and RBD.⁶ Moreover, an RBD mRNA directed vaccine was proven safe in a phase 1/2 clinical trial tested in the US and in Germany.^{27,33} In addition, RBD provides essential targetability for humoral and cellular immune responses. Piccoli et al. showed that 90% of the neutralizing activity of serum from an exposed patient targets the RBD.34 RBD is also an epitope for T cell responses against SARS-CoV-2 S protein.³⁵ A fast-spreading mutant with a mutation in the RBD (N501Y) has been identified in the UK (B.1.1.7) raising concerns about coverage of the current mRNA-based vaccines.6 This N501Y mutation does not seem to affect the efficacy of an RBD vaccination since mice vaccinated against the original RBD sequence were able to clear a SARS-CoV-2 variant containing the specific mutation.³⁶ However, recent data indicates that other clinically relevant RBD and non-RBD mutations can mediate escape from vaccine induced humoral immunity,^{13,37} highlighting the urgent need of flexible and rapidly adaptable vaccine platforms.

The safety data of mRNA+CpG-CART vaccination seems to be favorable. The detected changes in the white blood cell count and cytokine profile were mediated by CpG. However, CpG has a well-known safety record in clinical studies of other vaccines. We believe that the ability to formulate TLR activating molecules like CpG into our vaccine will aid in inducing a protective immune response in populations with less competent immune systems and that are more at risk for severe COVID19 symptoms. CpG directly activates pDCs and B cells, contributing to the induction of both innate and adaptive immune responses. The cascade of events initiated by CpG indirectly supports maturation, differentiation, and proliferation of natural killer cells, T cells, and monocytes/macrophages. ³⁸⁻⁴¹ B cells activated by CpG upregulate expression of their Fc receptor (FcR) and costimulatory molecules including MHC class II, CD40, CD80, and CD86.⁴²⁻⁴⁴ Subsequently, the CpG-stimulated B cells proliferate and differentiate into plasma cells and memory B cells.⁴⁵ The adjuvant effects of CpG are supported by our study where the addition of CpG resulted in a more rapid immune response, higher anti-RBD titers in the serum and bronchoalveolar lavage, more effective ACE2-RBD inhibition and pseudotyped virus neutralization, an increased T cell response, and more pronounced isotype switching.

In conclusion, our study demonstrates the potency and flexibility of this mRNA-CART vaccine platform against the clinically relevant SARS-CoV-2 RBD antigen. The robust induction of both B and T cell responses via different routes of administration warrants further exploration and its use as an alternative to the clinically approved lipid nanoparticles in the general development of mRNA-based therapeutics against infectious diseases.

Materials and Methods

Recombinant Proteins.

The pCAGGS plasmids coding for soluble RBD-His, residues 319-541, and spike-His, residues 1-1213 from the Wuhan-Hu-1 genome sequence (GenBank MN9089473), were a gift from Prof. Florian Krammer (Icahn School of Medicine at Mount Sinai).²³ The pcDNA3 plasmid coding for a soluble ACE2-hIgA FC fusion protein was purchased from addgene (ID 145154). Plasmids were expanded using One Shot TOP10 Chemically Competent Escherichia coli (ThermoFisher Scientific) and the ZymoPURE II Plasmid Maxiprep kit (Zymo Research). Recombinant proteins were produced using the Expi293F cells (Thermo Fisher Scientific) by transfecting 200×106 of these cells with purified DNA using the ExpiFectamine 293 transfection kit (Thermo Fisher Scientific). Supernatants from transfected cells were harvested 3 days post-transfection by centrifugation at 300 g for 10 min and filtration through a 22 μm filter. RBD-His and Spike-His containing supernatants were batch purified using the HisPur Ni-NTA resin (Thermo-Fisher Scientific). Supernatants were incubated with 6 mL of resin for 1 h at room temperature (RT). Then, the resin was recovered by centrifugation (2 min at 700 g), washed, and finally eluted per manufacturer recommendation. Elution fractions were analyzed by SDS-PAGE and Western blot to confirm RBD-His or Spike-His purification, and the positive fractions were pooled.

In Vitro Transcription.

The RBD-6his was cloned from the pCAGGS expression plasmid into the LF-pLMCT plasmid that contains a T7 promoter and a polyA sequence required for mRNA synthesis. The LF-pLMCT plasmid was a gift from Dr. Kris Thielemans (Free University of Belgium). The RBD-6his coding sequence was amplified by PCR using PHUSION polymerase (NEB) and TAAACTTAAGACAACCATGGTCGTGTTTCTGGTGC (SEQ ID NO:5) as a forward primer and GGGGATCCcGTCTTCCTCGAGTTATCAATGGTGATGGTGA (SEQ ID NO:6) as a reverse primer. The PCR product was then inserted into pLMCT by NcoI and XhoI. mRNA coding for RBD was synthesized per manufacturer recommendation using Hiscribe T7 (NEB) with cotranscriptional CleanCap AG (Trilink), N1-methyl-pseudouridine (Trilink), and 5-methyl-cytidine (Trilink). The template for in vitro transcription was a PCR amplicon from the pLMCT-RBD-6His produced using the PHUSION high-fidelity DNA polymerase (NEB) and TGTGGAATTGTGAGCGGATA (SEQ ID NO:7) as a forward primer and CTTCACTATTGTCGACAAAAAAAAAAAAAAAAAAAAAAAAAAAAAAAAAAAAA AAAAAAAAAAAAAAAAAAAAAAAAAAAAAAAAAAAAAAAAAAAAAAAAAAAA AAAAAAAAAAAAAAAAAAAAAA (SEQ ID NO:8) as a reverse primer.

CART Preparation and Characterization.

CART O6-stat-N₆: A₉, consisting of a first block of a 1:1 statistical mixture of oleyl and nonenyl-substituted carbonate monomers, followed by a block of a-amino ester monomer was prepared as previously reported.^{14,15} Briefly, to a mixture of nonenyl (29 mg, 1 mmol) and oleyl carbonate (40.5 mg, 1 mmol) in toluene (150 μL) were added TU, DBU, and BnOH (5 mol % TU/DBU, 0.2 mmol BnOH) in 50 μL of toluene. The reaction was stirred for 1.5 h; then, the morpholinone monomer (33.4 mg, 0.16 mmol) was added as a solid and then stirred for an additional 2.5 h. The reaction was quenched with AcOH then dialyzed overnight in DCM/MeOH (3.5 kDa Mw, cutoff). Concentration after dialysis afforded 85 mg of clear residue which was deprotected with TFA (0.85 mL) in dry DCM (8.5 mL) overnight. End group analysis of the deprotected polymer showed block lengths of 6 nonenyl and 6 oleyl carbonate units and 9 cationic aminoester units.

CART Oligonucleotide Formulation.

To prepare the CART-vaccine, CARTs were formulated with a mixture of CpG and RBD mRNA at a 10:1 cation: anion ratio assuming full protonation of the CART and full deprotonation of the oligonucleotides (1:1 mass ratio of CpG and mRNA nucleotides). Formulations were prepared by mixing the reagents for 20 s in acidic PBS (pH adjusted to 5.5 by addition of 0.1 M HCl) in a total volume of 50-100 μL, followed by a brief spin in a tabletop centrifuge. The formulation was used within 5 min for in vitro or in vivo experiments.

Mouse Vaccination.

Female BALB/c mice (8- to 12-week-old) were purchased from The Jackson Laboratory and housed in the Laboratory Animal Facility of the Stanford University Medical Center. All experiments were approved by the Stanford Administrative Panel on Laboratory Animal Care and were conducted in accordance with Stanford University Animal Facility and NIH guidelines. RBD-mRNA and CpG were formulated with CART polymer in PBS at pH 5.5 as described above. Mice were injected with 31.1 g of RBD-mRNA formulated with 2.6 μL of CART (5 mM) or 3 μg of CpG formulated with 2.6 μL of CART (5 mM) or 31.1 g of RBD-mRNA plus 3 μg of CpG formulated with 5.2 μL of CART (5 mM). Mice were vaccinated by IV, IM, or SC injection and were boosted as described in the experiment. CARTs are formulated at indicated concentrations of mRNA in 50-100 μL total volume. For IV administration, 100 μL of formulated CART was administered per tail vein injection. For IM injections, 50 μL of formulated CART was injected in the thigh muscle. SC injections were administered on the back of the mouse near the tail. At indicated time points, mice were bled, and serum was collected.

HeLa and 293F Transfection.

HeLa cells and 293F cells were plated at 10⁶ cells per well in a 12-well plate in Opti-MEM media (ThermoFisher Scientific). 2 μg of the RBD-his mRNA or GFP mRNA (Trilink) was formulated in 6.6 μL of PBS pH 5.5 with 1.37 μL of 5 mM CART and added to the cells. After 4 h of transfection, Opti-MEM media was replaced by RPMI media containing 10% FCS and penicillin-streptomycin 1000 U/mL. 12 hours post-transfection, RBD and GFP expression were monitored by Western blot and fluorescence microscopy, respectively.

Western Blot.

15 μL of media from HeLa or 293F transfected cells was mixed with 4× sample loading buffer (Invitrogen) and was loaded on a 4-12% NuPAGE gel (Invitrogen). Electrophoresis was performed in an MES buffer at 200 V for 35 min. Proteins were transferred to a cellulose membrane using the iBLOT system (Invitrogen). The membrane was stained with Ponceau red to verify protein transfer, and then, the membrane was blocked for 1 h in TBS buffer containing 0.1% Tween 20 (TBST) containing 5% nonfat dry milk. The membrane was washed 3 times in TBST and incubated in TBST containing 5% nonfat dry milk and 1:1000 mouse anti-His (Biolegend) overnight. After 3 washes in TBST, the membrane was incubated with 1:10000 antimouse Ig (Southern Biotech) in TBST containing 5% nonfat dry milk for 1 h. After 3 washes in TBST, the blot was revealed using the EC Prime Western blotting system (Sigma). The membrane was imaged using a Chemidoc MP imaging system from BioRad.

Serum Preparation.

For human samples, informed consent was obtained from the subjects prior to blood draw. Blood was collected in Eppendorf tubes and allowed to coagulate for 60 min at room temperature. After 10 min of centrifugation at 1000 g, the supernatant was collected. Serum was heat-inactivated at 56° C. for 30 min.

ELISA.

Nunc-Immuno MicroWell 96-well ELISA plates (MilliporeSigm) were coated overnight with 50 μL per well of 2 μg/mL RBD-His or Spike-His protein in carbonate buffer pH 9. After 3 washes in ELISA wash buffer (PBS with 0.1% Tween 20), plates were blocked using 100 μL of 5% nonfat dry milk diluted in TBS buffer containing 0.1% Tween 20 (TBST) for 1 h at room temperature. Serum, BAL, and antibody dilutions were prepared in TBST containing 1% nonfat dry milk. The blocking solution was removed, and 50 μL of each serial dilution was added to the plate for 1 h at room temperature. Plates were washed three times and incubated with HRP conjugated antihuman Ig (1:5000, BioSource), antimouse Ig (1:5000, Cell Signaling), antimouse IgG2a (1:5000, Southern Biotech), antimouse IgG2b (1:5000, Southern Biotech), antimouse IgG1 (1:5000, Southern Biotech), antimouse IgG3 (1:5000, Southern Biotech), antimouse IgA (1:5000, Invitrogene), or antimouse IgM (1:5000, Southern Biotech). Plates were washed three times, and 100 μL of TMB ELISA substrate (Abcam) was added to each well. ELISA was developed for 10 min, and then, the reaction was stopped by adding 50 μL of Stop Solution for TMB Substrates (ThermoFisher Scientific) to each well. In some assays, a human anti-RBD (Invivogene) of known antibody concentration was used as a standard. Optical density at 450 nm (OD450) was measured using a SpectraMax Paradigm microplate reader (Molecular devices).

RBD-ACE2 Interaction Blocking Assay ELISA.

The RBD-ACE2 interaction blocking assay was evaluated using three methods: a commercial kit from Genescript, an in house developed ELISA, and flow cytometry.

For the commercial kit, we used the SARS-CoV-2 surrogate virus neutralization test (sVNT) kit (Genescript) following the manufacturer's instructions. In short, samples and controls were diluted at indicated ratios with dilution buffer and preincubated with HRP-RBD in a 1:1 ratio for 30 min at 37° C. Samples were then added to the capture plate in wells precoated with hACE2. After 15 h of incubation at 37° C., wells were washed four times with wash buffer. TMB solution was added and incubated for 15 h at room temperature in the dark. After 15 h, stop solution was added to the wells and promptly analyzed. Optical density at 450 nm (OD450) was measured using a SpectraMax Paradigm microplate reader (Molecular devices).

For the in-house developed ELISA, Nunc-Immuno Micro-Well 96-well ELISA plates (Millipore) were coated overnight with 50 μL per well of 2 μg/mL RBD-His or Spike-His protein in carbonate buffer pH 9. After 3 washes in ELISA wash buffer (PBS with 0.1% Tween 20), plates were blocked using 100 μL of 5% nonfat dry milk diluted in TBS buffer containing 0.1% Tween 20 (TBST) for 1 h at room temperature. Serum, BAL, and antibody dilutions were prepared in TBST containing 1% nonfat dry milk. The blocking solution was removed, and 50 μL of each serial dilution was added to the plate for 1 h at room temperature. Plates were washed three times, and 50 μL of 2 times diluted ACE2-hIgA supernatant was added to each well for 1 h. After 3 washes, the plate was incubated with HRP conjugated antihuman IgA (1:1000, Thermo Scientific) for 1 h in TBST with 1% nonfat dry milk. Plates were washed three times, and 100 μL of TMB ELISA Substrate (Abcam) was added to each well. ELISA was allowed to develop for 10 min, and then, the reaction was stopped by adding 50 μL of Stop Solution for TMB Substrates (ThermoFisher Scientific) to each well. In some assays, human anti-RBD (Invivogen) of known antibody concentration was used as a standard. Optical density at 450 nm (OD450) was measured using a SpectraMax Paradigm microplate reader (Molecular devices).

For the flow cytometry assay, RBD-His 2 μg/mL was incubated with sera for 1 h. Then, the 4×10⁵ ACE2-expressing HEK293T cells were added to the RBD-His/sera mix and incubated at RT for 30 min. Cells were then washed 2 times in PBS containing 1% BSA. RBD was then detected using an Alexa Fluor 488 conjugated anti-His antibody (clone J099B1, Biolegend). Cells were analyzed by flow cytometry (BD).

Pseudovirus Assay.

Pseudotyped lentivirus expressing the Sars-Cov-2 spike protein and the luciferase was produced in HEK293T cells as previously described.^{46,47} One day before transfection, 6×106 HEK293T cells were seeded in a 10 cm culture plate in RPMI containing 10% FCS, 2 mM L-glutamine, streptomycin, and penicillin. Using TransIT (Mirus), cells were then transfected with 10 μg of the lentiviral packaging vector (pHAGE_Luc2_IRES_ZsGreen), the 3.4 μg of SARS-CoV-2 spike, and lentiviral helper plasmids (2.2 μg of HDM-Hgpm2, 2.2 μg of HDM-Tat1b, and 2.2 μg of pRC-CMV_Rev1b). The spike vector contained the full-length wild-type spike sequence from the Wuhan-Hu-1 strain of SARS-CoV-2 (GenBank NC_045512). These 5 plasmids were kindly provided by Dr. Jesse Bloom (Fred Hutch Seattle, University of Washington). 72 h after transfection, virus-containing supernatant was harvested, centrifuged at 300 g for 5 min, filtered on a 0.45 μm filter, aliquoted, and frozen at −80° C.

For viral neutralization assays, ACE2-expressing HEK293T⁴⁷ cells were plated in poly-L-lysine-coated, white-walled, clear-bottom 96-well plates at 12 500 cells/well 1 day prior to infection. Mouse serum was centrifuged at 2000 g for 15 min, heat-inactivated for 30 min at 56° C., and diluted in D10 media (DMEM medium supplemented with 10% FCS). Virus was diluted in D10 medium, supplemented with polybrene (5 μg/mL), and then added to serum dilutions. The virus/serum mix was preincubated for 1 h at 37° C. before it was added to the cells and incubated at 37° C. for ˜48 h. Cells were lysed by adding BriteLite assay readout solution (PerkinElmer), and luminescence values were measured with a SpectraMax Paradigm microplate reader (Molecular devices). As a positive control, a neutralizing human anti-SARS-Cov-2 IgG1 antibody was used (Acro).

Bronchoalveolar Lavage.

Mice were sacrificed, and lungs were inflated 2 times with 1 mL of PBS following a previously described procedure.⁴⁸ Lavage fractions were pooled and centrifuged at 1200 rpm for 5 min. Supernatant was collected and assayed for anti-RBD antibodies by ELISA.

T Cell Response Assay on Lungs.

Mouse lungs were harvested at indicated days after vaccination. To prepare lung single-cell suspensions, lungs were cut into small pieces and incubated at 37° C. in RPMI containing Collagenase D (2 mg/mL, Sigma) and DNase (50 μg/mL, Sigma) for 30 min. Then, digestion mix was diluted 5 times, and the lung preparations were processed through a 70 μm cell strainer. Red blood cells present in the spleen or lung single-cell suspensions were lysed using ACK buffer (ThermoFisher Scientific). Single-cell suspensions were kept on ice until further processing for the T cell response assay. Cells were cultured in 96-well plates (Corning, V-bottom) at 1×106 cells/well and stimulated for 48 h with 5 μg/mL RBD-His or hCD81-His or media alone (RPMI+5% FCS) in the presence of antimouse CD₂₈ antibody [0.5 g/mL] (Southern Biotech). As a positive control, cells were stimulated with antimouse CD3 [0.05 g/mL] (Southern Biotech). For intracellular staining, cells were treated with GolgiStop (BD Biosciences) for 5 h prior to staining. Following stimulation, cells were washed, stained with Aqua live/dead viability dye (Thermo Fisher) in PBS, washed two additional times, and stained with a cocktail of monoclonal antibodies and Fc block: CD16/32, CD4 BV605 RM4-5, CD8 FITC 53-6.7, CD44 APC IM7, CD69 PE-Cy7 H1.2F3, CD134 BV786 OX-86, CD137 PE 1AH2, and CD45R/B220 Per-CP 5.5 RA3-6B2 (all BD Bioscience). Cells were fixed and permeabilized according to the manufacturer's protocol (BD Biosciences) and stained with aTNFα BV650 MP6-XT22 (BD Bioscience). Cells were washed, fixed with 2% formaldehyde, acquired on a BD LSR II, and analyzed using Cytobank V7.3.0.

T Cell Response Assay on the Spleen.

Mouse spleens were harvested on D105 after vaccination, and single-cell suspensions were prepared by processing them through a 70 m cell strainer (BD Biosciences). Cells were then incubated in FACS tubes at 6×10⁵ cells per tube and stimulated for 18 h with 2 g/mL RBD peptide mix [PepMix SARS-CoV-2 (S-RBD) Protein ID: P0DTC2 PM-WCPV-S-RBD-1, JPT] or media alone. As a positive control, cells were stimulated with antimouse CD3 [0.05 g/mL] (Southern Biotech) and antimouse CD28 antibody [0.5 g/mL] (Southern Biotech). GolgiStop (BD Biosciences) was added for the last 10 h of the assay. Following stimulation, cells were washed, stained with Aqua live/dead viability dye (Thermo Fisher) in PBS, washed two additional times, and stained with a cocktail of monoclonal antibodies and Fc block: CD16/32, CD4 Ax700 RM4-5, CD8 APC-H7 53-6.7, and CD44 APC IM7 (all BD Bioscience). Cells were fixed and permeabilized according to the manufacturer's protocol (BD Biosciences) and stained for intracellular cytokines with IFNγ PE-Cy7 XMG1.2, TNFα BV650 MP6-XT22, and IL-4 BV786 11B11 (BD Bioscience). Cells were washed, fixed with 2% formaldehyde, acquired on a Cytek Aurora (Northern Lights), and analyzed using Cytobank V7.3.0.

IFNγ ELISpot.

The assay was performed following the manufacturer's instructions (R&D systems, mouse IFNγ kit cat. EL485). In short, IFNγ ELISpot analysis was performed ex vivo (without further in vitro culturing for expansion) using PBMCs depleted of CD4⁺ and enriched for CD8⁺ T cells or depleted of CD8+ and enriched for CD4+ T cells by MACS sort (Miltenyi CD4⁺ or CD8⁺ microbeads following the manufacturer instructions). Tests were performed in triplicates and with a positive control [anti-CD3 monoclonal antibody (0.05 g/mL; Southern Biotech)]. PVDF backed microplates precoated with IFNγ-specific antibodies (R&D systems, mouse IFNγ kit cat. EL485) were washed with PBS and blocked with RPMI medium (Corning) containing 5% FCS for 20 min at room temperature. Per well, 5×10⁵ effector cells were stimulated for 16 h with 2 g/mL RBD peptide mix [PepMix SARS-CoV-2 (S-RBD) Protein ID: P0DTC2 PM-WCPV-S-RBD-1, JPT]. After stimulation, wells were washed and incubated with a biotinylated anti-IFNγ antibody (R&D systems, mouse IFNγ kit cat. EL485) overnight at 4° C. The next day, wells were washed and incubated with streptavidin-AP (R&D systems, mouse IFNγ kit cat. EL485) for 2 h at RT. After washing, wells were incubated with a 5-bromo-4-chloro-3′-indolyl phosphate (BCIP)/nitro blue tetrazolium (NBT) substrate (R&D systems, mouse IFNγ kit cat. EL485). Plates were scanned and analyzed using an ImmunoSpot micro-analyzer.

In Vivo Bioluminescence Imaging.

For the biolumines-cence assessment, mice were anesthetized with isoflurane gas (2% isoflurane in oxygen, 1 L/min) during injection and imaging procedures. Intraperitoneal injections of D-luciferin (Biosynth AG) were done at a dose of 150 mg/kg, providing a saturating substrate concentration for Fluc enzyme (luciferin crosses the blood-brain barrier). Mice were imaged in a light-tight chamber using an in vivo optical imaging system (AMI HT; Spectral Instruments imaging) equipped with a cooled charge-coupled device camera. During image recording, mice inhaled isoflurane delivered via a nose cone, and their body temperature was maintained at 37° C. in the dark box of the camera system. Bioluminescence images were acquired between 10 and 20 min after luciferin administration. Mice usually recovered from anesthesia within 2 min of imaging.

White Blood Cell Count.

5 L of blood was harvested and mixed with 45 L of 3% acetic acid with methylene blue (Stemcell), and nuclei were counted using a hematocytometer.

Cytokine Analysis.

IP10, IFNa, TNFa, and IL6 were measured in serum samples using the LEGENDplex bead-based immunoassays from Biolegend per manufacturer protocol. The assay was analyzed on a BD FACSCalibur instrument.

Complete Blood Count.

A complete blood count (CBC) analysis was performed by the animal diagnostic lab at Stanford. Automated hematology was performed on an Sysmex XN-1000 V analyzer system. Blood smears were made for all CBC samples and reviewed by a clinical laboratory scientist. Manual differentials were performed as indicated by species and automated analysis.

Liver Enzyme Analysis.

Liver enzymes were analyzed by the animal diagnostic lab at Stanford. The chemistry analysis was performed on the Siemens Dimension EXL200/LOCI analyzer. A clinical laboratory scientist performed all testing, including dilutions and repeat tests as indicated, and reviewed all data.

Safety Statement.

For all mouse experiments, no unexpected or unusually high safety hazards were encountered.

### REFERENCES FOR EXAMPLE 2

(1) Padron-Regalado, E. Vaccines for SARS-CoV-2: Lessons from Other Coronavirus Strains. Infect Dis Ther 2020, 9, 255-274.
(2) Zhao, J.; Zhao, S.; Ou, J.; Zhang, J.; Lan, W.; Guan, W.; et al. COVID-19: Coronavirus Vaccine Development Updates. Front. Immunol. 2020, 11, 602256.
(3) Alberer, M.; Gnad-Vogt, U.; Hong, H. S.; Mehr, K. T.; Backert, L.; Finak, G.; et al. Safety and immunogenicity of a mRNA rabies vaccine in healthy adults: an open-label, non-randomised, prospective, first-in-human phase 1 clinical trial. Lancet 2017, 390, 1511-1520.
(4) Feldman, R. A.; Fuhr, R.; Smolenov, I.; Mick Ribeiro, A.; Panther, L.; Watson, M.; et al. mRNA vaccines against H10N8 and H7N9 influenza viruses of pandemic potential are immunogenic and well tolerated in healthy adults in phase 1 randomized clinical trials. Vaccine 2019, 37, 3326-3334.
(5) Anderson, E. J.; Rouphael, N. G.; Widge, A. T.; Jackson, L. A.; Roberts, P. C.; Makhene, M.; et al. Safety and Immunogenicity of SARS-CoV-2 mRNA-1273 Vaccine in Older Adults. N. Engl. J. Med. 2020, 383, 2427-2438.
(6) Walsh, E. E.; Frenck, R. W.; Falsey, A. R.; Kitchin, N.; Absalon, J.; Gurtman, A.; et al. Safety and Immunogenicity of Two RNA-Based Covid-19 Vaccine Candidates. N. Engl. J. Med. 2020, 383, 2439-2450.
(7) Ura, T.; Yamashita, A.; Mizuki, N.; Okuda, K.; Shimada, M. New vaccine production platforms used in developing SARS-CoV-2 vaccine candidates. Vaccine 2021, 39, 197-201.
(8) Dai, L.; Gao, G. F. Viral targets for vaccines against COVID-19. Nat. Rev. Immunol. 2021, 21, 73-82.
(9) Lee, W. S.; Wheatley, A. K.; Kent, S. J.; DeKosky, B. J. Antibody-dependent enhancement and SARS-CoV-2 vaccines and therapies. Nat. Microbiol 2020, 5, 1185-1191.
(10) Liang, F.; Lindgren, G.; Lin, A.; Thompson, E. A.; Ols, S.; Rohss, J.; et al. Efficient Targeting and Activation of Antigen-Presenting Cells In Vivo after Modified mRNA Vaccine Admin-istration in Rhesus Macaques. Mol. Ther. 2017, 25, 2635-2647.
(11) Zhu, Z.; Gao, P.; Hu, Y.; Wang, J.; Wang, H.; Yang, J.; et al. PEGylated versus non-PEGylated drugs: A cross-sectional analysis of adverse events in the FDA Adverse Event Reporting System (FAERS) Database. Int. J. Clin. Pharmacol. Ther. 2020, 58, 332-342.
(12) Castells, M. C.; Phillips, E. J. Maintaining Safety with SARS-CoV-2 Vaccines. N. Engl. J. Med. 2021, 384, 643-649.
(13) Chen, R. E.; Zhang, X.; Case, J. B.; Winkler, E. S.; Liu, Y.; VanBlargan, L. A.; et al. Resistance of SARS-CoV-2 variants to neutralization by monoclonal and serum-derived polyclonal antibod-ies. Nat. Med. 2021, 27, 717-726.
(14) McKinlay, C. J.; Vargas, J. R.; Blake, T. R.; Hardy, J. W.; Kanada, M.; Contag, C. H.; et al. Charge-altering releasable transporters (CARTs) for the delivery and release of mRNA in living animals. Proc. Natl. Acad. Sci. U.S.A. 2017, 114, E448-E456.
(15) McKinlay, C. J.; Benner, N. L.; Haabeth, O. A.; Waymouth, R. M.; Wender, P. A. Enhanced mRNA delivery into lymphocytes enabled by lipid-varied libraries of charge-altering releasable trans-porters. Proc. Natl. Acad. Sci. U.S.A. 2018, 115, E5859-E5866.
(16) Haabeth, O. A. W.; Blake, T. R.; McKinlay, C. J.; Waymouth, R. M.; Wender, P. A.; Levy, R. mRNA vaccination with charge-altering releasable transporters elicits human T cell responses and cures established tumors in mice. Proc. Natl. Acad. Sci. U.S.A. 2018, 115, E9153-E9161.
(17) Bode, C.; Zhao, G.; Steinhagen, F.; Kinjo, T.; Klinman, D. M. CpG DNA as a vaccine adjuvant. Expert Rev. Vaccines 2011, 10, 499-511.
(18) Amanat, F.; Krammer, F. SARS-CoV-2 Vaccines: Status Report. Immunity 2020, 52, 583-589.
(19) Zhang, N.-N.; Li, X.-F.; Deng, Y.-Q.; Zhao, H.; Huang, Y.-J.; Yang, G.; et al. A Thermostable mRNA Vaccine against COVID-19. Cell 2020, 182, 1271-1283.
(20) Huang, Q.; Ji, K.; Tian, S.; Wang, F.; Huang, B.; Tong, Z.; et al. A single-dose mRNA vaccine provides a long-term protection for hACE2 transgenic mice from SARS-CoV-2. Nat. Commun. 2021, 12, 12.
(21) Haabeth, O. A. W.; Blake, T. R.; McKinlay, C. J.; Tveita, A. A.; Sallets, A.; Waymouth, R. M.; et al. Local Delivery of Ox40l, Cd80, and Cd86 mRNA Kindles Global Anticancer Immunity. Cancer Res. 2019, 79, 1624-1634.
(22) Xue, H. Y.; Liu, S.; Wong, H. L. Nanotoxicity: a key obstacle to clinical translation of siRNA-based nanomedicine. Nanomedicine (London, U. K.) 2014, 9, 295-312.
(23) Amanat, F.; Stadlbauer, D.; Strohmeier, S.; Nguyen, T. H. O.; Chromikova, V.; McMahon, M.; et al. A serological assay to detect SARS-CoV-2 seroconversion in humans. Nat. Med. 2020, 26, 1033-1036.
(24) Vaidyanathan, S.; Azizian, K. T.; Hague, A. K. M. A.; Henderson, J. M.; Hendel, A.; Shore, S.; et al. Uridine Depletion and Chemical Modification Increase Cas9 mRNA Activity and Reduce Immunogenicity without HPLC Purification. Mol. Ther.-Nucleic Acids 2018, 12, 530-542.
(25) Toellner, K.-M.; Luther, S. A.; Sze, D M-Y; Choy, R K-W; Taylor, D. R.; MacLennan, I. C. M.; et al. T Helper 1 (Th1) and Th2 Characteristics Start to Develop During T Cell Priming and Are Associated with an Immediate Ability to Induce Immunoglobulin Class Switching. J. Exp. Med. 1998, 187, 1193-1204.
(26) Reinhardt, R. L.; Liang, H.-E.; Locksley, R. M. Cytokine-secreting follicular T cells shape the antibody repertoire. Nat. Immunol. 2009, 10, 385-393.
(27) Mulligan, M. J.; Lyke, K. E.; Kitchin, N.; Absalon, J.; Gurtman, A.; Lockhart, S.; et al. Phase I/II study of COVID-19 RNA vaccine BNT162b1 in adults. Nature 2020, 586, 589-593.
(28) Polack, F. P.; Thomas, S. J.; Kitchin, N.; Absalon, J.; Gurtman, A.; Lockhart, S.; et al. Safety and Efficacy of the BNT162b2 mRNA Covid-19 Vaccine. N. Engl. J. Med. 2020, 383, 2603-2615.
(29) Baden, L. R.; El Sahly, H. M.; Essink, B.; Kotloff, K.; Frey, S.; Novak, R.; et al. Efficacy and Safety of the mRNA-1273 SARS-CoV-2 Vaccine. N. Engl. J. Med. 2021, 384, 403-416.
(30) Darrah, P. A.; Zeppa, J. J.; Maiello, P.; Hackney, J. A.; Wadsworth, M. H.; Hughes, T. K.; et al. Prevention of tuberculosis in macaques after intravenous BCG immunization. Nature 2020, 577, 95-102.
(31) Grunwitz, C.; Salomon, N.; Vascotto, F.; Selmi, A.; Bukur, T.; Diken, M.; et al. HPV16 RNA-LPX vaccine mediates complete regression of aggressively growing HPV-positive mouse tumors and establishes protective T cell memory. Oncoimmunology 2019, 8, e1629259.
(32) Sahin, U.; Oehm, P.; Derhovanessian, E.; Jabulowsky, R. A.; Vormehr, M.; Gold, M.; et al. An RNA vaccine drives immunity in checkpoint-inhibitor-treated melanoma. Nature 2020, 585, 107-112.
(33) Sahin, U.; Muik, A.; Derhovanessian, E.; Vogler, I.; Kranz, L. M.; Vormehr, M.; et al. COVID-19 vaccine BNT162b1 elicits human antibody and TH1 T cell responses. Nature 2020, 586, 594-599.
(34) Piccoli, L.; Park, Y.-J.; Tortorici, M. A.; Czudnochowski, N.; Walls, A. C.; Beltramello, M.; et al. Mapping Neutralizing and Immunodominant Sites on the SARS-CoV-2 Spike Receptor-Binding Domain by Structure-Guided High-Resolution Serology. Cell 2020, 183, 1024-1042.
(35) Smith, T. R. F.; Patel, A.; Ramos, S.; Elwood, D.; Zhu, X.; Yan, J.; et al. Immunogenicity of a DNA vaccine candidate for COVID-19. Nat. Commun. 2020, 11, 2601.
(36) Gu, H.; Chen, Q.; Yang, G.; He, L.; Fan, H.; Deng, Y.-Q.; et al. Adaptation of SARS-CoV-2 in BALB/c mice for testing vaccine efficacy. Science 2020, 369, 1603-1607.
(37) Garcia-Beltran, W. F.; Lam, E. C.; St. Denis, K.; Nitido, A. D.; Garcia, Z. H.; Hauser, B. M.; et al. Multiple SARS-CoV-2 variants escape neutralization by vaccine-induced humoral immunity. Cell 2021, 184, 2372.
(38) Ballas, Z. K.; Rasmussen, W. L.; Krieg, A. M. Induction of NK activity in murine and human cells by CpG motifs in oligodeox-ynucleotides and bacterial DNA. J. Immunol 1996, 157 (5), 1840-1845.
(39) Klinman, D. M.; Yi, A. K.; Beaucage, S. L.; Conover, J.; Krieg, A. M. CpG motifs present in bacteria DNA rapidly induce lymphocytes to secrete interleukin 6, interleukin 12, and interferon gamma. Proc. Natl. Acad. Sci. U.S.A 1996, 93, 2879-2883.
(40) Sun, S.; Zhang, X.; Tough, D. F.; Sprent, J. Type I interferon-mediated stimulation of T cells by CpG DNA. J. Exp. Med. 1998, 188, 2335-2342.
(41) Stacey, K. J.; Sweet, M. J.; Hume, D. A. Macrophages ingest and are activated by bacterial DNA. J. Immunol 1996, 157 (5), 2116-2122.
(42) Krieg, A. M.; Yi, A. K.; Matson, S.; Waldschmidt, T. J.; Bishop, G. A.; Teasdale, R.; et al. CpG motifs in bacterial DNA trigger direct B-cell activation. Nature 1995, 374, 546-549.
(43) Davis, H. L.; Weeratna, R.; Waldschmidt, T. J.; Tygrett, L.; Schorr, J.; Krieg, A. M.; et al. CpG DNA is a potent enhancer of specific immunity in mice immunized with recombinant hepatitis B surface antigen. J. Immunol 1998, 160 (2), 870-876.
(44) Kobayashi, H.; Horner, A. A.; Takabayashi, K.; Nguyen, M. D.; Huang, E.; Cinman, N.; et al. Immunostimulatory DNA pre-priming: a novel approach for prolonged Th1-biased immunity. Cell. Immunol. 1999, 198, 69-75.
(45) Jung, J.; Yi, A.-K.; Zhang, X.; Choe, J.; Li, L.; Choi, Y. S. Distinct response of human B cell subpopulations in recognition of an innate immune signal, CpG DNA. J. Immunol. 2002, 169, 2368-2373.
(46) Crawford, K. H. D.; Eguia, R.; Dingens, A. S.; Loes, A. N.; Malone, K. D.; Wolf, C. R.; et al. Protocol and Reagents for Pseudotyping Lentiviral Particles with SARS-CoV-2 Spike Protein for Neutralization Assays. Viruses 2020, 12, 513.
(47) Rogers, T. F.; Zhao, F.; Huang, D.; Beutler, N.; Burns, A.; He, W.; et al. Isolation of potent SARS-CoV-2 neutralizing antibodies and protection from disease in a small animal model. Science 2020, 369, 956-963.
(48) Sun, F.; Xiao, G.; Qu, Z. Murine Bronchoalveolar Lavage. Bio Protoc 2017, 7, 7.

INFORMAL SEQUENCE LISTING
(ODN 1826):
SEQ ID NO: 1
tccatgacgttcctgacgtt
(ODN SD-101):
SEQ ID NO: 2
tcgaacgttcgaacgttcgaacgttcgaat
(mRNA encoding SARS-COV-2 RBD of spike
protein):
SEQ ID NO: 3
(atggTCGTGTTTCTGGTGCTGCTGCCTCTGGTGTCCAGCCAGCGGGTGC
AGCCCACCGAATCCATCGTGCGGTTCCCCAATATCACCAATCTGTGCCCC
TTCGGCGAGGTGTTCAATGCCACCAGATTCGCCTCTGTGTACGCCTGGAA
CCGGAAGCGGATCAGCAATTGCGTGGCCGACTACTCCGTGCTGTACAACT
CCGCCAGCTTCAGCACCTTCAAGTGCTACGGCGTGTCCCCTACCAAGCTG
AACGACCTGTGCTTCACAAACGTGTACGCCGACAGCTTCGTGATCCGGGG
AGATGAAGTGCGGCAGATTGCCCCTGGACAGACAGGCAAGATCGCCGACT
ACAACTACAAGCTGCCCGACGACTTCACCGGCTGTGTGATTGCCTGGAAC
AGCAACAACCTGGACTCCAAAGTCGGCGGCAACTACAATTACCTGTACCG
GCTGTTCCGGAAGTCCAATCTGAAGCCCTTCGAGCGGGACATCTCCACCG
AGATCTATCAGGCCGGCAGCACCCCTTGTAACGGCGTGGAAGGCTTCAAC
TGCTACTTCCCACTGCAGTCCTACGGCTTTCAGCCCACAAATGGCGTGGG
CTATCAGCCCTACAGAGTGGTGGTGCTGAGCTTCGAACTGCTGCATGCCC
CTGCCACAGTGTGCGGCCCTAAGAAAAGCACCAATCTCGTGAAGAACAAA
TGCGTGAACTTCcaccatcaccatcaccatTGATAA)
(mRNA encoding SARS-COV-2 spike protein):
SEQ ID NO: 4
atggTCGTGTTTCTGGTGCTGCTGCCTCTGGTGTCCAGCCAGTGTGTGAA
CCTGACCACAAGAACCCAGCTGCCTCCAGCCTACACCAACAGCTTTACCA
GAGGCGTGTACTACCCCGACAAGGTGTTCAGATCCAGCGTGCTGCACTCT
ACCCAGGACCTGTTCCTGCCTTTCTTCAGCAACGTGACCTGGTTCCACGC
CATCCACGTGTCCGGCACCAATGGCACCAAGAGATTCGACAACCCCGTGC
TGCCCTTCAACGACGGGGTGTACTTTGCCAGCACCGAGAAGTCCAACATC
ATCAGAGGCTGGATCTTCGGCACCACACTGGACAGCAAGACCCAGAGCCT
GCTGATCGTGAACAACGCCACCAACGTGGTCATCAAAGTGTGCGAGTTCC
AGTTCTGCAACGACCCCTTCCTGGGCGTCTACTATCACAAGAACAACAAG
AGCTGGATGGAAAGCGAGTTCCGGGTGTACAGCAGCGCCAACAACTGCAC
CTTCGAGTACGTGTCCCAGCCTTTCCTGATGGACCTGGAAGGCAAGCAGG
GCAACTTCAAGAACCTGCGCGAGTTCGTGTTCAAGAACATCGACGGCTAC
TTCAAGATCTACAGCAAGCACACCCCTATCAACCTCGTGCGGGATCTGCC
TCAGGGCTTCTCTGCTCTGGAACCCCTGGTGGATCTGCCCATCGGCATCA
ACATCACCCGGTTTCAGACACTGCTGGCCCTGCACAGAAGCTACCTGACA
CCTGGCGATAGCAGCAGCGGATGGACAGCTGGTGCCGCCGCTTACTATGT
GGGCTACCTGCAGCCTAGAACCTTTCTGCTGAAGTACAACGAGAACGGCA
CCATCACCGACGCCGTGGATTGTGCTCTGGATCCTCTGAGCGAGACAAAG
TGCACCCTGAAGTCCTTCACCGTGGAAAAGGGCATCTACCAGACCAGCAA
CTTCCGGGTGCAGCCCACCGAATCCATCGTGCGGTTCCCCAATATCACCA
ATCTGTGCCCCTTCGGCGAGGTGTTCAATGCCACCAGATTCGCCTCTGTG
TACGCCTGGAACCGGAAGCGGATCAGCAATTGCGTGGCCGACTACTCCGT
GCTGTACAACTCCGCCAGCTTCAGCACCTTCAAGTGCTACGGCGTGTCCC
CTACCAAGCTGAACGACCTGTGCTTCACAAACGTGTACGCCGACAGCTTC
GTGATCCGGGGAGATGAAGTGCGGCAGATTGCCCCTGGACAGACAGGCAA
GATCGCCGACTACAACTACAAGCTGCCCGACGACTTCACCGGCTGTGTGA
TTGCCTGGAACAGCAACAACCTGGACTCCAAAGTCGGCGGCAACTACAAT
TACCTGTACCGGCTGTTCCGGAAGTCCAATCTGAAGCCCTTCGAGCGGGA
CATCTCCACCGAGATCTATCAGGCCGGCAGCACCCCTTGTAACGGCGTGG
AAGGCTTCAACTGCTACTTCCCACTGCAGTCCTACGGCTTTCAGCCCACA
AATGGCGTGGGCTATCAGCCCTACAGAGTGGTGGTGCTGAGCTTCGAACT
GCTGCATGCCCCTGCCACAGTGTGCGGCCCTAAGAAAAGCACCAATCTCG
TGAAGAACAAATGCGTGAACTTCAACTTCAACGGCCTGACCGGCACCGGC
GTGCTGACAGAGAGCAACAAGAAGTTCCTGCCATTCCAGCAGTTTGGCCG
GGATATCGCCGATACCACAGACGCCGTTAGAGATCCCCAGACACTGGAAA
TCCTGGACATCACCCCTTGCAGCTTCGGCGGAGTGTCTGTGATCACCCCT
GGCACCAACACCAGCAATCAGGTGGCAGTGCTGTACCAGGACGTGAACTG
TACCGAAGTGCCCGTGGCCATTCACGCCGATCAGCTGACACCTACATGGC
GGGTGTACTCCACCGGCAGCAATGTGTTTCAGACCAGAGCCGGCTGTCTG
ATCGGAGCCGAGCACGTGAACAATAGCTACGAGTGCGACATCCCCATCGG
CGCTGGCATCTGTGCCAGCTACCAGACACAGACAAACAGCCCCGCCTCTG
TGGCCAGCCAGAGCATCATTGCCTACACAATGTCTCTGGGCGCCGAGAAC
AGCGTGGCCTACTCCAACAACTCTATCGCTATCCCCACCAACTTCACCAT
CAGCGTGACCACAGAGATCCTGCCTGTGTCCATGACCAAGACCAGCGTGG
ACTGCACCATGTACATCTGCGGCGATTCCACCGAGTGCTCCAACCTGCTG
CTGCAGTACGGCAGCTTCTGCACCCAGCTGAATAGAGCCCTGACAGGGAT
CGCCGTGGAACAGGACAAGAACACCCAAGAGGTGTTCGCCCAAGTGAAGC
AGATCTACAAGACCCCTCCTATCAAGGACTTCGGCGGCTTCAATTTCAGC
CAGATTCTGCCCGATCCTAGCAAGCCCAGCAAGCGGAGCTTCATCGAGGA
CCTGCTGTTCAACAAAGTGACACTGGCCGACGCCGGCTTCATCAAGCAGT
ATGGCGATTGTCTGGGCGACATTGCCGCCAGGGATCTGATTTGCGCCCAG
AAGTTTAACGGACTGACAGTGCTGCCTCCTCTGCTGACCGATGAGATGAT
CGCCCAGTACACATCTGCCCTGCTGGCCGGCACAATCACAAGCGGCTGGA
CATTTGGAGCTGGCGCCGCTCTGCAGATCCCCTTTGCTATGCAGATGGCC
TACCGGTTCAACGGCATCGGAGTGACCCAGAATGTGCTGTACGAGAACCA
GAAGCTGATCGCCAACCAGTTCAACAGCGCCATCGGCAAGATCCAGGACA
GCCTGAGCAGCACAGCAAGCGCCCTGGGAAAGCTGCAGGACGTGGTCAAC
CAGAATGCCCAGGCACTGAACACCCTGGTCAAGCAGCTGTCCTCCAACTT
CGGCGCCATCAGCTCTGTGCTGAACGATATCCTGAGCAGACTGGACcctc
ctGAAGCCGAGGTGCAGATCGACAGACTGATCACCGGAAGGCTGCAGTCC
CTGCAGACCTACGTTACCCAGCAGCTGATCAGAGCCGCCGAGATTAGAGC
CTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGC
TCAAGAGAAGAATTTCACCACCGCTCCAGCCATCTGCCACGACGGCAAAG
CCCACTTTCCTAGAGAAGGCGTGTTCGTGTCCAACGGCACCCATTGGTTC
GTGACCCAGCGGAACTTCTACGAGCCCCAGATCATCACCACCGACAACAC
CTTCGTGTCTGGCAACTGCGACGTCGTGATCGGCATTGTGAACAATACCG
TGTACGACCCTCTGCAGCCCGAGCTGGACAGCTTCAAAGAGGAACTGGAT
AAGTACTTTAAGAACCACACAAGCCCCGACGTGGACCTGGGCGATATCAG
CGGAATCAATGCCAGCGTCGTGAACATCCAGAAAGAGATCGACCGGCTGA
ACGAGGTGGCCAAGAATCTGAACGAGAGCCTGATCGACCTGCAAGAACTG
GGGAAGTACGAGCAGTACATCAAGTGGCCCAGCGGCCGCTTGGTCCCACG
TGGCTCACCCGGATCTGGATACATCCCGGAGGCCCCTAGGGACGGTCAAG
CTTACGTGAGAAAGGACGGCGAATGGGTTCTGCTGTCGACCTTCTTGGGA
CATCATCATCATCATCACTAATGA
(forward primer for BD-6his
coding sequence)
SEQ ID NO: 5
TAAACTTAAGACAACCATGGTCGTGTTTCTGGTGC
(reverse primer for BD-6his
coding sequence)
SEQ ID NO: 6
GGGGATCCcGTCTTCCTCGAGTTATCAATGGTGATGGTGA
(forward primer for amplicon
from pLMCT-RBD-6His)
SEQ ID NO: 7
TGTGGAATTGTGAGCGGATA
(reverse primer for amplicon
from pLMCT-RBD-6His)
SEQ ID NO: 8
CTTCACTATTGTCGACAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAA

1-13. (canceled)

14. A cell-penetrating complex comprising a ribonucleic acid comprising a sequence encoding a viral protein, a nucleic acid adjuvant and a cationic amphipathic polymer, wherein said cationic amphipathic polymer has the formula:
R^{1A}[L¹-[(LP¹)_{z1}-(LP²)_{z3}-(IM)_{z2}]_{z4}-L²-R^{2A}]_{z5} (I)
wherein
R^{1A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI ₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI ₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ and L² are independently a bond, —C(O)O—, —O—, —S—, —NH—, —C(O)NH—, —NHC(O)—, —S(O)₂—, —S(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
LP¹ and LP² are independently a lipophilic polymer domain;
IM is said pH-sensitive immolation domain;
z5 is an integer from 1 to 10;
z1 and z3 are independently integers from 0 to 100, wherein at least one of z1 or z3 is not 0;
z4 is an integer from 1 to 100; and
z2 is an integer from 2 to 100.

15. (canceled)

16. The cell-penetrating complex of claim 14, wherein LP¹ has the formula: 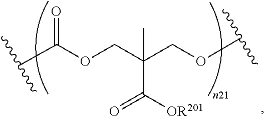
wherein
n21 is an integer from 1 to 100;
R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

17. The cell-penetrating complex of claim 16, wherein n21 is 5 and R²⁰¹ is unsubstituted C₁₈ alkenyl.

18. The cell-penetrating complex of claim 17, wherein said unsubstituted C₁₈ alkenyl is oleyl.

19. The cell-penetrating complex of claim 14, wherein LP² has the formula: 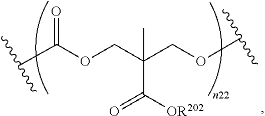
wherein
n22 is an integer from 1 to 100;
R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

20. The cell-penetrating complex of claim 19, wherein n22 is 5 and R²⁰² is unsubstituted C₉ alkenyl.

21. The cell-penetrating complex of claim 20, wherein said unsubstituted C₉ alkenyl is nonenyl.

22. The cell-penetrating complex of claim 14, wherein said cationic amphipathic polymer has the formula: 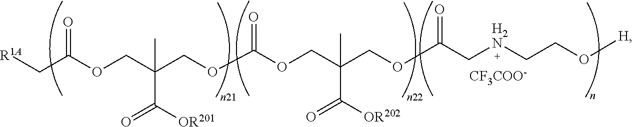
wherein n21 is 5, R²⁰¹ is oleyl, n22 is 5, R²⁰² is nonenyl and n is 7.

23-29. (canceled)

30. The cell penetrating complex of claim 14, wherein said cationic amphipathic polymer has the formula: 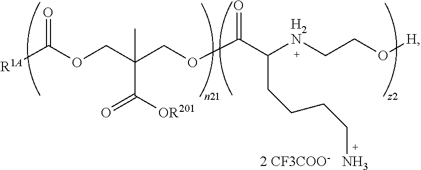
wherein n21 is an integer from 10 to 20;
R²⁰¹ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
z2 is independently an integer from 3-10.

31. The cell penetrating complex of claim 30, wherein n21 is 14, R²⁰¹ is dodecyl and z2 is 8.

32. The cell penetrating complex of claim 31, wherein said cationic amphipathic polymer has the formula: 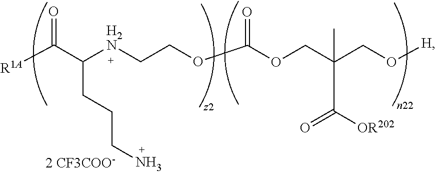
wherein n22 is an integer from 10 to 35;
R²⁰² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
z2 is independently an integer from 5-20.

33. The cell penetrating complex of claim 32, wherein n22 is 14, R²⁰² is dodecyl and z2 is 7.

34. The cell penetrating complex of claim 14, further comprising a second cationic amphipathic polymer, wherein said second cationic amphipathic polymer is different from said cationic amphipathic polymer.

35. The cell penetrating complex of claim 34, wherein said second cationic amphipathic polymer has the formula: 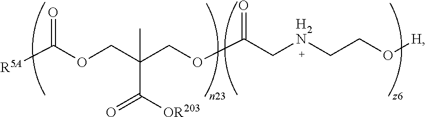
n23 is an integer from 1 to 100;
z6 is an integer from 5-15;
R^{3A} is independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R²⁰³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

36. The cell penetrating complex of claim 35, wherein n23 is 13, z6 is 11 and R²⁰³ is dodecyl.

37. A vaccine composition comprising a cell-penetrating complex of claim 14.

38. A pharmaceutical composition comprising a therapeutically effective amount of a cell-penetrating complex of claim 14 and a pharmaceutically acceptable excipient.

39. A method of treating or preventing a viral disease in a subject in need of such treatment or prevention, said method comprising administering a therapeutically or prophylactically effective amount of a cell-penetrating complex of claim 14 to said subject.

40-42. (canceled)

43. The method of claim 39, wherein the viral disease is SARS

44. The method of claim 39, wherein the viral disease is COVID-19.

45. (canceled)


SEQUENCE LISTING (8 sequences)

SEQ ID NO: 1 - DNA, 20 bp, Artificial Sequence
  Synthetic polynucleotide
tccatgacgttcctgacgtt

SEQ ID NO: 2 - DNA, 30 bp, Artificial Sequence
  Synthetic polynucleotide
tcgaacgttcgaacgttcgaacgttcgaat

SEQ ID NO: 3 - DNA, 735 bp, Severe acute respiratory syndrome coronavirus 2
atggtcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccattgataa

SEQ ID NO: 4 - DNA, 3774 bp, Severe acute respiratory syndrome coronavirus 2
atggtcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatga

SEQ ID NO: 5 - DNA, 35 bp, Artificial Sequence
  Synthetic polynucleotide
taaacttaagacaaccatggtcgtgtttctggtgc

SEQ ID NO: 6 - DNA, 40 bp, Artificial Sequence
  Synthetic polynucleotide
ggggatcccgtcttcctcgagttatcaatggtgatggtga

SEQ ID NO: 7 - DNA, 20 bp, Artificial Sequence
  Synthetic polynucleotide
tgtggaattgtgagcggata

SEQ ID NO: 8 - DNA, 127 bp, Artificial Sequence
  Synthetic polynucleotide
cttcactattgtcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa